(19) United States
(12) Patent Application Publication
(10) Pub. No.: US 2025/0127898 A1
(43) Pub. Date: Apr. 24, 2025

(54) PHOTODYNAMIC THERAPY AND DIAGNOSIS

(71) Applicant: RMW CHO Group Limited, Hong Kong (CN)

(72) Inventors: Sebastian M. Marcuccio, Melbourne (AU); Andrew N. Stephens, Melbourne (AU); Christopher D. Donner, Melbourne (AU); Honsue Cho, Melbourne (AU); Sacha Novakovic, Melbourne (AU)

(21) Appl. No.: 18/713,572

(22) PCT Filed: Nov. 28, 2022
(86) PCT No.: PCT/EP2022/083554

§ 371 Date: May 24, 2024

(30) Foreign Application Priority Data
Nov. 26, 2021 (GB) 2117140.0
Jun. 1, 2022 (GB) 2208167.3

(51) Int. Cl. A61K 41/00; A61K 39/395; A61P 35/00; C07D 487/22; C07H 15/26

(52) U.S. Cl. CPC A61K 41/0071; A61K 39/3955; A61P 35/00; C07D 487/22; C07H 15/26

(57) ABSTRACT

The present invention relates to reduced chlorin analogues and their pharmaceutically acceptable salts, and compositions comprising reduced chlorin analogues and their pharmaceutically acceptable salts. Reduced chlorin analogues and pharmaceutically acceptable salts thereof are suitable for use in photodynamic therapy, cytoluminescent therapy and photodynamic diagnosis, for example, for treating or detecting a tumour, or for antiviral treatment. The present invention also relates to the use of reduced chlorin analogues and pharmaceutically acceptable salts thereof in the manufacture of a phototherapeutic or photodiagnostic agent, and to a method of photodynamic therapy, cytoluminescent therapy or photodynamic diagnosis, for example, for treating or detecting a tumour, or for antiviral treatment.

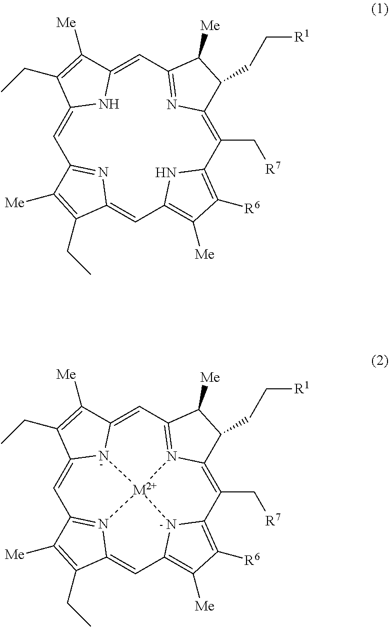

### TECHNICAL FIELD

The present invention relates to reduced chlorin analogues and their pharmaceutically acceptable salts, and compositions comprising reduced chlorin analogues and their pharmaceutically acceptable salts. Reduced chlorin analogues and pharmaceutically acceptable salts thereof are suitable for use in photodynamic therapy, cytoluminescent therapy and photodynamic diagnosis, for example, for treating or detecting a tumour, or for antiviral treatment. The present invention also relates to the use of reduced chlorin analogues and pharmaceutically acceptable salts thereof in the manufacture of a phototherapeutic or photodiagnostic agent, and to a method of photodynamic therapy, cytoluminescent therapy or photodynamic diagnosis, for example, for treating or detecting a tumour, or for antiviral treatment.

The structure of ‘phyllochlorin’ is shown below:

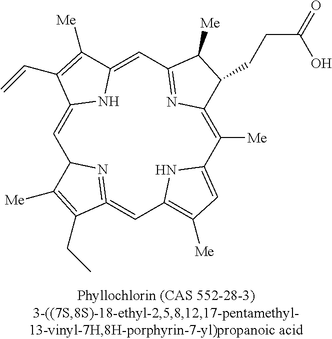

### BACKGROUND ART

Porphyrins and their analogues are known photosensitive chemical compounds, which can absorb light photons and emit them at higher wavelengths. There are many applications for such unique properties and PDT (photodynamic therapy) is one of them.

Presently, there are two generations of photosensitizers for PDT. The first generation comprises heme porphyrins (blood derivatives), and the second for the most part are chlorophyll analogues. The later compounds are known as chlorins and bacteriochlorins.

Chlorin e4 has been shown to display good photosensitive activity. It was indicated that chlorin e4 has a protective effect against indomethacin-induced gastric lesions in rats and TAA- or CCl₄-induced acute liver injuries in mice. It was therefore suggested that chlorin e4 may be a promising new drug candidate for anti-gastrelcosis and liver injury protection. WO 2009/040411 suggests the use of a chlorin e4 zinc complex in photodynamic therapy and WO 2014/091241 suggests the use of chlorin e4 disodium in photodynamic therapy.

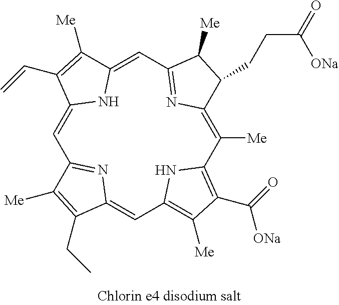

However, there is an ongoing need for better photosensitizers. There is a need for compounds that have a high singlet oxygen quantum yield and for compounds that have a strong photosensitizing ability, preferably in organic and aqueous media. There is also a need for compounds that have a high fluorescence quantum yield. In addition, there is a need for compounds and/or compositions which have a higher phototoxicity, a lower dark toxicity, good stability, good solubility, and/or are easily purified.

### SUMMARY OF THE INVENTION

A first aspect of the present invention provides a compound of formula (1) or a complex of formula (2):

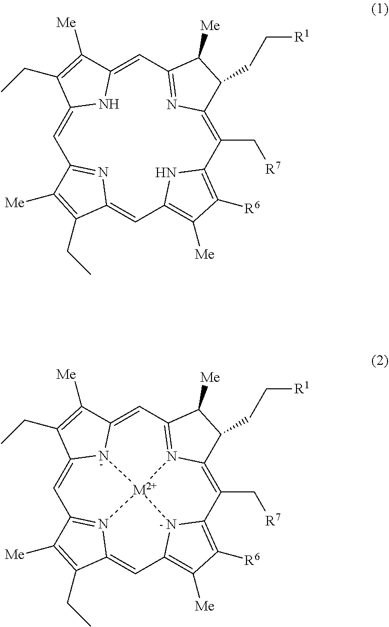

or a pharmaceutically acceptable salt thereof, wherein:
—R¹ is selected from —CH₂OR², —CH₂SR², —CH₂S(O)R², —CH₂S(O)₂R², —CH₂N(R²)₂, —R², —C(O)—OR³, —C(O)—SR³, —C(O)—N(R³)₂, —C(S)—OR³, —C(S)—SR³ or —C(S)—N(R³)₂;
—R², each independently, is selected from —H, —C(O)R⁴, —C(O)—OR⁴, —C(O)—SR⁴, —C(O)—N(R⁴)₂, —C(S)—OR⁴, —C(S)—SR⁴, —C(S)—N(R⁴)₂, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(Rβ)₂, —R^{α}—X, —R^{α}—[N(R⁵)₃]Y, —R^{α}—[P(R⁵)₃]Y, —R^{α}—[R⁸]Y, —R^{α}—[N(R⁵)₂(R^{5′})], —R^{α}—[P(R⁵)₂(R^{5′})] or —R^{α}—[R^{8′}];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁸]Y, —R^{α}—[N(R⁵)₂(R^{5′})], —R^{α}—[P(R⁵)₂(R^{5′})] or —R^{α}—[R^{8′}];
μR^{α}—, each independently, is selected from a C₁-C₄₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂⁻, wherein the phenyl or C₅-C₆ heteroaryl may optionally be further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁶ is selected from hydrogen, —C(O)—OR³, —C(O)—SR³, —C(O)—N(R³)₂, —C(S)—OR³, —C(S)—SR³ or —C(S)—N(R³)₂;
R⁷ is selected from hydrogen, —C(O)—OR³, —C(O)—SR³, —C(O)—N(R³)₂, —C(S)—OR³, —C(S)—SR³ or —C(S)—N(R³)₂; or —R⁶ and —R⁷ together form —C(O)—O—;
—R⁸ is —[NC₅H₅] optionally substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
R^{8′} is —[NC₅H₅] substituted with —CO₂ and optionally further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
n is 1, 2, 3, 4, 5 or 6;
X is a halo group;
Y is a counter anion;
Z is a counter cation; and
M²⁺ is a metal cation.

The first aspect of the present invention also provides a compound of formula (I) or a complex of formula (II):

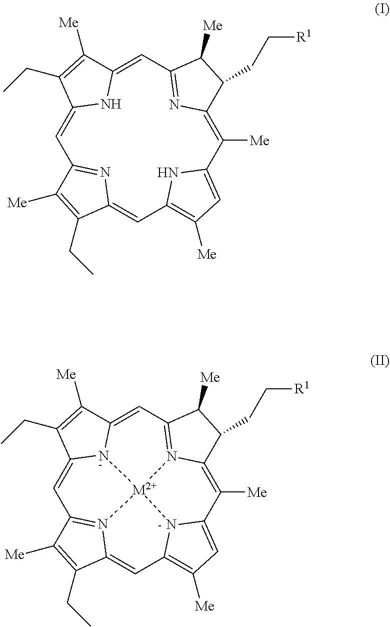

or a pharmaceutically acceptable salt thereof, wherein:
—R¹ is selected from —CH₂OR², —CH₂SR², —CH₂S(O)R², —CH₂S(O)₂R², —CH₂N(R²)₂, —R², —C(O)—OR³, —C(O)—SR³, —C(O)—N(R³)₂, —C(S)—OR³, —C(S)—SR³ or —C(S)—N(R³)₂;
—R², each independently, is selected from —H, —C(O)R⁴, —C(O)—OR⁴, —C(O)—SR⁴, —C(O)—N(R⁴)₂, —C(S)—OR⁴, —C(S)—SR⁴, —C(S)—N(R⁴)₂, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁸]Y, —R^{α}—[N(R⁵)₂(R^{5′})], —R^{α}—[P(R⁵)₂(R^{5′})] or —R^{α}—[R^{8′}];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁸]Y, —R^{α}—[N(R⁵)₂(R^{5′})], —R^{α}—[P(R⁵)₂(R^{5′})] or —R^{α}—[R^{8′}];
—μR^{α}—, each independently, is selected from a C₁-C₄₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂⁻, wherein the phenyl or C₅-C₆ heteroaryl may optionally be further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁸ is —[NC₅H₅] optionally substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{8′} is —[NC₅H₅] substituted with —CO₂ and optionally further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
n is 1, 2, 3, 4, 5 or 6;
X is a halo group;
Y is a counter anion;
Z is a counter cation; and
M²⁺ is a metal cation.

The first aspect of the present invention also provides a compound of formula (I) or a complex of formula (II):

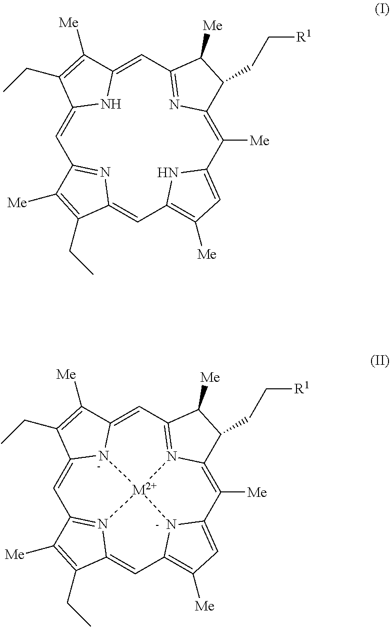

wherein
—R¹ is selected from —C(O)—OR³, —C(O)—SR³, —C(O)—N(R³)₂, —C(S)—OR³, —C(S)—SR³ or —C(S)—N(R³)₂;
—R³, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y or —R^{α}—[R⁸]Y;
—R^{α}—, each independently, is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O, S, P and Se in its carbon skeleton;
—R⁵, each independently, is selected from C₁-C₄ alkyl, C₁-C₄ haloalkyl, halo, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
—R⁸ is —[NC₅H₅] optionally substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
n is 1, 2, 3 or 4;
Y is a counter ion;
X is a halo group;
M²⁺ is a metal ion;
   or a pharmaceutically acceptable salt thereof.

Reduced phyllochlorin free acid has the structure:

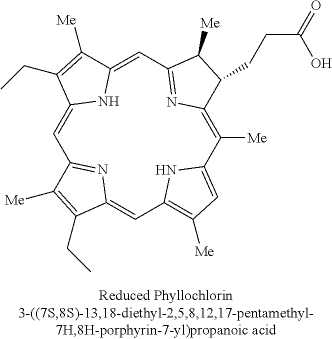 [SMILES: CCC1=C(C)c2cc3[nH]c(cc4nc(c(C)c5cc(C)c(cc1n2)[nH]5)[C@@H](CCC(=O)O)[C@@H]4C)c(C)c3CC]

In one embodiment of the first aspect of the invention, when the compound of formula (1) or (I) is reduced phyllochlorin free acid, reduced phyllochlorin methyl ester or a salt thereof, then the reduced phyllochlorin free acid, reduced phyllochlorin methyl ester or salt thereof is substantially enantiomerically pure.

In one embodiment of the first aspect of the invention, the compound of formula (1) or (I) is not reduced phyllochlorin free acid, reduced phyllochlorin iron salt, reduced phyllochlorin copper salt, or reduced phyllochlorin methyl ester.

In one embodiment of the first aspect of the invention, the compound of formula (1) or (I) is not reduced phyllochlorin free acid, reduced phyllochlorin methyl ester or a salt thereof.

A second aspect of the present invention provides a compound of formula (1) or a complex of formula (2):

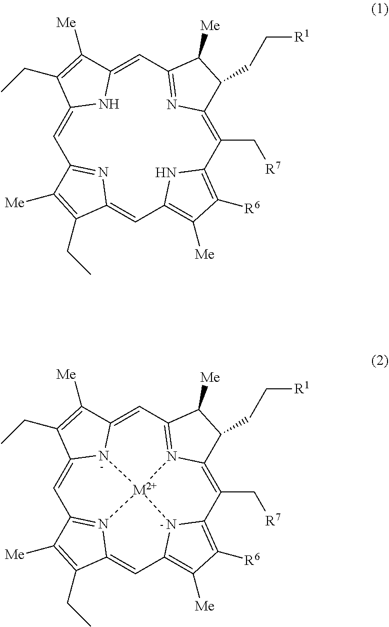

or a pharmaceutically acceptable salt thereof, for use in medicine, wherein:
—R¹ is selected from —CH₂OR², —CH₂SR², —CH₂S(O)R², —CH₂S(O)₂R², —CH₂N(R²)₂, —R², —C(O)—OR³, —C(O)—SR³, —C(O)—N(R³)₂, —C(S)—OR³, —C(S)—SR³ or —C(S)—N(R³)₂;
—R², each independently, is selected from —H, —C(O)R⁴, —C(O)—OR⁴, —C(O)—SR⁴, —C(O)—N(R⁴)₂, —C(S)—OR⁴, —C(S)—SR⁴, —C(S)—N(R⁴)₂, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁸]Y, —R^{α}—[N(R⁵)₂(R^{5′})], —R^{α}—[P(R⁵)₂(R^{5′})] or —R^{α}—[R^{8′}];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁸]Y, —R^{α}—[N(R⁵)₂(R^{5′})], —R^{α}—[P(R⁵)₂(R^{5′})] or —R^{α}—[R^{8′}];
—R^{α}—, each independently, is selected from a C₁-C₄₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)₁—H or —O—(CH₂CH₂O)₁—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂, wherein the phenyl or C₅-C₆ heteroaryl may optionally be further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁶ is selected from hydrogen, —C(O)—OR³, —C(O)—SR³, —C(O)—N(R³)₂, —C(S)—OR³, —C(S)—SR³ or —C(S)—N(R³)₂;
—R⁷ is selected from hydrogen, —C(O)—OR³, —C(O)—SR³, —C(O)—N(R³)₂, —C(S)—OR³, —C(S)—SR³ or —C(S)—N(R³)₂;
or —R⁶ and —R⁷ together form —C(O)—O—;
—R⁸ is —[NC₅H₅] optionally substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{8′} is —[NC₅H₅] substituted with —CO₂ and optionally further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
n is 1, 2, 3, 4, 5 or 6;
X is a halo group;
Y is a counter anion;
Z is a counter cation; and
M²⁺ is a metal cation.

The second aspect of the present invention also provides a compound of formula (I) or a complex of formula (II):

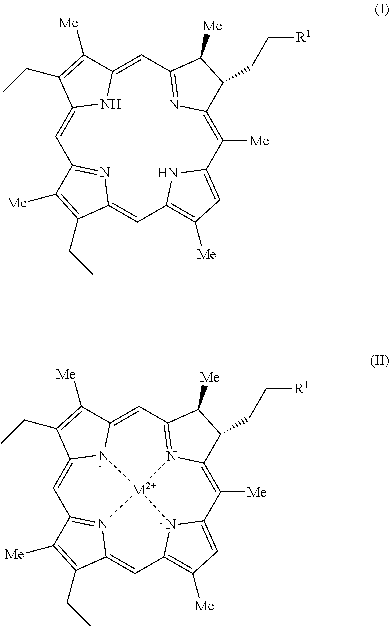

or a pharmaceutically acceptable salt thereof, for use in medicine, wherein:
—R¹ is selected from —CH₂OR², —CH₂SR², —CH₂S(O)R², —CH₂S(O)₂R², —CH₂N(R²)₂, —R², —C(O)—OR³, —C(O)—SR³, —C(O)—N(R³)₂, —C(S)—OR³, —C(S)—SR³ or —C(S)—N(R³)₂;
—R², each independently, is selected from —H, —C(O)R⁴, —C(O)—OR⁴, —C(O)—SR⁴, —C(O)—N(R⁴)₂, —C(S)—OR⁴, —C(S)—SR⁴, —C(S)—N(R⁴)₂, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁸]Y, —R^{α}—[N(R⁵)₂(R^{5′})], —R^{α}—[P(R⁵)₂(R^{5′})] or —R^{α}—[R^{8′}];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁸]Y, —R^{α}—[N(R⁵)₂(R^{5′})], —R^{α}—[P(R⁵)₂(R^{5′})] or —R^{α}—[R^{8′}];
R^{α}—, each independently, is selected from a C₁-C₄₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂⁻, wherein the phenyl or C₅-C₆ heteroaryl may optionally be further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁸ is —[NC₅H₅] optionally substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{8′} is —[NC₅H₅] substituted with —CO₂ and optionally further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
n is 1, 2, 3, 4, 5 or 6;
X is a halo group;
Y is a counter anion;
Z is a counter cation; and
M²⁺ is a metal cation.

The second aspect of the present invention also provides a compound of formula (I) or a complex of formula (II):

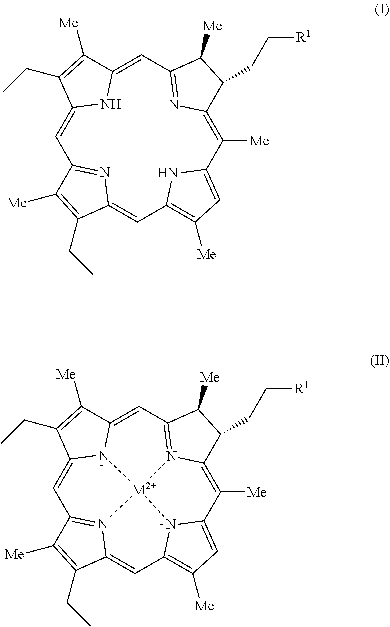

wherein
—R¹ is selected from —C(O)—OR³, —C(O)—SR³, —C(O)—N(R³)₂, —C(S)—OR³, —C(S)—SR³ or —C(S)—N(R³)₂;
—R³, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y or —R^{α}—[R⁸]Y;
—R^{α}—, each independently, is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O, S, P and Se in its carbon skeleton;
—R⁵, each independently, is selected from C₁-C₄ alkyl, C₁-C₄ haloalkyl, halo, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
—R⁸ is —[NC₅H₅] optionally substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
n is 1, 2, 3 or 4;
Y is a counter ion;
X is a halo group;
M²⁺ is a metal ion;
   or a pharmaceutically acceptable salt thereof;
   for use in medicine.

In some embodiments of the second aspect of the invention the compound is:
(1) reduced phyllochlorin free acid; or
(2) reduced phyllochlorin methyl ester.

In one embodiment of the first or second aspect of the present invention, the compound of formula (1), (2), (I), (II), (III), (IV), (V), (VI), (VII) or (VIII) is not:
(i) reduced phyllochlorin free acid;
(ii) reduced phyllochlorin iron salt;
(iii) reduced phyllochlorin copper salt;
(iv) reduced phyllochlorin methyl ester;
(v) 3-(13,18-diethyl-2,5,8,12,17-pentamethyl-7H,8H-porphyrin-7-yl)propanoate zinc complex;
(vi) 3-(13,18-diethyl-2,5,8,12,17-pentamethyl-7H,8H-porphyrin-7-yl)propanoic acid zinc complex;
(vii) (7-(3-((carboxymethyl)amino)-3-oxopropyl)-13,18-diethyl-2,5,8,12,17-pentamethyl-7H,8H-porphyrin-3-carbonyl)glycine;
(viii) 3-(7-(3-((carboxymethyl)amino)-3-oxopropyl)-13,18-diethyl-2,5,8,12,17-pentamethyl-7H,8H-porphyrin-3-carboxamido)propanoic acid;
(ix) (7-(3-((1-carboxyethyl)amino)-3-oxopropyl)-13,18-diethyl-2,5,8,12,17-pentamethyl-7H,8H-porphyrin-3-carbonyl)alanine;
   or an enantiomer of any thereof;
   or a racemic mixture of any thereof;
   or a salt of any thereof.

In one embodiment of the first or second aspect of the present invention, —R¹, —R⁶ and —R⁷ are not simultaneously selected from —CO₂H, —CO₂Me or —CONHR³.

In one embodiment of the first or second aspect of the present invention, when —R¹ and —R⁷ are both —CO₂Me, then —R⁶ is not —CO—NMe₂.

In one embodiment of the first or second aspect of the present invention, in the compound of formula (1) or (2), —R⁶ is hydrogen and —R⁷ is hydrogen.

In one embodiment of the first or second aspect of the present invention, in the compound of formula (1) or (2), —R⁶ is —CO₂H and —R⁷ is hydrogen.

In one embodiment of the first or second aspect of the present invention, in the compound of formula (1) or (2), —R⁶ is —CO₂H and —R⁷ is —CO₂H.

In one embodiment of the first or second aspect of the present invention, in the compound of formula (1) or (2), —R⁶ and —R⁷ together form —C(O)—O—.

In the context of the present specification, a “hydrocarbyl” substituent group or a hydrocarbyl moiety in a substituent group only includes carbon and hydrogen atoms but, unless stated otherwise, does not include any heteroatoms, such as N, O, S, P or Se in its carbon skeleton. A hydrocarbyl group/moiety may be saturated or unsaturated (including aromatic), and may be straight-chained or branched, or be or include cyclic groups wherein, unless stated otherwise, the cyclic group does not include any heteroatoms, such as N, O, S, P or Se in its carbon skeleton. Examples of hydrocarbyl groups include alkyl, alkenyl, alkynyl, cycloalkyl, cycloalkenyl and aryl groups/moieties and combinations of all of these groups/moieties. Typically a hydrocarbyl group is a C₁-C₆₀ hydrocarbyl group, more typically a C₁-C₄₀ hydrocarbyl group, more typically a C₁-C₂₀ hydrocarbyl group. More typically a hydrocarbyl group is a C₁-C₁₂ hydrocarbyl group. More typically a hydrocarbyl group is a C₁-C₁₀ hydrocarbyl group. A “hydrocarbylene” group is similarly defined as a divalent hydrocarbyl group.

An “alkyl” substituent group or an alkyl moiety in a substituent group may be linear (i.e. straight-chained) or branched. Examples of alkyl groups/moieties include methyl, ethyl, n-propyl, i-propyl, n-butyl, i-butyl, t-butyl and n-pentyl groups/moieties. Unless stated otherwise, the term “alkyl” does not include “cycloalkyl”. Typically an alkyl group is a C₁-C₁₂ alkyl group. More typically an alkyl group is a C₁-C₆ alkyl group. An “alkylene” group is similarly defined as a divalent alkyl group.

An “alkenyl” substituent group or an alkenyl moiety in a substituent group refers to an unsaturated alkyl group or moiety having one or more carbon-carbon double bonds. Examples of alkenyl groups/moieties include ethenyl, propenyl, 1-butenyl, 2-butenyl, 1-pentenyl, 1-hexenyl, 1,3-butadienyl, 1,3-pentadienyl, 1,4-pentadienyl and 1,4-hexadienyl groups/moieties. Unless stated otherwise, the term “alkenyl” does not include “cycloalkenyl”. Typically an alkenyl group is a C₂-C₁₂ alkenyl group. More typically an alkenyl group is a C₁-C₆ alkenyl group. An “alkenylene” group is similarly defined as a divalent alkenyl group.

An “alkynyl” substituent group or an alkynyl moiety in a substituent group refers to an unsaturated alkyl group or moiety having one or more carbon-carbon triple bonds. Examples of alkynyl groups/moieties include ethynyl, propargyl, but-1-ynyl and but-2-ynyl. Typically an alkynyl group is a C₂-C₁₂ alkynyl group. More typically an alkynyl group is a C₂-C₆ alkynyl group. An “alkynylene” group is similarly defined as a divalent alkynyl group.

A “cyclic” substituent group or a cyclic moiety in a substituent group refers to any hydrocarbyl ring, wherein the hydrocarbyl ring may be saturated or unsaturated (including aromatic) and may include one or more heteroatoms, e.g. N, O, S, P or Se in its carbon skeleton. Examples of cyclic groups include cycloalkyl, cycloalkenyl, heterocyclic, aryl and heteroaryl groups as discussed below. A cyclic group may be monocyclic, bicyclic (e.g. bridged, fused or spiro), or polycyclic. Typically, a cyclic group is a 3- to 12-membered cyclic group, which means it contains from 3 to 12 ring atoms. More typically, a cyclic group is a 3- to 7-membered monocyclic group, which means it contains from 3 to 7 ring atoms.

A “heterocyclic” substituent group or a heterocyclic moiety in a substituent group refers to a cyclic group or moiety including one or more carbon atoms and one or more (such as one, two, three or four) heteroatoms, e.g. N, O, S, P or Se in the ring structure. Examples of heterocyclic groups include heteroaryl groups as discussed below and non-aromatic heterocyclic groups such as azetidinyl, azetinyl, tetrahydrofuranyl, pyrrolidinyl, tetrahydrothiophenyl, tetrahydropyranyl, piperidinyl, piperazinyl, morpholinyl, thiomorpholinyl, oxetanyl, thietanyl, pyrazolidinyl, imidazolidinyl, dioxolanyl, oxathiolanyl, thianyl and dioxanyl groups.

A “cycloalkyl” substituent group or a cycloalkyl moiety in a substituent group refers to a saturated hydrocarbyl ring containing, for example, from 3 to 7 carbon atoms, examples of which include cyclopropyl, cyclobutyl, cyclopentyl and cyclohexyl. Unless stated otherwise, a cycloalkyl substituent group or moiety may include monocyclic, bicyclic or polycyclic hydrocarbyl rings.

A “cycloalkenyl” substituent group or a cycloalkenyl moiety in a substituent group refers to a non-aromatic unsaturated hydrocarbyl ring having one or more carbon-carbon double bonds and containing, for example, from 3 to 7 carbon atoms, examples of which include cyclopent-1-en-1-yl, cyclohex-1-en-1-yl and cyclohex-1,3-dien-1-yl.

Unless stated otherwise, a cycloalkenyl substituent group or moiety may include monocyclic, bicyclic or polycyclic hydrocarbyl rings.

An “aryl” substituent group or an aryl moiety in a substituent group refers to an aromatic hydrocarbyl ring. The term “aryl” includes monocyclic aromatic hydrocarbons and polycyclic fused ring aromatic hydrocarbons wherein all of the fused ring systems (excluding any ring systems which are part of or formed by optional substituents) are aromatic. Examples of aryl groups/moieties include phenyl, naphthyl, anthracenyl and phenanthrenyl. Unless stated otherwise, the term “aryl” does not include “heteroaryl”.

A “heteroaryl” substituent group or a heteroaryl moiety in a substituent group refers to an aromatic heterocyclic group or moiety. The term “heteroaryl” includes monocyclic aromatic heterocycles and polycyclic fused ring aromatic heterocycles wherein all of the fused ring systems (excluding any ring systems which are part of or formed by optional substituents) are aromatic. Examples of heteroaryl groups/moieties include the following:

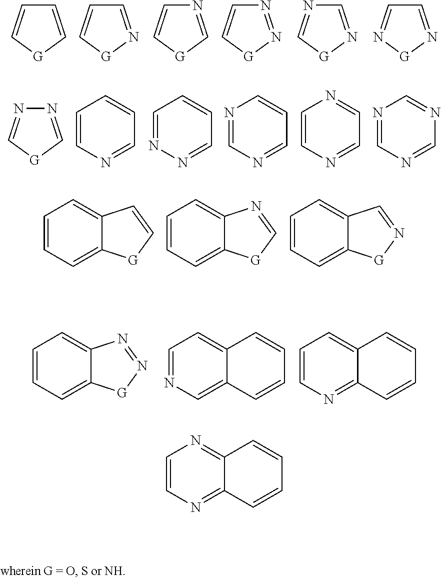

For the purposes of the present specification, where a combination of moieties is referred to as one group, for example, arylalkyl, arylalkenyl, arylalkynyl, alkylaryl, alkenylaryl or alkynylaryl, the last mentioned moiety contains the atom by which the group is attached to the rest of the molecule. An example of an arylalkyl group is benzyl.

For the purposes of the present specification, in an optionally substituted group or moiety (such as —R^{β}):
(i) each hydrogen atom may optionally be replaced by a monovalent substituent independently selected from halo; —CN; —NO₂; —N₃; —R^{x}; —OH; —OR^{x}; —R^{y}-halo; —R^{y}—CN; —R^{y}—NO₂; —R^{y}—N₃; —R^{y}—R^{x}; —R^{y}—OH; —R^{y}—OR^{x}; —SH; —SR^{x}; —SOR^{x}; —SO₂H; —SO₂R^{x}; —SO₂NH₂; —SO₂NHR^{x}; —SO₂N(R^{x})₂; —R^{y}—SH; —R^{y}—SR^{x}; —R^{y}—SOR^{x}; —R^{y}—SO₂H; —R^{y}—SO₂R^{x}; —R^{y}—SO₂NH₂; —R^{y}—SO₂NHR^{x}; —R^{y}—SO₂N(R^{x})₂; —NH₂; —NHR^{x}; —N(R^{x})₂; —N⁺(R^{x})₃; —R^{y}—NH₂; —R^{y}—NHR^{x}; —R^{y}—N(R^{x})₂; —R^{y}—N⁺(R^{x})₃; —CHO; —COR^{x}; —COOH; —COOR^{x}; —OCOR^{x}; —R^{y}—CHO; —R^{y}—COR^{x}; —R^{y}—COOH; —R^{y}—COOR^{x}; or —R^{y}—OCOR^{x}; and/or
(ii) any two hydrogen atoms attached to the same carbon atom may optionally be replaced by a r-bonded substituent independently selected from oxo (═O), ═S, ═NH, or ═NR^{x}; and/or
(iii) any two hydrogen atoms attached to the same or different atoms, within the same optionally substituted group or moiety, may optionally be replaced by a bridging substituent independently selected from —O—, —S—, —NH—, —N(R^{x})—, —N⁺(R^{x})₂— or —R^{y}—;
   wherein each —R^{y}— is independently selected from an alkylene, alkenylene or alkynylene group, wherein the alkylene, alkenylene or alkynylene group contains from 1 to 6 atoms in its backbone, wherein one or more carbon atoms in the backbone of the alkylene, alkenylene or alkynylene group may optionally be replaced by one or more heteroatoms N, O or S, and wherein the alkylene, alkenylene or alkynylene group may optionally be substituted with one or more halo and/or —R^{x} groups; and
   wherein each —R^{x} is independently selected from a C₁-C₆ alkyl, C₂-C₆ alkenyl, C₂-C₆ alkynyl or C₂-C₆ cyclic group, or wherein any two or three —R^{x} attached to the same nitrogen atom may, together with the nitrogen atom to which they are attached, form a C₂-C₇ cyclic group, and wherein any —R^{x} may optionally be substituted with one or more C₁-C₄ alkyl, C₁-C₄ haloalkyl, —O(C₁-C₄ alkyl), —O(C₁-C₄ haloalkyl), halo, —OH, —NH₂, —CN, or oxo (═O) groups.

Typically a substituted group comprises 1, 2, 3 or 4 substituents, more typically 1, 2 or 3 substituents, more typically 1 or 2 substituents, and more typically 1 substituent.

Unless stated otherwise, any divalent bridging substituent (e.g. —O—, —S—, —NH—, —N(R^{x})—, —N⁺(R^{x})₂— or —R^{y}—) of an optionally substituted group or moiety must only be attached to the specified group or moiety and may not be attached to a second group or moiety, even if the second group or moiety can itself be optionally substituted.

The term “halo” includes fluoro, chloro, bromo and iodo.

Unless stated otherwise, where a group is prefixed by the term “halo”, such as a haloalkyl or halomethyl group, it is to be understood that the group in question is substituted with one or more halo groups independently selected from fluoro, chloro, bromo and iodo. Typically, the maximum number of halo substituents is limited only by the number of hydrogen atoms available for substitution on the corresponding group without the halo prefix. For example, a halomethyl group may contain one, two or three halo substituents. A haloethyl or halophenyl group may contain one, two, three, four or five halo substituents. Similarly, unless stated otherwise, where a group is prefixed by a specific halo group, it is to be understood that the group in question is substituted with one or more of the specific halo groups. For example, the term “fluoromethyl” refers to a methyl group substituted with one, two or three fluoro groups.

Unless stated otherwise, where a group is said to be “halo-substituted”, it is to be understood that the group in question is substituted with one or more halo groups independently selected from fluoro, chloro, bromo and iodo. Typically, the maximum number of halo substituents is limited only by the number of hydrogen atoms available for substitution on the group said to be halo-substituted. For example, a halo-substituted methyl group may contain one, two or three halo substituents. A halo-substituted ethyl or halo-substituted phenyl group may contain one, two, three, four or five halo substituents.

Unless stated otherwise, any reference to an element is to be considered a reference to all isotopes of that element. Thus, for example, unless stated otherwise any reference to hydrogen is considered to encompass all isotopes of hydrogen including deuterium and tritium.

Unless stated otherwise, any reference to a compound or group is to be considered a reference to all tautomers of that compound or group.

Where reference is made to a hydrocarbyl or other group including one or more heteroatoms N, O, S, P or Se in its carbon skeleton, or where reference is made to a carbon atom of a hydrocarbyl or other group being replaced by an N, O, S, P or Se atom, what is intended is that:

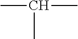

is replaced by

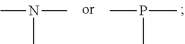

—CH₂— is replaced by —NH—, —PH—, —O—, —S— or —Se—;
—CH₃ is replaced by —NH₂, —PH₂, —OH, —SH or —SeH;
—CH═ is replaced by —N═ or —P═;
—CH₂═ is replaced by NH═, PH═, O═, S═ or Se═; or
CH≡ is replaced by N≡ or P≡;
provided that the resultant group comprises at least one carbon atom. For example, methoxy, dimethylamino and aminoethyl groups are considered to be hydrocarbyl groups including one or more heteroatoms N, O, S, P or Se in their carbon skeleton.

In the context of the present specification, unless otherwise stated, a Cₓ-C_{y} group is defined as a group containing from x to y carbon atoms. For example, a C₁-C₄ alkyl group is defined as an alkyl group containing from 1 to 4 carbon atoms. Optional substituents and moieties are not taken into account when calculating the total number of carbon atoms in the parent group substituted with the optional substituents and/or containing the optional moieties. For the avoidance of doubt, replacement heteroatoms, e.g. N, O, S, P or Se are to be counted as carbon atoms when calculating the number of carbon atoms in a Cₓ-C_{y} group. For example, a morpholinyl group is to be considered a C₆ heterocyclic group, not a C₄ heterocyclic group.

The a electrons of the chlorin ring are delocalised and therefore the chlorin ring can be depicted by more than one resonance structure. Resonance structures are different ways of drawing the same compound. Two of the resonance structures of the chlorin ring are depicted directly below:

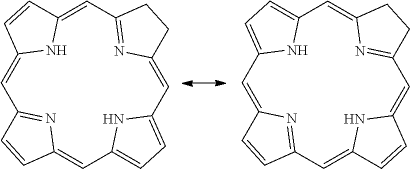 [SMILES: C1=Cc2cc3ccc(cc4nc(cc5ccc(cc1n2)[nH]5)CC4)[nH]3 ; C1=Cc2cc3ccc(cc4nc(cc5ccc(cc1n2)[nH]5)CC4)[nH]3]

Typically a complex comprises a central metal atom or ion known as the coordination centre and a bound molecule or ion which is known as a ligand. In the present specification, the bond between the coordination centre and the ligand is depicted as shown in the complex on the below left (where the attraction between an anionic ligand and a central metal cation is represented by four dashed lines), but equivalently it could be depicted as shown in the complex on the below right (where the attraction between a ligand molecule and a central metal atom is represented by two covalent bonds and two dashed lines):

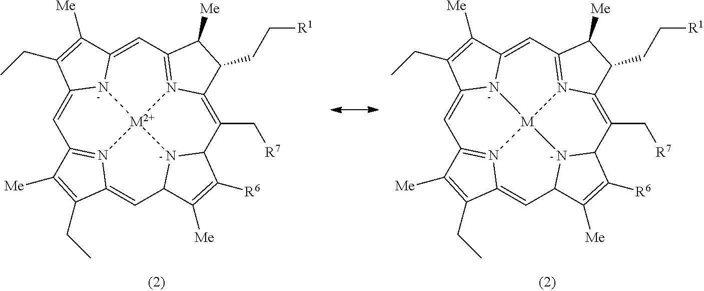

As used herein —[NC₅H₅]Y refers to:

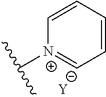

In one embodiment of the first or second aspect of the present invention, X is a halo group selected from fluoro, chloro, bromo, or iodo. In one embodiment, X is chloro or bromo.

In one embodiment of the first or second aspect of the present invention, there is provided a compound of formula (I).

In one embodiment of the first or second aspect of the present invention, Y is a counter anion selected from halides (for example fluoride, chloride, bromide, or iodide) or other inorganic anions (for example bisulfate, hexafluorophosphate (PF6), nitrate, perchlorate, phosphate, or sulfate) or organic anions (for example acetate, ascorbate, aspartate, benzoate, besylate (benzenesulfonate), bicarbonate, bis(trifluoromethanesulfonyl)imide (TFSI), bitartrate, butyrate, camsylate (camphorsulfonate), carbonate, citrate, decanoate, edetate, esylate (ethanesulfonate), fumarate, galactarate, gluceptate, gluconate, glutamate, glycolate, hexanoate, β-hydroxybutyrate, 2-hydroxyethanesulfonate, hydroxymaleate, hydroxynaphthoate, isethionate, lactate, lactobionate, malate, maleate, mandelate, mesylate (methanesulfonate), methylsulfate, mucate, napsylate (naphthalene-2-sulfonate), octanoate, oleate, ornithinate, pamoate, pantothenate, polygalacturonate, propanoate, propionate, salicylate, stearate, succinate, tartrate, teoclate, tetrakis[3,5-bis(trifluoromethyl)phenyl]borate (BARF), tetrakis(pentafluorophenyl)borate (F5-TPB), tetraphenylborate (TPB), tosylate (toluene-p-sulfonate), or triflate (trifluoromethanesulfonate)).

In another embodiment of the first or second aspect of the present invention, Y is a counter anion selected from halides (for example fluoride, chloride, bromide, or iodide) or other inorganic anions (for example bisulfate, nitrate, perchlorate, phosphate, or sulfate) or organic anions (for example acetate, aspartate, benzoate, besylate (benzenesulfonate), butyrate, camsylate (camphorsulfonate), citrate, esylate (ethanesulfonate), fumarate, galactarate, gluconate, glutamate, glycolate, 2-hydroxyethanesulfonate, hydroxymaleate, lactate, malate, maleate, mandelate, mesylate (methanesulfonate), napsylate (naphthalene-2-sulfonate), ornithinate, pamoate, pantothenate, propanoate, salicylate, succinate, tartrate, tosylate (toluene-p-sulfonate), or triflate (trifluoromethanesulfonate)). In one embodiment, Y is fluoride, chloride, bromide or iodide. In one embodiment, Y is chloride or bromide.

In one embodiment of the first or second aspect of the present invention, Z is a counter cation selected from inorganic cations (for example lithium, sodium, potassium, magnesium, calcium or ammonium cation) or organic cations (for example amine cations (for example choline or meglumine cation) or amino acid cations (for example arginine cation).

In one embodiment of the first or second aspect of the present invention, M²⁺ is a metal cation selected from Zn²⁺, Cu²⁺, Fe²⁺, Pd²⁺ or Pt²⁺. In one embodiment, M²⁺ is Zn²⁺.

In one embodiment of the first or second aspect of the present invention, —R¹ is selected from —C(O)—OR³, —C(O)—SR³, —C(O)—N(R³)₂, —C(S)—OR³, —C(S)—SR³ or —C(S)—N(R³)₂. In one embodiment, —R¹ is selected from —C(O)—OR³, —C(O)—SR³, —C(O)—N(R³)₂ or —C(S)—N(R³)₂. In one embodiment, —R¹ is selected from —C(O)—OR³, —C(O)—SR³ or —C(O)—N(R³)₂.

In one embodiment of the first or second aspect of the present invention, —R¹ is selected from —C(O)—OR³, —C(O)—SR³, —C(O)—N(R³)₂, —C(S)—OR³, —C(S)—SR³ or —C(S)—N(R³)₂, and each —R³ is C₁-C₄ alkyl (preferably methyl). In one embodiment, —R¹ is selected from —C(O)—OR³, —C(O)—SR³, —C(O)—N(R³)₂ or —C(S)—N(R³)₂, and each —R³ is C₁-C₄ alkyl (preferably methyl). In one embodiment, —R¹ is selected from —C(O)—OR³, —C(O)—SR³ or —C(O)—N(R³)₂, and each —R³ is C₁-C₄ alkyl (preferably methyl). In one embodiment, —R¹ is —C(O)—OR³ and —R³ is C₁-C₄ alkyl (preferably methyl).

In one embodiment of the first or second aspect of the present invention, —R¹ is selected from —C(O)—OR³, —C(O)—SR³, —C(O)—N(R³)₂, —C(S)—OR³, —C(S)—SR³ or —C(S)—N(R³)₂, and each —R³ is selected from —R^{α}—OR^{β} or —R^{α}—SR^{β}, and —R^{β} is a saccharidyl group. In one embodiment, —R¹ is selected from —C(O)—OR³, —C(O)—SR³ or —C(O)—N(R³)₂, and each —R³ is selected from —R^{α}—OR^{β} or —R^{α}—SR^{β}, and —R^{β} is a saccharidyl group. In one embodiment, —R¹ is selected from —C(O)—OR³ or —C(O)—SR³, and —R³ is selected from —R^{α}—OR^{β} or —R^{α}—SR^{β}, and —R^{β} is a saccharidyl group. Typically in these embodiments, —R^{α}— is a C₁-C₁₂ alkylene group (preferably a C₁-C₈ alkylene group, or a C₁-C₆ alkylene group), a —(CH₂CH₂O)ₘ—CH₂CH₂— group or a —(CH₂CH₂S)ₘ—CH₂CH₂— group, all optionally substituted, wherein m is 1, 2, 3 or 4.

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}), wherein —R² or —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}), wherein —R³ is selected from —R^{α}—OR^{β} or —R^{α}—SR^{β}, and —R^{β} is a saccharidyl group, and —R^{3′} is H or C₁-C₄ alkyl (preferably methyl). In one embodiment, —R¹ is —C(O)—N(R³)(R^{3′}), wherein —R³ is selected from —R^{α}—OR^{β} or —R^{α}—SR^{β}, and —R^{β} is a saccharidyl group, and —R^{3′} is H or C₁-C₄ alkyl (preferably methyl). Typically in these embodiments, —R^{α}— is selected from a C₁-C₁₂ alkylene group, wherein one, two, three or four carbon atoms in the backbone of the alkylene group may optionally be replaced by a heteroatom or group independently selected from O, S, NH or NMe. Alternatively, in these embodiments, —R^{α}— is a C₁-C₁₂ alkylene group (preferably a C₁-C₈ alkylene group, or a C₁-C₆ alkylene group), a —(CH₂CH₂O)ₘ—CH₂CH₂— group or a —(CH₂CH₂S)ₘ—CH₂CH₂— group, all optionally substituted, wherein m is 1, 2, 3 or 4.

In one embodiment of the first or second aspect of the present invention, —R¹ is selected from —R², —C(O)—OR³, —C(O)—SR³, —C(O)—N(R³)(R^{3′}) or —C(S)—N(R³)(R^{3′}), wherein —R² or —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}), wherein —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl (preferably methyl). In one embodiment, —R¹ is —C(O)—N(R³)(R^{3′}), wherein —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl (preferably methyl). In one embodiment, —R¹ is —C(O)—N(R³)(R^{3′}), wherein —R³ is selected from —R^{α}—OR^{β} or —R^{α}—SR^{β}, and —R^{β} is a saccharidyl group, and —R^{3′} is H or C₁-C₄ alkyl (preferably methyl). In one embodiment, —R¹ is —C(O)—N(R³)(R^{3′}), wherein —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is C₁-C₄ alkyl (preferably methyl). In one embodiment, —R¹ is —C(O)—N(R³)(R^{3′}), wherein —R³ is selected from —R^{α}—OR^{β} or —R^{α}—SR^{β}, and —R^{β} is a saccharidyl group, and —R^{3′} is C₁-C₄ alkyl (preferably methyl). Typically in these embodiments, —R^{α}— is selected from a C₁-C₁₂ alkylene group, wherein one, two, three or four carbon atoms in the backbone of the alkylene group may optionally be replaced by a heteroatom or group independently selected from O, S, NH or NMe. Alternatively, in these embodiments, —R^{α}— is a C₁-C₁₂ alkylene group (preferably a C₁-C₈ alkylene group, or a C₁-C₆ alkylene group), a —(CH₂CH₂O)ₘ—CH₂CH₂— group or a —(CH₂CH₂S)ₘ—CH₂CH₂— group, all optionally substituted, wherein m is 1, 2, 3 or 4.

An —R^{3′} group refers to an —R³ group attached to the same atom as another —R³ group. —R³ and —R^{3′} may be the same or different. Preferably —R³ and —R^{3′} are different.

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}), wherein —R² or —R³ is selected from —R^{α}—R^{β} or —R^{β}, and —R^{β} is a saccharidyl group, and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}), wherein —R³ is selected from —R^{α}—R^{β} or —R^{β}, and —R^{β} is a saccharidyl group, and —R^{3′} is H or C₁-C₄ alkyl (preferably methyl). In one embodiment, —R¹ is —C(O)—N(R³)(R^{3′}), wherein —R³ is selected from —R^{α}—R^{β} or —R^{β}, and —R^{β} is a saccharidyl group, and —R^{3′} is H or C₁-C₄ alkyl (preferably methyl). Typically in these embodiments, —R^{α}— is a C₁-C₁₂ alkylene group (preferably a C₁-C₈ alkylene group, or a C₁-C₆ alkylene group), a —(CH₂CH₂O)ₘ— group or a —(CH₂CH₂S)ₘ— group, all optionally substituted, wherein m is 1, 2, 3 or 4.

In any of the embodiments in the four preceding paragraphs, the saccharidyl group may optionally be substituted, for example, with a protecting group such as acetyl or a natural amino acid such as valine. Amino acids can be attached to saccharidyl groups, for example, by forming an ester between a carboxylic acid group of the amino acid and a hydroxyl group of the saccharidyl group.

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}), wherein —R² or —R³ is selected from —R^{α}—R^{β} or —R^{β}, and —R^{β} is a C₁-C₈ alkyl group optionally substituted with one or more (such as one, two, three, four, five, six, seven or eight) —OH or —OAc groups, and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}), wherein —R³ is selected from —R^{α}—R^{β} or —R^{β}, and —R^{β} is a C₁-C₈ alkyl group optionally substituted with one or more (such as one, two, three, four, five, six, seven or eight) hydroxyl groups, and —R^{3′} is H or C₁-C₄ alkyl (preferably methyl). In one embodiment, —R¹ is —C(O)—N(R³)(R^{3′}), wherein —R³ is selected from —R^{α}—R^{β} or —R^{β}, and —R^{β} is a C₁-C₈ alkyl group optionally substituted with one or more (such as one, two, three, four, five, six, seven or eight) hydroxyl groups, and —R^{3′} is H or C₁-C₄ alkyl (preferably methyl). Typically in these embodiments, —R^{α}— is an unsubstituted C₁-C₆ alkylene group, or an unsubstituted C₁-C₄ alkylene group, or an unsubstituted C₁-C₂ alkylene group.

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); wherein —R² or —R³ is selected from —R^{α}—H or —R^{α}—OH; —R^{α}— is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S; and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}); wherein —R³ is selected from —R^{α}—H or —R^{α}—OH; —R^{α}— is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S; and —R^{3′} is H or C₁-C₄ alkyl (preferably methyl). In one embodiment, —R¹ is —C(O)—N(R³)(R^{3′}); wherein —R³ is selected from —R^{α}—H or —R^{α}—OH; —R^{α}— is selected from a C₁-C₁₂ alkylene group, wherein one or more carbon atoms in the backbone of the alkylene group may optionally be replaced by one or more heteroatoms O or S; and —R^{3′} is H or C₁-C₄ alkyl (preferably methyl).

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); wherein —R² or —R³ is —R^{β}; —R^{β} is a C₁-C₁₂ alkyl or C₂-C₁₂ alkenyl group optionally substituted with one or more (such as one, two, three, four or five) substituents independently selected from halo, —CN, —NO₂, —N₃, —OH, —OR^{x}, —SH, —SR^{x}, —SOR^{x}, —SO₂H, —SO₂R^{x}, —SO₂NH₂, —SO₂NHR^{x}, —SO₂N(R^{x})₂, —NH₂, —NHR^{x}, —N(R^{x})₂, —N⁺(R^{x})₃, —CHO, —COR^{x}, —COOH, —COOR^{x}, —OCOR^{x}, or —NH—CO—CR^{z}—NH₂; each —R^{x} is independently selected from C₁-C₄ alkyl; —R^{z} is the side chain of a natural amino acid; and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}); wherein —R³ is —R^{β}; —R^{β} is a C₁-C₁₂ alkyl group optionally substituted with one or more (such as one, two, three, four or five) substituents independently selected from halo, —CN, —NO₂, —N₃, —OH, —OR^{x}, —SH, —SR^{x}, —SOR^{x}, —SO₂H, —SO₂R^{x}, —SO₂NH₂, —SO₂NHR^{x}, —SO₂N(R^{x})₂, —NH₂, —NHR^{x}, —N(R^{x})₂, —N⁺(R^{x})₃, —CHO, —COR^{x}, —COOH, —COOR^{x}, —OCOR^{x}, or —NH—CO—CR^{z}—NH₂; each —R^{x} is independently selected from C₁-C₄ alkyl; —R^{z} is the side chain of a natural amino acid; and —R^{3′} is H or C₁-C₄ alkyl (preferably methyl). In one embodiment, —R¹ is —C(O)—N(R³)(R^{3′}); wherein —R³ is —R^{β}; —R^{β} is a C₁-C₈ alkyl group optionally substituted with one or more (such as one, two or three) substituents independently selected from halo, —CN, —NO₂, —N₃, —OH, —OR^{x}, —SH, —SR^{x}, —SOR^{x}, —SO₂H, —SO₂R^{x}, —SO₂NH₂, —SO₂NHR^{x}, —SO₂N(R^{x})₂, —NH₂, —NHR^{x}, —N(R^{x})₂, —N⁺(R^{x})₃, —CHO, —COR^{x}, —COOH, —COOR^{x}, —OCOR^{x}, or —NH—CO—CR^{z}—NH₂; each —R^{x} is independently selected from C₁-C₄ alkyl; —R^{Z} is the side chain of a natural amino acid; and —R^{3′} is H or C₁-C₄ alkyl (preferably methyl).

In one embodiment of the first or second aspect of the present invention, —R¹ is selected from —CO—(NR^{zz}—CHR^{z}—CO)ᵥ—N(R^{zz})₂ and —CO—(NR^{zz}—CHR^{z}—CO)ᵥ—OR^{zz}; wherein each —R^{z} is independently selected from the side chains of natural amino acids; each —R^{zz} is independently selected from hydrogen and C₁-C₄ alkyl (preferably methyl); and v is 1, 2, 3, 4, 5, 6, 7 or 8.

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); wherein —R² or —R³ is —R^{β}; —R^{β} is selected from a C₁-C₂₀ alkyl group, wherein the alkyl group may optionally be substituted with one, two, three or four halo groups, and wherein one, two, three, four, five or six carbon atoms in the backbone of the alkyl group may optionally be replaced by a heteroatom or group independently selected from O, S, NH or NMe; and —R^{3′} is H or C₁-C₄ alkyl (preferably methyl).

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); —R^{3′} is H or C₁-C₄ alkyl (preferably methyl); and —R² or —R³ is selected from —R^{α}—[N(R⁵)₃]Y, —R^{α}—[P(R⁵)₃]Y, or —R^{α}—[R⁸]Y. In one embodiment, —R¹ is selected from —R², —C(O)—OR³, —C(O)—SR³, —C(O)—N(R³)(R^{3′}) or —C(S)—N(R³)(R^{3′}); —R^{3′} is H or C₁-C₄ alkyl (preferably methyl); —R² or —R³ is selected from —R^{α}—[N(R⁵)₃]Y, —R^{α}—[P(R⁵)₃]Y, or —R^{α}—[R⁸]Y; each —R⁵ is independently selected from C₁-C₄ alkyl or phenyl wherein the phenyl is optionally substituted with one, two or three C₁-C₄ alkyl or C₁-C₄ alkoxy groups; —R⁸ is —[NC₅H₅] optionally substituted with one, two or three C₁-C₄ alkyl or C₁-C₄ alkoxy groups; —R^{α}— is selected from a C₁-C₁₂ alkylene group, wherein one, two, three or four carbon atoms in the backbone of the alkylene group may optionally be replaced by a heteroatom or group independently selected from O, S, NH or NMe; and Y is a counter ion (preferably a halide).

In one embodiment of the first or second aspect of the present invention, —R¹ is selected from —R², —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); wherein —R² or —R³ is —R^{α}—[P(R⁵)₃]Y; each —R⁵ is independently selected from phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups; n is 1, 2, 3 or 4; Y is fluoride, chloride, bromide or iodide; and —R^{3′} is H or C₁-C₄ alkyl (preferably methyl). In one embodiment, —R¹ is selected from —C(O)—OR³, —C(O)—SR³, —C(O)—N(R³)(R^{3′}) or —C(S)—N(R³)(R^{3′}); wherein —R³ is —R^{α}—[P(R⁵)₃]Y; each —R⁵ is independently selected from phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups; n is 1, 2, 3 or 4; Y is fluoride, chloride, bromide or iodide; and —R^{3′} is H or C₁-C₄ alkyl (preferably methyl). In one embodiment, —R¹ is —C(O)—N(R³)(R^{3′}); wherein —R³ is —R^{α}—[P(R⁵)₃]Y; each —R⁵ is independently selected from phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups; n is 1, 2, 3 or 4; Y is fluoride, chloride, bromide or iodide; and —R^{3′} is H or C₁-C₄ alkyl (preferably methyl). Typically in these embodiments, —R^{α}— is a C₁-C₁₂ alkylene group (preferably a C₁-C₈ alkylene group, or a C₁-C₆ alkylene group), a —(CH₂CH₂O)ₘ—CH₂CH₂— group or a —(CH₂CH₂S)ₘ—CH₂CH₂— group, all optionally substituted, wherein m is 1, 2, 3 or 4.

In one embodiment of the first or second aspect of the present invention, —R¹ is —C(O)—OR³, wherein —R³ is selected from C₁-C₄ alkyl (preferably methyl) or a cation (such as a lithium, sodium, potassium, magnesium, calcium, ammonium, amine (such as choline or meglumine), or amino acid (such as arginine) cation).

In one embodiment of the first or second aspect of the present invention, —R¹ is —C(O)—N(R³)₂. In one embodiment, —R¹ is —C(O)—N(C₁-C₄ alkyl)(R³) or —C(O)—NHR³. In one embodiment, —R¹ is —C(O)—N(CH₃)(R³) or —C(O)—NHR³. In one embodiment, —R¹ is —C(O)—N(C₁-C₄ alkyl)(R³). In one embodiment, —R¹ is —C(O)—N(CH₃)(R³).

In one embodiment of the first or second aspect of the present invention, —R¹ is selected from —CH₂OR², —CH₂SR², —CH₂S(O)R², —CH₂S(O)₂R², —CH₂N(R²)₂, or —R². In one embodiment, —R¹ is selected from —CH₂OR², —CH₂SR², —CH₂N(R²)₂, or —R². In one embodiment, —R¹ is selected from —CH₂OR², —CH₂SR², or —CH₂N(R²)₂. In one embodiment, —R¹ is selected from —CH₂OR² or —CH₂SR². In one embodiment, —R¹ is —CH₂OR². In one embodiment, —R¹ is —R², and —R² is —R^{α}—X.

In one embodiment of the first or second aspect of the present invention, —R² is selected from —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, or —R^{α}—[NC₅H₅]Y. In one embodiment, —R² is selected from —R^{α}—OR^{β}, —R^{α}—SR^{β}, —R^{α}—S(O)R^{β} or —R^{α}—S(O)₂R^{β}. In one embodiment, —R² is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R² is selected from —R^{α}—OR^{β} or —R^{α}—SR^{β}. In one embodiment, —R² is selected from —R^{α}—OR^{β} or —R^{α}—SR^{β}, and —R^{β} is a saccharidyl group.

In one embodiment of the first or second aspect of the present invention, —R² is selected from —C(O)R⁴, —C(O)—OR⁴, —C(O)—SR⁴, —C(O)—N(R⁴)₂, —C(S)—OR⁴, —C(S)—SR⁴ or —C(S)—N(R⁴)₂. In one embodiment, —R² is selected from —C(O)R⁴, —C(O)—OR⁴, —C(O)—SR⁴, —C(O)—N(R⁴)₂ or —C(S)—N(R⁴)₂. In one embodiment, —R² is selected from —C(O)R⁴, —C(O)—OR⁴, —C(O)—SR⁴ or —C(O)—N(R⁴)₂.

In one embodiment of the first or second aspect of the present invention, —R² is —C(O)—N(R⁴)(R^{4′}), wherein —R⁴ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{4′} is H or C₁-C₄ alkyl (preferably methyl). In one embodiment, —R² is —C(O)—N(R⁴)(R^{4′}), wherein —R⁴ is selected from —R^{α}—OR^{β} or —R^{α}—SR^{β}, and —R^{β} is a saccharidyl group, and —R^{4′} is H or C₁-C₄ alkyl (preferably methyl). In one embodiment, —R² is —C(O)—N(R⁴)(R^{4′}), wherein —R⁴ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{4′} is C₁-C₄ alkyl (preferably methyl). In one embodiment, —R² is —C(O)—N(R⁴)(R^{4′}), wherein —R⁴ is selected from —R^{α}—OR^{β} or —R^{α}—SR^{β}, and —R^{β} is a saccharidyl group, and —R^{4′} is C₁-C₄ alkyl (preferably methyl).

An —R^{4′} group refers to an —R⁴ group attached to the same atom as another —R⁴ group. —R⁴ and —R^{4′} may be the same or different. Preferably —R⁴ and —R^{4′} are different.

In one embodiment of the first or second aspect of the present invention, —R² is —C(O)—N(R⁴)₂. In one embodiment, —R² is —C(O)—N(C₁-C₄ alkyl)(R⁴). In one embodiment, —R² is —C(O)—N(CH₃)(R⁴).

In one embodiment of the first or second aspect of the present invention, —R⁶ is hydrogen.

In one embodiment of the first or second aspect of the present invention, —R⁶ is selected from —C(O)—OR³, —C(O)—SR³, —C(O)—N(R³)₂, —C(S)—OR³, —C(S)—SR³ or —C(S)—N(R³)₂, and each —R³ is C₁-C₄ alkyl, preferably each —R³ is methyl. In one embodiment, —R⁶ is selected from —C(O)—OR³, —C(O)—SR³ or —C(O)—N(R³)₂, and each —R³ is C₁-C₄ alkyl, preferably each —R³ is methyl. In one embodiment, —R⁶ is —C(O)—OR³, and —R³ is C₁-C₄ alkyl, preferably —R³ is methyl.

In one embodiment of the first or second aspect of the present invention, —R⁶ is —C(O)—OR³, wherein —R³ is selected from hydrogen, C₁-C₄ alkyl (preferably methyl) or a cation (such as a lithium, sodium, potassium, magnesium, calcium, ammonium, amine (such as choline or meglumine), or amino acid (such as arginine) cation).

In one embodiment of the first or second aspect of the present invention, —R⁶ is selected from —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); wherein —R³ is —R^{β}; —R^{β} is selected from a C₁-C₂₀ alkyl group, wherein the alkyl group may optionally be substituted with one, two, three or four halo groups, and wherein one, two, three, four, five or six carbon atoms in the backbone of the alkyl group may optionally be replaced by a heteroatom or group independently selected from O, S, NH or NMe; and —R^{3′} is H or C₁-C₄ alkyl (preferably methyl).

In one embodiment of the first or second aspect of the present invention, —R⁶ is selected from —C(O)—OR³, —C(O)—SR³, —C(O)—N(R³)₂, —C(S)—OR³, —C(S)—SR³ or —C(S)—N(R³)₂, and each —R³ is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R⁶ is selected from —C(O)—OR³, —C(O)—SR³ or —C(O)—N(R³)₂, and each —R³ is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R⁶ is selected from —C(O)—OR³ or —C(O)—SR³, and —R³ is selected from —R^{α}—OR^{β} or —R^{α}—SR^{β}, and —R^{β} is a saccharidyl group. Typically in these embodiments, —R^{α}— is selected from a C₁-C₁₂ alkylene group, wherein one, two, three or four carbon atoms in the backbone of the alkylene group may optionally be replaced by a heteroatom or group independently selected from O, S, NH or NMe. Alternatively, in these embodiments, —R^{α}— is a C₁-C₁₂ alkylene group (preferably a C₁-C₈ alkylene group, or a C₁-C₆ alkylene group), a —(CH₂CH₂O)ₘ—CH₂CH₂— group or a —(CH₂CH₂S)ₘ—CH₂CH₂— group, all optionally substituted, wherein m is 1, 2, 3 or 4.

In one embodiment of the first or second aspect of the present invention, —R⁶ is selected from —C(O)—OR³, —C(O)—SR³, —C(O)—N(R³)(R^{3′}) or —C(S)—N(R³)(R^{3′}), wherein —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl (preferably methyl). In one embodiment, —R⁶ is selected from —C(O)—OR³, —C(O)—SR³ or —C(O)—N(R³)(R^{3′}), wherein —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl (preferably methyl). In one embodiment, —R⁶ is selected from —C(O)—OR³, —C(O)—SR³ or —C(O)—N(R³)(R^{3′}), wherein —R³ is selected from —R^{α}—OR^{β} or —R^{α}—SR^{β}, and —R^{β} is a saccharidyl group, and —R^{3′} is H or C₁-C₄ alkyl (preferably methyl). Typically in these embodiments, —R^{α}— is selected from a C₁-C₁₂ alkylene group, wherein one, two, three or four carbon atoms in the backbone of the alkylene group may optionally be replaced by a heteroatom or group independently selected from O, S, NH or NMe. Alternatively, in these embodiments, —R^{α}— is a C₁-C₁₂ alkylene group (preferably a C₁-C₈ alkylene group, or a C₁-C₆ alkylene group), a —(CH₂CH₂O)ₘ—CH₂CH₂— group or a —(CH₂CH₂S)ₘ—CH₂CH₂— group, all optionally substituted, wherein m is 1, 2, 3 or 4.

An —R^{3′} group refers to an —R³ group attached to the same atom as another —R³ group. —R³ and —R^{3′} may be the same or different. Preferably —R³ and —R^{3′} are different.

In one embodiment of the first or second aspect of the present invention, —R⁶ is —C(O)—N(R³)₂. In one embodiment, —R⁶ is —C(O)—N(C₁-C₄ alkyl)(R³) or —C(O)—NHR³. In one embodiment, —R⁶ is —C(O)—N(CH₃)(R³) or —C(O)—NHR³.

In one embodiment of the first or second aspect of the present invention, —R⁷ is hydrogen.

In one embodiment of the first or second aspect of the present invention, —R⁷ is selected from —C(O)—OR³, —C(O)—SR³, —C(O)—N(R³)₂, —C(S)—OR³, —C(S)—SR³ or —C(S)—N(R³)₂, and each —R³ is C₁-C₄ alkyl, preferably each —R³ is methyl. In one embodiment, —R⁷ is selected from —C(O)—OR³, —C(O)—SR³ or —C(O)—N(R³)₂, and each —R³ is C₁-C₄ alkyl, preferably each —R³ is methyl. In one embodiment, —R⁷ is —C(O)—OR³, and —R³ is C₁-C₄ alkyl, preferably —R³ is methyl.

In one embodiment of the first or second aspect of the present invention, —R⁷ is —C(O)—OR³, wherein —R³ is selected from hydrogen, C₁-C₄ alkyl (preferably methyl) or a cation (such as a lithium, sodium, potassium, magnesium, calcium, ammonium, amine (such as choline or meglumine), or amino acid (such as arginine) cation).

In one embodiment of the first or second aspect of the present invention, —R⁷ is selected from —C(O)—OR³, —C(O)—SR³, —C(O)—N(R³)₂, —C(O)—N(R³)(R^{3′}), —C(S)—OR³, —C(S)—SR³, —C(S)—N(R³)₂ or —C(S)—N(R³)(R^{3′}); wherein —R³ is —R^{β}; —R^{β} is selected from a C₁-C₂₀ alkyl group, wherein the alkyl group may optionally be substituted with one, two, three or four halo groups, and wherein one, two, three, four, five or six carbon atoms in the backbone of the alkyl group may optionally be replaced by a heteroatom or group independently selected from O, S, NH or NMe; and —R^{3′} is H or C₁-C₄ alkyl (preferably methyl).

In one embodiment of the first or second aspect of the present invention, —R⁷ is selected from —C(O)—OR³, —C(O)—SR³, —C(O)—N(R³)₂, —C(S)—OR³, —C(S)—SR³ or —C(S)—N(R³)₂, and each —R³ is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R⁷ is selected from —C(O)—OR³, —C(O)—SR³ or —C(O)—N(R³)₂, and each —R³ is selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, —R⁷ is selected from —C(O)—OR³ or —C(O)—SR³, and —R³ is selected from —R^{α}—OR^{β} or —R^{α}—SR^{β}, and —R^{β} is a saccharidyl group. Typically in these embodiments, —R^{α}— is selected from a C₁-C₁₂ alkylene group, wherein one, two, three or four carbon atoms in the backbone of the alkylene group may optionally be replaced by a heteroatom or group independently selected from O, S, NH or NMe. Alternatively, in these embodiments, —R^{α}— is a C₁-C₁₂ alkylene group (preferably a C₁-C₈ alkylene group, or a C₁-C₆ alkylene group), a —(CH₂CH₂O)ₘ—CH₂CH₂— group or a —(CH₂CH₂S)ₘ—CH₂CH₂— group, all optionally substituted, wherein m is 1, 2, 3 or 4.

In one embodiment of the first or second aspect of the present invention, —R⁷ is selected from —C(O)—OR³, —C(O)—SR³, —C(O)—N(R³)(R^{3′}) or —C(S)—N(R³)(R^{3′}), wherein —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl (preferably methyl). In one embodiment, —R⁷ is selected from —C(O)—OR³, —C(O)—SR³ or —C(O)—N(R³)(R^{3′}), wherein —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl (preferably methyl). In one embodiment, —R⁷ is selected from —C(O)—OR³, —C(O)—SR³ or —C(O)—N(R³)(R^{3′}), wherein —R³ is selected from —R^{α}—OR^{β} or —R^{α}—SR^{β}, and —R^{β} is a saccharidyl group, and —R^{3′} is H or C₁-C₄ alkyl (preferably methyl).

Typically in these embodiments, —R^{α}— is selected from a C₁-C₁₂ alkylene group, wherein one, two, three or four carbon atoms in the backbone of the alkylene group may optionally be replaced by a heteroatom or group independently selected from O, S, NH or NMe. Alternatively, in these embodiments, —R^{α}— is a C₁-C₁₂ alkylene group (preferably a C₁-C₈ alkylene group, or a C₁-C₆ alkylene group), a —(CH₂CH₂O)ₘ—CH₂CH₂— group or a —(CH₂CH₂S)ₘ—CH₂CH₂— group, all optionally substituted, wherein m is 1, 2, 3 or 4.

An —R^{3′} group refers to an —R³ group attached to the same atom as another —R³ group. —R³ and —R^{3′} may be the same or different. Preferably —R³ and —R^{3′} are different.

In one embodiment of the first or second aspect of the present invention, —R⁷ is —C(O)—N(R³)₂. In one embodiment, —R⁷ is —C(O)—N(C₁-C₄ alkyl)(R³) or —C(O)—NHR³. In one embodiment, —R⁷ is —C(O)—N(CH₃)(R³) or —C(O)—NHR³.

In one embodiment of the first or second aspect of the present invention, —R⁶ and —R⁷ together form —C(O)—O—.

In one embodiment of the first or second aspect of the present invention, each —R^{α}— is independently a C₁-C₁₂ alkylene group, a —(CH₂CH₂O)ₘ— group, a —(CH₂CH₂S)ₘ— group, a —(CH₂CH₂O)ₘ—CH₂CH₂— group or a —(CH₂CH₂S)ₘ—CH₂CH₂— group, all optionally substituted, wherein m is 1, 2, 3 or 4. In one embodiment, each —R^{α}— is independently a C₁-C₁₂ alkylene group, a —(CH₂CH₂O)ₘ— group or a —(CH₂CH₂S)ₘ— group, all optionally substituted, wherein m is 1, 2, 3 or 4. In one embodiment, each —R^{α}— is independently a C₁-C₁₂ alkylene group or a —(CH₂CH₂O)ₘ— group, both optionally substituted, wherein m is 1, 2, 3 or 4. In one embodiment, each —R^{α}— is independently an optionally substituted —(CH₂CH₂O)ₘ— group, wherein m is 1, 2, 3 or 4.

In one embodiment of the first or second aspect of the present invention, each —R^{α}— is independently a C₁-C₈ alkylene group, or a C₁-C₆ alkylene group, or a C₂-C₄ alkylene group, all optionally substituted.

In one embodiment of the first or second aspect of the present invention, each —R^{α}— is independently unsubstituted or substituted with one or more substituents independently selected from halo, C₁-C₄ alkyl, or C₁-C₄ haloalkyl. In one embodiment, each —R^{α}— is independently unsubstituted or substituted with one or two substituents independently selected from halo, C₁-C₄ alkyl, or C₁-C₄ haloalkyl. In one embodiment, each —R^{α}— is unsubstituted.

In one embodiment of the first or second aspect of the present invention, each —R^{β} is independently a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O and S in its carbon skeleton.

In one embodiment of the first or second aspect of the present invention, at least one —R^{β} is independently a C₁-C₆ alkyl group, or a C₁-C₄ alkyl group, or a methyl group, all optionally substituted. In one embodiment, each —R^{β} is independently a C₁-C₆ alkyl group, or a C₁-C₄ alkyl group, or a methyl group, all optionally substituted. In one embodiment, each —R^{β} is independently a C₁-C₆ alkyl group, or a C₁-C₄ alkyl group, or a methyl group.

In one embodiment of the first or second aspect of the present invention, at least one —R^{β} is independently a saccharidyl group. In one embodiment, each —R^{β} is independently a saccharidyl group.

In one embodiment of the first or second aspect of the present invention, each —R^{β} is independently unsubstituted or substituted with one or more substituents independently selected from halo, C₁-C₄ alkyl, or C₁-C₄ haloalkyl. In one embodiment, each —R^{β} is independently unsubstituted or substituted with one or two substituents independently selected from halo, C₁-C₄ alkyl, or C₁-C₄ haloalkyl. In one embodiment, each —R^{β} is unsubstituted.

In one embodiment of the first or second aspect of the present invention, each —R³ is independently selected from —R^{α}—H, —R^{β}, —R^{α}—R^{β}, —R^{α}—OH, —R^{α}—OR^{β}, —R^{α}—SH, —R^{α}—SR^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, or —R^{α}—[NC₅H₅]Y. In one embodiment, each —R³ is independently selected from —R^{α}—OR^{β} or —R^{α}—SR^{β}. In one embodiment, each —R³ is independently selected from —R, —OR^{β} or —R^{α}—SR^{β}, and —R^{β} is a saccharidyl group.

In one embodiment of the first or second aspect of the present invention, each —R³ is independently selected from —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{β}—X, —R^{α}—[N(R⁵)₃]Y, —R^{α}—[P(R⁵)₃]Y, or —R^{α}—[NC₅H₅]Y. In one embodiment, each —R³ is independently selected from —R^{α}—OR^{β}, —R^{α}—SR^{β}, —R^{α}—S(O)R^{β} or —R^{α}—S(O)₂R^{β}. In one embodiment, each —R³ is independently selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, each —R³ is independently selected from —R^{α}—OR^{β} or —R^{α}—SR^{β}. In one embodiment, each —R³ is independently selected from —R^{α}—OR^{β} or —R^{α}—SR^{β}, and —R^{β} is a saccharidyl group.

In one embodiment of the first or second aspect of the present invention, each —R⁴ is independently selected from —R^{α}—H, —R^{β}, —R^{α}—R^{β}, —R^{α}—OH, —R^{α}—OR^{β}, —R^{α}—SH, —R^{α}—SR^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, or —R^{α}—[NC₅H₅]Y. In one embodiment, each —R⁴ is independently selected from —R^{α}—OR^{β} or —R^{α}—SR^{β}. In one embodiment, each —R⁴ is independently selected from —R^{α}—OR^{β} or —R^{α}—SR^{β}, and —R^{β} is a saccharidyl group.

In one embodiment of the first or second aspect of the present invention, each —R⁴ is independently selected from —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, or —R^{α}—[NC₅H₅]Y. In one embodiment, each —R⁴ is independently selected from —R^{α}—OR^{β}, —R^{α}—SR^{β}, —R^{α}—S(O)R^{β} or —R^{α}—S(O)₂R^{β}. In one embodiment, each —R⁴ is independently selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, each —R⁴ is independently selected from —R^{α}—OR^{β} or —R^{α}—SR^{β}. In one embodiment, each —R⁴ is independently selected from —R^{α}—OR^{β} or —R^{α}—SR^{β}, and —R^{β} is a saccharidyl group.

In one embodiment of the first or second aspect of the present invention, at least one of —R², —R³ or —R⁴ is independently selected from —R^{α}—OR^{β}, —R^{α}—SR^{β}, —R^{α}—S(O)R^{β} or —R^{α}—S(O)₂R^{β}, and —R^{β} is a saccharidyl group. In one embodiment, at least one of —R², —R³ or —R⁴ is independently selected from —R^{α}—OR^{β} or —R^{α}—SR^{β}, and —R^{β} is a saccharidyl group.

For the purposes of the present invention, a “saccharidyl group” is any group comprising at least one monosaccharide subunit, wherein each monosaccharide subunit may optionally be substituted and/or modified. Typically, a saccharidyl group consist of one or more monosaccharide subunits, wherein each monosaccharide subunit may optionally be substituted and/or modified.

Typically, a carbon atom of a single monosaccharide subunit of each saccharidyl group is directly attached to the remainder of the compound, most typically via a single bond.

For the purposes of the present specification, where it is stated that a first atom or group is “directly attached” to a second atom or group it is to be understood that the first atom or group is covalently bonded to the second atom or group with no intervening atom(s) or group(s) being present. For example, for the group —(C═O)N(CH₃)₂, the carbon atom of each methyl group is directly attached to the nitrogen atom and the carbon atom of the carbonyl group is directly attached to the nitrogen atom, but the carbon atom of the carbonyl group is not directly attached to the carbon atom of either methyl group.

Typically, each saccharidyl group is derived from the corresponding saccharide by substitution of a hydroxyl group of the saccharide with the group defined by the remainder of the compound.

A single bond between an anomeric carbon of a monosaccharide subunit and a substituent is called a glycosidic bond. A glycosidic group is linked to the anomeric carbon of a monosaccharide subunit by a glycosidic bond. The bond between the saccharidyl group and the remainder of the compound may be a glycosidic or a non-glycosidic bond. Typically, the bond between the saccharidyl group and the remainder of the compound is a glycosidic bond, such that the saccharidyl group is a glycosyl group. Where the bond between the saccharidyl group and the remainder of the compound is a glycosidic bond, the glycosidic bond may be in the a or P configuration. Typically, such a glycosidic bond is in the P configuration.

For the purposes of the present invention, where a saccharidyl group “contains x monosaccharide subunits”, this means that the saccharidyl group has x monosaccharide subunits and no more. In contrast, where a saccharidyl group “comprises x monosaccharide subunits”, this means that the saccharidyl group has x or more monosaccharide subunits.

Each saccharidyl group may be independently selected from a monosaccharidyl, disaccharidyl, oligosaccharidyl or polysaccharidyl group. As will be understood, a monosaccharidyl group contains a single monosaccharide subunit. Similarly, a disaccharidyl group contains two monosaccharide subunits. As used herein, an “oligosaccharidyl group” contains from 2 to 9 monosaccharide subunits. Examples of oligosaccharidyl groups include trisaccharidyl, tetrasaccharidyl, pentasaccharidyl, hexasaccharidyl, heptasaccharidyl, octasaccharidyl and nonasaccharidyl groups. As used herein, a “polysaccharidyl group” contains 10 or more monosaccharide subunits (such as 10-50, or 10-30, or 10-20, or 10-15 monosaccharide subunits).

Each monosaccharide subunit within a disaccharidyl, oligosaccharidyl or polysaccharidyl group may be the same or different. Each monosaccharide subunit within a disaccharidyl, oligosaccharidyl or polysaccharidyl group may be connected to another monosaccharide subunit within the group via a glycosidic or a non-glycosidic bond. Typically each monosaccharide subunit within a disaccharidyl, oligosaccharidyl or polysaccharidyl group is connected to another monosaccharide subunit within the group via a glycosidic bond, which may be in the a or P configuration.

Each oligosaccharidyl or polysaccharidyl group may be a linear, branched or macrocyclic oligosaccharidyl or polysaccharidyl group. Typically, each oligosaccharidyl or polysaccharidyl group is a linear or branched oligosaccharidyl or polysaccharidyl group.

In one embodiment, at least one —R^{β} is a monosaccharidyl or disaccharidyl group.

In a further embodiment, at least one —R^{β} is a monosaccharidyl group. For example, at least one —R^{β} may be a glycosyl group containing a single monosaccharide subunit, wherein the monosaccharide subunit may optionally be substituted and/or modified. Typically at least one —R^{β} is a glycosyl group containing a single monosaccharide subunit, wherein the monosaccharide subunit may optionally be substituted. More typically, at least one —R^{β} is a glycosyl group containing a single monosaccharide subunit, wherein the monosaccharide subunit is unsubstituted.

In one embodiment, at least one —R^{β} is an aldosyl group, wherein the aldosyl group may optionally be substituted and/or modified. For example, at least one —R^{β} may be selected from a glycerosyl, aldotetrosyl (such as erythrosyl or threosyl), aldopentosyl (such as ribosyl, arabinosyl, xylosyl or lyxosyl) or aldohexosyl (such as allosyl, altrosyl, glucosyl, mannosyl, gulosyl, idosyl, galactosyl or talosyl) group, any of which may optionally be substituted and/or modified.

In another embodiment, at least one —R^{β} is a ketosyl group, wherein the ketosyl group may optionally be substituted and/or modified. For example, at least one —R^{β} may be selected from an erythrulosyl, ketopentosyl (such as ribulosyl or xylulosyl) or ketohexosyl (such as psicosyl, fructosyl, sorbosyl or tagatosyl) group, any of which may optionally be substituted and/or modified.

Each monosaccharide subunit may be present in a ring-closed (cyclic) or open-chain (acyclic) form. Typically, each monosaccharide subunit in at least one —R^{β} is present in a ring-closed (cyclic) form. For example, at least one —R^{β} may be a glycosyl group containing a single ring-closed monosaccharide subunit, wherein the monosaccharide subunit may optionally be substituted and/or modified. Typically in such a scenario, at least one —R^{β} is a pyranosyl or furanosyl group, such as an aldopyranosyl, aldofuranosyl, ketopyranosyl or ketofuranosyl group, any of which may optionally be substituted and/or modified. More typically, at least one —R^{β} is a pyranosyl group, such as an aldopyranosyl or ketopyranosyl group, any of which may optionally be substituted and/or modified.

In one embodiment, at least one —R^{β} is selected from a ribopyranosyl, arabinopyranosyl, xylopyranosyl, lyxopyranosyl, allopyranosyl, altropyranosyl, glucopyranosyl, mannopyranosyl, gulopyranosyl, idopyranosyl, galactopyranosyl or talopyranosyl group, any of which may optionally be substituted and/or modified.

In a further embodiment, at least one —R^{β} is a glucosyl group, such as a glucopyranosyl group, wherein the glucosyl or the glucopyranosyl group may optionally be substituted and/or modified. Typically, at least one —R^{β} is a glucosyl group, wherein the glucosyl group is optionally substituted. More typically, at least one —R^{β} is an unsubstituted glucosyl group.

Each monosaccharide subunit may be present in the D- or L-configuration. Typically, each monosaccharide subunit is present in the configuration in which it most commonly occurs in nature.

In one embodiment, at least one —R^{β} is a D-glucosyl group, such as a D-glucopyranosyl group, wherein the D-glucosyl or the D-glucopyranosyl group may optionally be substituted and/or modified. Typically, at least one —R^{β} is a D-glucosyl group, wherein the D-glucosyl group is optionally substituted. More typically, at least one —R^{β} is an unsubstituted D-glucosyl group.

For the purposes of the present invention, in a substituted monosaccharidyl group or monosaccharide subunit:
(a) one or more of the hydroxyl groups of the monosaccharidyl group or monosaccharide subunit are each independently replaced with —H, —F, —Cl, —Br, —I, —CF₃, —CCl₃, —CBr₃, —CI₃, —SH, —NH₂, —N₃, —NH═NH₂, —CN, —NO₂, —COOH, —R^{b}, —O—R^{b}, —S—R^{b}, —R^{a}—O—R^{b}, —R^{a}—S—R^{b}, —SO—R^{b}, —SO₂—R^{b}, —SO₂—OR^{b}, —O—SO—R^{b}, —O—SO₂—R^{b}, —O—SO₂—OR^{b}, —NR^{b}—SO—R^{b}, —NR^{b}—SO₂—R^{b}, —NR^{b}—SO₂—OR^{b}, —R^{a}—SO—R^{b}, —R^{a}—SO₂—R^{b}, —R^{a}—SO₂—OR^{b}, —SO—N(R^{b})₂, —SO₂—N(R^{b})₂, —O—SO—N(R^{b})₂, —O—SO₂—N(R^{b})₂, —NR^{b}—SO—N(R^{b})₂, —NR^{b}—SO₂—N(R^{b})₂, —R^{a}—SO—N(R^{b})₂, —R^{a}—SO₂—N(R^{b})₂, —N(R^{b})₂, —N(R^{b})₃+, —R^{a}—N(R^{b})₂, —R^{a}—N(R^{b})₃⁺, —P(R^{b})₂, —PO(R^{b})₂, —OP(R^{b})₂, —OPO(R^{b})₂, —R^{a}—P(R^{b})₂, —R^{a}—PO(R^{b})₂, —OSi(R^{b})₃, —R^{a}—Si(R^{b})₃, —CO—R^{b}, —CO—OR^{b}, —CO—N(R^{b})₂, —O—CO—R^{b}, —O—CO—OR^{b}, —O—CO—N(R^{b})₂, —NR^{b}—CO—R^{b}, —NR^{b}—CO—OR^{b}, —NR^{b}—CO—N(R^{b})₂, —R^{a}—CO—R^{b}, —R^{a}—CO—OR^{b}, or —R^{a}—CO—N(R^{b})₂; and/or
(b) one, two or three hydrogen atoms directly attached to a carbon atom of the monosaccharidyl group or monosaccharide subunit are each independently replaced with —F, —Cl, —Br, —I, —CF₃, —CCl₃, —CBr₃, —CI₃, —OH, —SH, —NH₂, —N₃, —NH═NH₂, —CN, —NO₂, —COOH, —R^{b}, —O—R^{b}, —S—R^{b}, —R^{a}—O—R^{b}, —R^{a}—S—R^{b}, —SO—R^{b}, —SO₂—R^{b}, —SO₂—OR^{b}, —O—SO—R^{b}, —O—SO₂—R^{b}, —O—SO₂—OR^{b}, —NR^{b}—SO—R^{b}, —NR^{b}—SO₂—R^{b}, —NR^{b}—SO₂—OR^{b}, —R^{a}—SO—R^{b}, —R^{a}—SO₂—R^{b}, —R^{a}—SO₂—OR^{b}, —SO—N(R^{b})₂, —SO₂—N(R^{b})₂, —O—SO—N(R^{b})₂, —O—SO₂—N(R^{b})₂, —NR^{b}—SO—N(R^{b})₂, —NR^{b}—SO₂—N(R^{b})₂, —R^{a}—SO—N(R^{b})₂, —R^{a}—SO₂—N(R^{b})₂, —N(R^{b})₂, —N(R^{b})₃+, —R^{a}—N(R^{b})₂, —R^{a}—N(R^{b})₃+, —P(R^{b})₂, —PO(R^{b})₂, —OP(R^{b})₂, —OPO(R^{b})₂, —R^{a}—P(R^{b})₂, —R^{a}—PO(R^{b})₂, —OSi(R^{b})₃, —R^{a}—Si(R^{b})₃, —CO—R^{b}, —CO—OR^{b}, —CO—N(R^{b})₂, —O—CO—R^{b}, —O—CO—OR^{b}, —O—CO—N(R^{b})₂, —NR^{b}—CO—R^{b}, —NR^{b}—CO—OR^{b}, —NR^{b}—CO—N(R^{b})₂, —R^{a}—CO—R^{b}, —R^{a}—CO—OR^{b}, or —R^{a}—CO—N(R^{b})₂; and/or
(c) one or more of the hydroxyl groups of the monosaccharidyl group or monosaccharide subunit, together with the hydrogen attached to the same carbon atom as the hydroxyl group, are each independently replaced with ═O, ═S, ═NR^{b}, or ═N(R^{b})₂⁺; and/or
(d) any two hydroxyl groups of the monosaccharidyl group or monosaccharide subunit are together replaced with —O—R^{c}—, —S—R^{c}—, —SO—R^{c}—, —SO₂—R^{c}—, or —NR^{b}—R^{c}—; wherein:
   each —R^{a}— is independently a substituted or unsubstituted alkylene, alkenylene or alkynylene group which optionally includes one or more heteroatoms each independently selected from O, N and S in its carbon skeleton and preferably comprises 1-10 carbon atoms;
   each —R^{b} is independently hydrogen, or a substituted or unsubstituted, straight-chained, branched or cyclic alkyl, alkenyl, alkynyl, aryl, arylalkyl, arylalkenyl, arylalkynyl, alkylaryl, alkenylaryl or alkynylaryl group which optionally includes one or more heteroatoms each independently selected from O, N and S in its carbon skeleton and preferably comprises 1-15 carbon atoms; and
   each —R^{c}— is independently a chemical bond, or a substituted or unsubstituted alkylene, alkenylene or alkynylene group which optionally includes one or more heteroatoms each independently selected from O, N and S in its carbon skeleton and preferably comprises 1-10 carbon atoms;
   provided that the monosaccharidyl group or monosaccharide subunit comprises at least one, preferably at least two or at least three, —OH, —O—R^{b}, —O—SO—R^{b}, —O—SO₂—R^{b}, —O—SO₂—OR^{b}, —O—SO—N(R^{b})₂, —O—SO₂—N(R^{b})₂, —OP(R^{b})₂, —OPO(R^{b})₂, —OSi(R^{b})₃, —O—CO—R^{b}, —O—CO—OR^{b}, —O—CO—N(R^{b})₂, or —O—R^{c}—.

Typically, in a substituted monosaccharidyl group or monosaccharide subunit:
(a) one or more of the hydroxyl groups of the monosaccharidyl group or monosaccharide subunit are each independently replaced with —H, —F, —CF₃, —SH, —NH₂, —N₃, —CN, —NO₂, —COOH, —R^{b}, —O—R^{b}, —S—R^{b}, —N(R^{b})₂, —OPO(R^{b})₂, —OSi(R^{b})₃, —O—CO—R^{b}, —O—CO—OR^{b}, —O—CO—N(R^{b})₂, —NR^{b}—CO—R^{b}, —NR^{b}—CO—OR^{b}, or —NR^{b}—CO—N(R^{b})₂; and/or
(b) one or two of the hydrogen atoms directly attached to a carbon atom of the monosaccharidyl group or monosaccharide subunit are each independently replaced with —F, —CF₃, —OH, —SH, —NH₂, —N₃, —CN, —NO₂, —COOH, —R^{b}, —O—R^{b}, —S—R^{b}, —N(R^{b})₂, —OPO(R^{b})₂, —OSi(R^{b})₃, —O—CO—R^{b}, —O—CO—OR^{b}, —O—CO—N(R^{b})₂, —NR^{b}—CO—R^{b}, —NR^{b}—CO—OR^{b}, or —NR^{b}—CO—N(R^{b})₂; and/or
(c) one hydroxyl group of the monosaccharidyl group or monosaccharide subunit, together with the hydrogen attached to the same carbon atom as the hydroxyl group, is replaced with ═O; and/or
(d) any two hydroxyl groups of the monosaccharidyl group or monosaccharide subunit are together replaced with —O—R^{c}— or —NR^{b}—R^{c}—;
wherein:
   each —R^{b} is independently hydrogen, or a substituted or unsubstituted, straight-chained, branched or cyclic alkyl, alkenyl, alkynyl, aryl, arylalkyl, arylalkenyl, arylalkynyl, alkylaryl, alkenylaryl or alkynylaryl group which optionally includes one, two or three heteroatoms each independently selected from O and N in its carbon skeleton and comprises 1-8 carbon atoms; and
   each —R^{c}— is independently a substituted or unsubstituted alkylene, alkenylene or alkynylene group which optionally includes one, two or three heteroatoms each independently selected from 0 and N in its carbon skeleton and comprises 1-8 carbon atoms;
   provided that the monosaccharidyl group or monosaccharide subunit comprises at least two, preferably at least three, —OH, —O—R^{b}, —OPO(R^{b})₂, —OSi(R^{b})₃, —O—CO—R^{b}, —O—CO—OR^{b}, —O—CO—N(R^{b})₂, or —O—R^{c}—.

In one embodiment, —R^{β} is a saccharidyl group and one or more of the hydroxyl groups of the saccharidyl group are each independently replaced with —O—CO—R^{b}, wherein each —R^{b} is independently C₁-C₄ alkyl, preferably methyl. In one embodiment, —R^{β} is a saccharidyl group and all of the hydroxyl groups of the saccharidyl group are each independently replaced with —O—CO—R^{b}, wherein each —R^{b} is independently C₁-C₄ alkyl, preferably methyl.

In a modified monosaccharidyl group or monosaccharide subunit:
(a) the ring of the modified monosaccharidyl group or monosaccharide subunit, or what would be the ring in the ring-closed form of the modified monosaccharidyl group or monosaccharide subunit, is partially unsaturated; and/or
(b) the ring oxygen of the modified monosaccharidyl group or monosaccharide subunit, or what would be the ring oxygen in the ring-closed form of the modified monosaccharidyl group or monosaccharide subunit, is replaced with —S— or —NR^{d}—, wherein —R^{d} is independently hydrogen, or a substituted or unsubstituted, straight-chained, branched or cyclic alkyl, alkenyl, alkynyl, aryl, arylalkyl, arylalkenyl, arylalkynyl, alkylaryl, alkenylaryl or alkynylaryl group which optionally includes one or more heteroatoms each independently selected from O, N and S in its carbon skeleton and preferably comprises 1-15 carbon atoms.

Alternately, where the modified monosaccharide subunit forms part of a disaccharidyl, oligosaccharidyl or polysaccharidyl group, —R^{d} may be a further monosaccharide subunit or subunits forming part of the disaccharidyl, oligosaccharidyl or polysaccharidyl group, wherein any such further monosaccharide subunit or subunits may optionally be substituted and/or modified.

Typically, in a modified monosaccharidyl group or monosaccharide subunit:
(a) the ring of the modified monosaccharidyl group or monosaccharide subunit, or what would be the ring in the ring-closed form of the modified monosaccharidyl group or monosaccharide subunit, contains a single C═C; and/or
(b) the ring oxygen of the modified monosaccharidyl group or monosaccharide subunit, or what would be the ring oxygen in the ring-closed form of the modified monosaccharidyl group or monosaccharide subunit, is replaced with —NR^{d}—, wherein —R^{d} is independently hydrogen, or a substituted or unsubstituted, straight-chained, branched or cyclic alkyl, alkenyl, alkynyl, aryl, arylalkyl, arylalkenyl, arylalkynyl, alkylaryl, alkenylaryl or alkynylaryl group which optionally includes one, two or three heteroatoms each independently selected from O and N in its carbon skeleton and comprises 1-8 carbon atoms.

Typical examples of substituted and/or modified monosaccharide subunits include those corresponding to:
(i) deoxy sugars, such as deoxyribose, fucose, fuculose and rhamnose, wherein a hydroxyl group of the monosaccharidyl group or monosaccharide subunit has been replaced by —H;
(ii) amino sugars, such as glucosamine and galactosamine, wherein a hydroxyl group of the monosaccharidyl group or monosaccharide subunit has been replaced by —NH₂, most typically at the 2-position; and
(iii) sugar acids, containing a —COOH group, such as aldonic acids (e.g. gluconic acid), ulosonic acids, uronic acids (e.g. glucuronic acid) and aldaric acids (e.g. gularic or galactaric acid).

In one embodiment of the first or second aspect of the present invention, at least one —R^{β} is a monosaccharidyl group selected from:

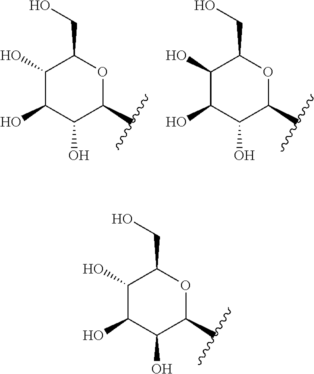 [SMILES: *[C@@H]1O[C@H](CO)[C@@H](O)[C@H](O)[C@H]1O ; *[C@@H]1O[C@H](CO)[C@H](O)[C@H](O)[C@H]1O ; *[C@@H]1O[C@H](CO)[C@@H](O)[C@H](O)[C@@H]1O]

Preferably in the compound or complex according to the first or second aspect of the present invention, at least one —R^{β} is:

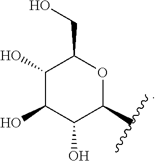 [SMILES: *[C@@H]1O[C@H](CO)[C@@H](O)[C@H](O)[C@H]1O]

In one embodiment of the first or second aspect of the present invention, at least one of —R², —R³ or —R⁴ is independently selected from —R^{α}—OR^{β}, —R^{α}—SR^{β}, —R^{α}—S(O)R^{β} or —R^{α}—S(O)₂R^{β} (preferably from —R^{α}—OR^{β} or —R^{α}—SR^{β}), and —R^{β} is selected from:

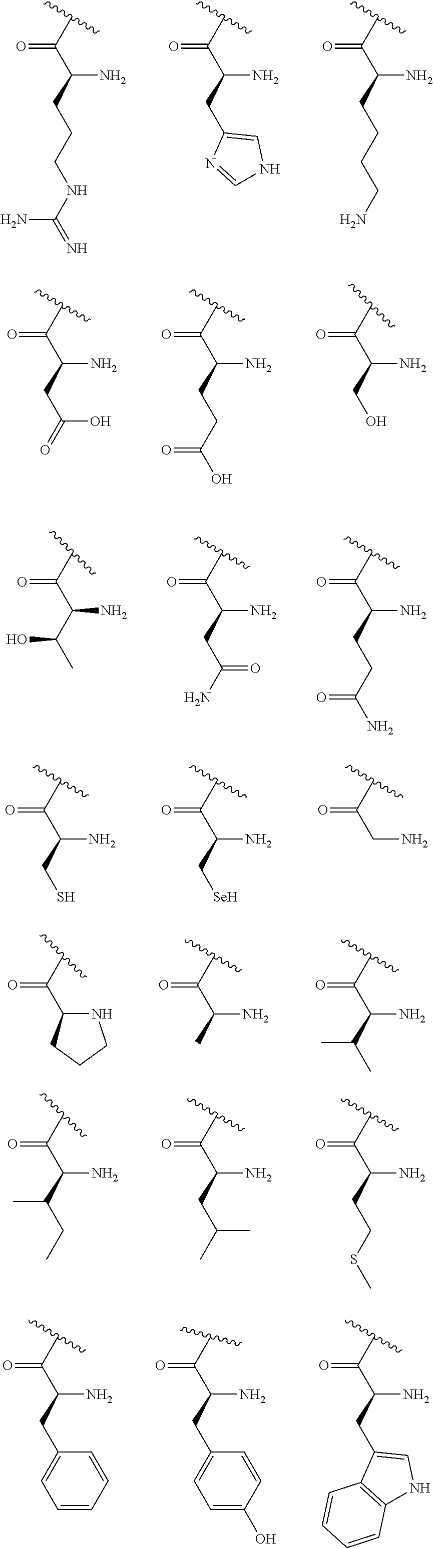

In one embodiment of the first or second aspect of the present invention, at least one of —R², —R³ or —R⁴ is independently selected from —R^{α}—[N(R⁵)₃]Y, —R^{α}—[P(R⁵)₃]Y, —R^{α}—[R⁸]Y, —R^{α}—[N(R⁵)₂(R^{5′})], —R^{α}—[P(R⁵)₂(R^{5′})], or —R^{α}—[R^{8′}]. In one embodiment, at least one of —R², —R³ or —R⁴ is independently selected from —R^{α}—[N(R⁵)₃]Y, —R^{α}—[P(R⁵)₃]Y, or —R^{α}—[R⁸]Y. In one embodiment, at least one of —R², —R³ or —R⁴ is independently selected from:

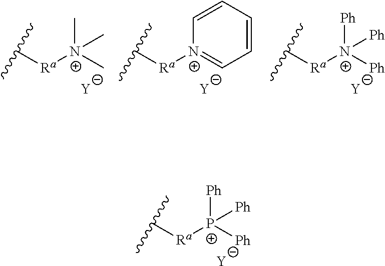

In the first or second aspect of the present invention, each —R⁵ may be the same or different. In a preferred embodiment, each —R⁵ is the same.

In one embodiment of the first or second aspect of the present invention, each —R⁵ is independently unsubstituted or substituted with one or two substituents. In one embodiment, each —R⁵ is unsubstituted.

In one embodiment of the first or second aspect of the present invention, —R⁸ is unsubstituted or substituted with one or two substituents. In one embodiment, —R⁸ is unsubstituted.

In one embodiment, —R⁸ is not substituted at the 4-position of the pyridine ring with a halo group. In one embodiment, —R⁸ is unsubstituted at the 4-position of the pyridine ring. In one embodiment, —R⁸ is unsubstituted.

In one embodiment of the first or second aspect of the present invention, —R¹ comprises from 1 to 100 atoms other than hydrogen, preferably from 1 to 80 atoms other than hydrogen, preferably from 1 to 60 atoms other than hydrogen, preferably from 1 to 50 atoms other than hydrogen, and preferably from 1 to 45 atoms other than hydrogen.

In a particularly preferred embodiment, the first or second aspect of the present invention provides a compound of formula (I) or a complex of formula (II):

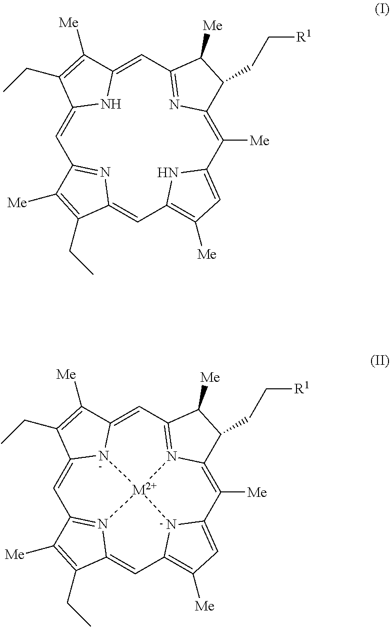

or a pharmaceutically acceptable salt thereof, wherein:
—R¹ is selected from —CH₂OR², —CH₂SR², —CH₂S(O)R², —CH₂S(O)₂R², —CH₂N(R²)(R^{2′}), —R², —C(O)—OR³, —C(O)—SR³, —C(O)—N(R³)(R^{3′}), —C(S)—OR³, —C(S)—SR³ or —C(S)—N(R³)(R^{3′}) [preferably —R¹ is selected from —C(O)—OR³, —C(O)—SR³, —C(O)—N(R³)(R^{3′}), —C(S)—OR³, —C(S)—SR³ or —C(S)—N(R³)(R^{3′}); more preferably —R¹ is —C(O)—N(R³)(R^{3′})];
—R² and —R³ are selected from —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁸]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{8′}];
—R^{2′} and —R^{3′} are selected from hydrogen or C₁-C₆ alkyl [preferably —R^{2′} and —R^{3′} are selected from hydrogen or C₁-C₃ alkyl; more preferably —R^{2′} and —R^{3′} are selected from hydrogen or methyl];
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂⁻, wherein the phenyl or C₅-C₆ heteroaryl may optionally be further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁸ is —[NC₅H₅] optionally substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{8′} is —[NC₅H₅] substituted with —CO₂ and optionally further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
Q is O, S, NH or NMe [preferably Q is O];
Y is a counter anion;
Z is a counter cation;
M²⁺ is a metal cation;
n is 1, 2, 3, 4, 5 or 6;
p is 0, 1, 2, 3 or 4;
r is 0, 1, 2, 3, 4, 5 or 6; and
s is 0, 1, 2, 3, 4, 5, 6, 7, 8, 9, 10, 11 or 12.

In a particularly preferred embodiment, the first or second aspect of the present invention provides a compound of formula (I) or a complex of formula (II):

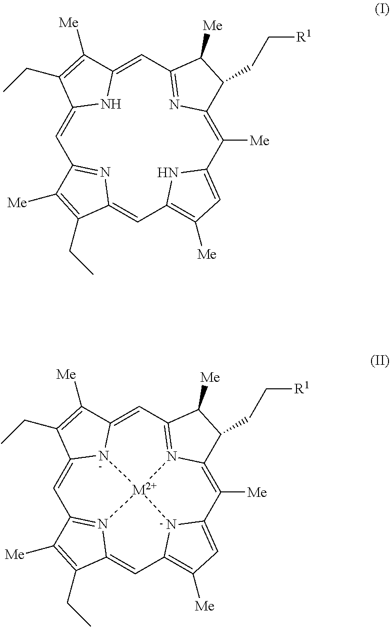

or a pharmaceutically acceptable salt thereof, wherein:
—R¹ is —C(O)—N(R³)(R^{3′});
—R³ is selected from —[(CH₂)ₚQ]ᵣ—(CH₂)ₛ [N(R⁵)₃]Y, —[(CH₂)ₚQ]ᵣ—(CH₂)ₛ [P(R⁵)₃]Y, —[(CH₂)ₚQ]ᵣ—(CH₂)ₛ—[R⁸]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{8′};
—R^{3′} is selected from hydrogen or C₁-C₃ alkyl (preferably hydrogen or methyl);
—R⁵, each independently, is selected from C₁-C₃ alkyl or phenyl, wherein the phenyl may optionally be substituted with one, two, three, four or five substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R^{5′} is selected from C₁-C₃ alkyl substituted with —CO₂ or phenyl substituted with —CO₂, wherein the phenyl may optionally be further substituted with one, two, three or four substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R⁸ is —[NC₅H₅] optionally substituted with one, two, three, four or five substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R^{8′} is —[NC₅H₅] substituted with —CO₂ and optionally further substituted with one, two, three or four substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
Q is O, S, NH or NMe (preferably O);
Y is a counter anion;
Z is a counter cation;
M²⁺ is a metal cation;
n is 1, 2, 3, 4, 5 or 6;
p is 0, 1, 2, 3 or 4;
r is 0, 1, 2, 3, 4, 5 or 6; and
s is 0, 1, 2, 3, 4, 5, 6, 7, 8, 9, 10, 11 or 12.

In these two preferred embodiments of the preceding paragraphs, each —R⁵ may be the same or different; preferably each —R⁵ is the same.

In another preferred embodiment of the first or second aspect of the present invention, the compound is a compound of formula (IA), (IB), (IC), (ID), (IE), (IF) or (IG):

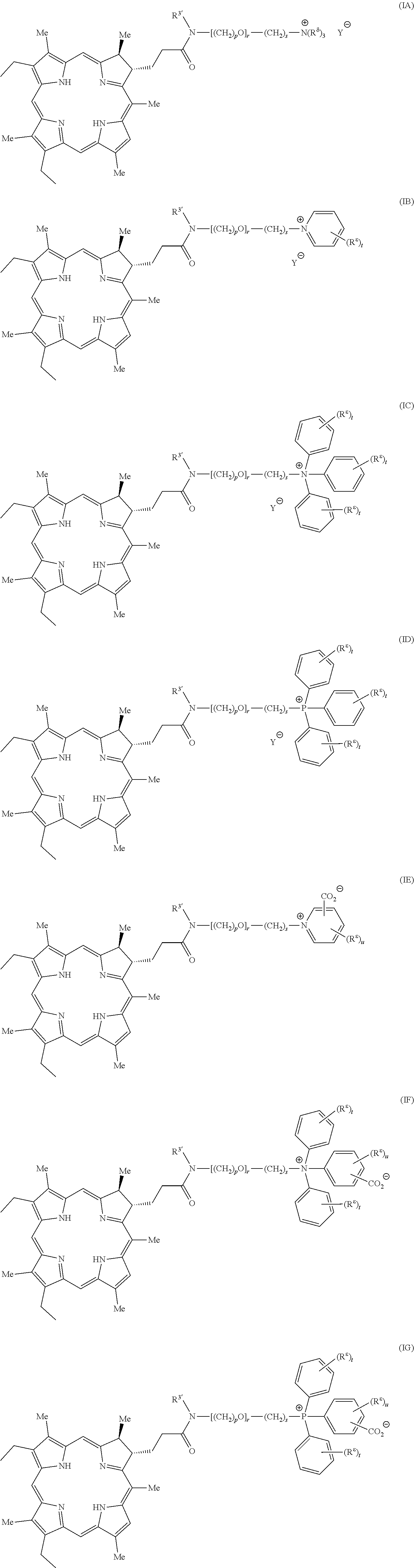

or a metal cation complex thereof, or a pharmaceutically acceptable salt thereof, wherein:
—R^{3′} is selected from hydrogen or C₁-C₃ alkyl (preferably hydrogen or methyl);
—R^{δ} is selected from C₁-C₃ alkyl;
—R^{ε} is selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
Y is a counter anion;
Z is a counter cation;
n is 1, 2, 3 or 4;
p is 0, 1, 2, 3 or 4;
r is 0, 1, 2, 3, 4, 5 or 6;
s is 0, 1, 2, 3, 4, 5, 6, 7, 8, 9, 10, 11 or 12;
t is 0, 1, 2, 3, 4 or 5; and
u is 0, 1, 2, 3 and 4.

The compounds of formula (IA), (IB), (IC), (ID), (IE), (IF), (IG) and complexes and salts thereof according to the first and second aspect of the present invention comprise a moiety —[(CH₂)ₚO]ᵣ—(CH₂)ₛ—, wherein:
p is 0, 1, 2, 3 or 4;
r is 0, 1, 2, 3, 4, 5 or 6; and
s is 0, 1, 2, 3, 4, 5, 6, 7, 8, 9, 10, 11 or 12.

In one embodiment, p is 2, 3 or 4; r is 1; and s is 2, 3 or 4. In a preferred embodiment, p is 3; r is 1; and s is 3; such that —[(CH₂)ₚO]ᵣ—(CH₂)ₛ is —(CH₂)₃—O—(CH₂)₃—.

In another embodiment, p is 2 or 3; r is 2 or 3; and s is 2 or 3. In a preferred embodiment, p is 2; r is 2; and s is 2; such that —[(CH₂)ₚO]ᵣ—(CH₂)ₛ— is —(CH₂CH₂O)₂—(CH₂)₂—.

In yet another embodiment, r is 0; and s is 1, 2, 3, 4, 5, 6, 7, 8, 9, 10, 11 or 12; such that —[(CH₂)ₚO]ᵣ—(CH₂)ₛ is —(CH₂)₁₋₁₂—.

Preferably in the compound or complex according to the first or second aspect of the present invention, the compound or complex is:

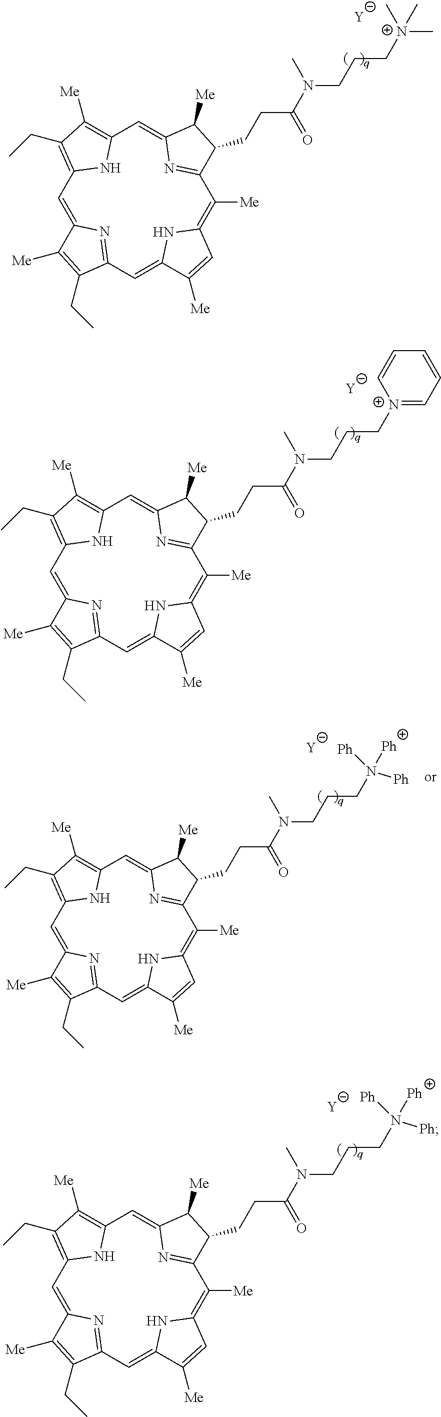

wherein Y is a counter anion, and q is 0, 1, 2, 3 or 4 (preferably q is 1); or a metal cation complex thereof, or a pharmaceutically acceptable salt thereof.

In a particularly preferred embodiment, the first or second aspect of the present invention provides a compound of formula (III) or a complex of formula (IV):

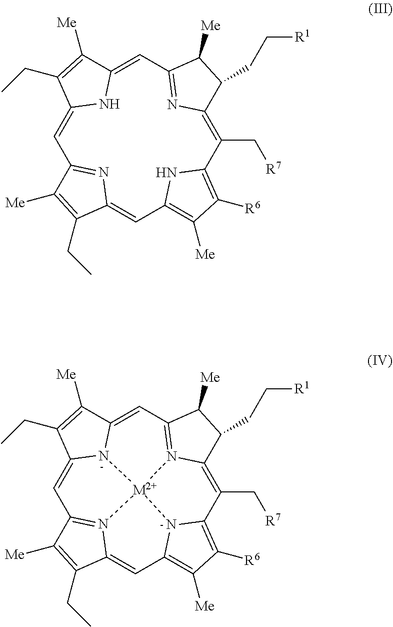

or a pharmaceutically acceptable salt thereof, wherein:
—R¹ is selected from —C(O)—OR³, —C(O)—SR³ or —C(O)—N(R³)(R^{3′}), —R³ is selected from —R^{α}—OR^{β} or —R^{α}—SR^{β} and —R^{β} is a saccharidyl group, and —R^{3′} is H or C₁-C₄ alkyl;
—R⁶ is selected from:
(a) —C(O)—OR³, —C(O)—SR³ or —C(O)—N(R³)₂, and —R³, each independently, is C₁-C₄ alkyl, preferably —R⁶ is —C(O)—OR³ and —R³ is C₁-C₄ alkyl; or
(b) —C(O)—OR³, —C(O)—SR³ or —C(O)—N(R³)(R^{3′}), wherein —R³ is selected from —R^{α}—OR^{β} or —R^{α}—SR^{β} and —R^{β} is a saccharidyl group, and —R^{3′} is H or C₁-C₄ alkyl;
—R^{α}— is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S; and
M²⁺ is a metal cation.

In a particularly preferred embodiment, the first or second aspect of the present invention provides a compound of formula (III) or a complex of formula (IV):

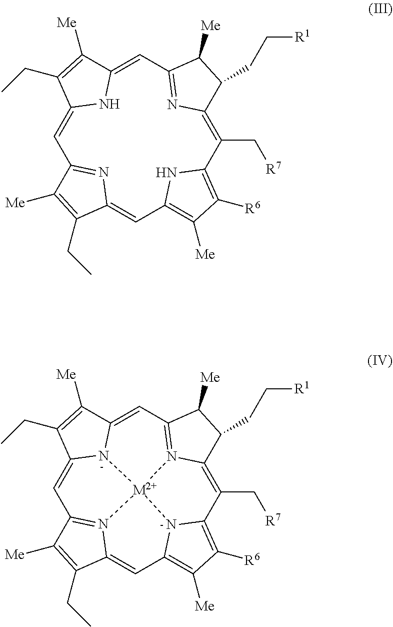

or a pharmaceutically acceptable salt thereof, wherein:
—R¹ is selected from:
(a) —C(O)—OR³, —C(O)—SR³ or —C(O)—N(R³)₂, and —R³, each independently, is —R^{β}, and —R^{β} is a C₁-C₄ alkyl group, more preferably, —R¹ is —C(O)—OR³ and —R³ is —R^{β}, and —R^{β} is a C₁-C₄ alkyl group; or
(b) —C(O)—OR³, —C(O)—SR³ or —C(O)—N(R³)(R^{3′}), —R³ is selected from —R^{α}—OR^{β} or —R^{α}—SR^{β} and —R^{β} is a saccharidyl group, and —R^{3′} is H or C₁-C₄ alkyl;
—R⁶ is selected from —C(O)—OR³, —C(O)—SR³ or —C(O)—N(R³)(R^{3′}), wherein —R³ is selected from —R^{α}—OR^{β} or —R^{α}—SR^{β} and —R^{β} is a saccharidyl group, and —R^{3′} is H or C₁-C₄ alkyl;
—R^{α}— is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S; and
M²⁺ is a metal cation.

In a particularly preferred embodiment the first or second aspect of the present invention provides a compound of formula (III) or a complex of formula (IV):

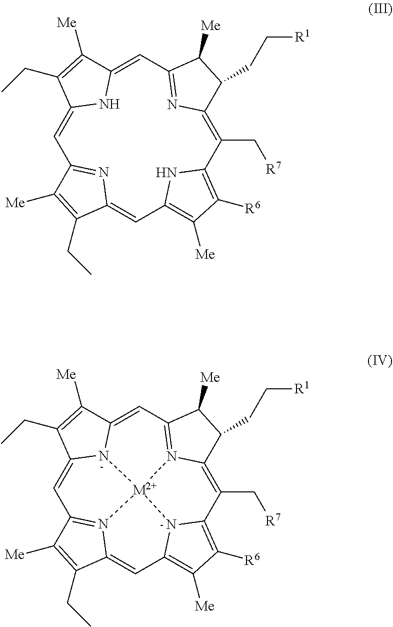

or a pharmaceutically acceptable salt thereof, wherein:
—R¹ is selected from —C(O)—OR³, —C(O)—SR³ or —C(O)—N(R³)(R^{3′}), wherein —R³ is selected from —R^{α}—OR^{β} or —R^{α}—SR^{β} and —R^{β} is a saccharidyl group, and —R^{3′} is H or C₁-C₄ alkyl;
—R⁶ is selected from —C(O)—OR³, —C(O)—SR³ or —C(O)—N(R³)(R^{3′}), wherein —R³ is selected from —R^{α}—OR^{β} or —R^{α}—SR^{β} and —R^{β} is a saccharidyl group, and —R^{3′} is H or C₁-C₄ alkyl;
—R^{α}— is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S; and
M²⁺ is a metal cation.

In a particularly preferred embodiment, the first or second aspect of the present invention provides a compound of formula (III) or a complex of formula (IV):

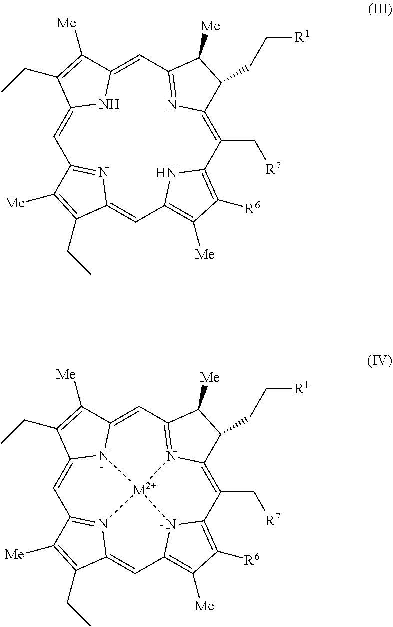

or a pharmaceutically acceptable salt thereof, wherein:
—R¹ is selected from —CH₂OR², —CH₂SR², —CH₂S(O)R², —CH₂S(O)₂R², —CH₂N(R²)(R^{2′}), —R², —C(O)—OR³, —C(O)—SR³, —C(O)—N(R³)(R^{3′}), —C(S)—OR³, —C(S)—SR³ or —C(S)—N(R³)(R^{3′}) [preferably —R¹ is selected from —C(O)—OR³, —C(O)—SR³, —C(O)—N(R³)(R^{3′}), —C(S)—OR³, —C(S)—SR³ or —C(S)—N(R³)(R^{3′}); more preferably —R¹ is —C(O)—N(R³)(R^{3′})];
—R² is selected from —H, —C(O)R⁴, —C(O)—OR⁴, —C(O)—SR⁴, —C(O)—N(R⁴)(R^{4′}), —C(S)—OR⁴, —C(S)—SR⁴, —C(S)—N(R⁴)(R^{4′}), —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}NH(R^{β}), —R^{α}—N(R^{β})₂, —R^{α}—X, —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁸]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{8′}];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁸]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{8′}];
wherein at least one of —R², —R³ and —R⁴ is selected from —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁸]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{8′}];
—R^{2′}, —R^{3′} and —R^{4′}, each independently, is selected from hydrogen or C₁-C₆ alkyl [preferably —R^{2′}, —R^{3′} and —R^{4′}, each independently, is selected from hydrogen or C₁-C₃ alkyl; more preferably —R^{2′}, —R^{3′} and —R^{4′}, each independently, is selected from hydrogen or methyl];
—R^{α}—, each independently, is selected from a C₁-C₄₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂, wherein the phenyl or C₅-C₆ heteroaryl may optionally be further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁶ is selected from —C(O)—OR³, —C(O)—SR³, —C(O)—N(R³)(R^{3′}), —C(S)—OR³, —C(S)—SR³ or —C(S)—N(R³)(R^{3′}) [preferably —R⁶ is —C(O)—N(R³)(R^{3′})];
—R⁸ is —[NC₅H₅] optionally substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{8′} is —[NC₅H₅] substituted with —CO₂ and optionally further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
Q is O, S, NH or NMe [preferably Q is O];
X is a halo group;
Y is a counter anion;
Z is a counter cation;
M²⁺ is a metal cation;
n is 1, 2, 3, 4, 5 or 6;
p is 0, 1, 2, 3 or 4;
r is 0, 1, 2, 3, 4, 5 or 6; and
s is 0, 1, 2, 3, 4, 5, 6, 7, 8, 9, 10, 11 or 12.

In a particularly preferred embodiment, the first or second aspect of the present invention provides a compound of formula (III) or a complex of formula (IV):

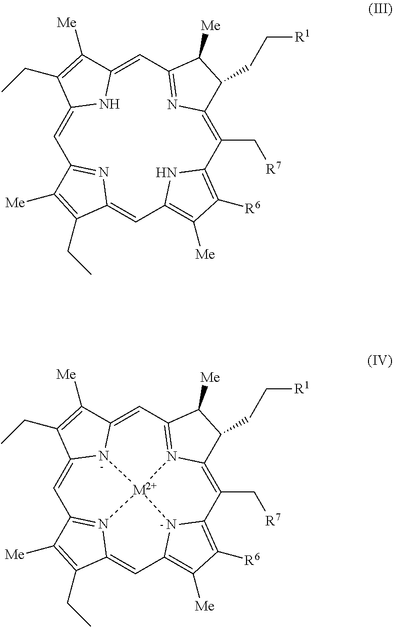

or a pharmaceutically acceptable salt thereof, wherein:
—R¹ is selected from —C(O)—OR³, —C(O)—SR³, —C(O)—N(R³)(R^{3′}), —C(S)—OR³, —C(S)—SR³ or —C(S)—N(R³)(R^{3′}) [preferably —R¹ is —C(O)—N(R³)(R^{3′})];
—R³, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —[(CH₂)ₚQ]ᵣ—(CH₂)ₛ [N(R⁵)₃]Y, —[(CH₂)ₚQ]ᵣ—(CH₂)ₛ [P(R⁵)₃]Y, —[(CH₂)ₚQ]ᵣ—(CH₂)ₛ—[R⁸]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{8′}]; wherein at least one —R³ is selected from —[(CH₂)ₚQ]ᵣ—(CH₂)ₛ [N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁸]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{8′}];
—R^{3′} is selected from hydrogen or C₁-C₃ alkyl [preferably —R^{3′} is hydrogen or methyl];
—R^{α}—, each independently, is selected from a C₁-C₄₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R⁵, each independently, is selected from C₁-C₃ alkyl or phenyl, wherein the phenyl may optionally be substituted with one, two, three, four or five substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R^{5′} is selected from C₁-C₃ alkyl substituted with —CO₂ or phenyl substituted with —CO₂⁻, wherein the phenyl may optionally be further substituted with one, two, three or four substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R⁶ is selected from —C(O)—OR³, —C(O)—SR³, —C(O)—N(R³)(R^{3′}), —C(S)—OR³, —C(S)—SR³ or —C(S)—N(R³)(R^{3′}) [preferably —R⁶ is —C(O)—N(R³)(R^{3′})];
—R⁸ is —[NC₅H₅] optionally substituted with one, two, three, four or five substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R^{8′} is —[NC₅H₅] substituted with —CO₂ and optionally further substituted with one, two, three or four substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
Q is O, S, NH or NMe [preferably Q is O];
X is a halo group;
Y is a counter anion;
Z is a counter cation;
M²⁺ is a metal cation;
n is 1, 2, 3, 4, 5 or 6;
p is 0, 1, 2, 3 or 4;
r is 0, 1, 2, 3, 4, 5 or 6; and
s is 0, 1, 2, 3, 4, 5, 6, 7, 8, 9, 10, 11 or 12.

In these two preferred embodiments of the preceding paragraphs, each —R⁵ may be the same or different; preferably each —R⁵ is the same.

In another preferred embodiment of the first or second aspect of the present invention, the compound is a compound of formula (IIIA), (IIIB), (IIIC), (IIID), (IIIE), (IIIF), (IIIG), (IIIH), (IIIJ), (IIIK), (IIIL), (IIIM), (IIIN) or (IIIO):

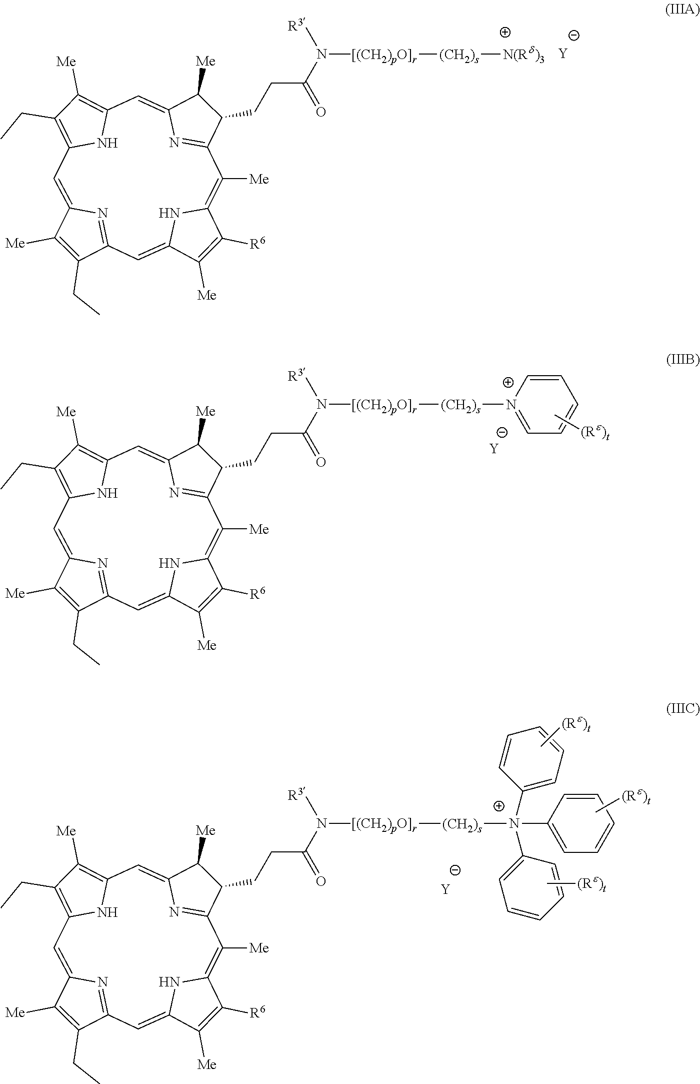

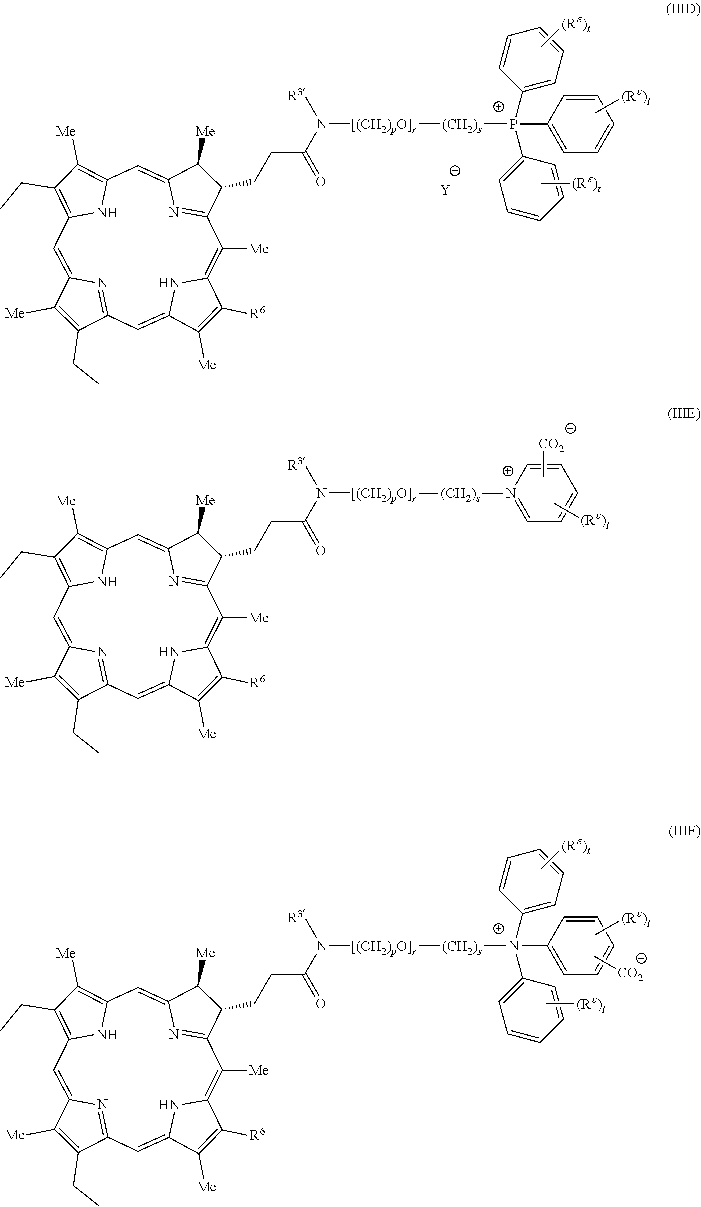

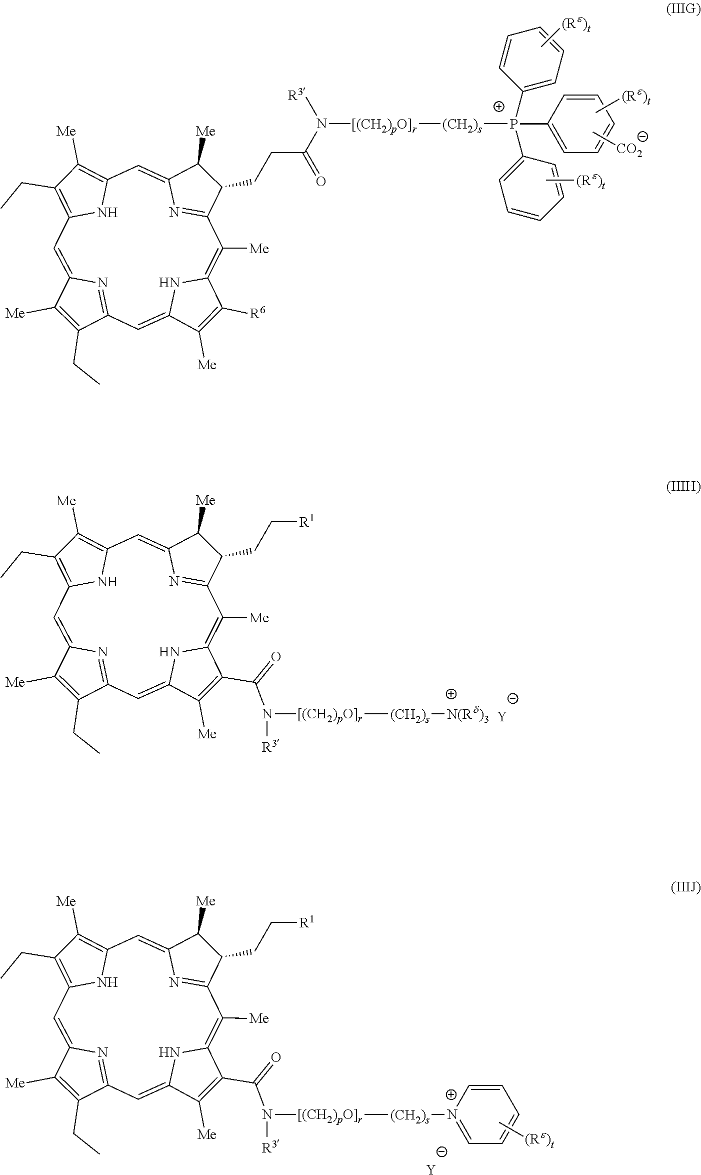

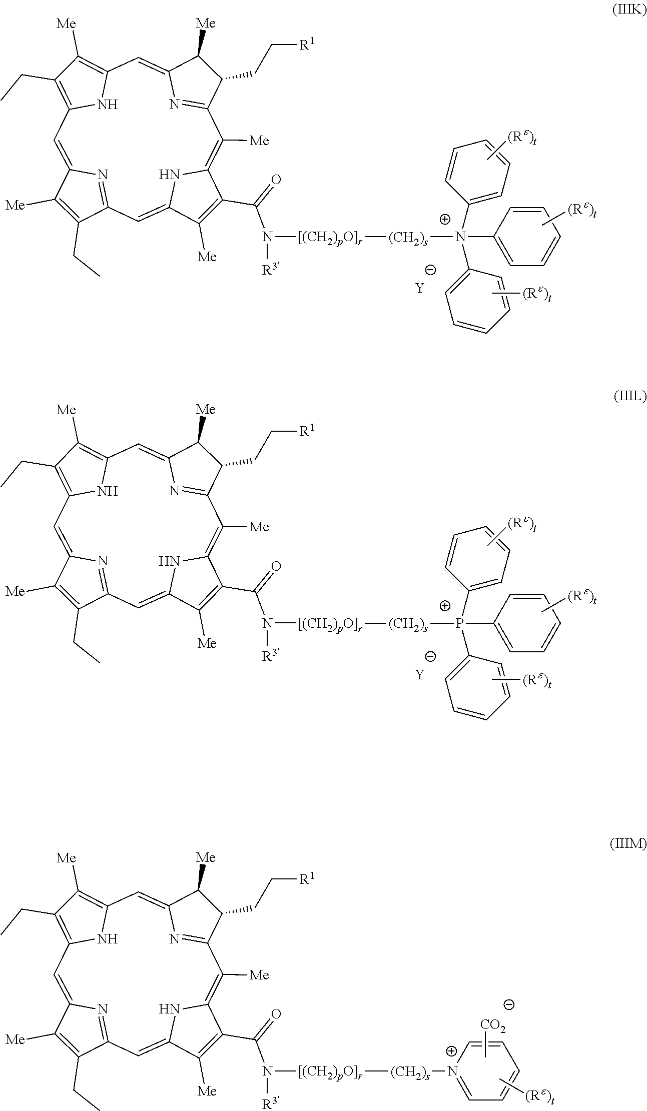

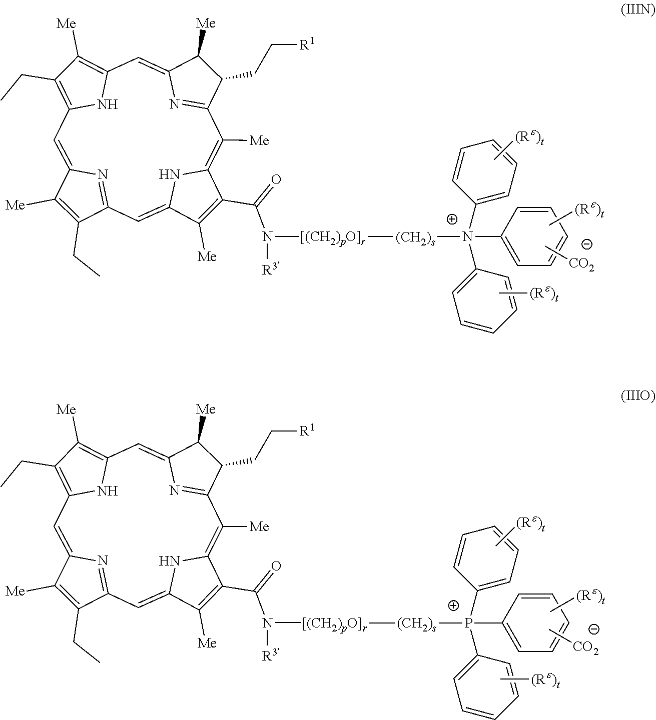

or a metal cation complex thereof, or a pharmaceutically acceptable salt thereof, wherein:
—R¹ is selected from —C(O)—OR³, —C(O)—SR³, —C(O)—N(R³)(R^{3′}), —C(S)—OR³, —C(S)—SR³ or —C(S)—N(R³)(R^{3′});
—R³ is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, or —R^{α}—X;
—R^{3′} is selected from hydrogen or C₁-C₃ alkyl (preferably hydrogen or methyl);
—R⁶ is selected from —C(O)—OR³, —C(O)—SR³, —C(O)—N(R³)(R^{3′}), —C(S)—OR³, —C(S)—SR³ or —C(S)—N(R³)(R^{3′});
—R^{α}—, each independently, is selected from a C₁-C₁₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R^{δ} is selected from C₁-C₃ alkyl;
—R^{ε} is selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
X is a halo group;
Y is a counter anion;
Z is a counter cation;
n is 1, 2, 3 or 4;
p is 0, 1, 2, 3 or 4;
r is 0, 1, 2, 3, 4, 5 or 6;
s is 0, 1, 2, 3, 4, 5, 6, 7, 8, 9, 10, 11 or 12;
t is 0, 1, 2, 3, 4 or 5; and
u is 0, 1, 2, 3 and 4.

The compounds of formula (IIIA), (IIIB), (IIIC), (IIID), (IIIE), (IIIF), (IIIG), (IIIH), (IIIJ), (IIIK), (IIIL), (IIIM), (IIIN), (IIIO) and complexes and salts thereof according to the first and second aspect of the present invention comprise a moiety —[(CH₂)ₚO]ᵣ—(CH₂)ₛ—, wherein:
p is 0, 1, 2, 3 or 4;
r is 0, 1, 2, 3, 4, 5 or 6; and
s is 0, 1, 2, 3, 4, 5, 6, 7, 8, 9, 10, 11 or 12.

In one embodiment, p is 2, 3 or 4; r is 1; and s is 2, 3 or 4. In a preferred embodiment, p is 3; r is 1; and s is 3; such that —[(CH₂)ₚO]ᵣ—(CH₂)ₛ is —(CH₂)₃—O—(CH₂)₃—.

In another embodiment, p is 2 or 3; r is 2 or 3; and s is 2 or 3. In a preferred embodiment, p is 2; r is 2; and s is 2; such that —[(CH₂)ₚO]ᵣ—(CH₂)ₛ— is —(CH₂CH₂O)₂—(CH₂)₂—.

In yet another embodiment, r is 0; and s is 1, 2, 3, 4, 5, 6, 7, 8, 9, 10, 11 or 12; such that —[(CH₂)ₚO]ᵣ(CH₂)ₛ— is —(CH₂)₁₋₁₂—.

Preferably in the compound or complex according to the first or second aspect of the present invention, the compound or complex is:

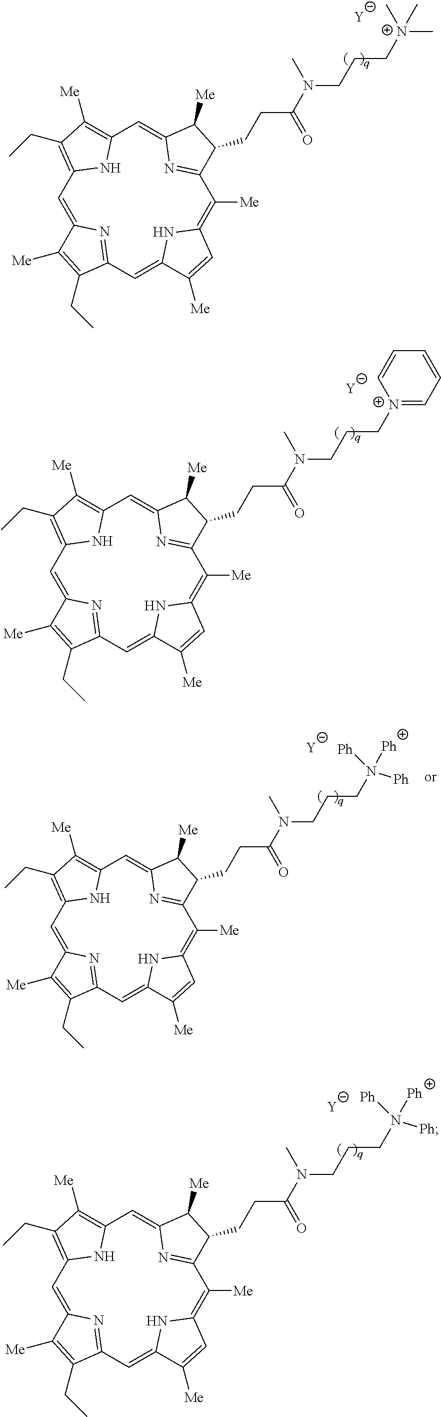

wherein Y is a counter anion, and q is 0, 1, 2, 3 or 4 (preferably q is 1);
or a metal cation complex thereof, or a pharmaceutically acceptable salt thereof.

In a particularly preferred embodiment, the first or second aspect of the present invention provides a compound of formula (V) or a complex of formula (VI):

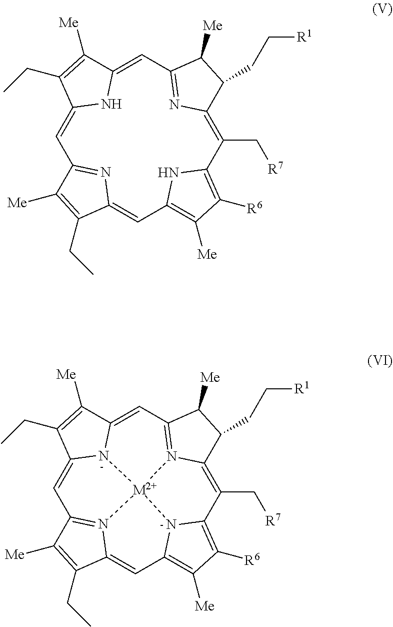

or a pharmaceutically acceptable salt thereof, wherein:
—R¹ is selected from —C(O)—OR³, —C(O)—SR³ or —C(O)—N(R³)(R^{3′}), —R³ is selected from —R^{α}—OR^{β} or —R^{α}—SR^{β} and —R^{β} is a saccharidyl group, and —R^{3′} is H or C₁-C₄ alkyl;
—R⁶ is selected from:
(a) —C(O)—OR³, —C(O)—SR³ or —C(O)—N(R³)₂, and —R³, each independently, is C₁-C₄ alkyl, preferably —R⁶ is —C(O)—OR³ and —R³ is C₁-C₄ alkyl; or
(b) —C(O)—OR³, —C(O)—SR³ or —C(O)—N(R³)(R^{3′}), wherein —R³ is selected from —R^{α}—OR^{β} or —R^{α}—SR^{β} and —R^{β} is a saccharidyl group, and —R^{3′} is H or C₁-C₄ alkyl;
—R⁷ is selected from:
(a) —C(O)—OR³, —C(O)—SR³ or —C(O)—N(R³)₂, and —R³, each independently, is C₁-C₄ alkyl, preferably —R⁷ is —C(O)—OR³ and —R³ is C₁-C₄ alkyl; or
(b) —C(O)—OR³, —C(O)—SR³ or —C(O)—N(R³)(R^{3′}), wherein —R³ is selected from —R^{α}—OR^{β} or —R^{α}—SR^{β} and —R^{β} is a saccharidyl group, and —R^{3′} is H or C₁-C₄ alkyl;
—R^{α}— is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S; and
M²⁺ is a metal cation.

In a particularly preferred embodiment, the first or second aspect of the present invention provides a compound of formula (V) or a complex of formula (VI):

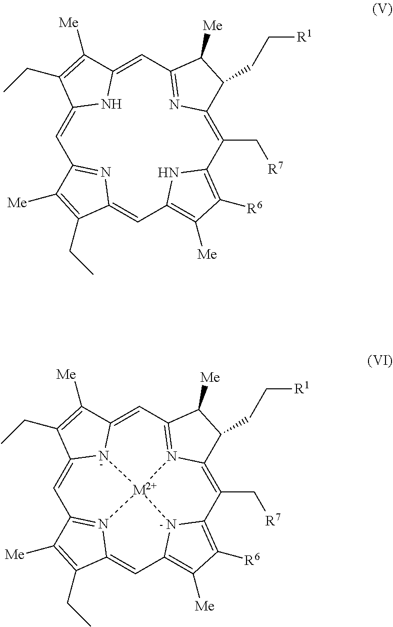

or a pharmaceutically acceptable salt thereof, wherein:
—R¹ is selected from:
(a) —C(O)—OR³, —C(O)—SR³ or —C(O)—N(R³)₂, and —R³, each independently, is —R^{β}, and —R^{β} is a C₁-C₄ alkyl group, more preferably, —R¹ is —C(O)—OR³ and —R³ is —R^{β}, and —R^{β} is a C₁-C₄ alkyl group; or
(b) —C(O)—OR³, —C(O)—SR³ or —C(O)—N(R³)(R^{3′}), —R³ is selected from —R^{α}—OR^{β} or —R^{α}—SR^{β} and —R^{β} is a saccharidyl group, and —R^{3′} is H or C₁-C₄ alkyl;
—R⁶ is selected from —C(O)—OR³, —C(O)—SR³ or —C(O)—N(R³)(R^{3′}), wherein —R³ is selected from —R^{α}—OR^{β} or —R^{α}—SR^{β} and —R^{β} is a saccharidyl group, and —R^{3′} is H or C₁-C₄ alkyl;
—R⁷ is selected from:
(a) —C(O)—OR³, —C(O)—SR³ or —C(O)—N(R³)₂, and —R³, each independently, is C₁-C₄ alkyl, preferably —R⁷ is —C(O)—OR³ and —R³ is C₁-C₄ alkyl; or
(b) —C(O)—OR³, —C(O)—SR³ or —C(O)—N(R³)(R^{3′}), wherein —R³ is selected from —R^{α}—OR^{β} or —R^{α}—SR^{β} and —R^{β} is a saccharidyl group, and —R^{3′} is H or C₁-C₄ alkyl;
—R^{α}— is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S; and
M²⁺ is a metal cation.

In a particularly preferred embodiment, the first or second aspect of the present invention provides a compound of formula (V) or a complex of formula (VI):

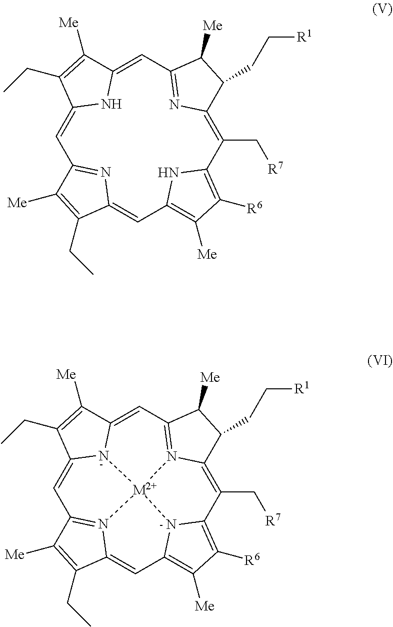

or a pharmaceutically acceptable salt thereof, wherein:
—R¹ is selected from:
(a) —C(O)—OR³, —C(O)—SR³ or —C(O)—N(R³)₂, and —R³, each independently, is —R^{β}, and —R^{β} is a C₁-C₄ alkyl group, more preferably, —R¹ is —C(O)—OR³ and —R³ is —R^{β}, and —R^{β} is a C₁-C₄ alkyl group; or
(b) —C(O)—OR³, —C(O)—SR³ or —C(O)—N(R³)(R^{3′}), —R³ is selected from —R^{α}—OR^{β} or —R^{α}—SR^{β} and —R^{β} is a saccharidyl group, and —R^{3′} is H or C₁-C₄ alkyl;
—R⁶ is selected from:
(a) —C(O)—OR³, —C(O)—SR³ or —C(O)—N(R³)₂, and —R³, each independently, is C₁-C₄ alkyl, preferably —R⁶ is —C(O)—OR³ and —R³ is C₁-C₄ alkyl; or
(b) —C(O)—OR³, —C(O)—SR³ or —C(O)—N(R³)(R^{3′}), wherein —R³ is selected from —R^{α}—OR^{β} or —R^{α}—SR^{β} and —R^{β} is a saccharidyl group, and —R^{3′} is H or C₁-C₄ alkyl;
—R⁷ is selected from —C(O)—OR³, —C(O)—SR³ or —C(O)—N(R³)(R^{3′}), wherein —R³ is selected from —R^{α}—OR^{β} or —R^{α}—SR^{β} and —R^{β} is a saccharidyl group, and —R^{3′} is H or C₁-C₄ alkyl;
—R^{α}— is selected from a C₁-C₁₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by one or more heteroatoms O or S; and
M²⁺ is a metal cation.

In a particularly preferred embodiment, the first or second aspect of the present invention provides a compound of formula (V) or a complex of formula (VI):

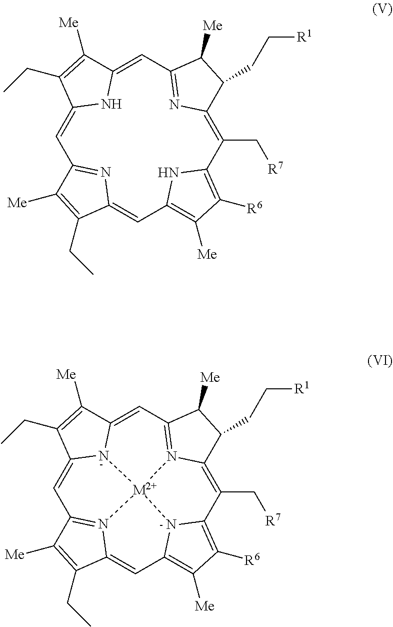

or a pharmaceutically acceptable salt thereof, wherein:
—R¹ is selected from —CH₂OR², —CH₂SR², —CH₂S(O)R², —CH₂S(O)₂R², —CH₂N(R²)(R^{2′}), —R², —C(O)—OR³, —C(O)—SR³, —C(O)—N(R³)(R^{3′}), —C(S)—OR³, —C(S)—SR³ or —C(S)—N(R³)(R^{3′}) [preferably —R¹ is selected from —C(O)—OR³, —C(O)—SR³, —C(O)—N(R³)(R^{3′}), —C(S)—OR³, —C(S)—SR³ or —C(S)—N(R³)(R^{3′}); more preferably —R¹ is —C(O)—N(R³)(R^{3′})];
—R² is selected from —H, —C(O)R⁴, —C(O)—OR⁴, —C(O)—SR⁴, —C(O)—N(R⁴)(R^{4′}), —C(S)—OR⁴, —C(S)—SR⁴, —C(S)—N(R⁴)(R^{4′}), —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —[(CH₂)_{P}Q]ᵣ—(CH₂)ₛ—[N(R⁵)₃]Y, —[(CH₂)_{P}Q]ᵣ—(CH₂)ₛ—[P(R⁵)₃]Y, —[(CH₂)_{P}Q]ᵣ—(CH₂)ₛ—[R⁸]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{8′}];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁸]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{8′}];
wherein at least one of —R², —R³ and —R⁴ is selected from —[(CH₂)ₚQ]ᵣ—(CH₂)ₛ—[N(R⁵)₃]Y, —[(CH₂)ₚQ]ᵣ—(CH₂)ₛ [P(R⁵)₃]Y, —[(CH₂)ₚQ]ᵣ—(CH₂)ₛ [R⁸]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{8′}];
—R^{2′}, —R^{3′} and —R^{4′}, each independently, is selected from hydrogen or C₁-C₆ alkyl [preferably —R^{2′}, —R^{3′} and —R^{4′}, each independently, is selected from hydrogen or C₁-C₃ alkyl; more preferably —R^{2′}, —R^{3′} and —R^{4′}, each independently, is selected from hydrogen or methyl];
—R^{α}—, each independently, is selected from a C₁-C₄₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂, wherein the phenyl or C₅-C₆ heteroaryl may optionally be further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁶ is selected from —C(O)—OR³, —C(O)—SR³, —C(O)—N(R³)(R^{3′}), —C(S)—OR³, —C(S)—SR³ or —C(S)—N(R³)(R^{3′}) [preferably —R⁶ is —C(O)—N(R³)(R^{3′})];
—R⁷ is selected from —C(O)—OR³, —C(O)—SR³, —C(O)—N(R³)(R^{3′}), —C(S)—OR³, —C(S)—SR³ or —C(S)—N(R³)(R^{3′}) [preferably —R⁷ is —C(O)—N(R³)(R^{3′})];
—R⁸ is —[NC₅H₅] optionally substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{8′} is —[NC₅H₅] substituted with —CO₂ and optionally further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
Q is O, S, NH or NMe [preferably Q is 0];
X is a halo group;
Y is a counter anion;
Z is a counter cation;
M²⁺ is a metal cation;
n is 1, 2, 3, 4, 5 or 6;
p is 0, 1, 2, 3 or 4;
r is 0, 1, 2, 3, 4, 5 or 6; and
s is 0, 1, 2, 3, 4, 5, 6, 7, 8, 9, 10, 11 or 12.

In a particularly preferred embodiment, the first or second aspect of the present invention provides a compound of formula (V) or a complex of formula (VI):

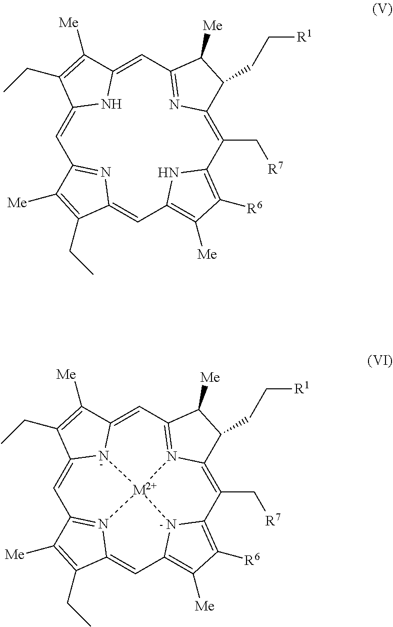

or a pharmaceutically acceptable salt thereof, wherein:
—R¹ is selected from —C(O)—OR³, —C(O)—SR³, —C(O)—N(R³)(R^{3′}), —C(S)—OR³, —C(S)—SR³ or —C(S)—N(R³)(R^{3′}) [preferably —R¹ is —C(O)—N(R³)(R^{3′})];
—R³, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{β}—NH(R^{β}), —R^{α}—N(R^{β})₂, —R^{α}—X, —[(CH₂)ₚQ]ᵣ—(CH₂)ₛ [N(R⁵)₃]Y, —[(CH₂)ₚQ]ᵣ—(CH₂)ₛ [P(R⁵)₃]Y, —[(CH₂)ₚQ]ᵣ—(CH₂)ₛ—[R⁸]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{8′}]; wherein at least one —R³ is selected from —[(CH₂)ₚQ]ᵣ—(CH₂)ₛ [N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁸]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{8′}];
—R^{3′} is selected from hydrogen or C₁-C₃ alkyl [preferably —R^{3′} is hydrogen or methyl];
—R^{α}—, each independently, is selected from a C₁-C₄₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R⁵, each independently, is selected from C₁-C₃ alkyl or phenyl, wherein the phenyl may optionally be substituted with one, two, three, four or five substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R^{5′} is selected from C₁-C₃ alkyl substituted with —CO₂ or phenyl substituted with —CO₂, wherein the phenyl may optionally be further substituted with one, two, three or four substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R⁶ is selected from —C(O)—OR³, —C(O)—SR³, —C(O)—N(R³)(R^{3′}), —C(S)—OR³, —C(S)—SR³ or —C(S)—N(R³)(R^{3′}) [preferably —R⁶ is —C(O)—N(R³)(R^{3′})];
—R⁷ is selected from —C(O)—OR³, —C(O)—SR³, —C(O)—N(R³)(R^{3′}), —C(S)—OR³, —C(S)—SR³ or —C(S)—N(R³)(R^{3′}) [preferably —R⁷ is —C(O)—N(R³)(R^{3′})];
—R⁸ is —[NC₅H₅] optionally substituted with one, two, three, four or five substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R^{8′} is —[NC₅H₅] substituted with —CO₂ and optionally further substituted with one, two, three or four substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
Q is O, S, NH or NMe [preferably Q is 0];
X is a halo group;
Y is a counter anion;
Z is a counter cation;
M²⁺ is a metal cation;
n is 1, 2, 3, 4, 5 or 6;
p is 0, 1, 2, 3 or 4;
r is 0, 1, 2, 3, 4, 5 or 6; and
s is 0, 1, 2, 3, 4, 5, 6, 7, 8, 9, 10, 11 or 12.

In these two preferred embodiments of the preceding paragraphs, each —R⁵ may be the same or different; preferably each —R⁵ is the same.

In another preferred embodiment of the first or second aspect of the present invention, the compound is a compound of formula (VA), (VB), (VC), (VD), (VE), (VF), (VG), (VH), (VJ), (VK), (VL), (VM), (VN), (VO), (VP), (VQ), (VR), (VS), (VT), (VU) or (VV):

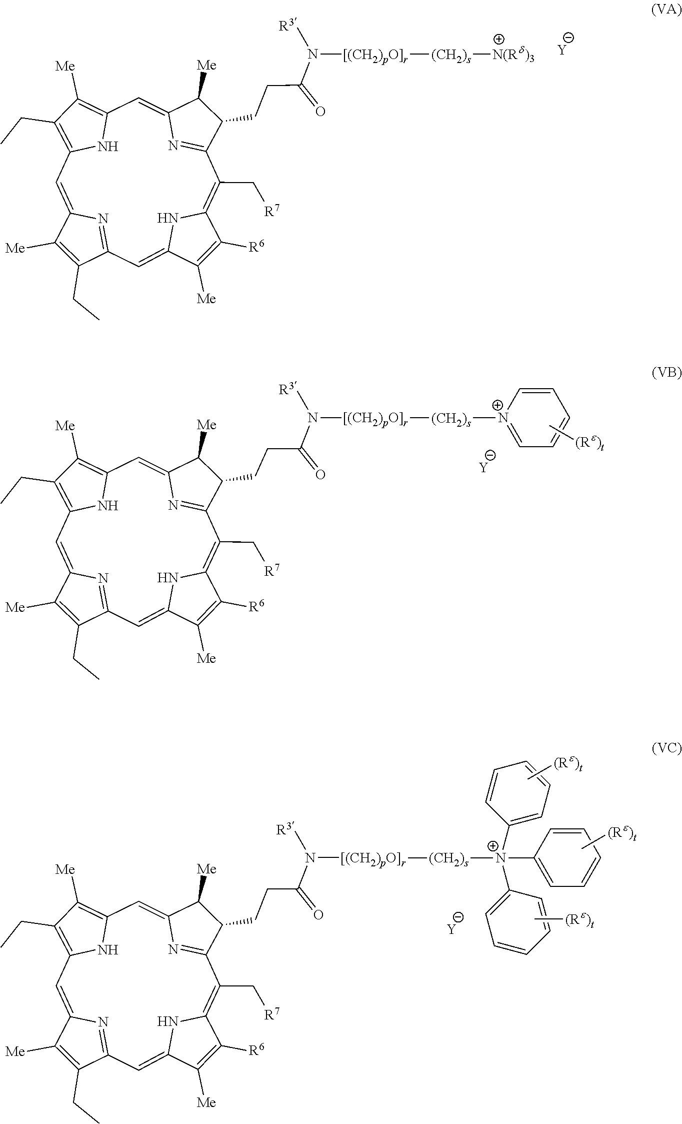

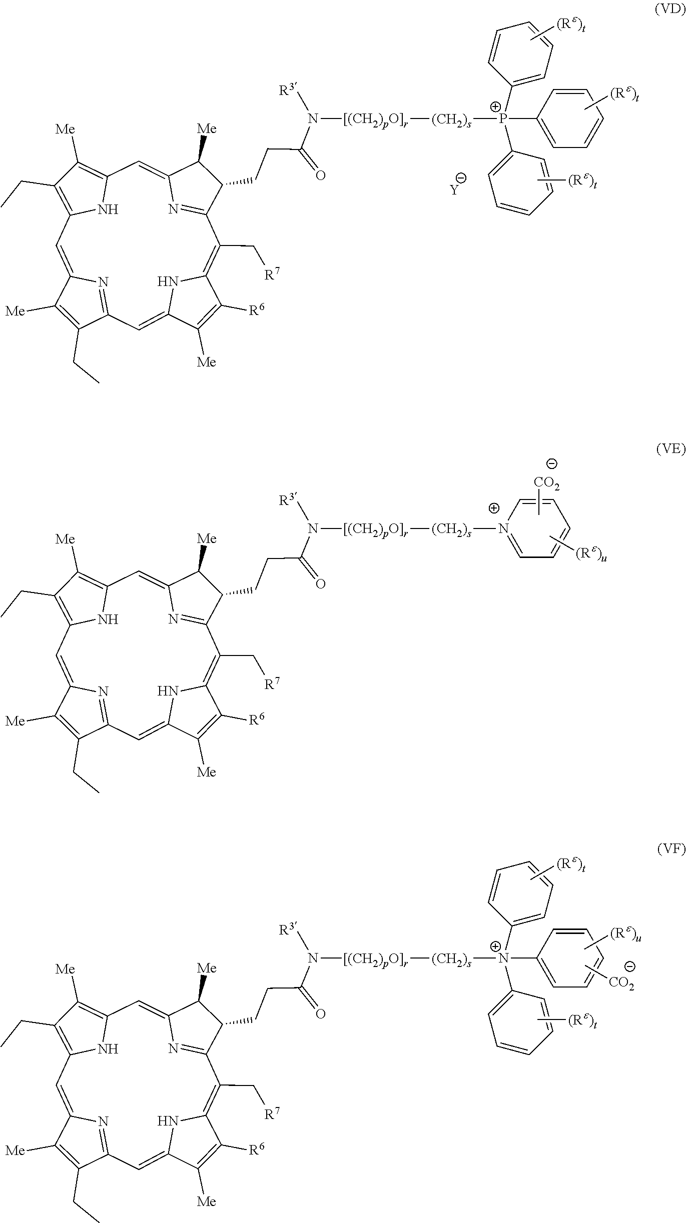

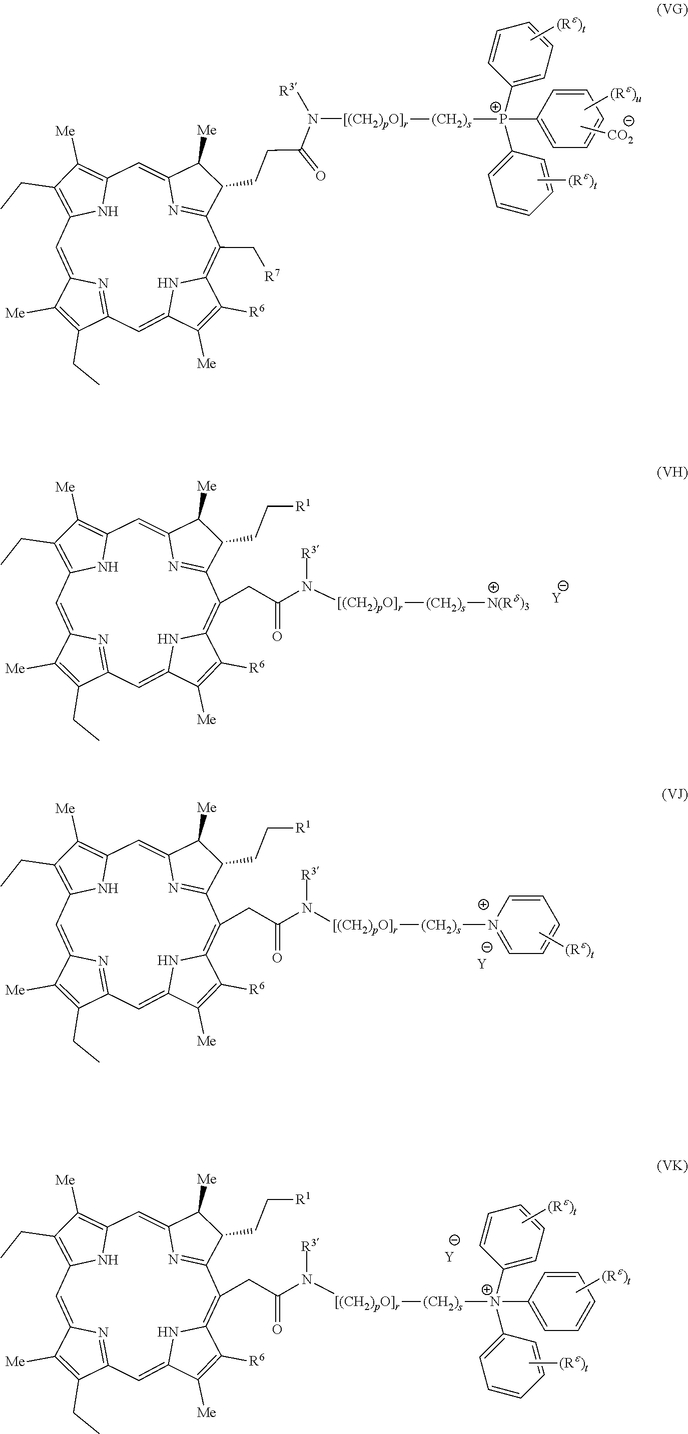

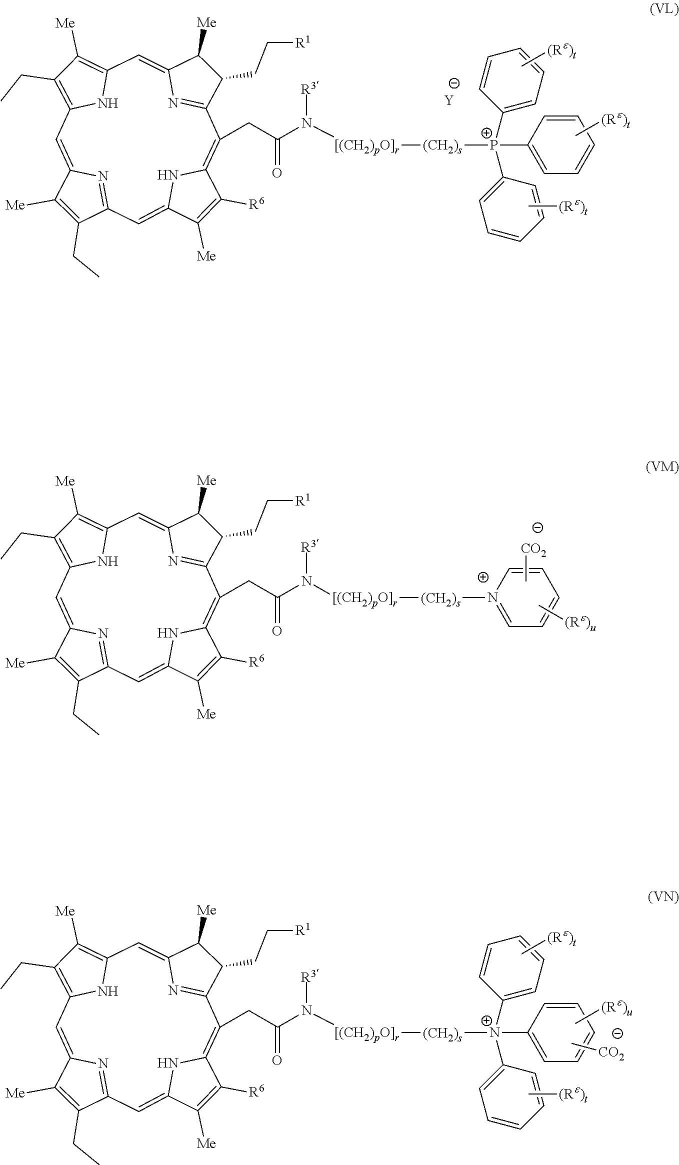

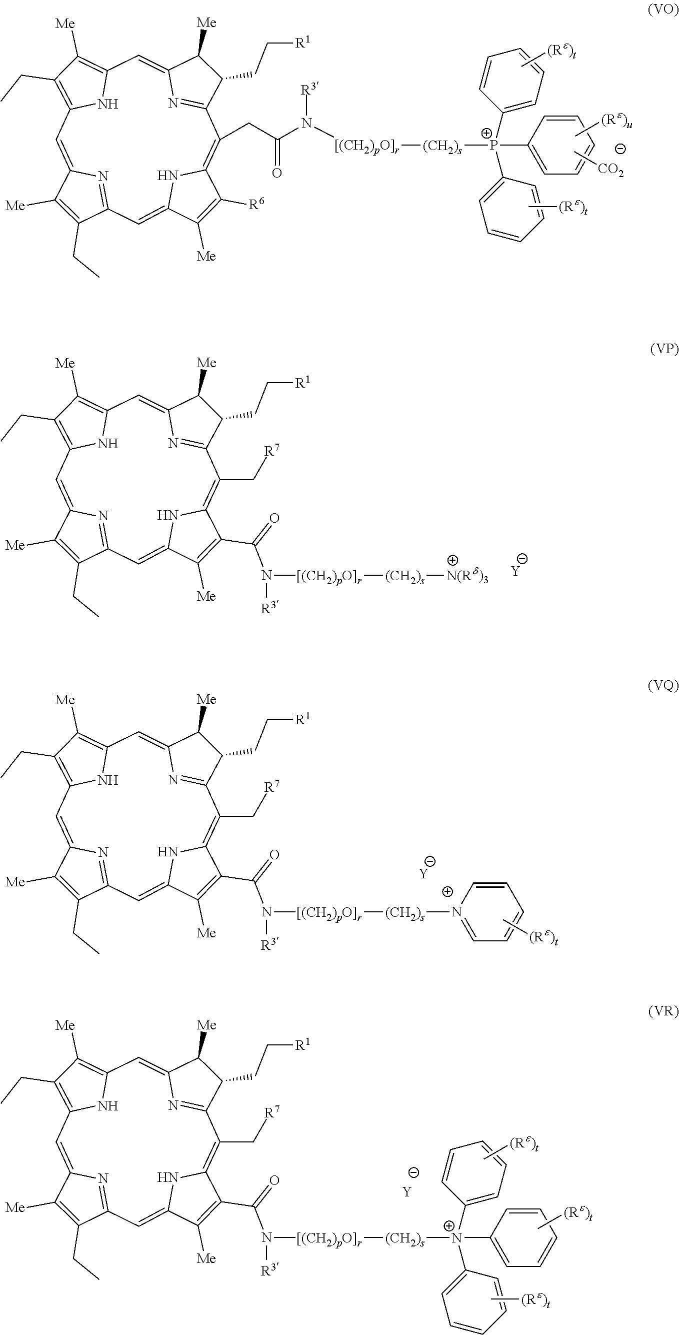

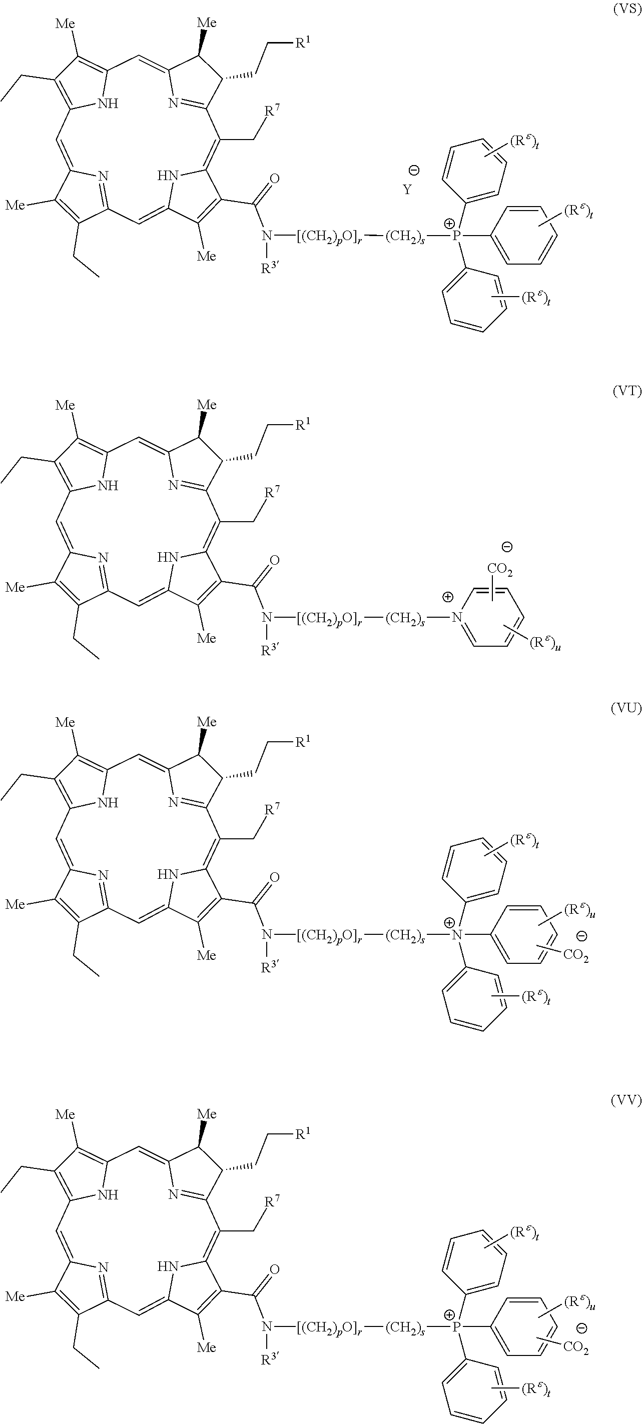

or a metal cation complex thereof, or a pharmaceutically acceptable salt thereof, wherein:
—R¹ is selected from —C(O)—OR³, —C(O)—SR³, —C(O)—N(R³)(R^{3′}), —C(S)—OR³, —C(S)—SR³ or —C(S)—N(R³)(R^{3′});
—R³, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, or —R^{α}—X;
—R^{3′} is selected from hydrogen or C₁-C₃ alkyl (preferably hydrogen or methyl);
—R⁶ is selected from —C(O)—OR³, —C(O)—SR³, —C(O)—N(R³)(R^{3′}), —C(S)—OR³, —C(S)—SR³ or —C(S)—N(R³)(R^{3′});
—R⁷ is selected from —C(O)—OR³, —C(O)—SR³, —C(O)—N(R³)(R^{3′}), —C(S)—OR³, —C(S)—SR³ or —C(S)—N(R³)(R^{3′});
—R^{α}—, each independently, is selected from a C₁-C₁₂ alkylene group, wherein the alkylene group may optionally be substituted with one or more (such as one, two, three, four or five) C₁-C₄ alkyl, C₁-C₄ haloalkyl or halo groups, and wherein one or more (such as one, two, three, four, five, six, seven, eight, nine or ten) carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more (such as one, two, three, four or five) heteroatoms N, O, S, P or Se in its carbon skeleton;
—R^{δ} is selected from C₁-C₃ alkyl;
—R^{ε} is selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
X is a halo group;
Y is a counter anion;
Z is a counter cation;
n is 1, 2, 3 or 4;
p is 0, 1, 2, 3 or 4;
r is 0, 1, 2, 3, 4, 5 or 6;
s is 0, 1, 2, 3, 4, 5, 6, 7, 8, 9, 10, 11 or 12;
t is 0, 1, 2, 3, 4 or 5; and
u is 0, 1, 2, 3 and 4.

The compounds of formula (VA), (VB), (VC), (VD), (VE), (VF), (VG), (VH), (VJ), (VK), (VL), (VM), (VN), (VO), (VP), (VQ), (VR), (VS), (VT), (VU), (VV) and complexes and salts thereof according to the first and second aspect of the present invention comprise a moiety —[(CH₂)ₚO]ᵣ—(CH₂)ₛ—, wherein:
p is 0, 1, 2, 3 or 4;
r is 0, 1, 2, 3, 4, 5 or 6; and
s is 0, 1, 2, 3, 4, 5, 6, 7, 8, 9, 10, 11 or 12.

In one embodiment, p is 2, 3 or 4; r is 1; and s is 2, 3 or 4. In a preferred embodiment, p is 3; r is 1; and s is 3; such that —[(CH₂)ₚO]ᵣ—(CH₂)ₛ is —(CH₂)₃—O—(CH₂)₃—.

In another embodiment, p is 2 or 3; r is 2 or 3; and s is 2 or 3. In a preferred embodiment, p is 2; r is 2; and s is 2; such that —[(CH₂)ₚO]ᵣ—(CH₂)ₛ— is —(CH₂CH₂O)₂—(CH₂)₂—.

In yet another embodiment, r is 0; and s is 1, 2, 3, 4, 5, 6, 7, 8, 9, 10, 11 or 12; such that —[(CH₂)ₚO]ᵣ—(CH₂)ₛ is —(CH₂)₁₋₁₂—.

Preferably in the compound or complex according to the first or second aspect of the present invention, the compound or complex is:

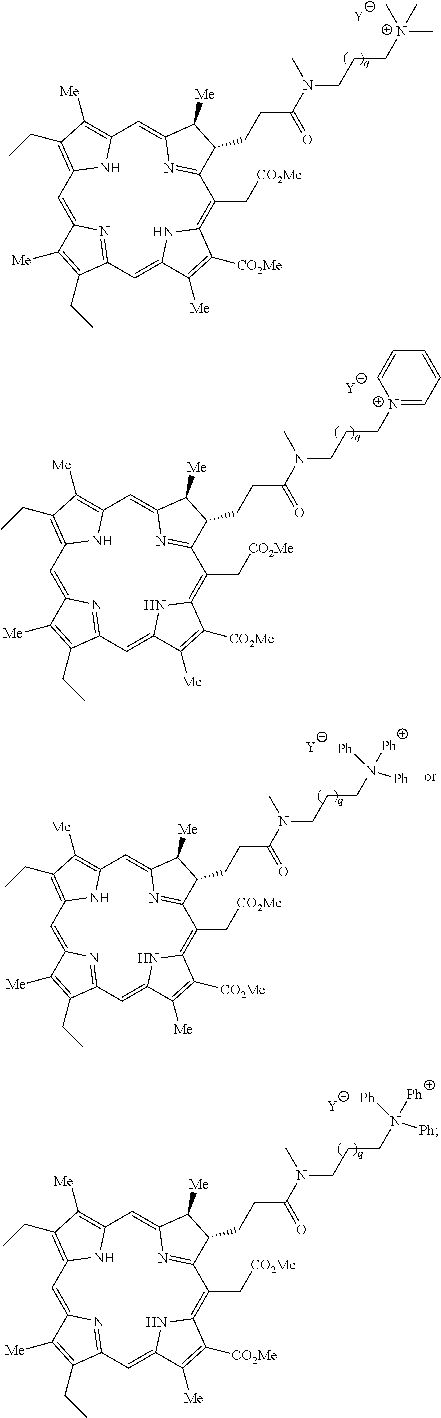

wherein Y is a counter anion, and q is 0, 1, 2, 3 or 4 (preferably q is 1);
or a metal cation complex thereof, or a pharmaceutically acceptable salt thereof.

In a particularly preferred embodiment, the first or second aspect of the present invention provides a compound of formula (VII) or a complex of formula (VIII):

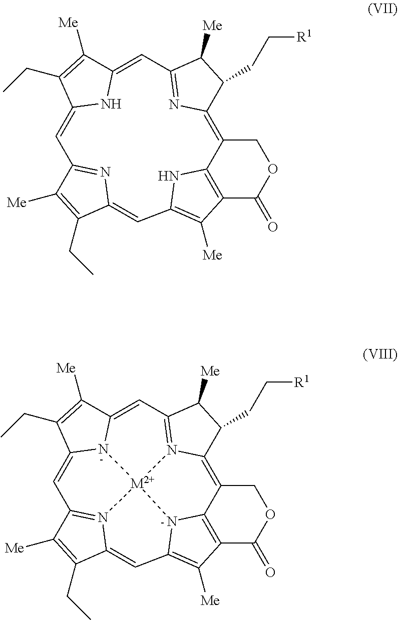

or a pharmaceutically acceptable salt thereof, wherein:
—R¹ is selected from —CH₂OR², —CH₂SR², —CH₂S(O)R², —CH₂S(O)₂R², —CH₂N(R²)(R^{2′}), —R², —C(O)—OR³, —C(O)—SR³, —C(O)—N(R³)(R^{3′}), —C(S)—OR³, —C(S)—SR³ or —C(S)—N(R³)(R^{3′}) [preferably —R¹ is selected from —C(O)—OR³, —C(O)—SR³, —C(O)—N(R³)(R^{3′}), —C(S)—OR³, —C(S)—SR³ or —C(S)—N(R³)(R^{3′}); more preferably —R¹ is —C(O)—N(R³)(R^{3′})];
—R² and —R³ are selected from —[(CH₂)ₚQ]ᵣ—(CH₂)ₛ—[N(R⁵)₃]Y, —[(CH₂)ₚQ]ᵣ—(CH₂)ₛ—[P(R⁵)₃]Y, —[(CH₂)ₚQ]ᵣ—(CH₂)ₛ—[R⁸]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{8′}];
—R^{2′} and —R^{3′} are selected from hydrogen or C₁-C₆ alkyl [preferably —R^{2′} and —R^{3′} are selected from hydrogen or C₁-C₃ alkyl; more preferably —R^{2′} and —R^{3′} are selected from hydrogen or methyl];
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂, wherein the phenyl or C₅-C₆ heteroaryl may optionally be further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁸ is —[NC₅H₅] optionally substituted with one or more (such as one, two, three, four or five) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{8′} is —[NC₅H₅] substituted with —CO₂ and optionally further substituted with one or more (such as one, two, three or four) C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
Q is O, S, NH or NMe [preferably Q is 0];
Y is a counter anion;
Z is a counter cation;
M²⁺ is a metal cation;
n is 1, 2, 3, 4, 5 or 6;
p is 0, 1, 2, 3 or 4;
r is 0, 1, 2, 3, 4, 5 or 6; and
s is 0, 1, 2, 3, 4, 5, 6, 7, 8, 9, 10, 11 or 12.

In a particularly preferred embodiment, the first or second aspect of the present invention provides a compound of formula (VII) or a complex of formula (VIII):

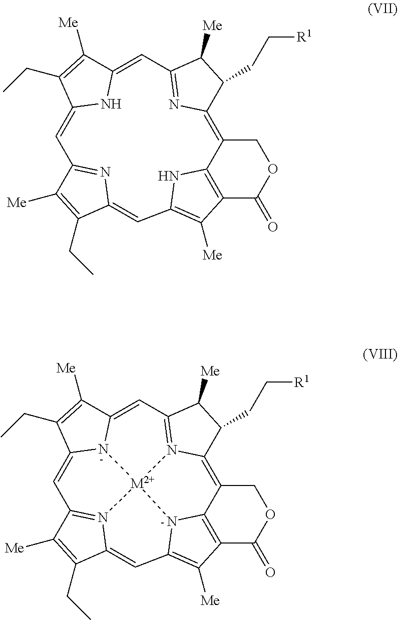

or a pharmaceutically acceptable salt thereof, wherein:
—R¹ is —C(O)—N(R³)(R^{3′});
—R³ is selected from —[(CH₂)ₚQ]ᵣ—(CH₂)ₛ [N(R⁵)₃]Y, —[(CH₂)ₚQ]ᵣ—(CH₂)ₛ [P(R⁵)₃]Y, —[(CH₂)ₚQ]ᵣ—(CH₂)ₛ—[R⁸]Y, —[(CH₂)ₚQ]ᵣ—(CH₂)ₛ—[N(R⁵)₂(R^{5′})], —[(CH₂)ₚQ]ᵣ—(CH₂)ₛ—[P(R⁵)₂(R^{5′})] or —[(CH₂)ₚQ]ᵣ—(CH₂)ₛ—[R^{8′}];
—R^{3′} is selected from hydrogen or C₁-C₃ alkyl (preferably hydrogen or methyl);
—R⁵, each independently, is selected from C₁-C₃ alkyl or phenyl, wherein the phenyl may optionally be substituted with one, two, three, four or five substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R^{5′} is selected from C₁-C₃ alkyl substituted with —CO₂ or phenyl substituted with —CO₂⁻, wherein the phenyl may optionally be further substituted with one, two, three or four substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R⁸ is —[NC₅H₅] optionally substituted with one, two, three, four or five substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
—R^{8′} is —[NC₅H₅] substituted with —CO₂ and optionally further substituted with one, two, three or four substituents independently selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
Q is O, S, NH or NMe (preferably O);
Y is a counter anion;
Z is a counter cation;
M²⁺ is a metal cation;
n is 1, 2, 3, 4, 5 or 6;
p is 0, 1, 2, 3 or 4;
r is 0, 1, 2, 3, 4, 5 or 6; and
s is 0, 1, 2, 3, 4, 5, 6, 7, 8, 9, 10, 11 or 12.

In these two preferred embodiments of the preceding paragraphs, each —R⁵ may be the same or different; preferably each —R⁵ is the same.

In another preferred embodiment of the first or second aspect of the present invention, the compound is a compound of formula (VIIA), (VIIB), (VIIC), (VIID), (VIIE), (VIIF) or (VIIG):

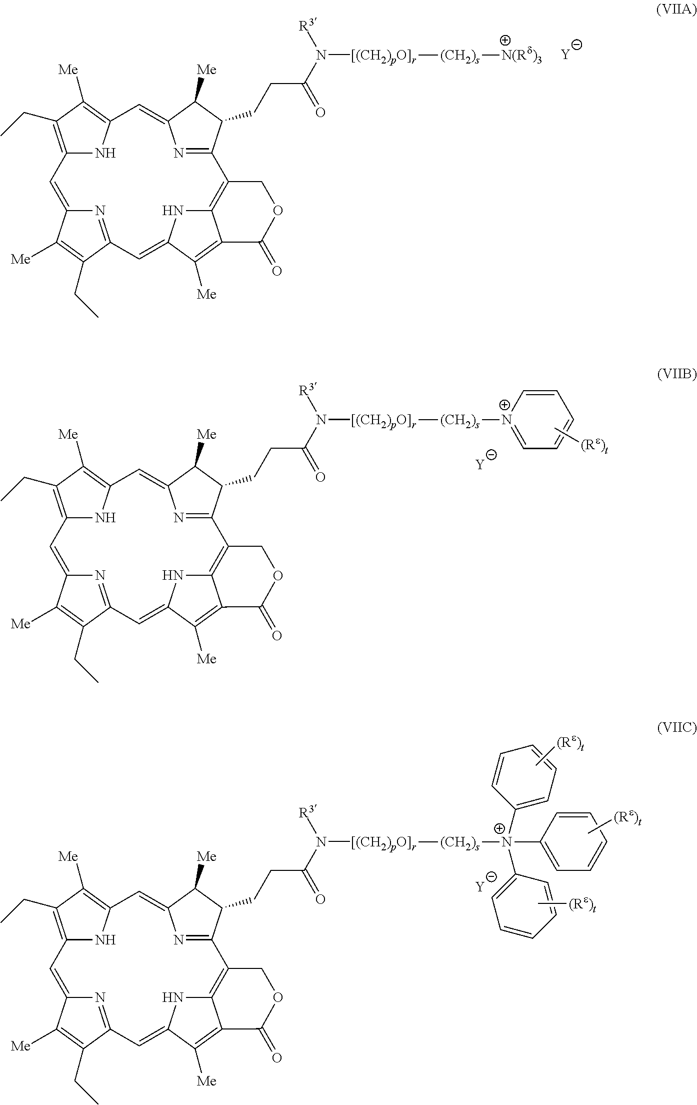

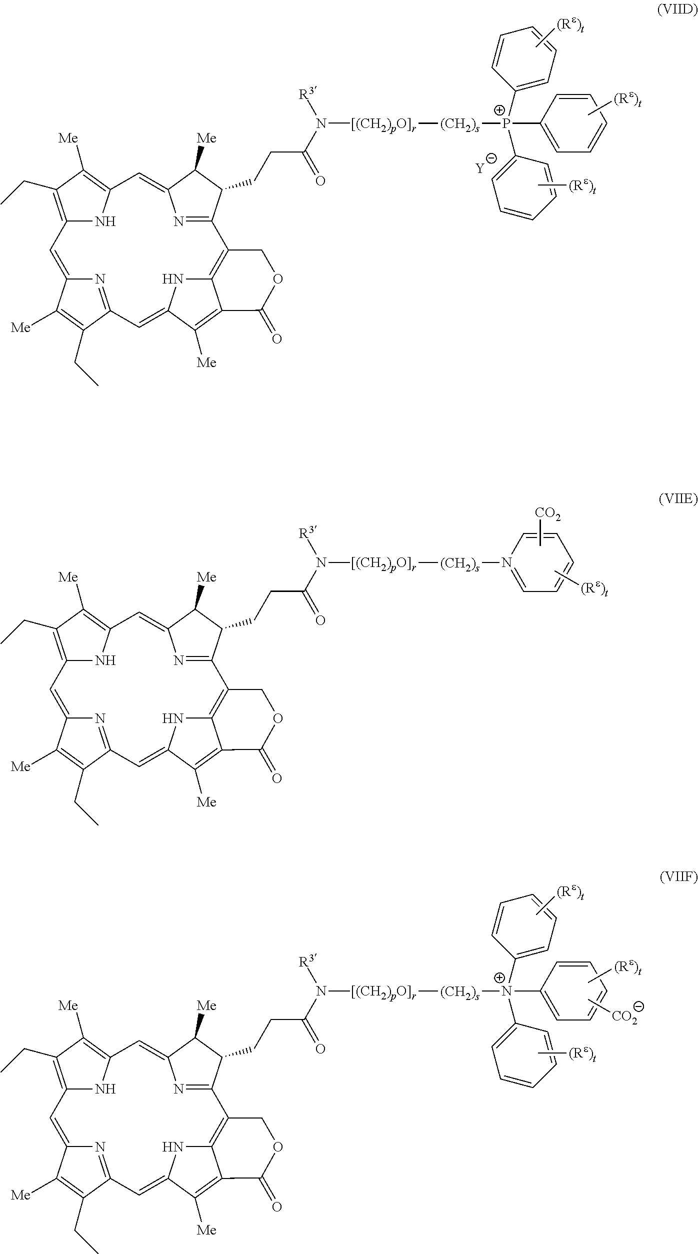

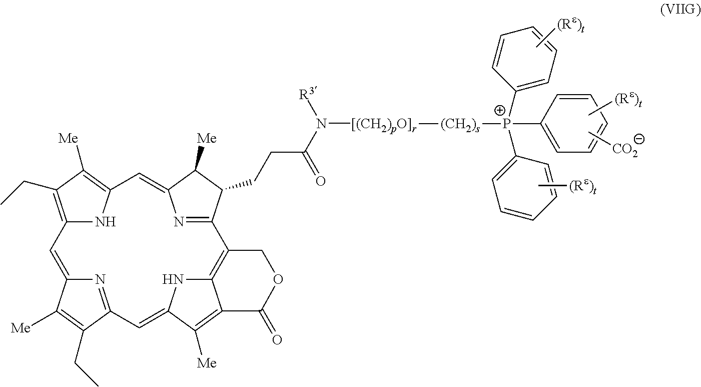

or a metal cation complex thereof, or a pharmaceutically acceptable salt thereof, wherein:
—R^{3′} is selected from hydrogen or C₁-C₃ alkyl (preferably hydrogen or methyl);
—R^{δ} is selected from C₁-C₃ alkyl;
—R^{ε} is selected from C₁-C₆ alkyl, —O(C₁-C₆ alkyl), —CO₂H, —CO₂Z, —CO₂NH₂, —O—(CH₂CH₂O)ₙ—H or —O—(CH₂CH₂O)ₙ—CH₃;
Y is a counter anion;
Z is a counter cation;
n is 1, 2, 3 or 4;
p is 0, 1, 2, 3 or 4;
r is 0, 1, 2, 3, 4, 5 or 6;
s is 0, 1, 2, 3, 4, 5, 6, 7, 8, 9, 10, 11 or 12;
t is 0, 1, 2, 3, 4 or 5; and
u is 0, 1, 2, 3 and 4.

The compounds of formula (VIIA), (VIIB), (VIIC), (VIID), (VIIE), (VIIF), (VIIG) and complexes and salts thereof according to the first and second aspect of the present invention comprise a moiety —[(CH₂)ₚO]ᵣ(CH₂)ₛ—, wherein:
p is 0, 1, 2, 3 or 4;
r is 0, 1, 2, 3, 4, 5 or 6; and
s is 0, 1, 2, 3, 4, 5, 6, 7, 8, 9, 10, 11 or 12.

In one embodiment, p is 2, 3 or 4; r is 1; and s is 2, 3 or 4. In a preferred embodiment, p is 3; r is 1; and s is 3; such that —[(CH₂)ₚO]ᵣ—(CH₂)ₛ is —(CH₂)₃—O—(CH₂)₃—.

In another embodiment, p is 2 or 3; r is 2 or 3; and s is 2 or 3. In a preferred embodiment, p is 2; r is 2; and s is 2; such that —[(CH₂)ₚO]ᵣ—(CH₂)ₛ— is —(CH₂CH₂O)₂—(CH₂)₂—.

In yet another embodiment, r is 0; and s is 1, 2, 3, 4, 5, 6, 7, 8, 9, 10, 11 or 12; such that —[(CH₂)ₚO]ᵣ—(CH₂)ₛ is —(CH₂)₁₋₁₂—.

Preferably in the compound or complex according to the first or second aspect of the present invention, the compound or complex is:

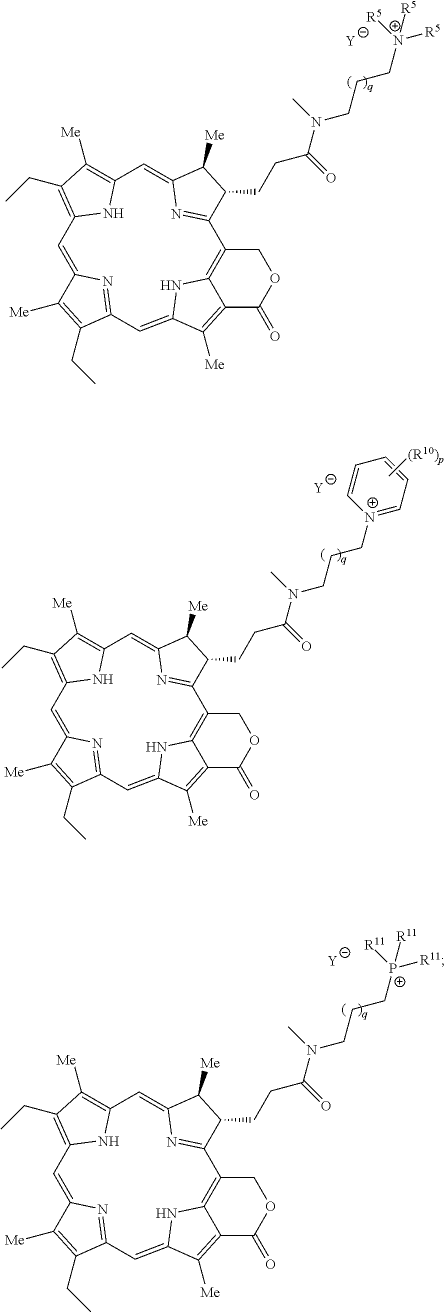

wherein
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
—R¹⁰, each independently, is selected from C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
—R¹¹, each independently, is phenyl optionally substituted with one or more C₁-C₄ alkyl, C₁-C₄ haloalkyl, —O(C₁-C₄ alkyl), —O(C₁-C₄ haloalkyl), halo, —O—(CH₂CH₂O)ₙ—H or —O—(CH₂CH₂O)ₙ—CH₃ groups;
n is 1, 2, 3 or 4;
p is 0, 1, 2, 3 or 4;
q is 0, 1, 2, 3 or 4 (preferably q is 1); and
Y is a counter anion;
   or a metal cation complex thereof, or a pharmaceutically acceptable salt thereof.

Preferably in the compound or complex according to the first or second aspect of the present invention, the compound or complex is:

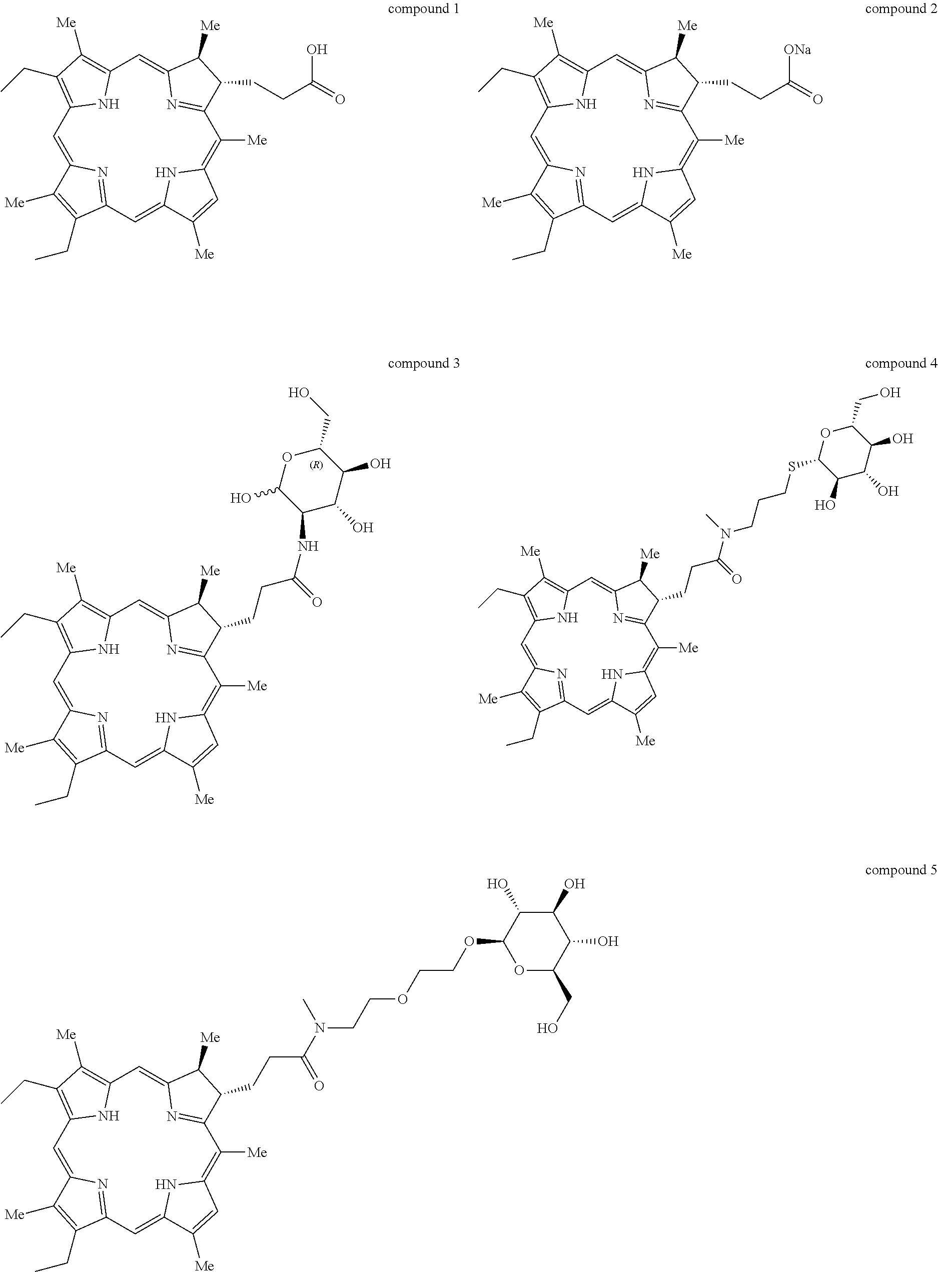

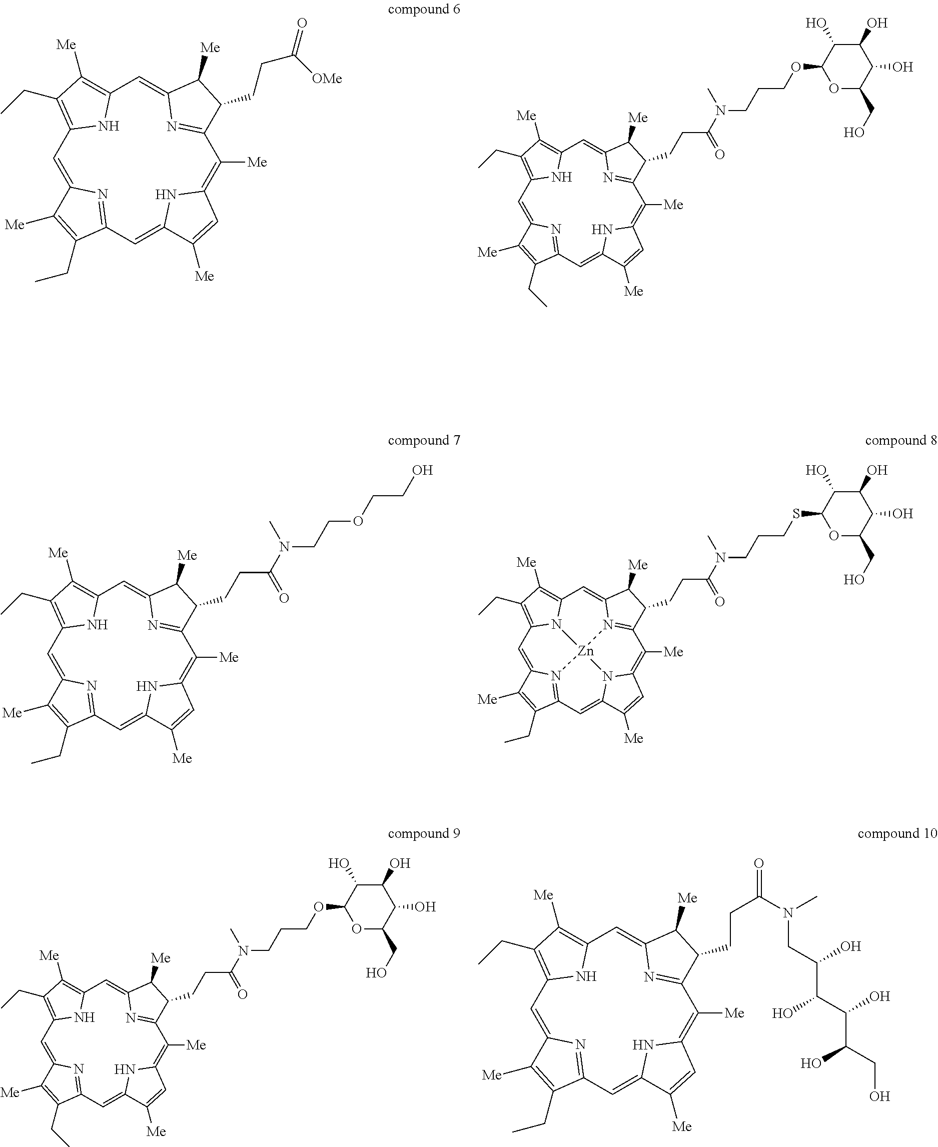

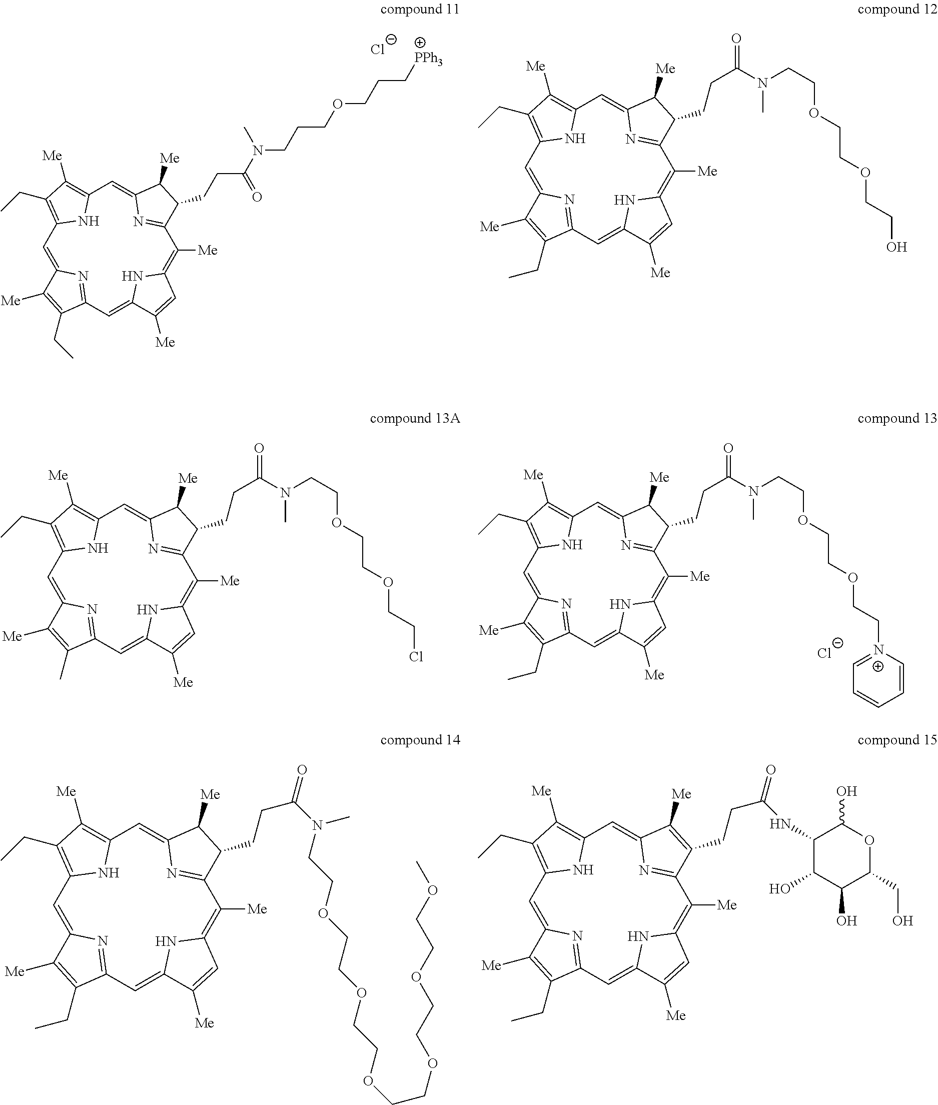

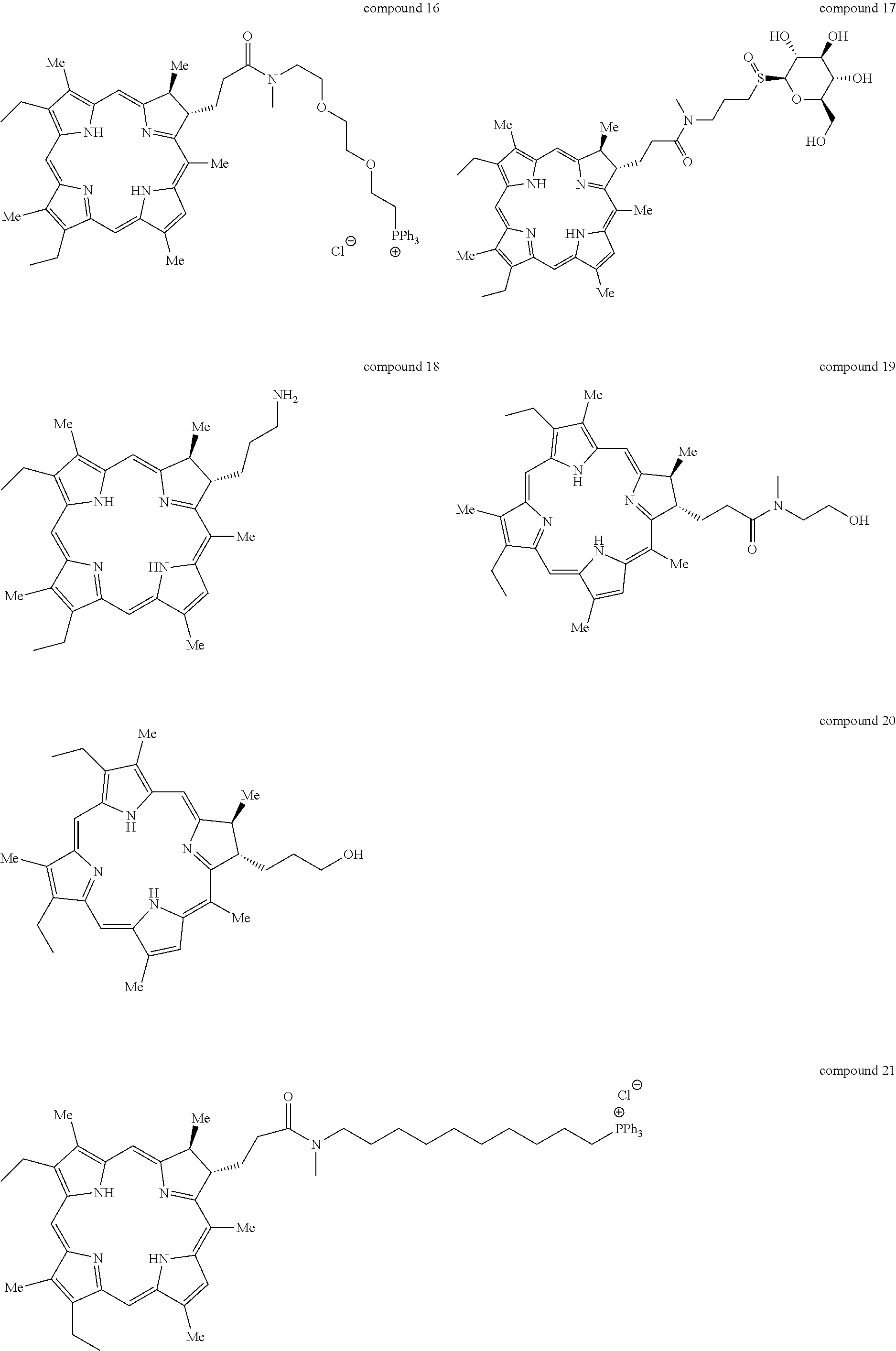

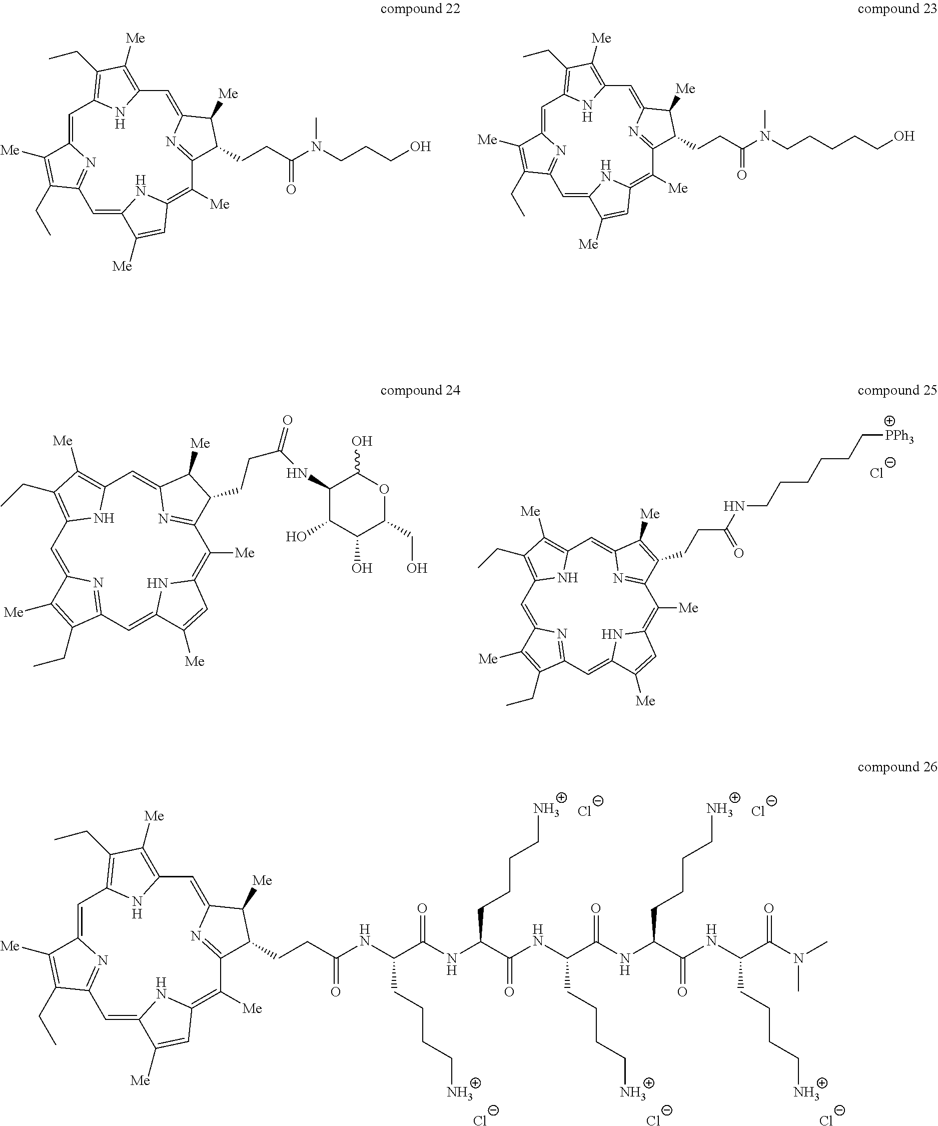

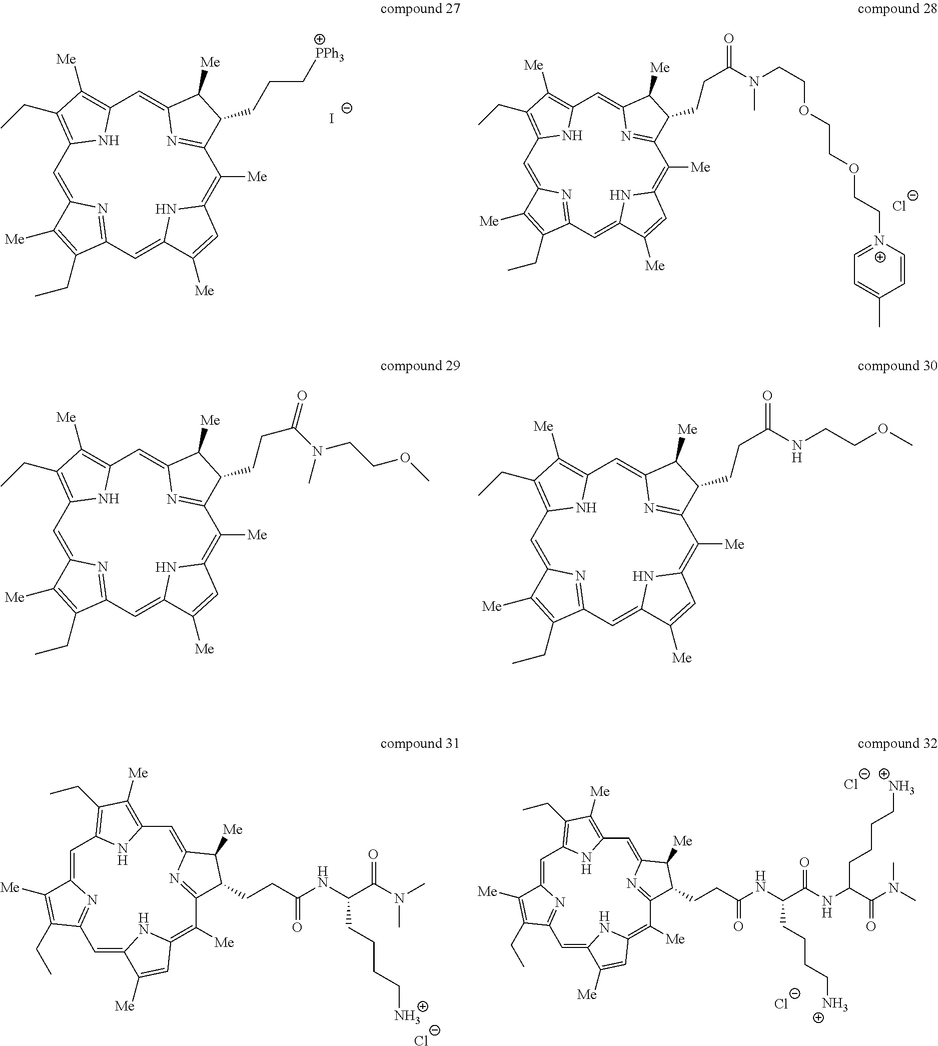

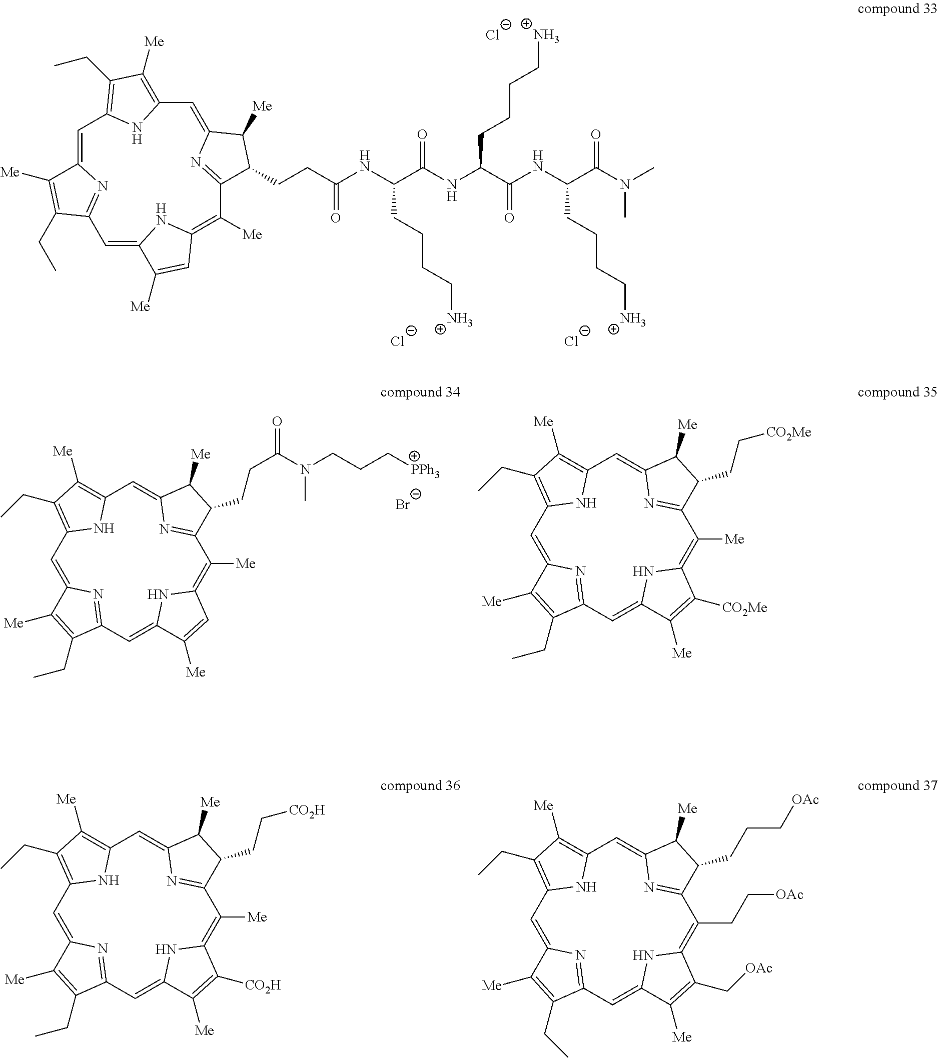

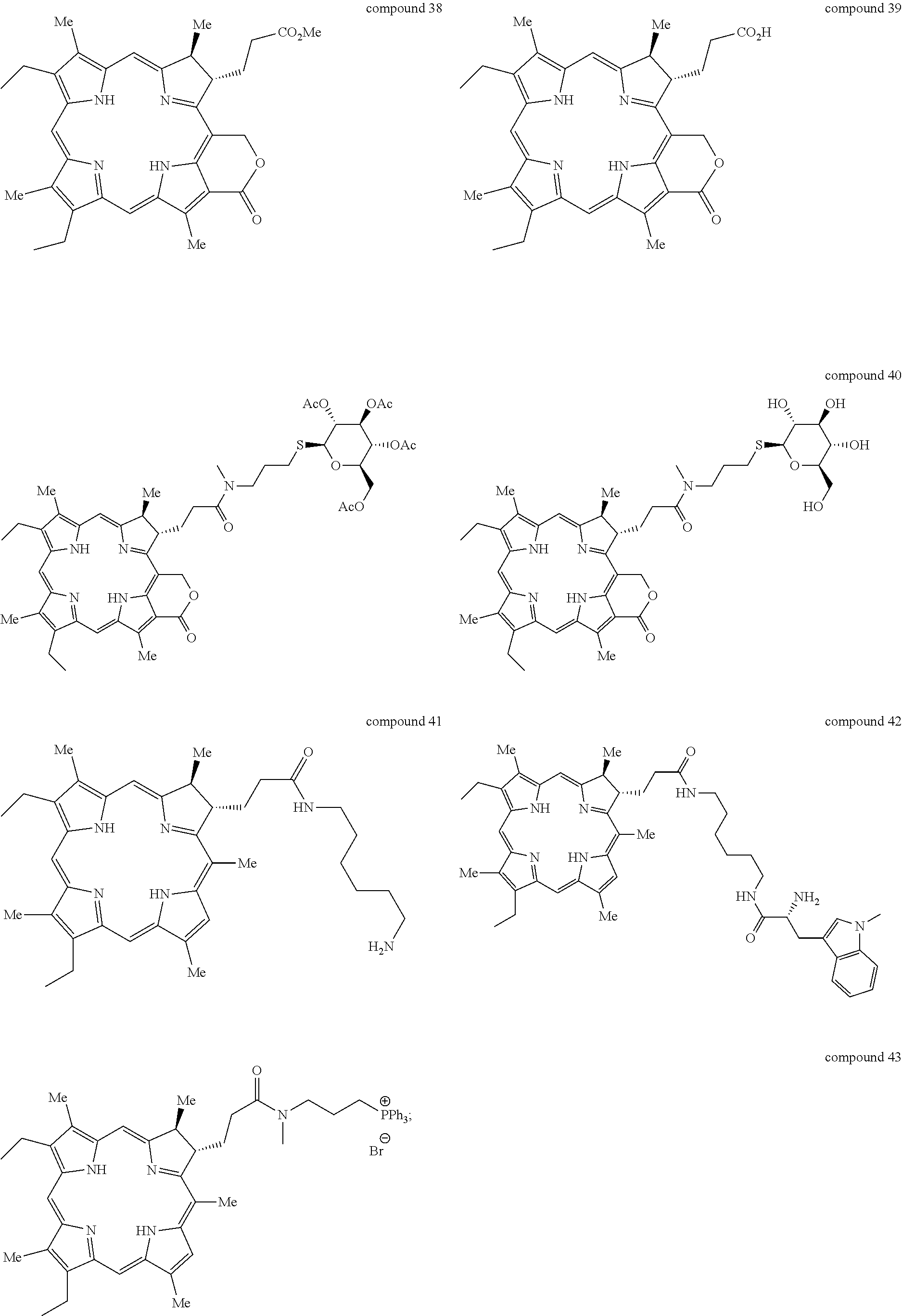

or a metal cation complex thereof, or a pharmaceutically acceptable salt thereof.

In one embodiment, the compound or complex according to the first or second aspect of the invention is in the form of a pharmaceutically acceptable salt. In one embodiment, the compound or complex is in the form of an inorganic salt such as a lithium, sodium, potassium, magnesium, calcium or ammonium salt. In one embodiment, the compound or complex is in the form of a sodium or potassium salt. In one embodiment, the compound is in the form of a sodium salt. In another embodiment, the compound or complex is in the form of an organic salt such as an amine salt (for example a choline or meglumine salt) or an amino acid salt (for example an arginine salt).

In one embodiment, the compound or complex according to the first or second aspect is reduced phyllochlorin in the form of a pharmaceutically acceptable salt. In one embodiment, the compound or complex is reduced phyllochlorin in the form of a pharmaceutically acceptable inorganic salt such as a lithium, sodium, potassium, magnesium, calcium or ammonium salt. In one embodiment, the compound or complex is reduced phyllochlorin mono-sodium or reduced phyllochlorin mono-potassium. In one embodiment, the compound or complex is reduced phyllochlorin mono-sodium.

The compound or complex according to the first or second aspect of the invention has at least two chiral centres. The compound or complex of the first or second aspect of the invention is preferably substantially enantiomerically pure, which means that the compound or complex comprises less than 10% of other stereoisomers, preferably less than 5%, preferably less than 3%, preferably less than 2%, preferably less than 1%, preferably less than 0.5%, all by weight, as measured by XRPD or SFC.

Preferably, the compound or complex according to the first or second aspect of the invention has a HPLC purity of more than 97%, more preferably more than 98%, more preferably more than 99%, more preferably more than 99.5%, more preferably more than 99.8%, and most preferably more than 99.9%. As used herein the percentage HPLC purity is measured by the area normalisation method.

A third aspect of the invention provides a composition comprising a compound or complex according to the first or second aspect of the invention and a pharmaceutically acceptable carrier or diluent.

In one embodiment, the composition according to the third aspect of the invention further comprises polyvinylpyrrolidone (PVP). In one embodiment, the composition comprises 0.01-10% w/w PVP as percentage of the total weight of the composition, preferably 0.1-5% w/w PVP as a percentage of the total weight of the composition, preferably 0.5-5% w/w PVP as a percentage of the total weight of the composition. In one embodiment, the PVP is K30.

In one embodiment, the composition according to the third aspect of the invention further comprises dimethylsulfoxide (DMSO). In one embodiment, the composition comprises 0.01-99% w/w DMSO as percentage of the total weight of the composition, preferably 40-99% w/w DMSO as a percentage of the total weight of the composition, preferably 65-99% w/w DMSO as a percentage of the total weight of the composition.

In one embodiment, the composition according to the third aspect of the invention further comprises an immune checkpoint inhibitor. In one embodiment, the immune checkpoint inhibitor is an inhibitor of PD-1 (programmed cell death protein 1), PD-L1 (programmed death ligand 1) or CTLA4 (cytotoxic T-lymphocyte associated protein 4). In one embodiment, the immune checkpoint inhibitor is selected from Pembrolizumab, Nivolumab, Cemiplimab, Atezolizumab, Avelumab, Durvalumab or Ipilimumab.

Preferably the compound or complex according to the first or second aspect of the present invention and the pharmaceutical composition according to the third aspect of the present invention are suitable for use in photodynamic therapy or cytoluminescent therapy.

Preferably the compound or complex according to the first or second aspect of the present invention and the pharmaceutical composition according to the third aspect of the present invention are suitable for the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the compound or complex according to the first or second aspect of the present invention and the pharmaceutical composition according to the third aspect of the present invention are suitable for the treatment of a disease characterised by benign or malignant cellular hyperproliferation or by areas of neovascularisation.

Preferably the compound or complex according to the first or second aspect of the present invention and the pharmaceutical composition according to the third aspect of the present invention are suitable for the treatment of a benign or malignant tumour.

Preferably the compound or complex according to the first or second aspect of the present invention and the pharmaceutical composition according to the third aspect of the present invention are suitable for the treatment of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the compound or complex according to the first or second aspect of the present invention and the pharmaceutical composition according to the third aspect of the present invention are suitable for use in photodynamic diagnosis.

Preferably the compound or complex according to the first or second aspect of the present invention and the pharmaceutical composition according to the third aspect of the present invention are suitable for the detection of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the compound or complex according to the first or second aspect of the present invention and the pharmaceutical composition according to the third aspect of the present invention are suitable for the detection of an area that is affected by benign or malignant cellular hyperproliferation or by neovascularisation.

Preferably the compound or complex according to the first or second aspect of the present invention and the pharmaceutical composition according to the third aspect of the present invention are suitable for the detection of a benign or malignant tumour.

Preferably the compound or complex according to the first or second aspect of the present invention and the pharmaceutical composition according to the third aspect of the present invention are suitable for the detection of early cancer; cervical dysplasia; soft tissue sarcoma; a germ cell tumour; retinoblastoma; age-related macular so degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the compound or complex according to the first or second aspect of the present invention and the pharmaceutical composition according to the third aspect of the present invention are suitable for the fluorescent or phosphorescent detection of the diseases listed above, preferably for the fluorescent or phosphorescent detection and quantification of the said diseases.

Preferably the compound or complex according to the first or second aspect of the present invention and the pharmaceutical composition according to the third aspect of the present invention are adapted for administration simultaneous with or prior to administration of irradiation or sound, preferably for administration prior to administration of irradiation.

If the compound or complex according to the first or second aspect of the present invention or the pharmaceutical composition according to the third aspect of the present invention are for use in photodynamic therapy or cytoluminescent therapy, then they are preferably adapted for administration 5 to 100 hours before the irradiation, preferably 6 to 72 hours before the irradiation, preferably 24 to 48 hours before the irradiation.

If the compound or complex according to the first or second aspect of the present invention or the pharmaceutical composition according to the third aspect of the present invention are for use in photodynamic diagnosis, then they are preferably adapted for administration 3 to 60 hours before the irradiation, preferably 8 to 40 hours before the irradiation.

Preferably the irradiation used in the photodynamic therapy, cytoluminescent therapy or photodynamic diagnosis is electromagnetic radiation with a wavelength in the range of from 500 nm to 1000 nm, preferably from 550 nm to 750 nm, preferably from 600 nm to 700 nm, preferably from 640 nm to 670 nm. The electromagnetic radiation may be administered for about 5-60 minutes, preferably for about 15-20 minutes, at about 0.1-5 W, preferably at about 1 W. In one embodiment of the present invention, two sources of electromagnetic radiation are used (for example a laser light and an LED light), both sources adapted to provide irradiation with a wavelength in the range of from 550 nm to 750 nm, preferably from 600 nm to 700 nm, preferably from 640 nm to 670 nm. In another embodiment of the present invention, the irradiation may be provided by a prostate, anal, vaginal, mouth and nasal device for insertion into a body cavity. In another embodiment of the present invention, the irradiation may be provided by interstitial light activation, for example, using a fine needle to insert an optical fibre laser into the lung, liver, lymph nodes or breast. In another embodiment of the present invention, the irradiation may be provided by endoscopic light activation, for example, for delivering light to the lung, stomach, colon, bladder or neck.

The pharmaceutical composition according to the third aspect of the present invention may be in a form suitable for oral, parenteral (including intravenous, subcutaneous, intramuscular, intradermal, intratracheal, intraperitoneal, intratumoral, intraarticular, intraabdominal, intracranial and epidural), transdermal, airway (aerosol), rectal, vaginal or topical (including buccal, mucosal and sublingual) administration. The pharmaceutical composition may also be in a form suitable for administration by enema or for administration by injection into a tumour. Preferably the pharmaceutical composition is in a form suitable for oral, parenteral (such as intravenous, intraperitoneal, and intratumoral) or airway administration, preferably in a form suitable for oral or parenteral administration, preferably in a form suitable for oral administration.

In one preferred embodiment, the pharmaceutical composition is in a form suitable for oral administration. Preferably the pharmaceutical composition is provided in the form of a tablet, capsule, hard or soft gelatine capsule, caplet, troche or lozenge, as a powder or granules, or as an aqueous solution, suspension or dispersion. More preferably the pharmaceutical composition is provided in the form of an aqueous solution, suspension or dispersion for oral administration, or alternatively in the form of a freeze-dried powder which can be mixed with water before administration to provide an aqueous solution, suspension or dispersion for oral administration. Preferably the pharmaceutical composition is in a form suitable for providing 0.01 to 10 mg/kg/day of the compound or complex according to the first or second aspect of the invention, preferably 0.1 to 2 mg/kg/day, preferably about 1 mg/kg/day.

In another preferred embodiment, the pharmaceutical composition is in a form suitable for parenteral administration. Preferably the pharmaceutical composition is in a form suitable for intravenous administration. Preferably the pharmaceutical composition is provided in the form of an aqueous solution for parenteral administration, or alternatively in the form of a freeze-dried powder which can be mixed with water before administration to provide an aqueous solution for parenteral administration.

Preferably the pharmaceutical composition is an aqueous solution or suspension having a pH of from 6 to 8.5. Preferably the pharmaceutical composition is in a form suitable for providing 0.01 to 10 mg/kg/day of the compound or complex according to the first or second aspect of the invention, preferably 0.1 to 2 mg/kg/day, preferably about 1 mg/kg/day.

In another preferred embodiment, the pharmaceutical composition is in a form suitable for airway administration. Preferably the pharmaceutical composition is provided in the form of an aqueous solution, suspension or dispersion for airway administration, or alternatively in the form of a freeze-dried powder which can be mixed with water before administration to provide an aqueous solution, suspension or dispersion for airway administration. Preferably the pharmaceutical composition is in a form suitable for providing 0.01 to 10 mg/kg/day of the compound or complex according to the first or second aspect of the invention, preferably 0.1 to 2 mg/kg/day, preferably about 1 mg/kg/day.

A fourth aspect of the present invention provides use of a compound or complex according to the first or second aspect of the present invention in the manufacture of a medicament for the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

The fourth aspect of the present invention also provides use of a compound or complex according to the first or second aspect of the present invention in the manufacture of a phototherapeutic agent for use in photodynamic therapy or cytoluminescent therapy. Preferably the phototherapeutic agent is suitable for the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the medicament or the phototherapeutic agent of the fourth aspect of the present invention is suitable for the treatment of a disease characterised by benign or malignant cellular hyperproliferation or by areas of neovascularisation.

Preferably the medicament or the phototherapeutic agent of the fourth aspect of the present invention is suitable for the treatment of a benign or malignant tumour.

Preferably the medicament or the phototherapeutic agent of the fourth aspect of the present invention is suitable for the treatment of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

The fourth aspect of the present invention also provides use of a compound or complex according to the first or second aspect of the present invention in the manufacture of a photodiagnostic agent for use in photodynamic diagnosis.

Preferably the photodiagnostic agent of the fourth aspect of the present invention is suitable for the detection of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the photodiagnostic agent of the fourth aspect of the present invention is suitable for the detection of an area that is affected by benign or malignant cellular hyperproliferation or by neovascularisation.

Preferably the photodiagnostic agent of the fourth aspect of the present invention is suitable for the detection of a benign or malignant tumour.

Preferably the photodiagnostic agent of the fourth aspect of the present invention is so suitable for the detection of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the photodiagnostic agent of the fourth aspect of the present invention is suitable for the fluorescent or phosphorescent detection of the said diseases, preferably the fluorescent or phosphorescent detection and quantification of the said diseases.

Preferably the medicament, the phototherapeutic agent or the photodiagnostic agent is adapted for administration simultaneous with or prior to administration of irradiation or sound, preferably for administration prior to administration of irradiation.

If the medicament or the phototherapeutic agent is for use in photodynamic therapy or cytoluminescent therapy, then it is preferably adapted for administration 5 to 100 hours before the irradiation, preferably 6 to 72 hours before the irradiation, preferably 24 to 48 hours before the irradiation.

If the photodiagnostic agent is for use in photodynamic diagnosis, then it is preferably adapted for administration 3 to 60 hours before the irradiation, preferably 8 to 40 hours before the irradiation.

Preferably the irradiation used in the photodynamic therapy, cytoluminescent therapy or photodynamic diagnosis is electromagnetic radiation with a wavelength in the range of from 500 nm to 1000 nm, preferably from 550 nm to 750 nm, preferably from 600 nm to 700 nm, preferably from 640 nm to 670 nm. The electromagnetic radiation may be administered for about 5-60 minutes, preferably for about 15-20 minutes, at about 0.1-5 W, preferably at about 1 W. In one embodiment of the present invention, two sources of electromagnetic radiation are used (for example a laser light and an LED light), both sources adapted to provide irradiation with a wavelength in the range of from 550 nm to 750 nm, preferably from 600 nm to 700 nm, preferably from 640 nm to 670 nm. In another embodiment of the present invention, the irradiation may be provided by a prostate, anal, vaginal, mouth and nasal device for insertion into a body cavity. In another embodiment of the present invention, the irradiation may be provided by interstitial light activation, for example, using a fine needle to insert an optical fibre laser into the lung, liver, lymph nodes or breast. In another embodiment of the present invention, the irradiation may be provided by endoscopic light activation, for example, for delivering light to the lung, stomach, colon, bladder or neck.

A fifth aspect of the present invention provides a method of treating atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; so early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas; the method comprising administering a therapeutically effective amount of a compound or complex according to the first or second aspect of the present invention to a human or animal in need thereof.

The fifth aspect of the present invention also provides a method of photodynamic therapy or cytoluminescent therapy of a human or animal disease, the method comprising administering a therapeutically effective amount of a compound or complex according to the first or second aspect of the present invention to a human or animal in need thereof. Preferably the human or animal disease is atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid so artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

Preferably the method of the fifth aspect of the present invention is a method of treating benign or malignant cellular hyperproliferation or areas of neovascularisation.

Preferably the method of the fifth aspect of the present invention is a method of treating a benign or malignant tumour.

Preferably the method of the fifth aspect of the present invention is a method of treating early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

The fifth aspect of the present invention also provides a method of photodynamic diagnosis of a human or animal disease, the method comprising administering a diagnostically effective amount of a compound or complex according to the first or second aspect of the present invention to a human or animal. Preferably the human or animal disease is atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas. Preferably the human or animal disease is characterised by benign or malignant cellular hyperproliferation or by areas of neovascularisation. Preferably the human or animal disease is a benign or malignant tumour. Preferably the human or animal disease is early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas. Preferably the method of photodynamic diagnosis is suitable for the fluorescent or phosphorescent detection of the said diseases, preferably for the fluorescent or phosphorescent detection and quantification of the said diseases.

In any of the methods of the fifth aspect of the present invention, the human or animal is preferably further subjected to irradiation or sound simultaneous with or after the administration of the compound or complex according to the first or second aspect of the invention. Preferably the human or animal is subjected to irradiation after the administration of the compound or complex according to the first or second aspect of the invention.

If the method is a method of photodynamic therapy or cytoluminescent therapy, then the human or animal is preferably subjected to irradiation 5 to 100 hours after administration of the compound or complex according to the first or second aspect of the invention, preferably 6 to 72 hours after administration, preferably 24 to 48 hours after administration.

If the method is a method of photodynamic diagnosis, then the human or animal is preferably subjected to irradiation 3 to 60 hours after administration of the compound or complex according to the first or second aspect of the invention, preferably 8 to 40 hours after administration.

Preferably the irradiation is electromagnetic radiation with a wavelength in the range of from 500 nm to 1000 nm, preferably from 550 nm to 750 nm, preferably from 600 nm to 700 nm, preferably from 640 nm to 670 nm. The electromagnetic radiation may be administered for about 5-60 minutes, preferably for about 15-20 minutes, at about 0.1-5 W, preferably at about 1 W. In one embodiment of the present invention, two sources of electromagnetic radiation are used (for example a laser light and an LED light), both sources adapted to provide irradiation with a wavelength in the range of from 550 nm to 750 nm, preferably from 600 nm to 700 nm, preferably from 640 nm to 670 nm. In another embodiment of the present invention, the irradiation may be provided by a prostate, anal, vaginal, mouth and nasal device for insertion into a body cavity. In another embodiment of the present invention, the irradiation may be provided by interstitial light activation, for example, using a fine needle to insert an optical fibre laser into the lung, liver, lymph nodes or breast. In another embodiment of the present invention, the irradiation may be provided by endoscopic light activation, for example, for delivering light to the lung, stomach, colon, bladder or neck.

In any of the methods of the fifth aspect of the present invention, preferably the human or animal is a human.

A sixth aspect of the present invention provides a pharmaceutical combination or kit comprising:
(a) a compound or complex according to the first or second aspect of the present invention; and
(b) an immune checkpoint inhibitor.

In one embodiment, the immune checkpoint inhibitor is an inhibitor of PD-1 (programmed cell death protein 1), PD-L1 (programmed death ligand 1) or CTLA4 (cytotoxic T-lymphocyte associated protein 4). In one embodiment, the immune checkpoint inhibitor is selected from Pembrolizumab, Nivolumab, Cemiplimab, Atezolizumab, Avelumab, Durvalumab or Ipilimumab.

Preferably, the combination or kit of the sixth aspect is for use in the treatment of a disease, disorder or condition, wherein the disease, disorder or condition is responsive to PD-1, PD-L1 or CTLA4 inhibition. Preferably, the combination or kit of the sixth aspect is for use in the treatment of cancer. In one embodiment, the cancer is melanoma, lung cancer (e.g. non small cell lung cancer), kidney cancer, bladder cancer, head and neck cancer, or Hodgkin's lymphoma.

The sixth aspect also provides a use of the combination or kit of the sixth aspect of the invention in the manufacture of a medicament for the treatment of a disease, disorder or condition which is responsive to PD-1, PD-L1 or CTLA4 inhibition. The sixth aspect also provides a use of the combination or kit of the sixth aspect of the invention in the manufacture of a medicament for the treatment of cancer. In one embodiment, the cancer is melanoma, lung cancer (e.g. non small cell lung cancer), kidney cancer, bladder cancer, head and neck cancer, or Hodgkin's lymphoma.

The sixth aspect of the invention also provides a method of treating a disease, disorder or condition which is responsive to PD-1, PD-L1 or CTLA4 inhibition, the method comprising administering a therapeutically effective amount of the combination or kit of the sixth aspect of the present invention to a human or animal in need thereof. The sixth aspect of the invention also provides a method of treating cancer, the method comprising administering a therapeutically effective amount of the combination or kit of the sixth aspect of the present invention to a human or animal in need thereof. In one embodiment, the cancer is melanoma, lung cancer (e.g. non small cell lung cancer), kidney cancer, bladder cancer, head and neck cancer, or Hodgkin's lymphoma.

For the combination or kit of the sixth aspect of the invention, the compound or complex according to the first or second aspect of the invention, and the immune checkpoint inhibitor may be provided together in one pharmaceutical composition or separately in two pharmaceutical compositions. If provided in two pharmaceutical compositions, these may be administered at the same time or at different times.

Preferably the combination or kit of the sixth aspect is adapted for administration simultaneous with or prior to administration of irradiation or sound, preferably for administration prior to administration of irradiation. In one embodiment, the combination or kit of the sixth aspect is adapted for administration 5 to 100 hours before the irradiation, preferably 6 to 72 hours before the irradiation, preferably 24 to 48 hours before the irradiation.

Preferably the irradiation used in the photodynamic therapy or cytoluminescent therapy is electromagnetic radiation with a wavelength in the range of from 500 nm to 1000 nm, preferably from 550 nm to 750 nm, preferably from 600 nm to 700 nm, preferably from 640 nm to 670 nm. The electromagnetic radiation may be administered for about 5-60 minutes, preferably for about 15-20 minutes, at about 0.1-5 W, preferably at about 1 W. In one embodiment of the present invention, two sources of electromagnetic radiation are used (for example a laser light and an LED light), both sources adapted to provide irradiation with a wavelength in the range of from 550 nm to 750 nm, preferably from 600 nm to 700 nm, preferably from 640 nm to 670 nm. In another embodiment of the present invention, the irradiation may be provided by a prostate, anal, vaginal, mouth and nasal device for insertion into a body cavity. In another embodiment of the present invention, the irradiation may be provided by interstitial light activation, for example, using a fine needle to insert an optical fibre laser into the lung, liver, lymph nodes or breast. In another embodiment of the present invention, the irradiation may be provided by endoscopic light activation, for example, for delivering light to the lung, stomach, colon, bladder or neck.

For the avoidance of doubt, insofar as is practicable any embodiment of a given aspect of the present invention may occur in combination with any other embodiment of the same aspect of the present invention. In addition, insofar as is practicable it is to be understood that any preferred or optional embodiment of any aspect of the present invention should also be considered as a preferred or optional embodiment of any other aspect of the present invention.

### SYNTHETIC EXPERIMENTAL DETAILS

### Synthesis Example 1—Synthesis of Reduced Phyllochlorin (Compound 1)

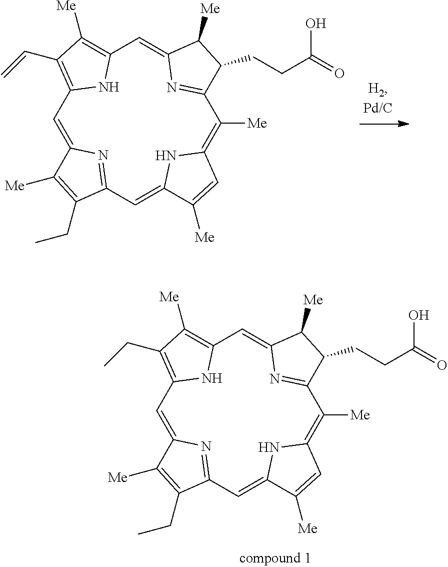 [SMILES: C=Cc1c(C)c2cc3nc(c(C)c4cc(C)c(cc5nc(cc1[nH]2)C(C)=C5CC)[nH]4)[C@@H](CCC(=O)O)[C@@H]3C ; CCC1=C(C)c2cc3[nH]c(cc4nc(c(C)c5cc(C)c(cc1n2)[nH]5)[C@@H](CCC(=O)O)[C@@H]4C)c(C)c3CC]

A 3-neck 250 mL RBF was charged with phyllochlorin (4.0 g, 7.86 mmol, 1 eq), 10% Pd/C (200 mg), ethyl acetate (200 mL), methanol (40 mL) and a stirrer bar (˜25 mm).

A hydrogen balloon was connected to the middle joint of the flask via a short length air condenser and the side-arm was connected to a 3-way tap. The setup was evacuated and then re-filled with nitrogen (3 times), evacuated and re-filled with hydrogen. The resulting solution was stirred under the hydrogen atmosphere with heating at 35° C. overnight. A sample of the mixture was removed, filtered through cotton wool, concentrated under reduced pressure and analysed by HPLC, which showed that the reaction was complete. The solution was filtered through Celite® (1×4 cm), the Celite® pad washed with methanol (˜10 mL), and the solvent removed under reduced pressure to give the crude product, which was purified using column chromatography (silica, 4×25 cm) eluting with 2-4% MeOH/DCM to provide compound 1 as a metallic blue/green flaky solid (2.35 g, 59%) (HPLC purity: 98.8%).

¹H NMR (400 MHz, CDCl₃) δ 9.74 (s, 1H), 9.56 (s, 1H), 8.79 (m, 2H), 4.60-4.55 (m, 1H), 4.50-4.45 (m, 1H), 3.97-3.90 (m, 5H), 3.85 (q, 2H), 3.62 (s, 3H), 3.39 (s, 3H), 3.37 (s, 3H), 2.55-2.30 (m, 2H), 2.12-1.95 (m, 2H), 1.79-1.70 (m, 9H), −2.25 (br s, 1H).

### Synthesis Example 2—Synthesis of Reduced Phyllochlorin Sodium Salt (Compound 2)

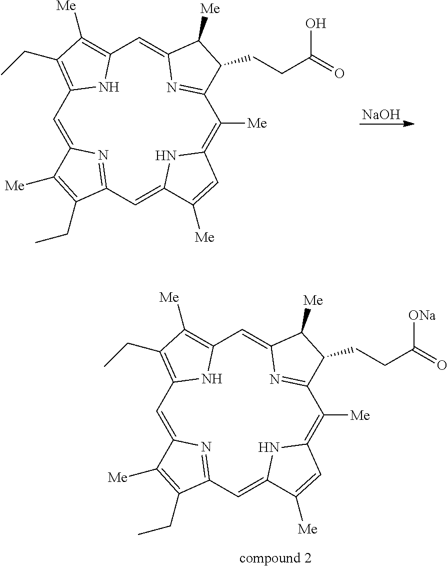

To a 1-neck 25 mL RBF containing reduced phyllochlorin (compound 1) (50 mg, 0.098 mmol, 1 eq) and a small stirrer bar was added water (1.5 mL) and the suspension stirred at 400 rpm. Sodium hydroxide (0.103 M, 0.86 mL, 0.088 mmol, 0.9 eq) was added and the mixture stirred for 15 minutes under nitrogen. The solution was then sonicated for 15 minutes, by which time most of the solid appeared to be soluble. The solution was stirred, under nitrogen, for a further 2 hours. The solvent was then removed using a rotary evaporator and the product dried on a rotary evaporator at 50° C. on full vacuum for 1 hour to provide compound 2 as a dark green/blue solid (37 mg, 71%) (HPLC purity: 94.8%).

¹H NMR (400 MHz, DMSO-d₆) δ 9.71 (s, 1H), 9.58 (s, 1H), 8.98 (m, 2H), 4.63-4.52 (m, 2H), 3.97-3.90 (m, 5H), 3.81 (q, 2H), 3.60 (s, 3H), 3.37 (s, 3H), 3.32 (s, 3H), 2.47-2.37 (m, 2H), 2.11-2.04 (m, 1H), 1.84-1.75 (m, 1H), 1.72-1.66 (m, 9H), −2.34 (brs, 1H), −2.39 (brs, 1H).

### Synthesis Example 3—Synthesis of Reduced Phyllochlorin 2-deoxyglucosamine-propylamide (Compound 3)

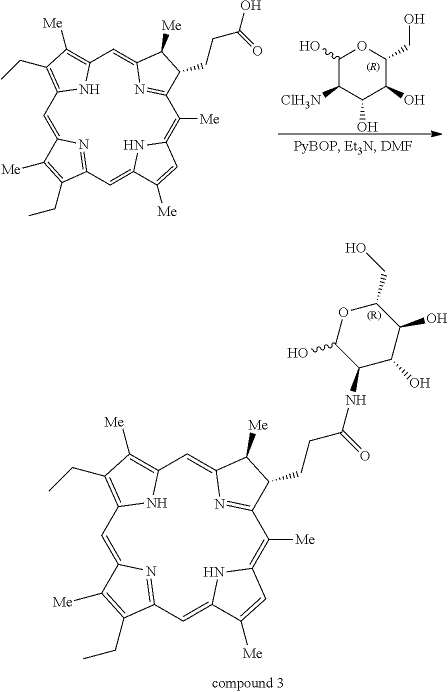

To a 10 mL RBF containing a stirrer bar was added reduced phyllochlorin (compound 1) (105.0 mg, 0.2056 mmol, 1 eq), PyBOP (117.7 mg, 0.2262 mmol, 1.1 eq), DMF (1 mL) and triethylamine (62.7 μL, 0.4523 mmol, 2.2 eq). The resultant mixture was stirred (420 rpm) under nitrogen at ambient temperature for 5 minutes, then a suspension of (D)-glucosamine hydrochloride (48.8 mg, 0.2262 mmol, 1.1 eq) in DMF (0.5 mL) was added in one portion with further DMF (0.5 mL) used for quantitative transfer. The resultant mixture was stirred for 30 minutes, by which time the reaction was complete, as monitored by HPLC. The reaction mixture was concentrated using a rotary evaporator to obtain a black residue. The crude product was purified by column chromatography using a stepped column (27/40 mm), a silica height of 30 cm and a gradient of 10%→15%→20% MeOH/DCM to give a black solid (158.1 mg) containing compound 3 and a triethylammonium impurity. The solid was slurried in distilled water (3 mL) overnight, collected on a sinter and washed with distilled water (2×3 mL). Drying in a vacuum oven at 60° C. for 2 hours gave compound 3 as a black solid (96.9 mg, 70%) (HPLC purity: 95.4%).

¹H NMR (400 MHz, DMSO-d₆) δ 9.74 (s, 1H), 9.60 (s, 1H), 9.00 (m, 2H), 5.24-4.90 (m, 3H), 4.67-4.45 (m, 3H), 4.28 (dd, 1H), 3.98 (m, 5H), 3.82 (q, 2H), 3.62 (s, 3H), 3.40 (s, 3H), 3.22-3.15 (m, 2H), 3.14-3.04 (m, 3H), 2.90 (s, 2H), 2.81 (s, 1H), 2.70-2.52 (m, 2H), 2.48-2.35 (m, 1H), 1.81 (m, 1H), 1.76-1.63 (m, 11H), −2.35 (s, 1H), −2.40 (s, 1H).

The product in solution exists as a mixture of epimers which causes two sets of signals in an ˜2:1 ratio.

### Synthesis Example 4—Synthesis of Reduced Phyllochlorin β-D-1-thioglucose-N-methylpropylamide (Compound 4)

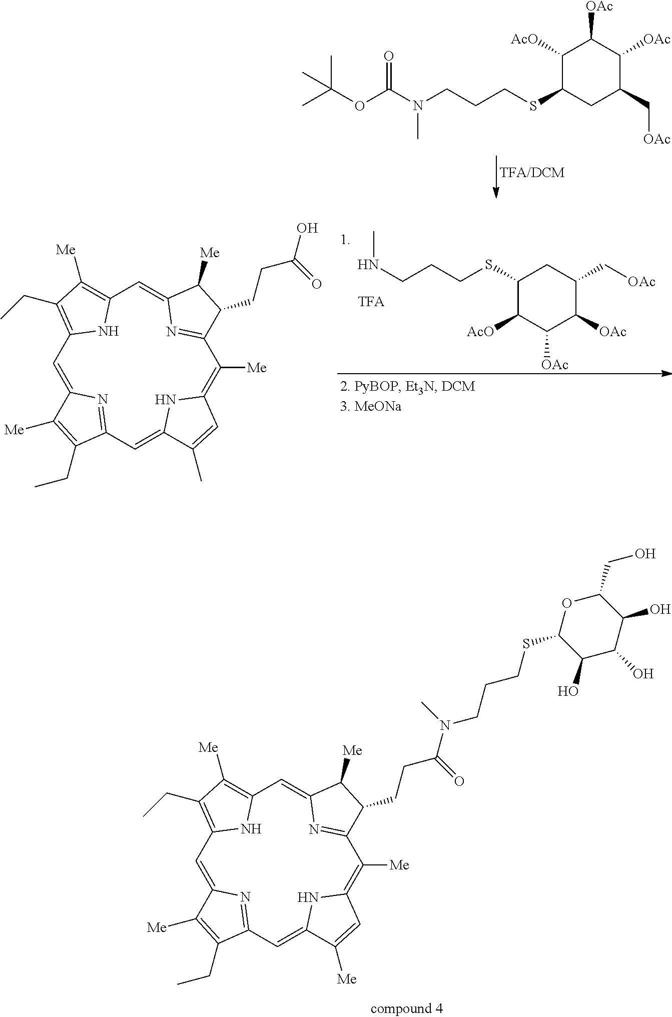 [SMILES: CC(=O)OC[C@H]1C[C@@H](SCCCN(C)C(=O)OC(C)(C)C)[C@H](OC(C)=O)[C@@H](OC(C)=O)[C@@H]1OC(C)=O ; CCC1=C(C)c2cc3[nH]c(cc4nc(c(C)c5cc(C)c(cc1n2)[nH]5)[C@@H](CCC(=O)O)[C@@H]4C)c(C)c3CC ; CNCCCS[C@@H]1C[C@H](COC(C)=O)[C@@H](OC(C)=O)[C@H](OC(C)=O)[C@H]1OC(C)=O ; CCC1=C(C)c2cc3[nH]c(cc4nc(c(C)c5cc(C)c(cc1n2)[nH]5)[C@@H](CCC(=O)N(C)CCCS[C@@H]1O[C@H](CO)[C@@H](O)[C@H](O)[C@H]1O)[C@@H]4C)c(C)c3CC]

Step 1: To a solution of (2R,3R,4S,5R,6S)-2-(acetoxymethyl)-6-((3-((tert-butoxycarbonyl)(methyl)amino)propyl)thio)tetrahydro-2H-pyran-3,4,5-triyl triacetate (274 mg, 0.511 mmol, 1.3 eq) in DCM (4 mL) was added TFA (1 mL). The resultant solution was stirred (420 rpm) for 1 hour at ambient temperature, then concentrated on a rotary evaporator. The residue was resuspended and concentrated twice from chloroform (2×5 mL) to give a viscous oil that was dissolved in DCM (2 mL) for the subsequent coupling reaction.

Step 2: To a 25 mL RBF was added reduced phyllochlorin (compound 1) (200 mg, 0.393 mmol, 1 eq), PyBOP (246 mg, 0.472 mmol, 1.2 eq), DCM (2 mL) and triethylamine (327 μL, 2.36 mmol, 6 eq). The resultant mixture was stirred (420 rpm) under nitrogen at ambient temperature for 30 minutes, then the solution of the deprotected amine in DCM (2 mL) prepared in step 1 was added in one portion, washing with further DCM (18 mL). After stirring the resultant mixture for 30 minutes, the reaction was complete, as monitored by HPLC. The reaction mixture was transferred to a separatory funnel and washed with 1M HCl (2×5 mL), then pH 7 buffer (1×10 mL). The organic phase was dried (Na₂SO₄) and concentrated by rotary evaporation to give a bubbly blue-black film. The residue was purified by silica column chromatography (3×22 cm). The crude product was dissolved in 1.5% MeOH/DCM and eluted using a gradient of 1.5%→3% MeOH/DCM to give reduced phyllochlorin β-D-1-thioglucose-N-methylpropylamide peracetate as a blue/black flaky solid (320 mg).

Step 3: To a solution of reduced phyllochlorin β-D-1-thioglucose-N-methylpropylamide peracetate (310 mg, 0.334 mmol, 1 eq) in MeOH (5 mL) and DCM (5 mL) was added NaOMe (4.6M in MeOH, 0.36 mL, 1.670 mmol, 5 eq), and the mixture stirred (420 rpm) under nitrogen for 1 hour. TLC analysis showed conversion to the deacetylated product (10% MeOH/DCM, R_{f} (starting material)=0.80, R_{f} (product)=0.30). The reaction was quenched with AcOH (100 mg, 5 eq) and concentrated by rotary evaporation to give a black film. The residue was purified by silica column chromatography (3×21 cm). The crude product was dissolved in 5% MeOH/DCM and eluted using a gradient of 5%→7%→8%→10% MeOH/DCM to give compound 4 as a blue/black flaky solid (213 gm).

¹H NMR (400 MHz, DMSO-d₆) δ 9.74 (s, 1H), 9.60 (s, 1H), 9.00 (m, 2H), 5.24-4.90 (m, 3H), 4.67-4.45 (m, 3H), 4.28 (dd, 1H), 3.98 (m, 5H), 3.82 (q, 2H), 3.62 (s, 3H), 3.40 (s, 3H), 3.22-3.15 (m, 2H), 3.14-3.04 (m, 3H), 2.90 (s, 2H), 2.81 (s, 2H), 2.75-2.50 (m, 3H), 2.48-2.30 (m, 3H), 1.81 (m, 1H), 1.76-1.63 (m, 11H), −2.35 (s, 1H), −2.40 (s, 1H).

Alternatively reduced phyllochlorin β-D-1-thioglucose-N-methylpropylamide (compound 4) can be prepared as follows:

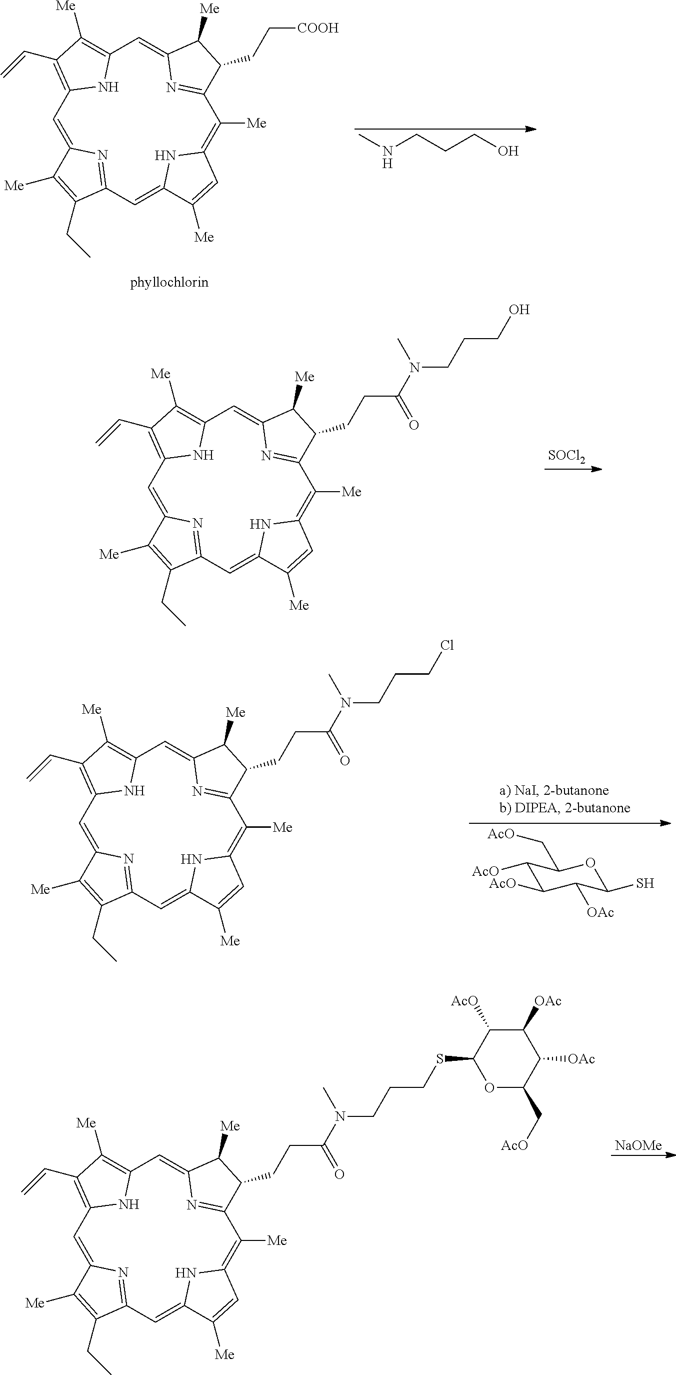

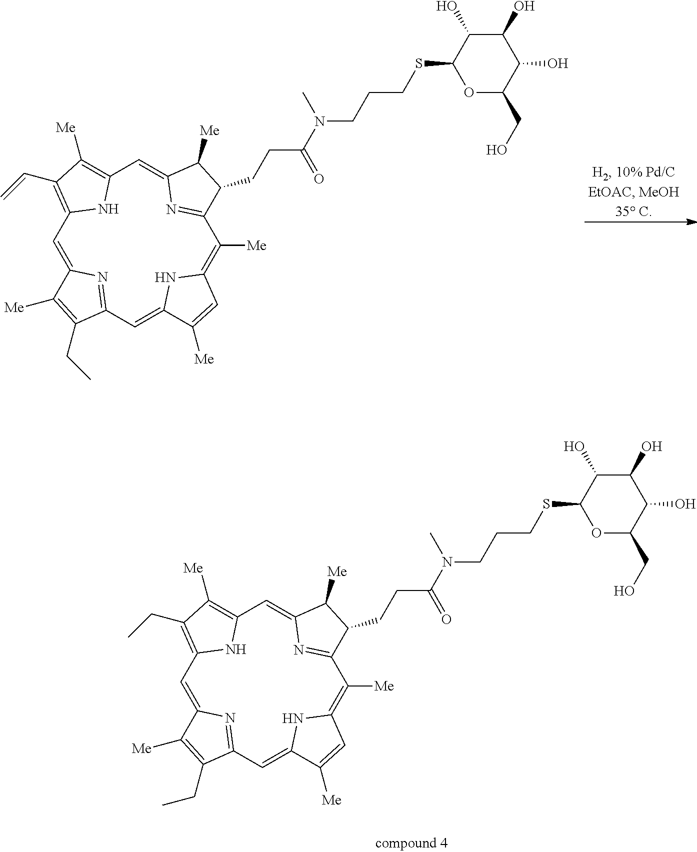 [SMILES: C=Cc1c(C)c2cc3nc(c(C)c4cc(C)c(cc5nc(cc1[nH]2)C(C)=C5CC)[nH]4)[C@@H](CCC(=O)N(C)CCCS[C@@H]1O[C@H](CO)[C@@H](O)[C@H](O)[C@H]1O)[C@@H]3C ; "H2, 10% Pd/CEtOAC, MeOH35° C." ; CCC1=C(C)c2cc3[nH]c(cc4nc(c(C)c5cc(C)c(cc1n2)[nH]5)[C@@H](CCC(=O)N(C)CCCS[C@@H]1O[C@H](CO)[C@@H](O)[C@H](O)[C@H]1O)[C@@H]4C)c(C)c3CC]

Step 1: Into a 100 mL RBF fitted with a nitrogen inlet and containing a small stirrer bar was added phyllochlorin (2.00 g, 3-93 mmol, 1 eq), dichloromethane (50 mL), PyBOP (2.26 mg, 1.1 eq), triethylamine (1.64 mL, 3 eq) and 3-(methylamino)-propanol (0.42 g, 1.2 eq). The mixture was stirred at room temperature for 3 hours. Analysis by HPLC showed the reaction to be complete. The reaction mixture was transferred to a separatory funnel and washed with water (2×30 mL). The organic layer was dried (Na₂SO₄) and concentrated by rotary evaporation to give the crude product as a blue/brown film (5.1 g). The crude mixture was loaded directly onto a silica column and eluted with 1.5-2% MeOH/DCM. Pure fractions containing a green/blue spot by TLC with R_{f} 0.30 (5% MeOH/DCM) were combined to give phyllochlorin N-3-hydroxypropyl-N-methyl propylamide (1.29 g, 57%).

¹H NMR (400 MHz, CDCl₃) δ 9.75-9.70 (m, 2H), 8.85 (brs, 1H), 8.16 (dd, 1H), 6.38 (dd, 1H), 6.15 (dd, 1H), 4.70-4.65 (m, 1H), 4.58-4.50 (m, 1H), 4.00 (s, 3H), 3.89-3.82 (m, 3H), 3.65 (s, 3H), 3.53 (s, 3H), 3.37 (s, 3H), 3.31-3.15 (m, 4H), 2.65-2.53 (m, 2H), 2.37-2.22 (m, 3H), 2.16 (3, 3H), 1.80-1.70 (m, 7H), 1.48-1.40 (m, 2H), −2.10 (s, 1H), −2.22 (m, 1H).

Step 2: A single-neck 50 mL RBF was charged with phyllochlorin N-3-hydroxypropyl-N-methyl propylamide (1.25 g, 2.156 mmol, 1 eq), dichloroethane (10 mL) and DMF (1 drop). Thionyl chloride (0.23 mL, 3.233 mmol, 1.5 eq) was added and the resultant solution was stirred (350 rpm) under N₂ at 40° C. After 2 hours, the flask was heated at 60° C. for a further 2 hours. The reaction was then cooled using an ice/water bath and pH=7 phosphate buffer (10 mL) was added. The mixture was extracted with DCM (3×15 mL), dried (Na₂SO₄) and the solvent was removed under reduced pressure to give ˜1.4 g of the crude product. The residual blue solid was purified by column chromatography using 2-4% MeOH/DCM and fractions containing the first dark band to elute were combined to give phyllochlorin N-3-chloropropyl-N-methyl propylamide as a blue/green solid (0.875 g, 67.8%).

Step 3: A single-neck 25 mL RBF was charged with phyllochlorin N-3-chloropropyl-N-methyl propylamide (145 mg, 0.242 mmol, 1 eq), 2-butanone (5 mL) and sodium iodide (73 mg). The resultant solution was stirred (350 rpm) under N₂ at 90° C. TLC after 3 hours indicated that the reaction was complete and the flask was then cooled using an ice/water bath. To the crude iodide was added thioglucose tetraacetate (106 mg, 0.291 mmol, 1.2 eq) and DIPEA (38 mg, 0.291 mmol, 1.2 eq) and the solution was stirred at 25° C. overnight. TLC analysis showed some starting material was present and the solution was heated at 50° C. for 3 hours. The solvent was removed and the residual blue solid was purified by column chromatography using 2-5% MeOH/DCM and fractions containing the darkest band (R_{f}˜0.6, 5% MeOH/DCM) were combined to give phyllochlorin β-1-thioglucose N-methyl propylamide peracetate as a blue/green solid (145 mg) that was used directly in the next step.

Step 4: To a solution of phyllochlorin β-1-thioglucose N-methyl propylamide peracetate (140 mg, 0.1512 mmol, 1 eq) in MeOH (2 mL)/DCM (2 mL) was added NaOMe (4.6 M in MeOH, 0.16 mL, 0.756 mmol, 5 eq), and the mixture was stirred (250 rpm) under N₂ for 30 minutes. TLC analysis showed conversion to the deacetylated product (5% MeOH/DCM, R_{f} (starting material)=0.6, R_{f} (product)=0). The reaction was quenched with acetic acid (10 drops) and concentrated by rotary evaporation. The residue was purified by column chromatography (2-8% MeOH/DCM) to elute phyllochlorin β-D-1-thioglucose-N-methylpropylamide as a dark blue solid (30 mg, 16%—over 2 steps from the chloride).

¹H NMR (400 MHz, d₆-DMSO) δ 9.76 (s, 1H), 9.74 (s, 1H), 9.10 (d, J=5.7 Hz, 1H), 9.07 (s, 1H), 8.35 (dd, J=17.8, 11.6 Hz, 1H), 6.45 (dd, J=17.8, 1.6 Hz, 1H), 6.18 (dd, J=11.6, 1.5 Hz, 1H), 5.24-4.89 (m, 3H), 4.66 (p, J=7.4 Hz, 1H), 4.59-4.44 (m, 2H), 4.28 (dd, J=32.2, 9.6 Hz, 1H), 3.98 (d, J=6.1 Hz, 3H), 3.83 (q, J=7.6 Hz, 2H), 3.63 (d, J=1.0 Hz, 3H), 3.55 (d, J=1.6 Hz, 3H), 3.26-3.01 (m, 3H), 2.90 (s, 2H), 2.81 (s, 1H), 2.72-2.52 (m, 1H), 2.42 (dt, J=20.1, 5.8 Hz, 1H), 1.81 (ddd, J=22.3, 14.6, 6.9 Hz, 1H), 1.75-1.61 (m, 7H), −2.26 (s, 1H), −2.42 (d, J=2.2 Hz, 1H).

Step 5: A 3-necked 250 mL RBF was charged with phyllochlorin β-D-1-thioglucose-N-methylpropylamide (2.10 g, 0.941 mmol, 1 eq), 10% Pd/C (300 mg), ethyl acetate (90 mL), methanol (30 mL) and a stirrer bar. A hydrogen balloon was connected to the middle joint of the flask via a short length air condenser and the side-arm was connected to a 3-way tap. The setup was evacuated and then re-filled with nitrogen (3 times), evacuated and refilled with hydrogen (2 times). The resulting solution was then stirred (350 rpm) under the hydrogen atmosphere with heating at 35° C. for 5 hours. The solution was filtered through Celite® (0.5×3 cm), washing with methanol (˜25 mL) and the solvent was then removed under reduced pressure to give the crude product. The residual crude solid was purified using column chromatography (silica, 4×7 cm) eluting with 10% MeOH/DCM to provide compound 4 as a metallic blue/green solid (1.90 g, 95%) (HPLC purity: 98.8%).

¹H NMR (400 MHz, DMSO-d₆) δ 9.74 (s, 1H), 9.61 (s, 1H), 9.03-8.97 (m, 2H), 5.24-4.88 (m, 3H), 4.68-4.60 (m, 1H), 4.58-4.50 (m, 2H), 4.28 (dd, 1H), 3.99-3.92 (m, 5H), 3.82 (q, 2H), 3.62 (s, 3H), 3.41 (s, 3H), 3.22-3.04 (m, 5H), 2.90 (s, 2H), 2.81 (s, 2H), 2.70-2.52 (m, 3H), 2.48-2.35 (m, 3H), 1.90-1.80 (m, 1H), 1.76-1.63 (m, 11H), −2.34 (s, 1H), −2.40 (s, 1H).

### Synthesis Example 5—Synthesis of Reduced Phyllochlorin β-D-1-glucose-N-methyl-2-ethoxy-N-ethanamine (Compound 5)

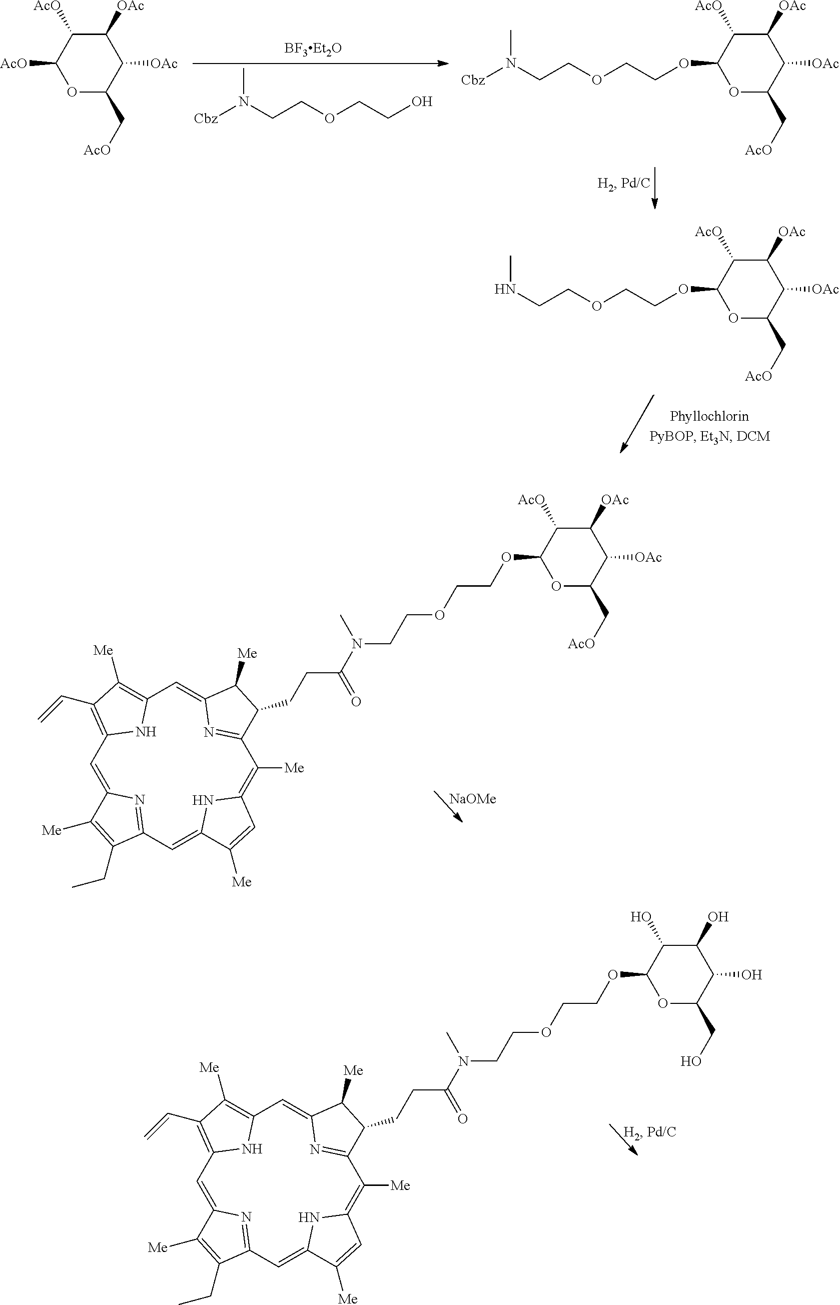

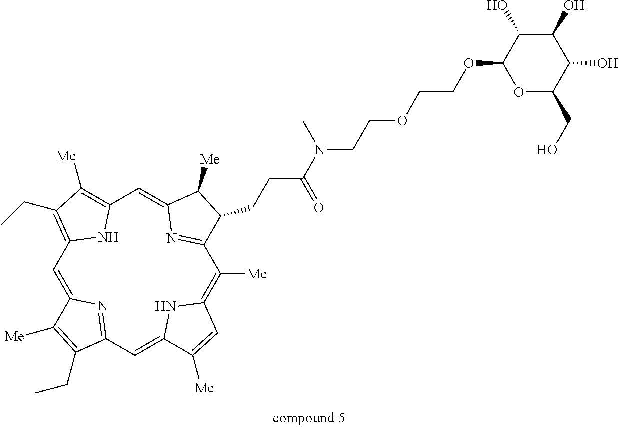 [SMILES: CCC1=C(C)c2cc3[nH]c(cc4nc(c(C)c5cc(C)c(cc1n2)[nH]5)[C@@H](CCC(=O)N(C)CCOCCO[C@@H]1O[C@H](CO)[C@@H](O)[C@H](O)[C@H]1O)[C@@H]4C)c(C)c3CC]

Step 1: A solution of N-Cbz-2-(2-methylamino-ethoxy)-ethanol (1.35 g, 5.33 mmol, 1 eq) and penta-acetyl glucose (2.29 g, 1.1 eq) in DCM (30 mL) under nitrogen was cooled in an ice/water bath and BF₃·Et₂O (3.78 g, 3.29 mL, 5 eq) was added dropwise via syringe. The solution was stirred (420 rpm) at 0-5° C. (external) for 1 hour and then at room temperature overnight. The reaction progress was checked by NMR. On completion of the reaction, the solution was washed with saturated NaHCO₃ (2×20 mL) and then the combined aqueous washes were extracted with DCM (20 mL). The combined organic layers were dried (MgSO₄) and evaporated to give Cbz-protected PEG glucose as a pale yellow oil (˜4 g) which was partially purified by column chromatography (4×25 cm of silica) eluting using a gradient of 0.5-3.5% MeOH/DCM. The resulting oil (2.85 g) was used directly in the next step.

Step 2: To a 3-neck 250 mL RBF was added Cbz-protected PEG glucose (2.85 g), methanol (100 mL) and 10% Pd/C (140 mg, 5% w/w). A hydrogen balloon was attached to the flask and the flask was evacuated and re-filled with nitrogen three times. The flask was then evacuated and re-filled with hydrogen. The solution was stirred at 325 rpm overnight. After evacuating the flask and re-filling with nitrogen the solution was filtered (Celite®), washed with methanol (20 mL) and concentrated under reduced pressure. The concentrated residue was taken up in DCM (25 mL), washed with water (2×20 mL), dried (MgSO₄) and evaporated to give the amine PEG glucose (1.5 g) as a yellow oil which was used without further purification.

Step 3: To a 50 mL RBF was added phyllochlorin (1.04 g, 2.05 mmol, 1 eq), PyBOP (1.28 g, 2.46 mmol, 1.2 eq), DCM (15 mL) and triethylamine (1.92 mL, 13.9 mmol, 6.7 eq). The resultant mixture was stirred (250 rpm) under nitrogen at ambient temperature for 30 minutes, then the amine PEG glucose in DCM (10 mL) was added in one portion. The resultant mixture was stirred overnight in the dark under nitrogen. The reaction mixture was transferred to a separatory funnel and washed with 1M HCl (2×30 mL), then pH 7 buffer (1×30 mL). The organic phase was dried (Na₂SO₄) and concentrated by rotary evaporation to give a blue/black oil which was purified by column chromatography (4×26 cm of silica) eluting using a gradient of 1%→2.5% MeOH/DCM. Fractions containing phyllochlorin β-D-1-glucose-N-methyl ethoxyethyl amide tetraacetate (R_{f}˜0.4 in 5% MeOH/DCM) were combined to give a blue/black oil (˜0.7 g) which was further purified using Biotage autocolumn chromatography. Fractions with R_{f}˜0.4 in 5% MeOH/DCM were combined to give phyllochlorin β-D-1-glucose-N-methyl ethoxyethyl amide tetraacetate (0.38 g) which was deprotected in the next step without further purification.

Step 4: To a solution of phyllochlorin β-D-1-glucose-N-methyl ethoxyethyl amide tetraacetate (350 mg, 0.372 mmol, 1 eq) in MeOH (5 mL) and DCM (5 mL) was added NaOMe (4.6M in MeOH, 0.40 mL, 1.862 mmol, 5 eq) and the mixture stirred (420 rpm) under nitrogen for 1 hour. TLC analysis showed conversion to the deacetylated product (10% MeOH/DCM, R_{f} (starting material)=0.85, R_{f} (product)=0.25). The reaction was quenched with AcOH (112 mg, 1.862 mmol, 5 eq) and concentrated by rotary evaporation to give a black film which was purified by column chromatography (3×21 cm of silica) eluting using a gradient of 3-10% MeOH/DCM to give phyllochlorin β-D-1-glucose-N-methyl-2-ethoxy-N-ethanamine as a blue/black solid (204 mg, 71%) (HPLC purity: 91%).

¹H NMR (400 MHz, DMSO-d₆) δ 9.75 (s, 1H), 9.73 (s, 1H), 9.10 (s, 1H), 9.06 (s, 1H), 8.33 (dd, 1H), 6.44 (d, 1H), 6.18 (d, 1H), 5.30-4.70 (br m, 3H), 4.65 (m, 1H), 4.60-4.53 (m, 2H), 4.13 (m, 1H), 3.98 (d, 3H), 3.81 (m, 4H), 3.68-3.62 (m, 2H), 3.62 (s, 3H), 3.54 (d, 3H), 3.50-3.30 (m, 14H), 3.18-3.05 (m, 4H), 3.00-2.94 (m, 3H), 2.88-2.78 (m, 2H), 2.55-2.45 (m, 1H), 2.45-2.38 (m, 1H), 1.75-1.65 (m, 7H), −2.25 (s, 1H), −2.42 (s, 1H).

Step 5: A 3-neck 100 mL RBF was charged with phyllochlorin β-D-1-glucose-N-methyl-2-ethoxy-N-ethanamine (80 mg, 0.104 mmol, 1 eq), 10% Pd/C (10 mg), methanol (4 mL), ethyl acetate (12 mL) and a stirrer bar (˜15 mm). A hydrogen balloon was connected to the middle joint of the flask via a short length air condenser and the side-arm was connected to a 3-way tap. The setup was evacuated and then re-filled with nitrogen (3 times), evacuated and re-filled with hydrogen (2 times). The resulting solution was stirred (525 rpm) under the hydrogen atmosphere at room temperature overnight. A sample of the mixture was removed, filtered through cotton wool, concentrated under reduced pressure and analysed by HPLC, which showed the reaction was complete. The solution was filtered through Celite® (1×3 cm), the Celite® pad washed with methanol (˜3 mL), and the solvent removed under reduced pressure to give the crude product as a dark blue/green solid which was purified using column chromatography (silica, 3×16 cm) eluting with 5-11% MeOH/DCM to provide compound 5 as a dark blue/green solid (62 mg, 78%) (HPLC purity: 94.1%).

¹H NMR (400 MHz, DMSO-d₆) δ 9.74 (s, 1H), 9.61 (s, 1H), 9.02 (s, 1H), 8.99 (s, 1H), 5.05-4.87 (br m, 3H), 4.61 (m, 1H), 4.58-4.45 (m, 2H), 4.12 (m, 2H), 3.96 (m, 5H), 3.81 (m, 3H), 3.67-3.60 (m, 5H), 3.62 (s, 3H), 3.58-3.50 (m, 2H), 3.50-3.37 (m, 12H), 3.18 (s, 3H), 3.15-3.10 (m, 1H), 3.08-3.00 (m, 2H), 2.95 (m, 3H), 2.83 (s, 2H), 1.75-1.65 (m, 10H), −2.35 (s, 1H), −2.40 (s, 1H).

### Synthesis Example 6—Synthesis of Reduced Phyllochlorin Methyl Ester (Compound 6)

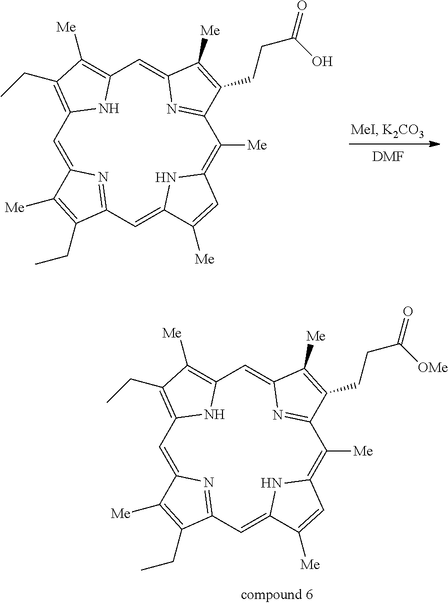

To a 10 mL RBF under nitrogen was added reduced phyllochlorin (compound 1) (250 mg, 0.4895 mmol, 1 eq), potassium carbonate (81.2 mg, 0.5875 mmol, 1.2 eq) and DMF (3 mL). Methyl iodide (39.6 μL, 0.6364 mmol, 1.3 eq) was then added, and the resultant mixture stirred (420 RPM) for 6 hours, by which time the reaction was complete as monitored by HPLC. DCM (3 mL) was added and the solution stirred for 15 minutes before being filtered through a pad of Celite® and washed with additional DCM (2×3 mL) until the filtrate became colourless. The solvents were removed on a rotary evaporator to give an almost black syrup which was then taken up in EtOAc (12.5 mL) and washed with water (3×10 mL), dried (Na₂SO₄) and concentrated under reduced pressure to give crude product as a dark blue/green solid (241.1 mg). The crude product was purified by Biotage autocolumn chromatography to give compound 6 as a black/green solid (151.6 mg).

¹H NMR (400 MHz, CDCl₃) δ 9.73 (s, 1H), 9.56 (s, 1H), 8.82 (s, 1H), 8.78 (s, 1H), 4.55 (d, J=9.1 Hz, 1H), 4.49 (q, J=7.3 Hz, 1H), 3.97 (s, 3H), 3.95 (d, J=7.7 Hz, 2H), 3.85 (q, J=7.6 Hz, 2H), 3.64 (d, J=1.0 Hz, 3H), 3.57 (s, 3H), 3.41 (s, 3H), 3.38 (s, 3H), 2.62-2.43 (m, 2H), 2.17-1.99 (m, 2H), 1.82-1.71 (m, 9H), −2.19 (s, 2H).

### Synthesis Example 7—Synthesis of Reduced Phyllochlorin N-(2-(2-hydroxyethoxy)ethyl)-N-methylpropylamide (Compound 7)

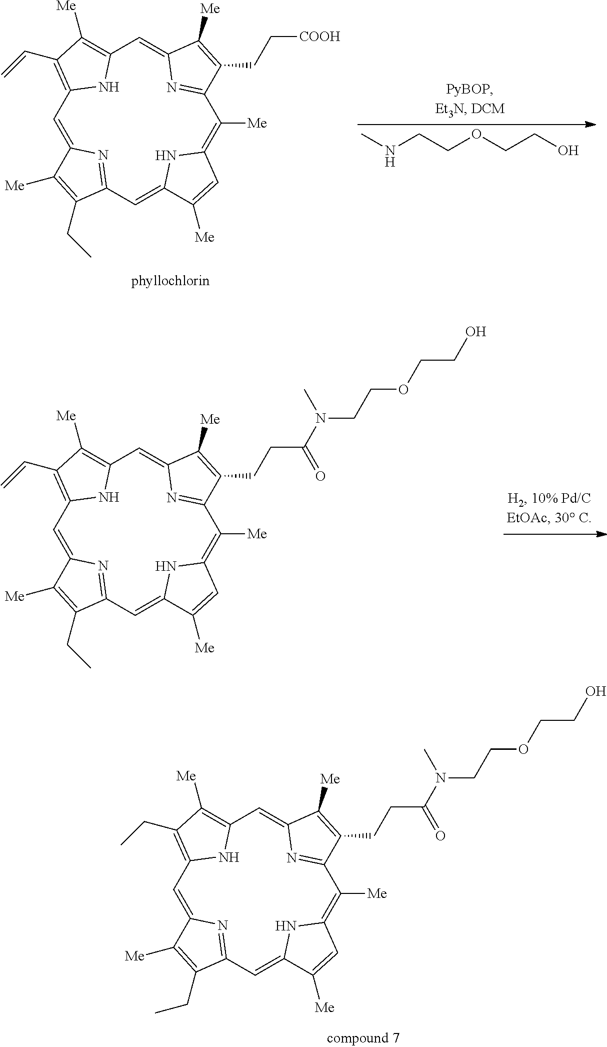

Step 1: Into a 100 mL RBF fitted with a nitrogen inlet and containing a stirrer bar was added phyllochlorin (2.00 g, 3.93 mmol, 1 eq), dichloromethane (40 mL), PyBOP (2.46 g, 4.72 mmol, 1.2 eq) and triethylamine (0.87 mL, 3 eq). The mixture was stirred at room temperature for 30 minutes and then 2-(2-methylaminoethoxy)ethanol (0.61 g, 1.3 eq) in DCM (5 mL) was added in one portion. The mixture was stirred at room temperature overnight. Analysis by HPLC showed the reaction to be complete. The reaction mixture was transferred to a separatory funnel and washed with 1M HCl (2×50 mL), then pH 7 buffer (2×50 mL). The organic layer was dried (Na₂SO₄) and concentrated by rotary evaporation to give the crude product as a blue/green film (4.2 g) which was purified by column chromatography (5×25 cm of silica) eluting with 1-3% MeOH/DCM. Pure fractions by TLC(R_{f} 0.40 in 5% MeOH/DCM) were combined to give phyllochlorin N-(2-(2-hydroxyethoxy)ethyl)-N-methylpropylamide (1.40 g, 58%) (HPLC purity: 94.4%).

¹H NMR (400 MHz, CDCl₃) δ 9.72 (m, 2H), 8.87-8.82 (m, 1H), 8.16 (dd, 1H), 6.39 (dd, 1H), 6.15 (dd, 1H), 4.72-4.65 (m, 1H), 4.59-4.50 (m, 1H), 4.01 (s, 3H), 3.85 (q, 2H), 3.65 (s, 3H), 3.60-3.55 (m, 2H), 3.53 (s, 3H), 3.41-3.38 (m, 2H), 3.37 (s, 3H), 3.35-3.30 (m, 2H), 2.76-2.71 (m, 1H), 2.65-2.50 (m, 4H), 2.50-2.40 (m, 1H), 2.38-2.30 (m, 3H), 2.30-2.20 (m, 2H), 1.90-1.80 (m, 1H), 1.80-1.73 (m, 7H), −2.09 (br s, 1H), −2.22 (br s, 1H).

Step 2: A 3-necked 100 mL RBF was charged with phyllochlorin N-(2-(2-hydroxyethoxy)ethyl)-N-methylpropylamide (100 mg, 0.164 mmol, 1 eq), 10% Pd/C (10 mg), ethyl acetate (20 mL) and a stirrer bar (˜15 mm). A hydrogen balloon was connected to the middle joint of the flask via a short length air condenser and the side-arm was connected to a 3-way tap. The setup was evacuated and then re-filled with nitrogen (3 times), evacuated and refilled with hydrogen (2 times). The resulting solution was then stirred (550 rpm) under the hydrogen atmosphere with heating at 30° C. for 90 minutes. The solution was filtered through Celite® (0.5×3 cm), washing with ethyl acetate (˜10 mL) and the solvent was then removed under reduced pressure to give crude product (˜0.15 g). The residual solid was purified using column chromatography (silica, 3×19 cm) eluting with 3-4% MeOH/DCM to provide compound 7 as a metallic blue/green flaky solid (84 mg, 84%) (HPLC purity: 97.7%).

¹H NMR (400 MHz, CDCl₃) δ 9.73 (s, 1H), 9.56 (s, 1H), 8.82 (brs, 1H), 8.79 (m, 1H), 4.70-4.62 (m, 1H), 4.55-4.47 (m, 1H), 4.00 (m, 3H), 3.95 (q, 2H), 3.86 (q, 2H), 3.64 (s, 3H), 3.60-3.56 (m, 2H), 3.43-3.37 (m, 9H), 3.35-3.30 (m, 1H), 2.70-2.66 (m, 1H), 2.65-2.50 (m, 4H), 2.42-2.35 (m, 1H), 2.33 (s, 2H), 2.30-2.15 (m, 2H), 1.90-1.80 (m, 1H), 1.80-1.73 (m, 10H), −2.18 (brm, 2H).

### Synthesis Example 8—Synthesis of Reduced Phyllochlorin β-D-1-thioglucose-N-methylpropylamide Zinc Complex (Compound 8)

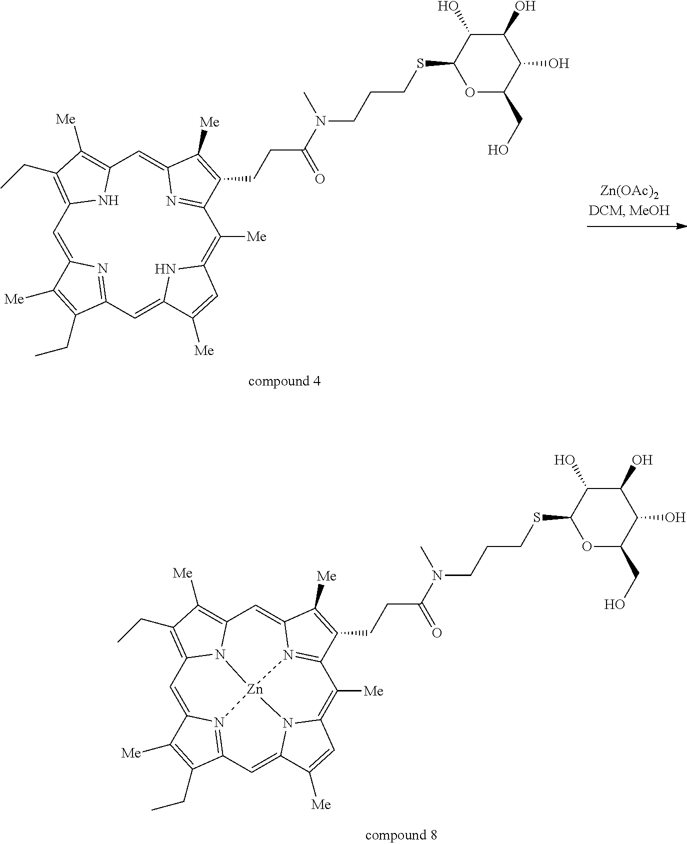

To a 25 mL 1-necked RBF was added reduced phyllochlorin β-D-1-thioglucose-N-methylpropylamide (compound 4) (50 mg, 0.0658 mmol, 1 eq) and DCM (2 mL). A solution of zinc acetate (24 mg, 0.1316 mmol, 2 eq) in methanol (1 mL) was added and the mixture was stirred under nitrogen in the dark for 2 hours. The solution was then diluted with DCM (20 mL) and washed with water (3×25 mL), dried (Na₂SO₄) and concentrated to give compound 8 (57 mg, quantitative) (HPLC purity: 99.3%) as a so blue solid.

¹H NMR (400 MHz, CDCl₃) δ 9.45 (brs, 1H), 9.37 (brs, 1H), 8.65 (brs, 1H), 8.51 (brs, 1H), 4.15-4.00 (brm, 2H), 3.90-3.70 (brm, 4H), 3.60-3.50 (brm, 3H), 3.45-3.30 (brm, 9H), 3.20 (brm, 1H), 3.15-3.00 (brm, 1H), 2.80-2.45 (brm, 2H), 2.40-2.20 (brm, 4H), 2.15-2.00 (brm, 3H), 1.95-1.70 (brm, 12H), 1.60 (brm, 2H), 1.50 (brs, 4H), 1.00-1.20 (brm, 3H).

### Synthesis Example 9—Synthesis of Reduced Phyllochlorin β-D-glucosyl-N-methylpropylamide (Compound 9)

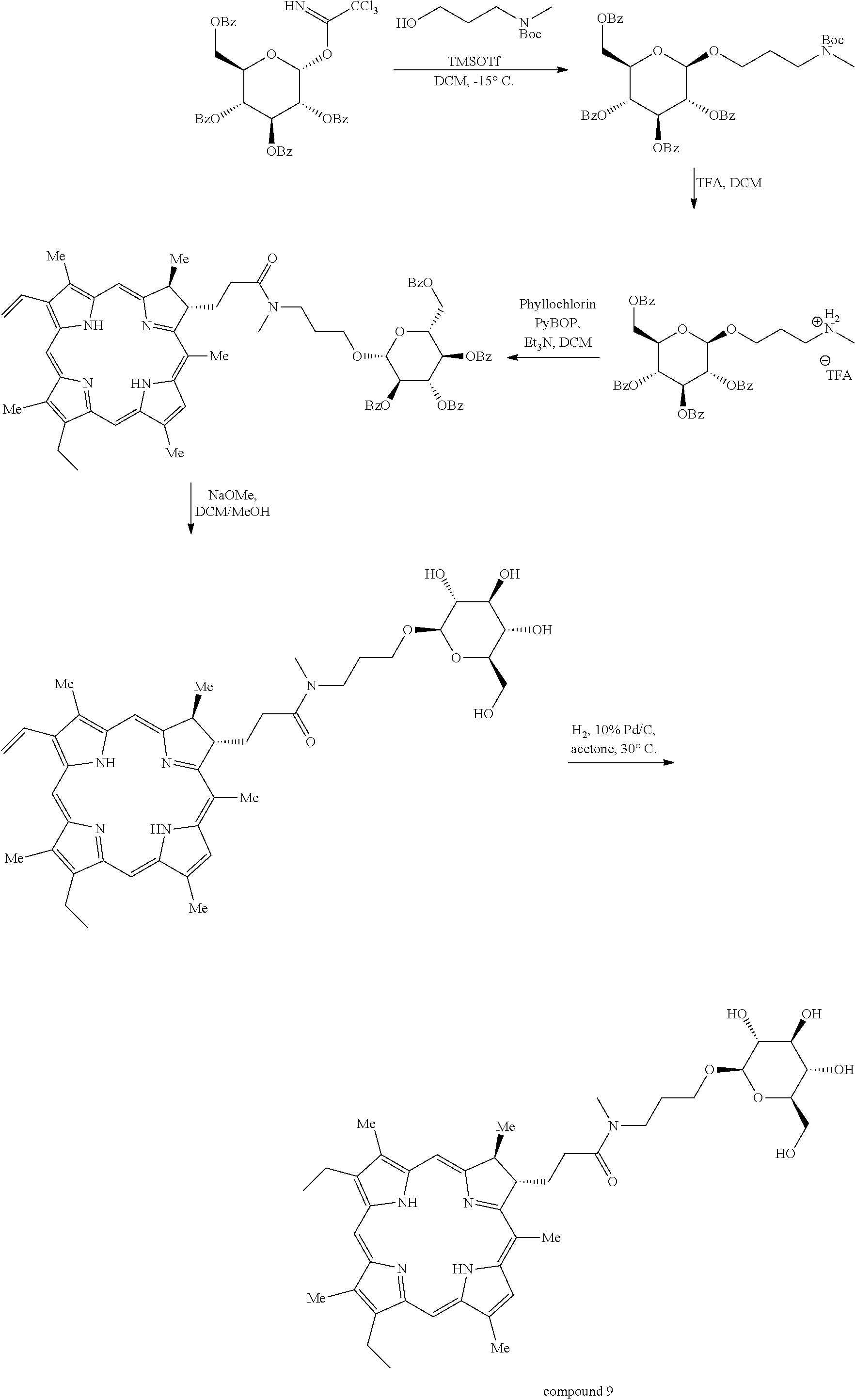

Step 1: A 250 mL 3-neck RBF fitted with an internal thermometer, nitrogen inlet and rubber septum was charged with tert-butyl (3-hydroxypropyl)(methyl)carbamate (2.68 g, 14.17 mmol, 1.05 eq), a stirrer bar, 2,3,4,6-tetra-O-benzoyl-α-D-glucopyranosyl trichloroacetimidate (10.00 g, 13.5 mmol, 1 eq), dry DCM (100 mL) and ground 4 A molecular sieves (5.0 g). The resultant suspension was placed under an atmosphere of nitrogen and stirred (420 rpm) for 0.5 hours before cooling to −15° C. (internal temperature) with the aid of an EtOH/ice/NaCl bath. A solution of TMSOTf (0.2M in DCM, 3.3 mL, 0.67 mmol) was added dropwise over the course of a minute, and stirring continued at low temperature for 0.5 hours, at which point TLC analysis indicated complete reaction (25% EtOAc/hexanes, R_{f} (starting material)=0.4, R_{f} (product)=0.2, visualised by UV). The reaction was quenched with triethylamine (8 drops) and filtered through Celite®, washing through with a further portion of DCM. The filtrate was concentrated to give the crude glycosylated product as a yellow syrup (15.4 g) which was dissolved in minimum DCM and the solution purified by column chromatography (4×22 cm of silica) using a gradient solvent of 25-35% EtOAc in hexane to give 2,3,4,6-tetra-O-benzoyl-β-D-glucopyranose-N-Boc-N-methylproploxyamine as a colourless syrup (7.95 g, 77%) (R_{f} 0.4 in 35% EtOAc in hexane).

¹H NMR (400 MHz, CDCl₃) δ 8.05-7.99 (m, 2H), 7.98-7.93 (m, 2H), 7.92-7.88 (m, 2H), 7.86-7.79 (m, 2H), 7.59-7.46 (m, 3H), 7.46-7.24 (m, 9H), 5.90 (dd, J=9.7, 9.7 Hz, 1H), 5.68 (dd, J=9.7, 9.7 Hz, 1H), 5.52 (dd, J=9.7, 7.8 Hz, 1H), 4.84 (d, J=7.8 Hz, 1H), 4.64 (dd, J=12.2, 3.2 Hz, 1H), 4.49 (dd, J=12.2, 5.2 Hz, 1H), 4.20-4.13 (m, 1H), 3.95 (ddd, J=9.7, 5.2, 3.2 Hz, 1H), 3.61-3.50 (m, 1H), 3.24-2.99 (m, 2H), 2.66 (s, 3H), 1.82-1.68 (m, 2H), 1.39 (s, 9H).

Step 2: To a 500 mL RBF containing 2,3,4,6-tetra-O-benzoyl-β-D-glucopyranose-N-Boc-N-methylproploxyamine (7.95 g, 10.35 mmol, 1 eq) was added DCM (50 mL) and a stirrer bar. The mixture was stirred (250 rpm) briefly until a solution had formed before TFA (10 mL) was added. The mixture was stirred for 0.5 hours and monitored by TLC (30% EtOAc/hexanes, R_{f} (starting material)=0.4, R_{f} (product)=0), visualised by UV). The reaction was concentrated by rotary evaporation and then reconcentrated from CHCl₃ (2×30 mL) to give 2,3,4,6-tetra-O-benzoyl-β-D-glucopyranose-N-methylpropyloxyammonium trifluoroacetate as lightly coloured syrup (11.0 g) that was used without further purification.

¹H NMR (400 MHz, CDCl₃) δ 8.69 (br s, 1H), 8.15 (br s, 1H), 8.08-8.02 (m, 2H), 7.99-7.88 (m, 4H), 7.87-7.80 (m, 2H), 7.67 (br s, 1H), 7.63-7.48 (m, 3H), 7.52-7.24 (m, 9H), 5.99 (dd, J=9.8, 9.8 Hz, 1H), 5.71 (dd, J=9.8, 9.8 Hz, 1H), 5.38 (dd, J=9.8, 7.8 Hz, 1H), 4.82 (d, J=7.8 Hz, 1H), 4.74 (dd, J=12.3, 2.8 Hz, 1H), 4.47 (dd, J=12.3, 5.0 Hz, 1H), 4.22-4.06 (m, 2H), 3.73 (app. p, J=5.1 Hz, 1H), 3.31-3.18 (m, 1H), 3.14-3.01 (m, 1H), 2.79 (t, J=5.3 Hz, 3H), 2.04 (app. p, J=5.4 Hz, 2H).

Step 3: A 500 mL RBF was charged with a stirrer bar, phyllochlorin (4.05 g, 7.96 mmol, 1 eq) and DCM (60 mL). 2,3,4,6-Tetra-O-benzoyl-β-D-glucopyranose-N-methylpropyloxyammonium trifluoroacetate (6.91 g, 10.35 mmol, 1.3 eq) was dissolved in DCM (20 mL) and transferred into the RBF. Triethylamine (6.62 mL, 47.8 mmol, 6 eq) was added, followed by PyBOP (4.97 g, 9.55 mmol, 1.2 eq), and the mixture stirred for 0.5 hours. TLC analysis showed consumption of the starting material and the presence of the product (5% MeOH/DCM, R_{f} (phyllochlorin)=0.25, R_{f} (product)=0.95, visualised by UV). The mixture was transferred to a separatory funnel and washed with 1M HCl (2×75 mL), then pH 7 phosphate buffer (100 mL), before being dried (Na₂SO₄) and concentrated by rotary evaporation to give the crude product as a green film (16.3 g). The crude product was purified by Biotage autocolumn chromatography (0-2% MeOH/DCM) to give phyllochlorin β-D-glucosyl-N-methylpropylamide tetrabenzoate as a green film (4.75 g, 52%) (HPLC purity: 95.9%).

Step 4: To a 500 mL RBF containing phyllochlorin β-D-glucosyl-N-methylpropylamide tetrabenzoate (4.65 g, 4.01 mmol, 1 eq) was added DCM (50 mL), MeOH (50 mL) and a stirrer bar. The mixture was stirred (300 rpm) briefly until a dark solution had formed, whereupon a solution of NaOMe (4.6M in MeOH, 0.44 mL, 2.01 mmol, 0.5 eq) was added, and the mixture stirred for 2 hours. TLC analysis at this point showed complete reaction (10% MeOH/DCM, R_{f} (starting material)=0.95, R_{f} (product)=0). The reaction mixture was concentrated and the residue purified by Biotage autocolumn chromatography (5-12% MeOH/DCM) to give phyllochlorin β-D-glucosyl-N-methylpropylamide as a blue/green flaky solid (2.34 g, 79%) (HPLC purity: 99.4%).

Step 5: A 3-necked 250 mL RBF fitted with a stopper and 3-way tap connected to a nitrogen line/vacuum pump was charged with phyllochlorin β-D-glucosyl-N-methylpropylamide (2.25 g, 3.03 mmol, 1 eq), a stirrer bar and acetone (100 mL). The mixture was stirred briefly until a dark green solution had formed, and then 10% Pd/C (100 mg) added under positive pressure of nitrogen. A hydrogen balloon was connected to the middle joint of the flask via a short length air condenser and the side-arm was connected to a 3-way tap. The setup was evacuated and then re-filled with nitrogen (3 times), evacuated and refilled with hydrogen (2 times). The resulting solution was then stirred (550 rpm) under the hydrogen atmosphere with heating at 30° C. for 5 hours. The reaction mixture was filtered through a small plug of Celite® and washed through with acetone. The filtrate was concentrated to give the crude hydrogenated compound as a blue-green film (2.30 g). The crude material was purified by Biotage autocolumn chromatography to give compound 9 as a black solid (2.02 g, 89%) (HPLC purity: 99.7%).

¹H NMR (400 MHz, CD₃OD) δ 9.65 (brs, 1H), 9.49 (m, 1H), 8.91 (m, 1H), 8.87 (m, 1H), 4.59 (m, 2H), 3.95 (m, 3H), 3.47 (m, 2H), 3.73 (m, 3H), 6.63 (m, 1H), 3.55 (m, 4H), 3.48 (m, 3H), 3.26 (m, 5H), 3.20-3.03 (m, 4H), 2.87 (m, 1H), 2.78 (m, 1H), 2.58 (s, 1H), 2.55-2.40 (m, 2H), 2.39 (s, 2H), 2.20-1.95 (m, 2H), 1.80-1.68 (m, 9H), 1.47 (m, 1H), 1.20 (m, 1H).

### Synthesis Example 10—Synthesis of Reduced Phyllochlorin N-meglumine-propylamide (Compound 10)

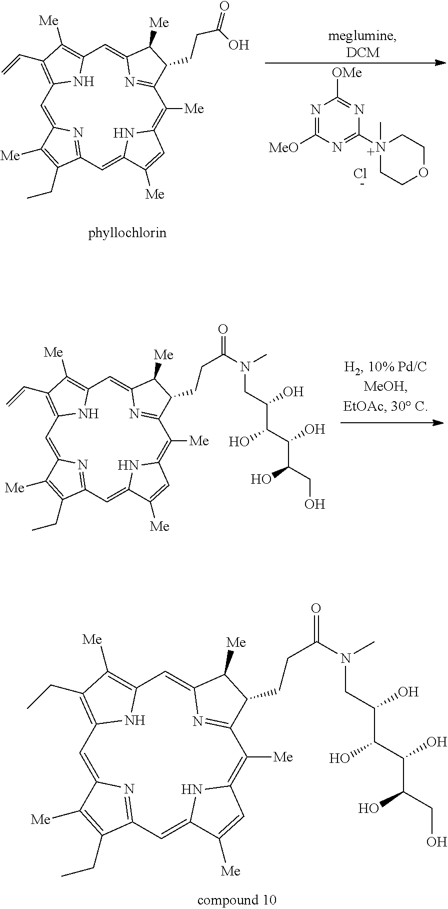

Step 1: To a 50 mL RBF was added phyllochlorin (0.50 g, 0.983 mmol, 1 eq), DMTMM (0.30 g, 1.081 mmol, 1.1 eq), DCM (15 mL) and meglumine (0.23 g, 1.179 mmol, 1.2 eq). The resultant mixture was stirred under nitrogen at ambient temperature for 1 hour. A further portion of meglumine (0.10 g, 0.51 mmol, 0.5 eq) was added and the solution stirred for a further 3 hours. The reaction mixture was transferred to a separatory funnel, diluted with chloroform (30 mL) and washed with 0.5M HCl (50 mL). The aqueous layer was re-extracted with chloroform and the combined organics washed with pH 7 buffer. The organic phase was dried (Na₂SO₄) and concentrated by rotary evaporation to give a blue/black film, which was purified by column chromatography (silica) using a gradient of 5% MeOH/DCM (50 mL), then 7% MeOH/DCM (100 mL), then 9% MeOH/DCM (100 mL), and then 10% MeOH/DCM (200 mL) to give phyllochlorin N-meglumine-propylamide as a dark blue/green solid (432 mg, 64%).

¹H NMR (400 MHz, d₆-DMSO) δ 9.74 (s, 1H), 9.72 (s, 1H), 9.11 (s, 1H), 9.07 (s, 1H), 8.34 (dd, 1H), 6.44 (d, 1H), 6.17 (d, 1H), 5.10 & 4.75 (2×d, 1H), 4.65-4.40 (m, 5H), 4.30 (m, 1H), 4.00 (m, 3H), 3.90 (m, 1H), 3.80 (m, 2H), 3.68 (m, 1H), 3.62 (s, 3H), 3.60-3.50 (m, 5H), 3.50-3.40 (m, 2H), 3.30-3.08 (m, 1H), 3.00 (s, 1H), 2.90 (s, 2H), 2.86-2.60 (m, 1H), 2.45-2.35 (m, 1H), 1.75-1.65 (m, 6H), 1.75-1.50 (m, 1H), −2.26 (s, 1H), −2.42 (s, 1H).

Step 2: A 3-necked 50 mL RBF was charged with phyllochlorin N-meglumine-propylamide (200 mg, 0.292 mmol, 1 eq), 10% Pd/C (25 mg), methanol (4 mL), ethyl acetate (4 mL) and a stirrer bar. A hydrogen balloon was connected to the middle joint of the flask via a short length air condenser and the side-arm was connected to a 3-way tap. The setup was evacuated and then re-filled with nitrogen (3 times), evacuated and refilled with hydrogen (2 times). The resulting solution was then stirred (500 rpm) under the hydrogen atmosphere with heating at 30° C. for 4 hours. The solution was filtered through Celite® (0.5×1.5 cm), washing with methanol (˜10 mL) and the solvent was then removed under reduced pressure to give compound 10 as a metallic blue/green solid (190 mg, 95%) (HPLC purity: 99.4%).

¹H NMR (400 MHz, DMSO-d₆) δ 9.74 (s, 1H), 9.61 (s, 1H), 9.02 (s, 1H), 8.99 (s, 1H), 5.10 & 4.75 (2×m, 1H), 4.65-4.40 (m, 5H), 4.40-4.30 (m, 1H), 4.10 (m, 1H), 4.00-3.88 (m, 6H), 3.80 (m, 2H), 3.70-3.50 (m, 7H), 3.40 (s, 3H), 3.18 (s, 3H), 3.00 & 2.90 (2×s, 3H), 2.86-2.60 (m, 1H), 2.50-2.38 (m, 1H), 1.75-1.65 (m, 9H), 1.65-1.50 (m, 1H), −2.35 (s, 1H), −2.40 (s, 1H).

### Synthesis Example 11—Synthesis of Reduced Phyllochlorin ((3-(3-(methylamino)propoxy) propyl)triphenylphosphonium Chloride Propylamide (Compound 11)

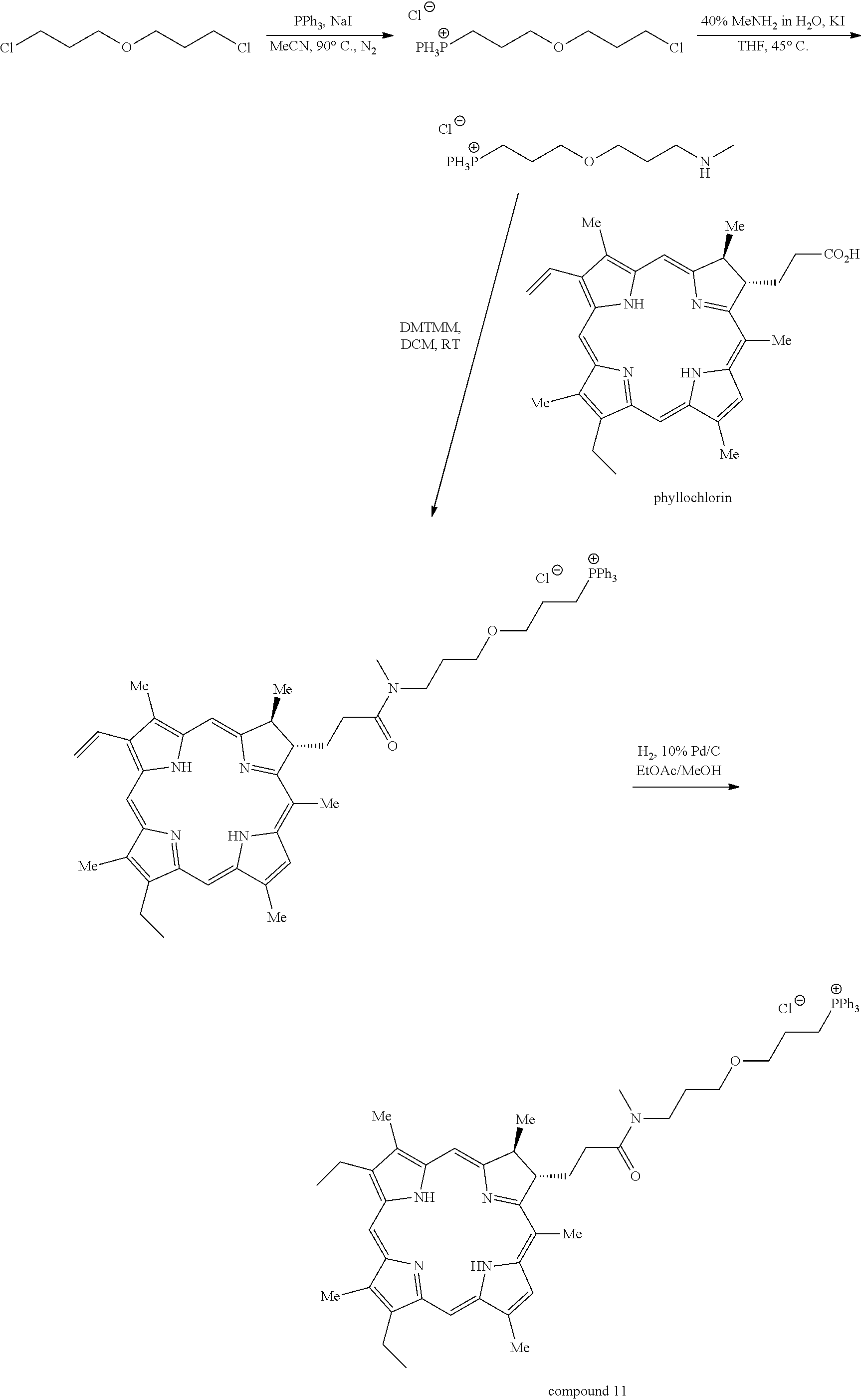

Step 1: A 500 mL 3-neck RBF was charged with bis(3-chloropropyl)ether (20.00 g, 70.15 mmol, 1.66 eq), triphenyl phosphine (18.40 g, 70.15 mmol, 1 eq), sodium iodide (7.01 g, 46.77 mmol, 0.66 eq) and acetonitrile (340 mL). The reaction was stirred at 90° C. for 72 hours. The reaction flask was then cooled to room temperature and the suspension was filtered through a 2 cm plug of Celite®, washing through with an extra 250 mL of acetonitrile. The faint yellow solution was then evaporated to dryness to leave a dark yellow oil (44.40 g). The crude oil was first purified by column chromatography (column length: 12 cm; column width: 9 cm; mobile phase: 6% MeOH in DCM; fraction size: ˜75 mL; loading method: dissolved in 20 mL of the mobile phase; R_{f} (prod.)=0.35, R_{f} (dimer)=0.06, visualised by UV). The fractions containing the product were concentrated by rotary evaporation and the resultant crude product put through a second column (column length: 7 cm; column width: 9 cm; loading method: dissolved in 10 mL of DCM). The column was first run with DCM then swapped to 10% MeOH in DCM to give 3-(3-chloropropoxy)propyl)triphenyl-phosphonium chloride as a faint red solid (18.74 g, 62%) (mp: 131-134° C.).

¹H NMR (400 MHz, CDCl₃) δ 7.87-7.76 (m, 9H), 7.75-7.63 (m, 6H), 3.93-3.80 (m, 2H), 3.75 (td, J=5.7, 1.3 Hz, 2H), 3.58 (q, J=6.3 Hz, 4H), 2.05-1.88 (m, 4H).

Step 2: A 50 mL pressure vessel fitted with a Teflon insert was charged with (3-(3-chloropropoxy)propyl)triphenylphosphonium chloride (3.00 g, 6.92 mmol, 1 eq), methylamine (40% w/w in H₂O, 5.99 mL, 69.23 mmol, 10 eq) and potassium iodide (11.5 mg, 69.22 μmol, 0.01 eq). The vessel was then heated to 45° C. and the mixture was left to stir (500 rpm) for 24 hours. Reaction progress was checked via NMR. The reaction mixture was then diluted with H₂O (60 mL) and extracted with DCM (3×60 mL). The combined organic layers were dried over MgSO₄, filtered and concentrated by rotary evaporation to give (3-(3-(methylamino)propoxy)propyl)triphenylphosphonium chloride as a viscous light beige syrup (2.96 g, quant, ˜63% purity by NMR analysis) (mp: 131-134° C.).

¹H NMR (400 MHz, CDCl₃) δ 7.87-7.75 (m, 9H), 7.75-7.65 (m, 6H), 3.98-3.85 (m, 2H), 3.74 (t, J=5.2 Hz, 2H), 3.55 (t, J=6.1 Hz, 2H), 2.70 (t, J=6.8 Hz, 2H), 2.43 (s, 3H), 1.94 (ddt, J=16.5, 8.7, 5.6 Hz, 2H), 1.86-1.77 (m, 2H).

Step 3: Into a 50 mL RBF fitted with a nitrogen inlet and containing a small stirrer bar was added phyllochlorin (500 mg, 0.983 mmol, 1 eq), dichloromethane (20 mL), 4-(4,6-dimethoxy-1,3,5-triazin-2-yl)-4-methyl-morpholinium chloride (DMTMM) (354 mg, 1.28 mmol, 1.3 eq) and (3-(3-(methylamino)propoxy)propyl)triphenyl-phosphonium chloride (504 mg, 1.18 mmol, 1.2 eq). The mixture was stirred at 25° C. for 1 hour at which time HPLC analysis showed that ˜6% phyllochlorin was still present in the reaction mixture. After stirring for another 1 hour, DMTMM (53 mg, 0.197 mmol, 0.2 eq) and (3-(3-(methylamino)propoxy)propyl)triphenylphosphonium chloride (133 mg, 0.197 mmol, 0.2 eq) were added and the reaction mixture stirred at 25° C. for 1 hour. The reaction mixture was then diluted with DCM (50 mL), washed with 0.1M HCl (50 mL) then H₂O (50 mL). The organic layer was then dried over Na₂SO₄, filtered on a sintered glass frit and concentrated by rotary evaporation to give the crude product as a dark blue solid. The crude mixture was loaded directly onto a silica column (4×20 cm, pre-equilibrated with 1% MeOH in DCM) and eluted with 1% to 5% MeOH in DCM. Fractions containing a green/blue spot by TLC with R_{f}=0.42 (5% MeOH in DCM) were collected and concentrated by rotary evaporation then dried in a vacuum oven at 60° C. for 2 hours to give phyllochlorin ((3-(3-(methylamino)propoxy)propyl)triphenyl-phosphonium chloride propylamide as a dark blue solid (407 mg, 45%) (HPLC purity: 95-4%).

¹H NMR (400 MHz, CDCl₃) δ 9.75-9.62 (m, 2H), 8.91-8.78 (m, 2H), 8.15 (m, 1H), 7.64 (m, 4H), 7.51-7.40 (m, 8H), 7.39-7.31 (m, 2H), 6.36 (m, 1H), 6.13 (dd, J=11.5, 1.5 Hz, 1H), 4.66-4.47 (m, 2H), 3.98 (s, 1H), 3.95 (s, 2H), 3.89-3.77 (m, 2H), 3.74-3.66 (m, 1H), 3.63 (t, J=1.2 Hz, 3H), 3.58 (m, 1H), 3.52 (d, J=2.9 Hz, 3H), 3.36 (s, 2H), 3.34-3.28 (m, 3H), 3.15-3.03 (m, 1H), 2.90-2.78 (m, 1H), 2.72 (s, 1H), 2.57 (s, 2H), 2.54-2.37 (m, 1H), 2.18-1.96 (m, 1H), 1.85-1.69 (m, 5H), 1.62 (m, 6H), 1.43-1.29 (m, 1H).

Step 4: A 3-necked 50 mL RBF was charged with phyllochlorin ((3-(3-(methylamino)propoxy)propyl)triphenylphosphonium chloride propylamide (60 mg, 0.065 mmol, 1 eq), 10% Pd/C (10 mg), methanol (4 mL), ethyl acetate (4 mL) and a stirrer bar. A hydrogen balloon was connected to the middle joint of the flask via a short length air condenser and the side-arm was connected to a 3-way tap. The setup was evacuated and then re-filled with nitrogen (3 times), evacuated and refilled with hydrogen (2 times). The resulting solution was then stirred (450 rpm) under the hydrogen atmosphere for 3 hours. The solution was filtered through Celite® (0.5×1.5 cm), washing with methanol (˜10 mL) and the solvent was then removed under reduced pressure to give compound 11 as a dark green solid (62 mg, quantitative) (HPLC purity: 96.1%).

¹H NMR (400 MHz, CDCl₃) δ 9.73-9.69 (m, 1H), 9.55-9.42 (m, 1H), 8.80-8.77 (m, 2H), 7.68-7.61 (m, 4H), 7.49-7.41 (m, 9H), 7.39-7.34 (m, 2H), 4.62-4.45 (m, 2H), 3.96-3.90 (m, 5H), 3.88-3.80 (m, 2H), 3.75-3.66 (m, 1H), 3.62 (s, 3H), 3.57 (td, 1H), 3.39 (m, 3H), 3.37 (m, 2H), 3.32-3.28 (m, 5H), 3.09 (m, 1H), 2.88-2.80 (m, 1H), 2.71 (s, 1H), 2.53 (s, 2H), 2.50-2.40 (m, 2H), 2.12-1.95 (m, 2H), 1.82-1.70 (m, 10H), 1.60 (m, 5H), 1.42-1.28 (m, 2H), −2.18 (brs, 1H), −2.19 (brs, 1H).

### Synthesis Example 12—Synthesis of Reduced Phyllochlorin N-(2-(2-(2-hydroxyethoxy)ethoxy)ethyl)-N-methyl-3-amide (Compound 12)

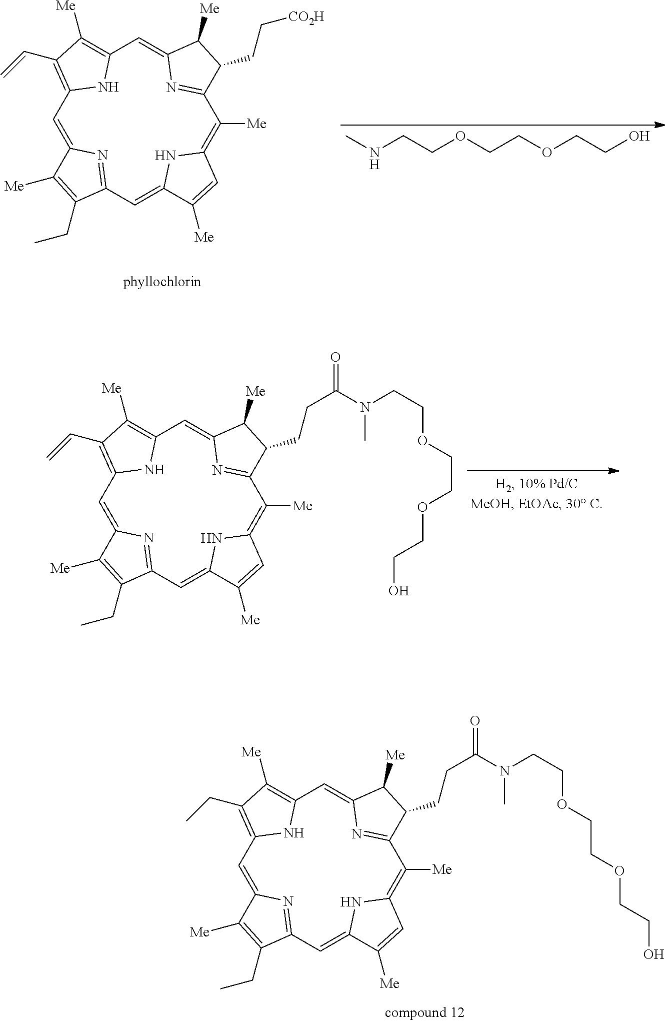 [SMILES: C=Cc1c(C)c2cc3nc(c(C)c4cc(C)c(cc5nc(cc1[nH]2)C(C)=C5CC)[nH]4)[C@@H](CCC(=O)O)[C@@H]3C ; CNCCOCCOCCO ; C=Cc1c(C)c2cc3nc(c(C)c4cc(C)c(cc5nc(cc1[nH]2)C(C)=C5CC)[nH]4)[C@@H](CCC(=O)N(C)CCOCCOCCO)[C@@H]3C ; CCC1=C(C)c2cc3[nH]c(cc4nc(c(C)c5cc(C)c(cc1n2)[nH]5)[C@@H](CCC(=O)N(C)CCOCCOCCO)[C@@H]4C)c(C)c3CC]

Step 1: Into a 50 mL RBF fitted with a nitrogen inlet and containing a small stirrer bar was added phyllochlorin (500 mg, 0.983 mmol, 1 eq), dichloromethane (15 mL), PyBOP (563 mg, 1.1 eq), triethylamine (409 μL, 3 eq) and 2-(2-(2-(methylamino)ethoxy)ethoxy)ethanol (193 mg, 1.2 eq). The mixture was stirred at room temperature for 1 hour. Analysis by HPLC showed the reaction to be complete. The reaction mixture was transferred to a separatory funnel and washed with water (2×10 mL) and the organic layer dried (Na₂SO₄) and concentrated by rotary evaporation to give the crude product as a blue/brown film (1.10 g). The crude mixture was loaded directly onto a silica column and eluted with 3-7% MeOH/DCM. Pure fractions containing a green/blue compound by TLC(R_{f} 0.20 in 5% MeOH/DCM) were combined to give phyllochlorin N-(2-(2-(2-hydroxyethoxy)ethoxy)ethyl)-N-methyl-3-amide.

¹H NMR (400 MHz, CDCl₃) δ 9.71 (m, 2H), 8.83 (m, 2H), 8.16 (dd, 1H), 6.38 (dd, 1H), 6.13 (dd, 1H), 4.66 (m, 1H), 4.54 (m, 1H), 4.01 (s, 3H), 3.84 (q, 2H), 3.63 (s, 3H), 3.55-3.50 (m, 4H), 3.47-3.30 (m, 10H), 3.04 (m, 1H), 2.77-2.50 (m, 6H), 2.40-2.20 (m, 4H), 1.93-1.35 (m, 1H), 1.30-1.22 (m, 6H), −2.10 (br, 1H), −2.24 (br, 1H).

Step 2: A 3-necked 100 mL RBF was charged with phyllochlorin N-(2-(2-(2-hydroxyethoxy)ethoxy)ethyl)-N-methyl-3-amide (500 mg, 0.765 mmol, 1 eq), 10% Pd/C (25 mg), ethyl acetate (15 mL), MeOH (5 mL) and a stirrer bar (˜15 mm). A hydrogen balloon was connected to the middle joint of the flask via a short length air condenser and the side-arm was connected to a 3-way tap. The setup was evacuated and then re-filled with nitrogen (3 times), evacuated and refilled with hydrogen (2 times). The resulting solution was then stirred (500 rpm) under the hydrogen atmosphere with heating at 30° C. for 2 hours. The solution was filtered through Celite® (0.5×2 cm), washing with ethyl acetate (˜10 mL) and the solvent was then removed under reduced pressure to give compound 12 as a dark green solid (507 mg, quantitative) (HPLC purity: 98.700).

¹H NMR (400 MHz, CDCl₃) δ 9.73 (m, 2H), 8.83 (brs, 1H), 8.78 (s, 1H), 4.68-4.62 (m, 1H), 4.57-4.49 (m, 1H), 4.00 (s, 3H), 3.96 (q, 2H), 3.86 (q, 2H), 3.65 (s, 3H), 3.51 (m, 1H), 3.46 (m, 1H), 3.45-3.38 (m, 9H), 3.37-3.34 (m, 2H), 3.00 (m, 1H), 2.72-2.68 (m, 1H), 2.67 (s, 1H), 2.65-2.50 (m, 3H), 2.40 (s, 2H), 2.38-2.20 (m, 3H), 1.94-1.84 (m, 1H), 1.81-1.74 (m, 9H), −2.15 (brm, 2H).

### Synthesis Example 13—Synthesis of Reduced Phyllochlorin N-(2-(2-(2-pyridiniumethoxy)ethoxy)ethyl)-N-methyl-3-amide chloride (Compound 13)

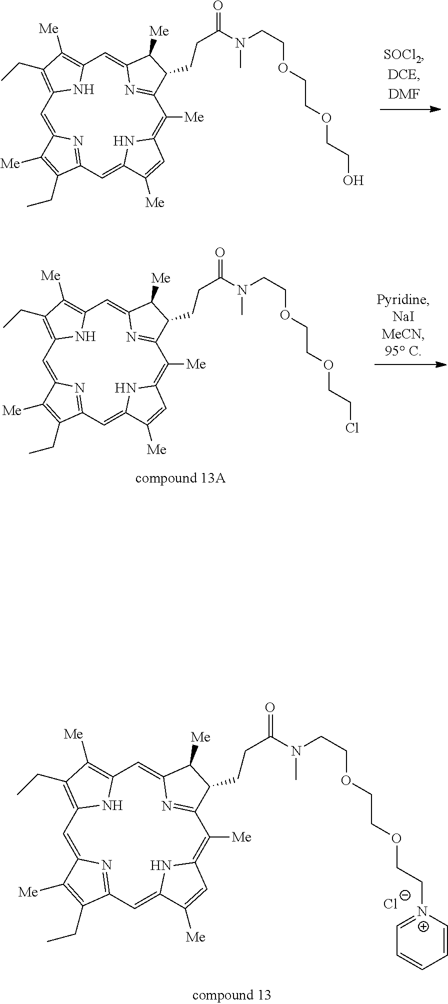 [SMILES: CCC1=C(C)c2cc3[nH]c(cc4nc(c(C)c5cc(C)c(cc1n2)[nH]5)[C@@H](CCC(=O)N(C)CCOCCOCCO)[C@@H]4C)c(C)c3CC ; CCC1=C(C)c2cc3[nH]c(cc4nc(c(C)c5cc(C)c(cc1n2)[nH]5)[C@@H](CCC(=O)N(C)CCOCCOCCCl)[C@@H]4C)c(C)c3CC ; CCC1=C(C)c2cc3[nH]c(cc4nc(c(C)c5cc(C)c(cc1n2)[nH]5)[C@@H](CCC(=O)N(C)CCOCCOCC[n+]1ccccc1)[C@@H]4C)c(C)c3CC ; [Cl-]]

Step 1: A 1-neck 50 mL RBF was charged with reduced phyllochlorin N-(2-(2-(2-hydroxyethoxy)ethoxy)ethyl)-N-methyl-3-amide (compound 12) (200 mg, 0.305 mmol, 1 eq), dichloroethane (4 mL) and thionyl chloride (54 mg, 0.457 mmol, 1.5 eq). The resultant solution was cooled using a water bath and stirred (350 rpm) under a nitrogen atmosphere. DMF (1 drop) was added and the mixture then stirred at room temperature for 1 hour and then heated at 40° C. for a further 1 hour. The reaction was then cooled using an ice/water bath and pH=7 phosphate buffer (10 mL) and water (10 mL) were added. The mixture was then extracted with DCM (2×15 mL), dried (Na₂SO₄) and the solvent was removed under reduced pressure to give the crude product as a dark green oil (0.6 g). The residue was purified by column chromatography (3×18 cm) eluting using a gradient of 2-4% MeOH/DCM. Fractions containing the major dark green spot (Rf=0.5 in 5% MeOH/DCM) were combined to give reduced phyllochlorin N-(2-(2-(2-chloroethoxy)ethoxy)ethyl)-N-methyl-3-amide (compound 13A) as a dark green solid (157 mg, 76%).

¹H NMR (400 MHz, CDCl₃) δ 9.71 (m, 1H), 9.53 (m, 1H), 8.81 (brs, 1H), 8.76 (s, 1H), 4.64 (m, 1H), 4.51 (m, 1H), 4.00 (m, 3H), 3.94 (m, 2H), 3.86 (q, 2H), 3.64 (s, 3H), 3.57 (m, 1H), 3.47 (m, 2H), 3.45-3.39 (m, 5H), 3.38-3.34 (m, 5H), 3.18 (t, 1H), 3.03 (t, 1H), 2.73-2.65 (m, 3H), 2.62-2.50 (m, 2H), 2.44 (s, 2H), 2.42-2.35 (m, 2H), 2.31-2.17 (m, 2H), 1.95-1.86 (m, 1H), 1.80-1.73 (m, 9H), −2.14 (brs, 1H), −2.17 (brs, 1H).

Step 2: To a sealed tube (2.5×20 cm with Teflon screw cap) containing reduced phyllochlorin N-(2-(2-(2-chloroethoxy)ethoxy)ethyl)-N-methyl-3-amide (compound 13A) (0.100 g, 0.148 mmol, 1 eq) and a stirrer bar was added MeCN (5 mL), pyridine (117 mg, 1.483 mmol, 10 eq) and NaI (44 mg, 0.297 mmol, 2 eq). After flushing the tube with nitrogen and sealing the tube, the dark green mixture was stirred (300 rpm) at 95° C. for 17 hours. The mixture was cooled and diluted with DCM (25 mL), washed with 0.1 M HCl (20 mL), pH=7 phosphate buffer (20 mL), dried (Na₂SO₄) and concentrated by rotary evaporation to give the crude product as a dark green oil. The residue was purified by column chromatography (3×15 cm) eluting using a gradient of 3-8% MeOH/DCM. Fractions containing the major dark green spot (Rf<0.1 in 5% MeOH/DCM) were combined to give compound 13 as a dark green solid (38 mg, 34%) (HPLC purity: 96.9%).

¹H NMR (400 MHz, CDCl₃) δ 9.68 (d, 1H), 9.51 (s, 1H), 8.80 (m, 2H), 7.43 (d, 1H), 6.95-6.75 (m, 2H), 6.33 (t, 1H), 6.21 (t, 1H), 4.63-4.48 (m, 2H), 3.98-3.91 (m, 5H), 3.82 (m, 2H), 3.63 (s, 3H), 3.41 (m, 3H), 3.37 (m, 3H), 3.32 (m, 1H), 3.27-3.18 (m, 2H), 3.10-3.02 (m, 3H), 3.0-2.95 (m, 1H), 2.83-2.76 (m, 2H), 2.60 (m, 3H), 2.55-2.40 (m, 3H), 2.35-2.20 (m, 2H), 2.18-2.00 (m, 2H), 1.81-1.67 (m, 13H), −2.33 (brs, 1H), −2.44 (brs, 1H).

### Synthesis Example 14—Synthesis of Reduced Phyllochlorin N-methyl-N-3,6,9,12,15,18-hexaoxanonadecyl propylamide (Compound 14)

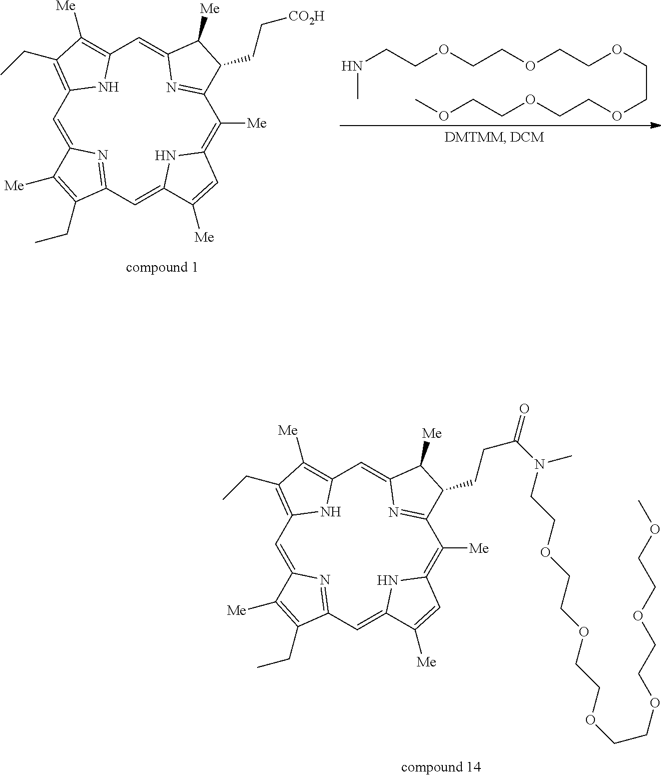 [SMILES: CCC1=C(C)c2cc3[nH]c(cc4nc(c(C)c5cc(C)c(cc1n2)[nH]5)[C@@H](CCC(=O)O)[C@@H]4C)c(C)c3CC ; CNCCOCCOCCOCCOCCOCCOC ; CCC1=C(C)c2cc3[nH]c(cc4nc(c(C)c5cc(C)c(cc1n2)[nH]5)[C@@H](CCC(=O)N(C)CCOCCOCCOCCOCCOCCOC)[C@@H]4C)c(C)c3CC]

Into a 50 mL RBF fitted with a nitrogen inlet and containing a small stirrer bar was added reduced phyllochlorin (compound 1) (100 mg, 0.194 mmol, 1 eq), dichloromethane (4 mL), 4-(4,6-dimethoxy-1,3,5-triazin-2-yl)-4-methyl-morpholinium chloride (DMTMM) (81 mg, 0.294 mmol, 1.5 eq) and N-methyl-2,5,8,11,14,17-hexaoxanonadecan-19-amine (91 mg, 0.294 mmol, 1.5 eq). The mixture was stirred at room temperature for 2 hours, monitoring by HPLC. The reaction mixture was transferred to a separatory funnel, diluted with DCM (20 mL) and washed with 0.5 M HCl (20 mL) and pH=7 phosphate buffer (20 mL). The organic layer was dried (Na₂SO₄) and concentrated by rotary evaporation to give the crude product as a dark green film (0.40 g). The residue was purified by column chromatography (3×16 cm) eluting using a gradient of 2-3% MeOH/DCM. Fractions containing the major dark green spot (Rf=0.25 in 5% MeOH/DCM) were combined to give compound 14 as a dark green oil (164 mg, quantitative) (HPLC purity: 94.4%).

¹H NMR (400 MHz, CDCl₃) δ 9.72 (m, 1H), 9.55 (m, 1H), 8.82 (s, 1H), 8.78 (s, 1H), 4.65 (m, 1H), 4.52 (m, 1H), 4.00 (m, 3H), 3.98-3.91 (m, 4H), 3.90-3.78 (m, 3H), 3.70-3.60 (m, 8H), 3.58-3.52 (m, 7H), 3.50-3.43 (m, 12H), 3.42-3.36 (m, 9H), 3.34 (m, 4H), 3.22 (m, 2H), 2.97 (m, 1H), 2.93-2.89 (m, 1H), 2.86-2.78 (m, 1H), 2.74 (s, 1H), 2.65-2.50 (m, 1H), 2.46 (s, 2H), 2.45-2.34 (m, 1H), 2.25-2.12 (m, 1H), 2.06-1.85 (m, 1H), 1.80-1.72 (m, 9H), −2.18 (s, 2H).

### Synthesis Example 15—Synthesis of Reduced Phyllochlorin 2-deoxy-D-mannosamine-propylamide (Compound 15)

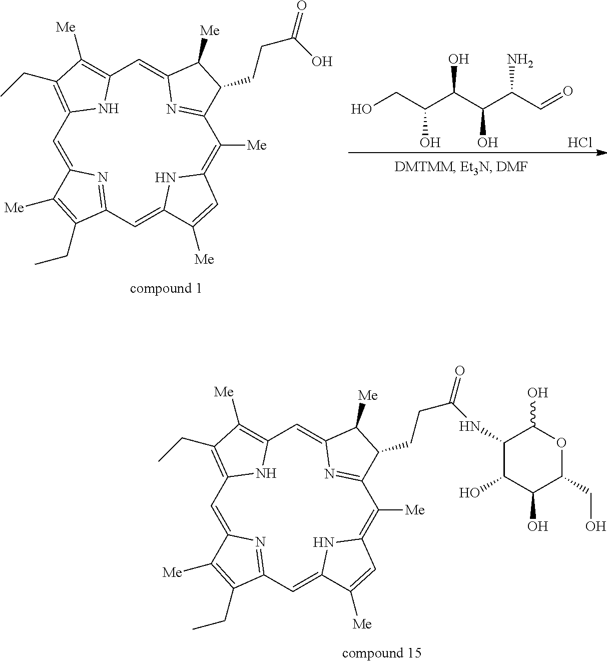 [SMILES: CCC1=C(C)c2cc3[nH]c(cc4nc(c(C)c5cc(C)c(cc1n2)[nH]5)[C@@H](CCC(=O)O)[C@@H]4C)c(C)c3CC ; N[C@H](C=O)[C@@H](O)[C@H](O)[C@H](O)CO ; Cl ; CCC1=C(C)c2cc3[nH]c(cc4nc(c(C)c5cc(C)c(cc1n2)[nH]5)[C@@H](CCC(=O)N[C@@H]1C(O)O[C@H](CO)[C@@H](O)[C@@H]1O)[C@@H]4C)c(C)c3CC]

To a 25 mL RBF containing a stirrer bar was added reduced phyllochlorin (compound 1) (100 mg, 0.196 mmol, 1 eq), DMTMM (70 mg, 0.255 mmol, 1.3 eq), DMF (1.5 mL), triethylamine (26 mg, 0.255 mmol, 1.3 eq) and (D)-mannosamine hydrochloride (55 mg, 0.255 mmol, 1.3 eq). The resultant mixture was stirred for 1 hour. The reaction mixture was concentrated using a rotary evaporator to give a black residue. The residue was purified by column chromatography (3×14 cm) eluting using a gradient of 5-12% MeOH/DCM. Fractions containing the major dark green spot (Rf=0.2 in 10% MeOH/DCM) were combined to give compound 15 as a dark green solid (97 mg, 73%).

¹H NMR (400 MHz, DMSO-d₆) δ 9.73 (s, 1H), 9.60 (s, 1H), 9.02 (s, 1H), 8.98 (m, 1H), 7.52 & 7.18 (2×d, 1H), 6.60 & 6.46 (2×d, 1H), 4.92 (m, 1H), 4.72-4.50 (m, 4H), 4.32-4.25 (m, 1H), 4.01-3.90 (m, 6H), 3.87-3.80 (m, 3H), 3.78-3.70 (m, 1H), 3.65-3.60 (m, 4H), 3.58-3.52 (m, 2H), 2.68-2.55 (m, 1H), 2.45-2.37 (m, 1H), 2.35-2.28 (m, 1H), 2.20-2.08 (m, 1H), 1.85-1.60 (m, 1H), −2.34 (brs, 1H), −2.40 (brs, 1H).

### Synthesis Example 16—Synthesis of Reduced Phyllochlorin N-(2-(2-(2-triphenylphosphoniumethoxy)ethoxy)ethyl)-N-methyl-3-amide chloride (Compound 16)

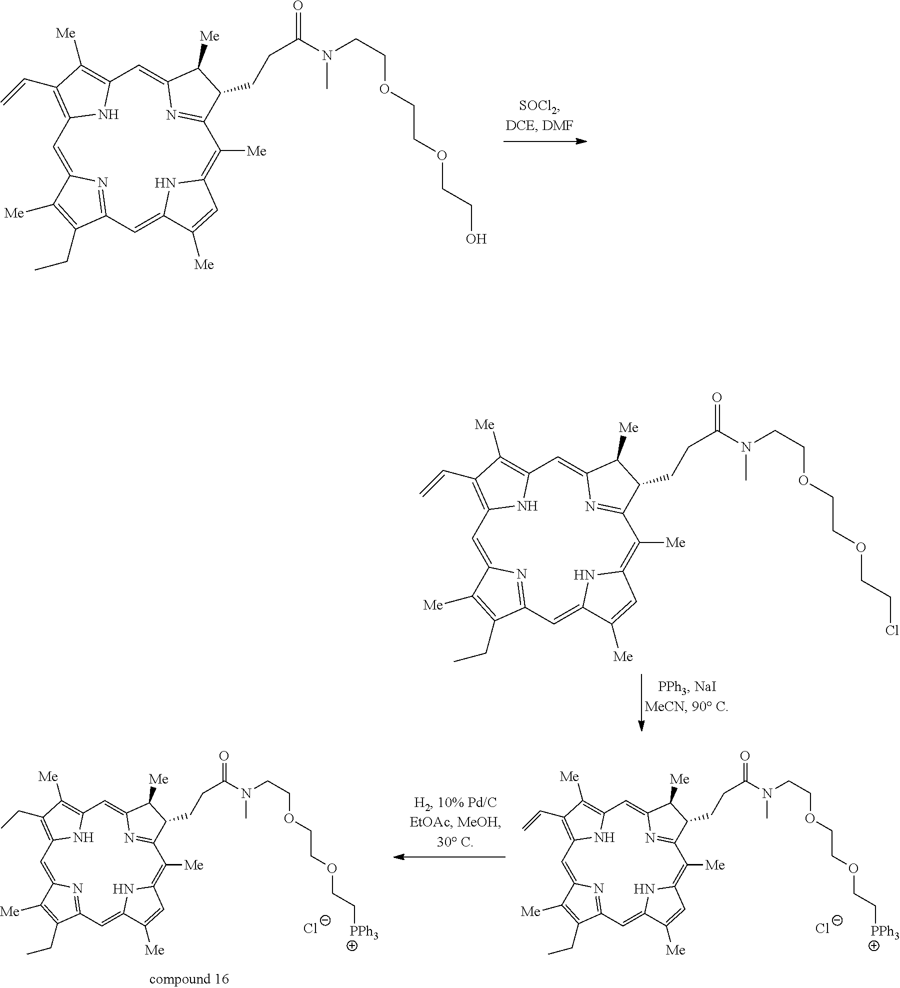

Step 1: A 1-necked 50 mL RBF was charged with phyllochlorin N-(2-(2-(2-hydroxyethoxy)ethoxy)ethyl)-N-methyl-3-amide (1.20 g, 1.84 mmol, 1 eq), dichloroethane (10 mL) and DMF (1 drop) and cooled using a water bath. Thionyl chloride (328 mg, 2.76 mmol, 1.5 eq) was dissolved in dichloroethane (2 mL) and the thionyl chloride solution added to the reaction over 1 minute. The resultant solution was stirred under a nitrogen atmosphere at room temperature for 2 hours with monitoring by HPLC. The reaction was then cooled using an ice/water bath and pH=7 phosphate buffer (30 mL) and water (30 mL) were added. The mixture was then extracted with DCM (2×30 mL), dried (Na₂SO₄) and the solvent was removed under reduced pressure to give 2.07 g of the crude product. The residual blue-green crude product was purified by column chromatography (4×18 cm of silica) using 1-2% MeOH in DCM as eluent. Fractions with spots at R_{f}=0.7 by TLC (5% MeOH/DCM eluent) were combined to give phyllochlorin N-(2-(2-(2-chloroethoxy)ethoxy)ethyl)-N-methyl-3-amide as a dark blue-green solid (924 mg, 75%) (HPLC purity: 97.2%).

¹H NMR (400 MHz, CDCl₃) δ 9.71 (m, 2H), 8.86 (m, 2H), 8.17 (dd, 1H), 6.38 (dd, 1H), 6.15 (dd, 1H), 4.66 (m, 1H), 4.54 (m, 1H), 4.01 (m, 3H), 3.86 (q, 2H), 3.64 (m, 3H), 3.59-3.50 (m, 4H), 3.48-3.45 (m, 2H), 3.44-3.39 (m, 2H), 3.40-3.30 (m, 5H), 3.22 (t, 1H), 3.09 (t, 1H), 2.77-2.65 (m, 3H), 2.62-2.50 (m, 2H), 2.48-2.43 (m, 2H), 2.43-2.15 (m, 2H), 1.98-1.83 (m, 1H), 1.80-1.72 (m, 6H), −2.07 (brs, 1H), −2.21 (brs, 1H).

Step 2: To a sealed tube (2.5×20 cm with Teflon screw cap) containing phyllochlorin N-(2-(2-(2-chloroethoxy)ethoxy)ethyl)-N-methyl-3-amide (150 mg, 0.223 mmol, 1 eq) and a stirrer bar was added MeCN (5 mL), triphenylphosphine (64 mg, 0.245 mmol, 1.1 eq) and sodium iodide (40 mg, 0.266 mmol, 1.2 eq). After flushing the tube with nitrogen and sealing the tube, the dark green mixture was stirred (300 rpm) at 90° C. for 70 hours with monitoring by HPLC. The mixture was cooled, transferred to a RBF and concentrated by rotary evaporation to give a dark green oil. The residue was purified by column chromatography (3×16 cm) using a gradient of 2-4% MeOH/DCM. Fractions containing the major dark green spot (Rf=0.1 in 5% MeOH/DCM) were combined to give phyllochlorin N-(2-(2-(2-triphenylphosphoniumethoxy)ethoxy)ethyl)-N-methyl-3-amide chloride (125 mg, 60%) (HPLC purity: 96.2%) as a dark green solid.

¹H NMR (400 MHz, CDCl₃) δ 9.69 (m, 2H), 8.84 (m, 2H), 8.14 (m, 1H), 7.55 (m, 6H), 7.44 (m, 5H), 7.23 (m, 2H), 7.06 (m, 2H), 6.35 (dd, 1H), 6.13 (m, 1H), 4.63 (m, 1H), 4.53 (m, 1H), 3.99 (m, 3H), 3.82 (q, 2H), 3.78-3.68 (m, 2H), 3.63 (m, 4H), 3.58-3.50 (m, 4H), 3.36-3.32 (m, 3H), 3.23-3.10 (m, 1H), 3.08 (m, 2H), 3.00 (m, 1H), 2.72-2.59 (m, 3H), 2.58-2.45 (m, 2H), 2.45-2.38 (m, 1H), 2.37 (s, 2H), 2.32-2.14 (m, 3H), 1.97-1.88 (m, 1H), 1.80-1.68 (m, 6H), −2.11 (brs, 1H), −2.23 (brs, 1H).

Step 3: A 3-necked 50 mL RBF was charged with phyllochlorin N-(2-(2-(2-triphenylphosphoniumethoxy)ethoxy)ethyl)-N-methyl-3-amide chloride (80 mg, 0.086 mmol, 1 eq), 10% Pd/C (10 mg), ethyl acetate (3 mL), MeOH (3 mL) and a stirrer bar. A hydrogen balloon was connected to the middle joint of the flask via a short length air condenser and the side-arm was connected to a 3-way tap. The setup was evacuated and then re-filled with nitrogen (3 times), evacuated and refilled with hydrogen (2 times). The resulting solution was then stirred (450 rpm) under the hydrogen atmosphere with heating at 30° C. for 22 hours. The solution was filtered through Celite® (0.5×2 cm), washing with methanol (˜10 mL) and the solvent was then removed under reduced pressure to give compound 16 as a dark green solid (63 mg, 79%) (HPLC purity: 95.5%).

¹H NMR (400 MHz, CDCl₃) δ 9.69 (m, 1H), 9.50 (m, 1H), 8.79 (m, 2H), 7.54 (m, 5H), 7.44 (m, 5H), 7.23 (m, 2H), 7.01 (m, 2H), 4.63 (m, 1H), 4.52 (m, 1H), 3.98 (m, 3H), 3.92 (m, 2H), 3.82 (m, 2H), 3.75-3.48 (m, 6H), 3.40 (s, 3H), 3.36-3.33 (m, 3H), 3.23-2.97 (m, 5H), 2.78-2.57 (m, 3H), 2.55-2.35 (m, 4H), 2.38 (m, 2H), 2.35-2.07 (m, 5H), 1.98-1.88 (m, 1H), 1.80-1.68 (m, 12H), −2.18 (brs, 2H).

### Synthesis Example 17—Synthesis of Reduced Phyllochlorin β-D-1-thioglucose-N-methylpropylamide sulfoxide (Compound 17)

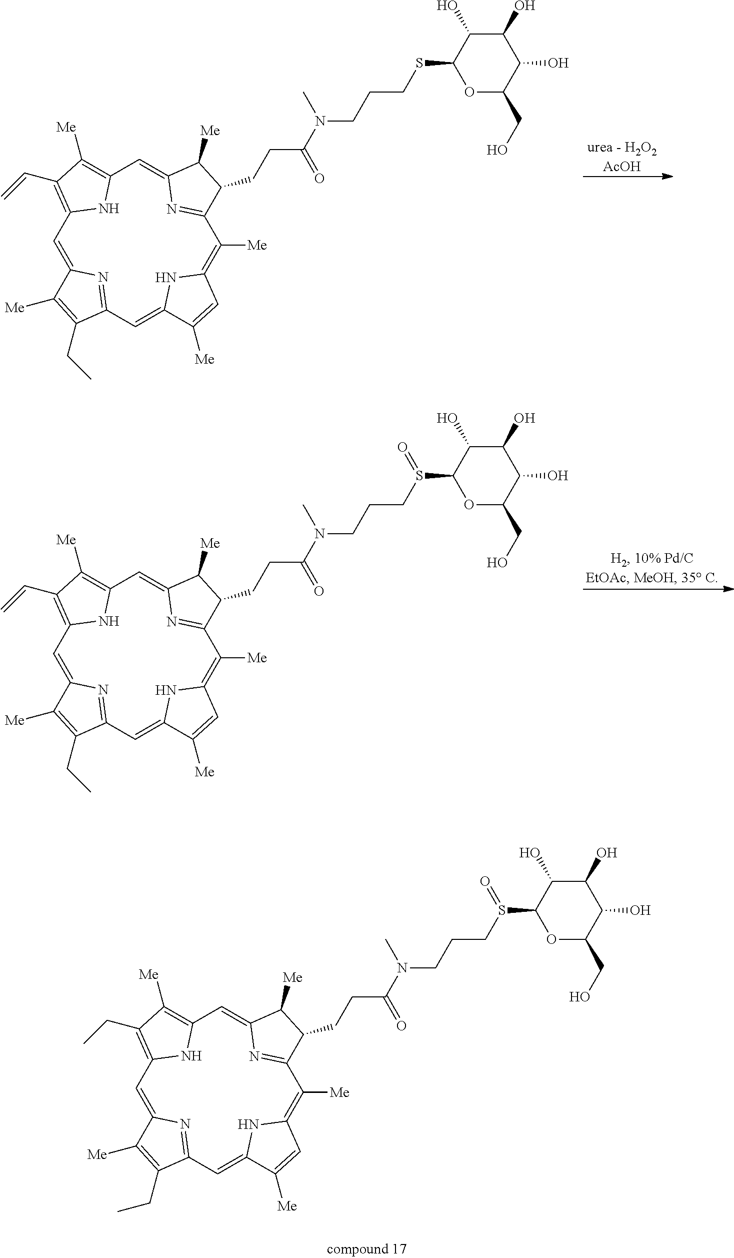 [SMILES: C=Cc1c(C)c2cc3nc(c(C)c4cc(C)c(cc5nc(cc1[nH]2)C(C)=C5CC)[nH]4)[C@@H](CCC(=O)N(C)CCCS[C@@H]1O[C@H](CO)[C@@H](O)[C@H](O)[C@H]1O)[C@@H]3C ; "urea - H2O2AcOH" ; C=Cc1c(C)c2cc3nc(c(C)c4cc(C)c(cc5nc(cc1[nH]2)C(C)=C5CC)[nH]4)[C@@H](CCC(=O)N(C)CCCS(=O)[C@@H]1O[C@H](CO)[C@@H](O)[C@H](O)[C@H]1O)[C@@H]3C ; "H2, 10% Pd/CEtOAc, MeOH, 35° C." ; CCC1=C(C)c2cc3[nH]c(cc4nc(c(C)c5cc(C)c(cc1n2)[nH]5)[C@@H](CCC(=O)N(C)CCCS(=O)[C@@H]1O[C@H](CO)[C@@H](O)[C@H](O)[C@H]1O)[C@@H]4C)c(C)c3CC]

Step 1: Into a single-neck 25 mL RBF was added phyllochlorin β-D-1-thioglucose-N-methylpropylamide (100 mg, 0.132 mmol, 1 eq), urea-hydrogen peroxide (18.6 mg, 0.198 mmol, 1.5 eq) and acetic acid (1.5 mL). The solution was stirred at 55° C. (external temperature, oil bath) for 2 hours. The acetic acid was removed under reduced pressure (rotary evaporator, 40° C., full vacuum) to leave a dark-green viscous oil. The crude product was purified by silica column chromatography (3×20 cm) using 10-16% MeOH/DCM. Fractions containing the product (R_{f} 0.3 in 10% MeOH/DCM) were combined to give phyllochlorin β-D-1-thioglucose-N-methylpropylamide sulfoxide as a dark green/blue flaky solid (91 mg, 89%).

¹H NMR (400 MHz, d₆-DMSO) δ 9.76-9.72 (m, 2H), 9.11-9.05 (m, 2H), 8.35 (dd, 1H), 6.44 (dd, 1H), 6.18 (dd, 1H), 5.70-5.30 (m, 1H), 5.24-5.02 (m, 2H), 4.80-4.60 (m, 2H), 4.60-4.52 (m, 1H), 4.31-4.10 (m, 1H), 3.99-3.95 (m, 3H), 3.81 (q, 2H), 3.75-3.65 (m, 1H), 3.62 (s, 3H), 3.55 (s, 3H), 3.51-3.39 (m, 3H), 3.33 (s, 3H), 3.17 (m, 1H), 3.16-2.97 (m, 2H), 2.90 (m, 2H), 2.84 (s, 1H), 2.82-2.55 (m, 2H), 2.47-2.38 (m, 1H), 1.99-1.75 (m, 2H), 1.73-1.66 (m, 7H), −2.25 (s, 1H), −2.43 (s, 1H).

Step 2: A 3-necked 50 mL RBF was charged with phyllochlorin β-D-1-thioglucose-N-methylpropylamide sulfoxide (75 mg, 0.097 mmol, 1 eq), 10% Pd/C (10 mg), ethyl acetate (3 mL), MeOH (2 mL) and a stirrer bar. A hydrogen balloon was connected to the middle joint of the flask via a short length air condenser and the side-arm was connected to a 3-way tap. The setup was evacuated and then re-filled with nitrogen (3 times), evacuated and refilled with hydrogen (2 times). The resulting solution was then stirred (500 rpm) under the hydrogen atmosphere with heating at 35° C. for 4 hours. The solution was filtered through Celite® (0.5×2 cm), washing with ethyl acetate (˜10 mL) and the solvent then removed under reduced pressure to give compound 17 as a dark green solid (67 mg, 89%) (HPLC purity: 98.3%).

¹H NMR (400 MHz, DMSO-d₆) δ 9.73 (s, 1H), 9.60 (s, 1H), 9.02-8.98 (m, 2H), 5.70-5.30 (m, 1H), 5.69-5.28 (m, 1H), 5.22-5.00 (m, 2H), 4.80-4.60 (m, 2H), 4.53 (m, 1H), 4.30-4.20 (m, 1H), 3.99-3.93 (m, 5H), 3.81 (q, 2H), 3.75-3.65 (m, 1H), 3.61 (s, 3H), 3.55-3.40 (m, 3H), 3.40 (m, 4H), 3.33 (s, 3H), 3.25 (m, 1H), 3.13-3.00 (m, 2H), 2.90 (m, 2H), 2.86 (s, 1H), 2.82-2.60 (m, 2H), 2.47-2.35 (m, 1H), 1.99-1.75 (m, 2H), 1.74-1.66 (m, 10H), −2.33 (s, 1H), −2.38 (s, 1H).

### Synthesis Example 18—Synthesis of Reduced Phyllochlorin 3-aminopropyl (Compound 18)

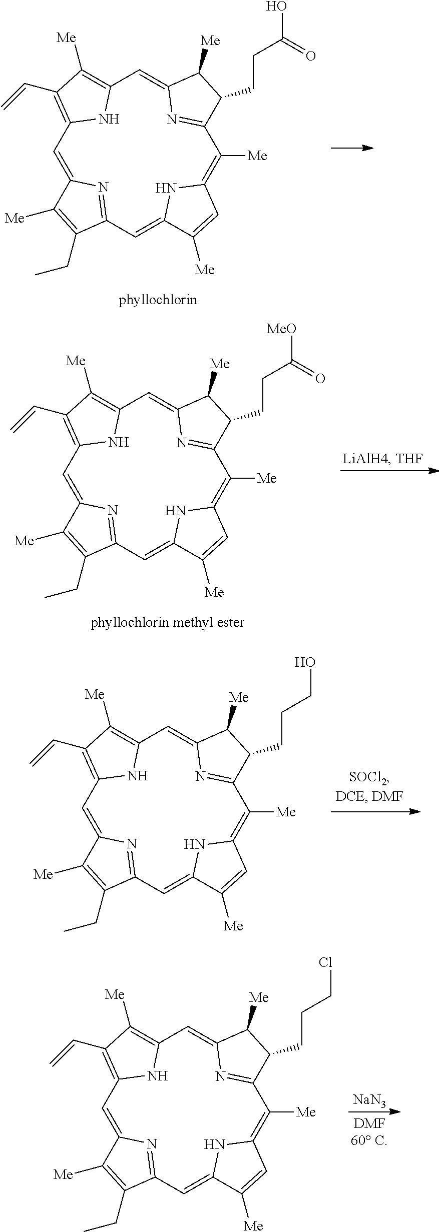 [SMILES: C=Cc1c(C)c2cc3nc(c(C)c4cc(C)c(cc5nc(cc1[nH]2)C(C)=C5CC)[nH]4)[C@@H](CCC(=O)O)[C@@H]3C ; C=Cc1c(C)c2cc3nc(c(C)c4cc(C)c(cc5nc(cc1[nH]2)C(C)=C5CC)[nH]4)[C@@H](CCC(=O)OC)[C@@H]3C ; C=Cc1c(C)c2cc3nc(c(C)c4cc(C)c(cc5nc(cc1[nH]2)C(C)=C5CC)[nH]4)[C@@H](CCCO)[C@@H]3C ; C=Cc1c(C)c2cc3nc(c(C)c4cc(C)c(cc5nc(cc1[nH]2)C(C)=C5CC)[nH]4)[C@@H](CCCCl)[C@@H]3C ; "DMF60° C." ; "NaN3"]

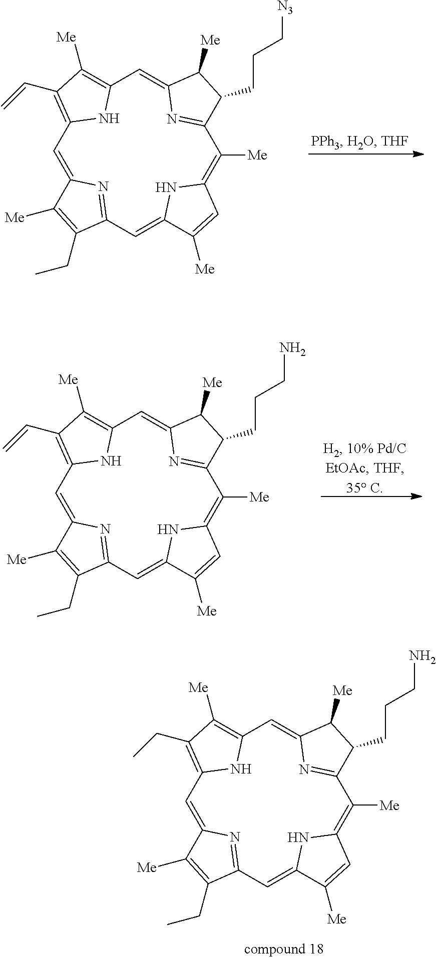

Step 1: Into a single-neck 100 mL RBF was added phyllochlorin (2.40 g, 4.72 mmol, 1 eq), potassium carbonate (0.78 g, 5.66 mmol, 1.2 eq), DMF (30 mL) and a small sized stirrer bar. The flask was placed under nitrogen and stirred at 300 rpm. Methyl iodide (382 μL, 6.13 mmol, 1.3 eq) was then added and the flask stirred at room temperature. HPLC analysis was undertaken after 2 hours and after stirring over the weekend, and confirmed the reaction was complete. The solution was diluted with DCM (30 mL) and filtered through Celite® (1 cm depth) washing with DCM until no more colour eluted. The solvent was removed under reduced pressure to give ˜4 g of a blue solid. The crude material was dissolved in EtOAc (125 mL) and washed with water (2×100 mL), dried (Na₂SO₄) and concentrated under reduced pressure to give crude product as a dark blue/green solid (2.7 g). The crude material was purified by column chromatography (silica, 4×23 cm, graduated solvent of DCM to 3% MeOH/DCM) to give phyllochlorin methyl ester as a dark blue/green solid (1.60 g, 64.8%).

¹H NMR (400 MHz, CDCl₃) δ −2.22 (br, 1H), −2.10 (br, 1H), 1.72-1.80 (m, 6H), 2.20-2.10 (m, 1H), 2.10-2.00 (m, 1H), 2.48-2.62 (m, 2H), 3.38 (s, 3H), 3.53 (s, 3H), 3.58 (s, 3H), 3.64 (s, 3H), 3.84 (q, 2H), 3.98 (s, 3H), 4.50 (q, 1H), 4.56 (d, 1H), 6.13 (dd, 1H), 6.37 (dd, 1H), 8.16 (dd, 1H), 8.83 (br s, 2H), 9.71 (br s, 2H).

Step 2: Into a 2-neck 500 mL RBF was added lithium aluminium hydride (670 mg, 16.8 mmol, 5 eq) and THF (200 mL). The solution was stirred (350 rpm) and cooled using an ice/water bath for 10 minutes under nitrogen. Phyllochlorin methyl ester (1.75 g, 3.35 mmol, 1 eq) was added in portions over 5 minutes. After 30 minutes, further lithium aluminium hydride (670 mg, 16.8 mmol, 5 eq) was added and stirring was continued for 90 minutes during which the reaction was allowed to warm slightly. TLC analysis showed the reaction to be complete. The flask was cooled (ice/water bath) and water (1.3 mL) was added, followed by 4M NaOH (1.3 mL). After stirring for 10 minutes, further water (4.0 mL) was added and the solution was warmed to room temperature and stirred for 15 minutes. Sodium sulfate was added and the mixture stirred for 10 minutes before being filtered, rinsing with DCM until no more colour eluted. The solvent was then removed under reduced pressure to give ˜2.3 g of crude product (still containing some solvent). HPLC analysis at this stage indicated a purity of 97.3% for the crude product. The crude material was purified by column chromatography (silica, 4×11 cm, 2-4% MeOH/DCM) to give phyllochlorin 3-hydroxypropyl as a dark blue/green solid (1.60 g, 97%) (HPLC purity: 97.6%).

¹H NMR (400 MHz, CDCl₃) δ 9.72 (m, 2H), 8.88 (s, 1H), 8.84 (m, 1H), 8.16 (dd, 1H), 6.48 (dd, 1H), 6.14 (dd, 1H), 4.51 (m, 2H), 3.94 (s, 3H), 3.85 (q, 2H), 3.63 (s, 3H), 3.60-3.50 (m, 5H), 3.38 (s, 3H), 2.21-2.612 (m, 1H), 1.85-1.72 (m, 8H), 1.52-1.40 (m, 2H), 1.12-1.05 (m, 1H), −2.10 (br, 1H), −2.22 (br, 1H).

Step 3: A single-neck 25 mL RBF was charged with phyllochlorin 3-hydroxypropyl (0.50 g, 1.01 mmol, 1 eq), dichloroethane (4 mL) and thionyl chloride (200 mg, 1.68 mmol, 1.65 eq). The resultant solution was cooled using an ice/water bath and stirred (350 rpm) under a nitrogen atmosphere. DMF (1 drop) was added and the mixture then stirred at room temperature for 1 hour, then at 40° C. for a further 1 hour at which point HPLC analysis showed the reaction was complete. The reaction was then cooled using an ice/water bath and pH 7 phosphate buffer (10 mL) was added. The mixture was extracted with DCM (2×15 mL), dried (Na₂SO₄) and concentrated under reduced pressure to give the crude chloride. Column chromatography (3×10 cm of silica) using 1% MeOH/DCM gave phyllochlorin 3-chloropropyl as a dark blue/green solid (450 mg, 87%).

¹H NMR (400 MHz, CDCl₃) δ 9.71 (m, 2H), 8.85 (s, 1H), 8.84 (m, 1H), 8.17 (dd, 1H), 6.37 (dd, 1H), 6.13 (dd, 1H), 4.51 (m, 2H), 3.96 (s, 3H), 3.84 (q, 2H), 3.64 (s, 3H), 3.53 (s, 3H), 3.49-3.42 (m, 2H), 3.38 (s, 3H), 2.35-2.25 (m, 1H), 2.08-1.95 (m, 1H), 1.94-1.85 (m, 1H), 1.80-1.72 (m, 6H), 1.71-1.60 (m, 1H), 1.04-1.01 (m, 3H), −2.10 (br, 1H), −2.23 (br, 1H).

Step 4: To a 100 mL RBF was added phyllochlorin 3-chloropropyl (3.14 g, 6.12 mmol, 1 eq) and DMF (30 mL). Sodium azide (477 mg, 7.34 mmol, 1.2 eq) was then added and the resultant mixture stirred overnight at 60° C. A further portion of sodium azide (119 mg, 1.84 mmol, 0.3 eq) was added, and the reaction stirred for a further 3 hours at 60° C. by which time it was complete as monitored by HPLC. The solvent was removed by rotary evaporation to give crude product as a dark residue. The crude product was purified by column chromatography using a stepped column (40/57 mm), a silica height of 35 cm and mobile phase of 0-5% MeOH in DCM. Fractions containing the product were combined to give phyllochlorin 3-azidopropyl as a blue/black solid (878 mg, 28%).

¹H NMR (400 MHz, CDCl₃) δ 9.73 (d, J=2.8 Hz, 2H), 8.87 (s, 1H), 8.84 (s, 1H), 8.16 (dd, J=17.8, 11.5 Hz, 1H), 6.37 (dd, J=17.8, 1.5 Hz, 1H), 6.14 (dd, J=11.6, 1.5 Hz, 1H), 4.57-4.48 (m, 2H), 3.96 (s, 3H), 3.85 (q, J=7.6 Hz, 2H), 3.65 (d, J=1.0 Hz, 3H), 3.54 (s, 3H), 3.37 (s, 3H), 3.21 (t, J=6.5 Hz, 2H), 2.27-2.16 (m, 1H), 1.90-1.81 (m, 1H), 1.79 (d, J=7.3 Hz, 3H), 1.75 (t, J=7.6 Hz, 3H), 1.52 (s, 2H), −2.11 (s, 1H), −2.22 (s, 1H).

Step 5: A 10 mL RBF was charged with phyllochlorin 3-azidopropyl (628 mg, 1.21 mmol, 1 eq), THF (2 mL) and water (0.4 mL). Triphenylphosphine (380 mg, 1.45 mmol, 1.2 eq) was then added in one portion and the resultant mixture stirred for 3 hours, by which time the reaction was complete as monitored by TLC. The solvent was removed by rotary evaporation. The residue was taken up in DCM (10 mL), dried over Na₂SO₄, filtered and concentrated to a dark residue which was purified by column chromatography using a stepped column (27/40 mm), a silica height of 30 cm and a gradient of 10%→60% MeOH/DCM. The main product fractions were combined to give phyllochlorin 3-aminopropyl as a blue/black solid (393 mg, 66%).

¹H NMR (400 MHz, d₆-DMSO) δ 9.76 (d, J=8.4 Hz, 2H), 9.12 (s, 1H), 9.09 (d, J=1.8 Hz, 1H), 8.36 (dd, J=17.9, 11.6 Hz, 1H), 7.10 (s, 2H), 6.46 (dd, J=17.8, 1.6 Hz, 1H), 6.19 (dd, J=11.6, 1.5 Hz, 1H), 4.59 (d, J=7.6 Hz, 3H), 3.94 (s, 3H), 3.83 (q, J=7.6 Hz, 2H), 3.64 (s, 3H), 3.56 (s, 3H), 2.75 (s, 1H), 2.70-2.58 (m, 1H), 2.19-2.06 (m, 1H), 1.87-1.64 (m, 9H), 1.23 (d, J=9.5 Hz, 1H), −2.27 (s, 1H), −2.43 (d, J=2.2 Hz, 1H).

Step 6: A 3-necked 50 mL RBF was charged with phyllochlorin 3-aminopropyl (60 mg, 0.122 mmol, 1 eq), 10% Pd/C (10 mg), ethyl acetate (2 mL), THF (3 mL) and a stirrer bar. A hydrogen balloon was connected to the middle joint of the flask via a short length air condenser and the side-arm was connected to a 3-way tap. The setup was evacuated and then re-filled with nitrogen (3 times), evacuated and refilled with hydrogen (2 times). The resulting solution was then stirred (500 rpm) under the hydrogen atmosphere with heating at 35° C. for 2 hours. The solution was filtered through Celite® (0.5×2 cm), washing with ethyl acetate (˜10 mL) and the solvent then removed under reduced pressure to give compound 18 as a dark green solid (48 mg, 80%) (HPLC purity: 93.1%).

¹H NMR (400 MHz, CDCl₃) δ 9.73 (s, 1H), 9.55 (s, 1H), 8.84-8.76 (m, 2H), 4.51-4.40 (m, 2H), 3.99-3.93 (m, 2H), 3.92-3.84 (m, 5H), 3.64 (m, 3H), 3.45-3.38 (m, 6H), 2.62-2.54 (m, 1H), 2.53-2.45 (m, 1H), 2.30-1.98 (m, 4H), 1.97-1.85 (m, 1H), 1.82-1.75 (m, 9H), 1.70-1.62 (m, 2H), −2.15 (brs, 2H).

### Synthesis Example 19—Synthesis of Reduced Phyllochlorin N-2-hydroxyethyl-N-methyl propylamide (Compound 19)

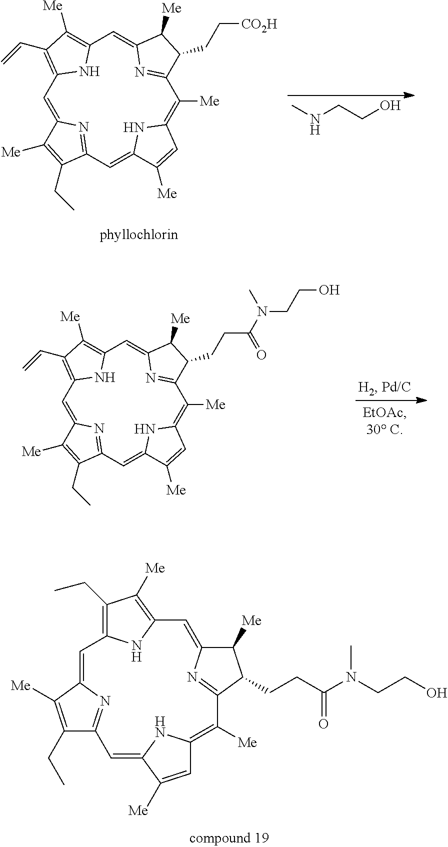

Step 1: Into a 250 mL RBF fitted with a nitrogen inlet and containing a stirrer bar was added phyllochlorin (3.00 g, 5.90 mmol, 1 eq), dichloromethane (70 mL), PyBOP (3.30 g, 1.1 eq), triethylamine (2.46 mL, 3 eq) and 2-(methylamino)-ethanol (0.53 g, 1.2 eq). The mixture was stirred at room temperature for 3 hours. Analysis by TLC showed the reaction to be complete. The reaction mixture was transferred to a separatory funnel and washed with water (2×30 mL). The organic layer was dried (Na₂SO₄), filtered through Celite® and concentrated by rotary evaporation to give the crude product as a blue/brown film (6.0 g). The crude product was loaded directly onto a silica column and eluted with 1-3% MeOH/DCM. Pure fractions containing a green/blue spot by TLC (R_{f} 0.40 in 5% MeOH/DCM) were combined to give phyllochlorin N-2-hydroxyethyl-N-methyl propylamide (0.62 g, 25%).

¹H NMR (400 MHz, CDCl₃) δ 9.78-9.70 (m, 2H), 8.85 (m, 2H), 8.20-8.10 (m, 1H), 6.38 (d, 1H), 6.14 (d, 1H), 4.70-4.65 (m, 1H), 4.58-4.50 (m, 1H), 4.01 (m, 3H), 3.89-3.82 (m, 2H), 3.65 (s, 3H), 3.53 (s, 3H), 3.37 (m, 4H), 3.31-3.22 (m, 1H), 3.14-3.08 (m, 2H), 2.65-2.51 (m, 3H), 2.37-2.22 (m, 3H), 2.09 (s, 2H), 1.88-1.70 (m, 8H), 1.62-1.50 (br s, 2H), −2.05-−2.32 (m, 2H).

Step 2: A 3-necked 100 mL RBF was charged with phyllochlorin N-2-hydroxyethyl-N-methyl propylamide (200 mg, 0.354 mmol, 1 eq), 10% Pd/C (15 mg), ethyl acetate (20 mL) and a stirrer bar (˜15 mm). A hydrogen balloon was connected to the middle joint of the flask via a short length air condenser and the side-arm was connected to a 3-way tap. The setup was evacuated and then re-filled with nitrogen (3 times), evacuated and refilled with hydrogen (2 times). The resulting solution was then stirred (600 RPM) under the hydrogen atmosphere with heating at 30° C. for 2 hours. The reaction progress was monitored by HPLC. The solution was filtered through Celite® (0.5×3 cm), washing with ethyl acetate (˜20 mL) and the solvent was then removed under reduced pressure to give 202 mg of crude product. The residual solid was purified by passage through a silica gel plug (3×3 cm) eluting with 5% MeOH/DCM to give compound 19 as a dark blue solid (162 mg, 81%) (HPLC purity: 98.7%).

¹H NMR (400 MHz, CDCl₃) δ 9.85 (s, 1H), 9.73 (s, 1H), 9.55 (s, 1H), 8.96-8.85 (m, 1H), 8.83-8.80 (m, 1H), 8.77 (s, 1H), 4.82-4.70 (m, 1H), 4.69-4.63 (m, 1H), 4.61-4.55 (m, 1H), 4.50 (q, J=7.3 Hz, 1H), 4.07-4.01 (m, 2H), 3.98 (s, 3H), 3.97-3.91 (m, 2H), 3.85 (q, J=7.5 Hz, 2H), 3.68-3.62 (m, 4H), 3.41 (d, J=3.6 Hz, 6H), 3.38 (s, 5H), 3.31-3.23 (m, 1H), 3.14-3.07 (m, 2H), 2.63 (s, 1H), 2.53 (s, 2H), 2.48-2.18 (m, 2H), 2.08 (s, 3H), 1.90-1.69 (m, 11H), 1.56 (s, 4H), −2.19 (s, 2H).

### Synthesis Example 20—Synthesis of Reduced Phyllochlorin 3-hydroxypropyl (Compound 20)

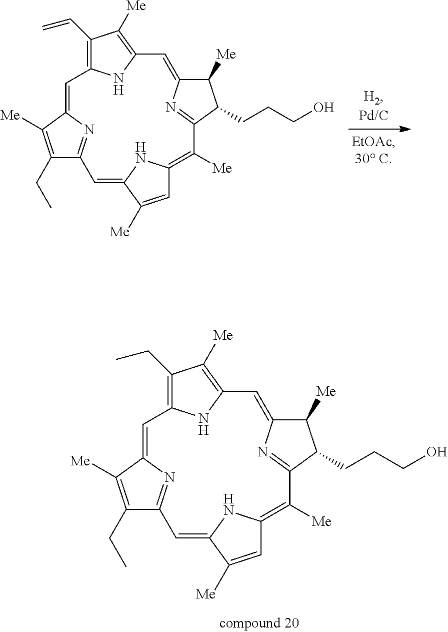 [SMILES: C=Cc1c(C)c2cc3nc(c(C)c4cc(C)c(cc5nc(cc1[nH]2)C(C)=C5CC)[nH]4)[C@@H](CCCO)[C@@H]3C ; CCC1=C(C)c2cc3[nH]c(cc4nc(c(C)c5cc(C)c(cc1n2)[nH]5)[C@@H](CCCO)[C@@H]4C)c(C)c3CC]

A 3-necked 100 mL RBF was charged with phyllochlorin 3-hydroxypropyl (100 mg, 0.202 mmol, 1 eq), 10% Pd/C (10 mg), ethyl acetate (20 mL) and a stirrer bar (˜15 mm). A hydrogen balloon was connected to the middle joint of the flask via a short length air condenser and the side-arm was connected to a 3-way tap. The setup was evacuated and then re-filled with nitrogen (3 times), evacuated and refilled with hydrogen (2 times). The resulting solution was then stirred (600 RPM) under the hydrogen atmosphere with heating at 30° C. for 2 hours. The solution was filtered through Celite® (1×3 cm), washing with ethyl acetate (˜10 mL) and the solvent was then removed under reduced pressure to give 170 mg of crude product. The crude product was purified by passage through a plug of silica gel (3×3 cm) eluting with 5% MeOH/DCM to give compound 20 as a dark blue solid (94 mg, 94%) (HPLC purity: 98.2%).

¹H NMR (400 MHz, CDCl₃) δ 9.73 (s, 1H), 9.56 (s, 1H), 8.84-8.74 (m, 2H), 4.51 (q, J=7.2 Hz, 2H), 3.99-3.91 (m, 5H), 3.85 (q, J=7.6 Hz, 2H), 3.66-3.62 (m, 3H), 3.57-3.45 (m, 2H), 3.41 (s, 3H), 3.38 (s, 3H), 2.23-2.12 (m, 1H), 1.86-1.71 (m, 10H), 1.52 (s, 2H), 1.47-1.33 (m, 1H), 1.10 (s, 1H), −2.20 (s, 2H).

### Synthesis Example 21—Synthesis of Reduced Phyllochlorin ((methylamino)-10-decyl)triphenylphosphonium Chloride Propylamide (Compound 21)

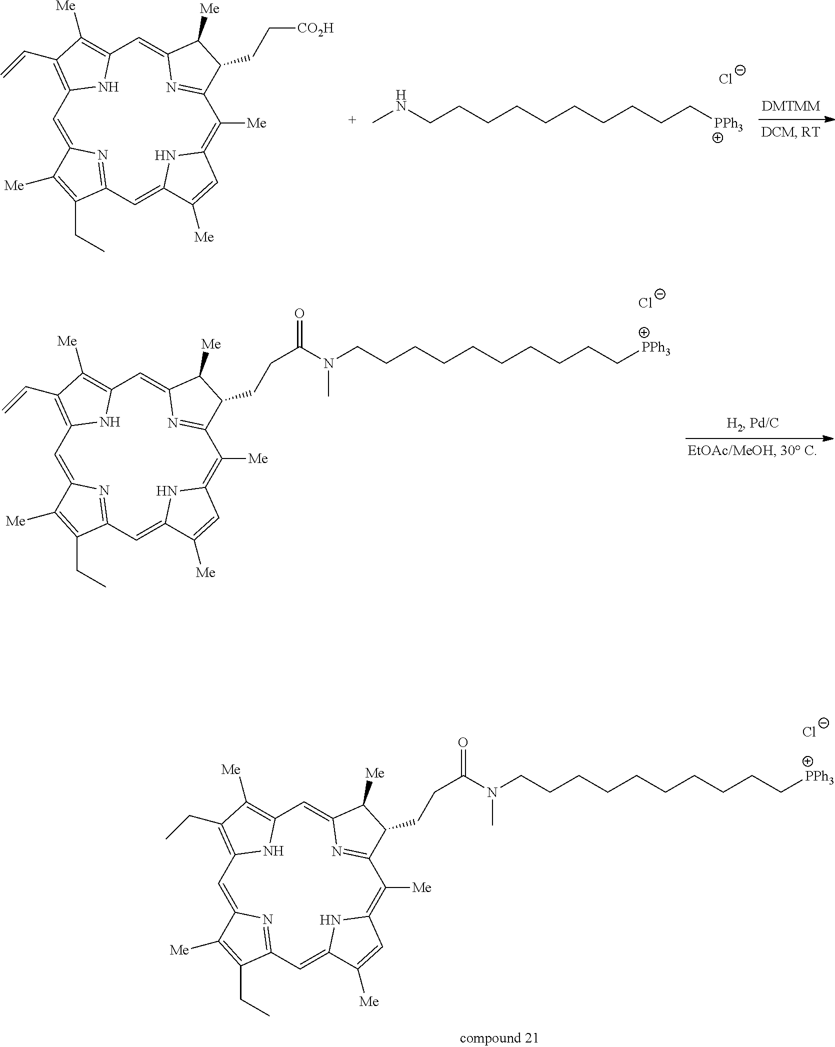

Step 1: Into a 50 mL RBF fitted with a nitrogen inlet and containing a small stirrer bar was added phyllochlorin (227 mg, 0.446 mmol, 1 eq), dichloromethane (15 mL), DMTMM (160 mg, 0.580 mmol, 1.3 eq) and (10-(methylamino)decyl)triphenyl-phosphonium chloride (417 mg, 0.891 mmol, 2 eq). The mixture was stirred at room temperature for 1 hour. The reaction mixture was then concentrated by rotary evaporation to give the crude product as a dark blue viscous syrup. The crude mixture was loaded directly onto a silica column (4×20 cm, pre-equilibrated with 1% MeOH in DCM) and eluted with 1% to 3% MeOH in DCM. Fractions containing a green/blue spot by TLC with R_{f}=0.37 (5% MeOH in DCM) were combined and concentrated by rotary evaporation to give a dark blue solid (422 mg). The compound was then dissolved in DCM (50 mL), washed with 0.1M HCl (50 mL) then H₂O (50 mL). The organic layer was then dried over Na₂SO₄, filtered on a sintered glass frit and concentrated by rotary evaporation. The residue was dried in a vacuum oven at 60° C. for 2 hours to give phyllochlorin ((methylamino)-10-decyl)triphenylphosphonium chloride propylamide as a dark blue solid (278 mg, 65%) (HPLC purity: 94.5%).

¹H NMR (400 MHz, CDCl₃) δ 9.73-9.64 (m, 2H), 8.87-8.77 (m, 2H), 8.21-8.08 (m, 1H), 7.85-7.66 (m, 10H), 7.65-7.57 (m, 6H), 6.36 (dd, J=17.8, 6.3, 1.6 Hz, 1H), 6.12 (dd, J=11.5, 6.8, 1.5 Hz, 1H), 4.65-4.48 (m, 2H), 4.00 (d, J=7.6 Hz, 3H), 3.88-3.77 (m, 2H), 3.62 (s, 3H), 3.52 (d, J=5.0 Hz, 3H), 3.35 (d, J=7.3 Hz, 3H), 3.22-3.14 (m, 1H), 2.71 (s, 1H), 2.66-2.51 (m, 1H), 2.46 (s, 2H), 2.15-2.06 (m, 1H), 2.06-1.95 (m, 1H), 1.80-1.69 (m, 6H), 1.68-1.58 (m, 3H), 1.58-1.43 (m, 4H), 1.37-0.99 (m, 7H), 0.98-0.78 (m, 2H), 0.75-0.63 (m, 1H), −2.08 (s, 1H), −2.21 (s, 1H).

Step 2: A 3-necked 100 mL RBF was charged with phyllochlorin ((methylamino)-10-decyl)triphenylphosphonium chloride propylamide (70 mg, 0.0729 mmol, 1 eq), 10% Pd/C (10 mg), ethyl acetate (7 mL), methanol (7 mL) and a stirrer bar (˜15 mm). A hydrogen balloon was connected to the middle joint of the flask via a short length air condenser and the side-arm was connected to a 3-way tap. The setup was evacuated and then re-filled with nitrogen (3 times), evacuated and refilled with hydrogen (2 times). The resulting solution was then stirred (600 RPM) under the hydrogen atmosphere with heating at 30° C. for 2 hours. The solution was filtered through Celite® (1×3 cm), washing with methanol (˜10 mL) and the solvent was then removed under reduced pressure to give 81 mg of crude product. The residual solid was purified by passage through a plug of silica gel (3×3 cm) eluting with 5% MeOH/DCM to give compound 21 as a dark blue solid (60 mg, 86%) (HPLC purity: 95.0%).

¹H NMR (400 MHz, CDCl₃) δ 9.70 (d, J=7.0 Hz, 1H), 9.53 (d, J=8.5 Hz, 1H), 8.85-8.71 (m, 2H), 7.86-7.67 (m, 9H), 7.67-7.58 (m, 6H), 4.62 (d, J=9.1, 2.8 Hz, 1H), 4.56-4.44 (m, 1H), 3.99 (d, J=6.9 Hz, 3H), 3.97-3.89 (m, 1H), 3.84 (p, J=7.5 Hz, 2H), 3.67-3.49 (m, 3H), 3.38 (dd, J=12.2, 5.4 Hz, 6H), 3.22-3.13 (m, 1H), 2.71 (s, 1H), 2.64 (t, J=7.6 Hz, 1H), 2.60-2.50 (m, 1H), 2.44 (s, 2H), 2.17-2.04 (m, 1H), 2.04-1.91 (m, 1H), 1.80-1.68 (m, 9H), 1.62 (s, 5H), 1.52 (s, 4H), 1.37-1.01 (m, 8H), 0.99-0.82 (m, 1H), 0.78-0.65 (m, 1H), −2.19 (s, 2H).

### Synthesis Example 22—Synthesis of Reduced Phyllochlorin N-3-hydroxypropyl-N-methyl Propylamide (Compound 22)

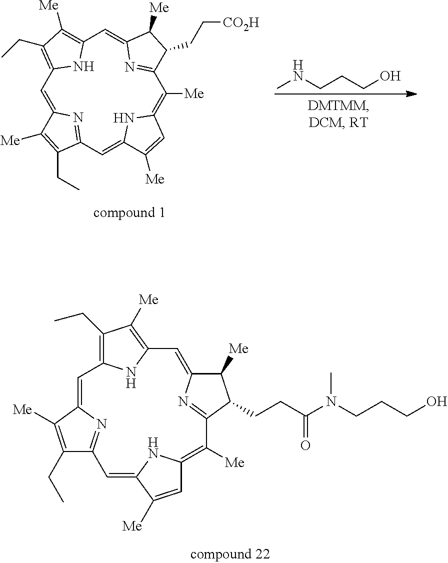

To a 25 mL RBF containing a stirrer bar was added reduced phyllochlorin (compound 1) (100 mg, 0.196 mmol, 1 eq), DMTMM (71 mg, 0.255 mmol, 1.3 eq), DCM (4 mL) and 3-(Methylamino)-1-propanol (26 mg, 0.294 mmol, 1.5 eq). The resultant mixture was stirred for 1 hour, at which point HPLC analysis showed the reaction to be complete. The reaction mixture was diluted with the addition of DCM (20 mL) and then washed with saturated aqueous NaHCO₃ solution (2×20 mL) and brine (20 mL). The organic layer was then dried with Na₂SO₄, filtered on a sintered glass frit and concentrated by rotary evaporation to give the crude product as a dark blue solid. The residue was subjected to column chromatography (graduated column, silica gel, 4×8 cm then 2×15 cm) using 1-1.5% MeOH in DCM as eluent. Fractions containing the product (Major dark green spot, R_{f}=0.43 in 5% MeOH/DCM) were combined and the solvent removed by rotary evaporation to give compound 22 as a dark blue solid (39 mg, 34%) (HPLC purity: 96.9%).

¹H NMR (400 MHz, CDCl₃) δ 9.84-9.72 (m, 1H), 9.67-9.50 (m, 1H), 8.88-8.67 (m, 2H), 4.76-4.61 (m, 1H), 4.51 (q, J=7.3 Hz, 1H), 4.05-3.91 (m, 5H), 3.91-3.80 (m, 2H), 3.68-3.62 (m, 3H), 3.39 (d, J=10.6 Hz, 6H), 3.32-3.11 (m, 3H), 2.63-2.49 (m, 1H), 2.45 (s, 1H), 2.27 (d, J=9.1 Hz, 1H), 2.14 (s, 2H), 1.93-1.69 (m, 10H), 1.55 (s, 2H), 1.46-1.38 (m, 2H), 1.26 (s, 1H), −2.18 (s, 2H).

### Synthesis Example 23—Synthesis of Reduced Phyllochlorin N-5-hydroxypentyl-N-methyl Propylamide (Compound 23)

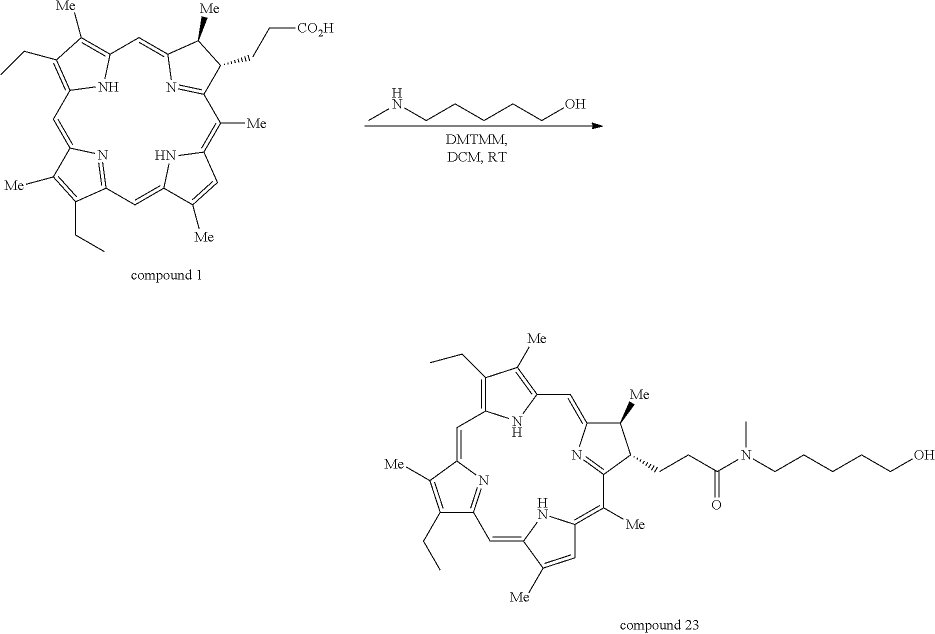

To a 25 mL RBF containing a stirrer bar was added reduced phyllochlorin (compound 1) (100 mg, 0.196 mmol, 1 eq), DMTMM (71 mg, 0.255 mmol, 1.3 eq), DCM (4 mL) and 3-(Methylamino)-1-propanol (34 mg, 0.294 mmol, 1.5 eq). The resultant mixture was stirred for 1 hour, at which point HPLC analysis showed the reaction to be complete. The reaction mixture was diluted with the addition of DCM (20 mL) and then washed with saturated aqueous NaHCO₃ solution (2×20 mL) and brine (20 mL). The organic layer was then dried with Na₂SO₄, filtered on a sintered glass frit and concentrated by rotary evaporation to give the crude product as a dark blue solid. The crude product was subjected to column chromatography (graduated column, silica gel, 4×8 cm then 2×15 cm) using 1-1.5% MeOH in DCM as eluent. Fractions containing the product (Major dark green spot, R_{f}=0.36 in 5% MeOH/DCM) were combined and the solvent removed by rotary evaporation to give compound 23 as a dark blue solid (57 mg, 48%) (HPLC purity: 97.6%).

¹H NMR (400 MHz, CDCl₃) δ 9.74 (s, 1H), 9.56 (s, 1H), 8.88-8.73 (m, 2H), 4.76-4.60 (m, 1H), 4.57-4.41 (m, 1H), 3.99 (s, 3H), 3.96-3.91 (m, 2H), 3.85 (q, J=7.5 Hz, 2H), 3.63 (s, 3H), 3.52 (t, J=6.5 Hz, 1H), 3.40 (s, 3H), 3.38 (s, 3H), 3.17-3.04 (m, 1H), 2.55 (s, 2H), 2.43 (s, 1H), 2.23 (s, 2H), 2.11-1.98 (m, 2H), 1.80-1.68 (m, 9H), 1.56 (s, 5H), 1.52-1.41 (m, 1H), 1.40-1.14 (m, 3H), 0.59-0.45 (m, 1H), 0.29-0.15 (m, 1H), 0.12-−0.03 (m, 2H), −2.21 (s, 2H).

### Synthesis Example 24—Synthesis of Reduced Phyllochlorin 2-deoxy-D-galactosamine-propylamide (Compound 24)

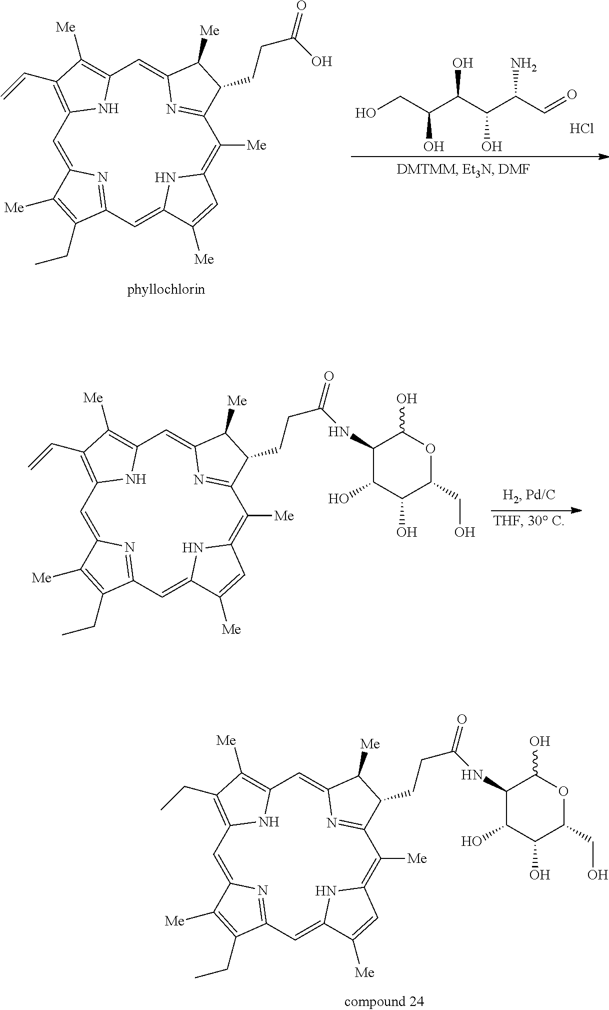 [SMILES: C=Cc1c(C)c2cc3nc(c(C)c4cc(C)c(cc5nc(cc1[nH]2)C(C)=C5CC)[nH]4)[C@@H](CCC(=O)O)[C@@H]3C ; N[C@H](C=O)[C@H](O)[C@H](O)[C@@H](O)CO ; Cl ; C=Cc1c(C)c2cc3nc(c(C)c4cc(C)c(cc5nc(cc1[nH]2)C(C)=C5CC)[nH]4)[C@@H](CCC(=O)N[C@H]1C(O)O[C@H](CO)[C@H](O)[C@@H]1O)[C@@H]3C ; CCC1=C(C)c2cc3[nH]c(cc4nc(c(C)c5cc(C)c(cc1n2)[nH]5)[C@@H](CCC(=O)N[C@H]1C(O)O[C@H](CO)[C@H](O)[C@@H]1O)[C@@H]4C)c(C)c3CC]

Step 1: To a 25 mL RBF containing a stirrer bar was added phyllochlorin (250 mg, 0.491 mmol, 1 eq), DMTMM (149 mg, 0.541 mmol, 1.1 eq), DMF (3 mL), triethylamine (75 μL, 0.541 mmol, 1.1 eq) and D-(+)-galactosamine hydrochloride (116 mg, 0.541 mmol, 1.1 eq). The resultant mixture was stirred for 30 minutes, at which point HPLC analysis showed ˜4% phyllochlorin remained. Further DMTMM (14 mg, 0.1 eq), triethylamine (7 μL, 0.1 eq) and D-(+)-galactosamine hydrochloride (11 mg, 0.1 eq) were added and the solution stirred for a further 30 minutes. The reaction mixture was concentrated by rotary evaporation to give a black residue. The residue was subjected to column chromatography. The column was eluted using a gradient of 10% MeOH/DCM (300 mL), then 15% MeOH/DCM (300 mL), and then 20% MeOH/DCM. Fractions containing the product were combined and the solvent removed by rotary evaporation. The solid was dried under vacuum at 60° C. to give phyllochlorin 2-deoxy-D-galactosamine-propylamide as a dark blue solid (275 mg, 84%).

¹H NMR (400 MHz, d₆-DMSO) δ 9.74 (d, J=6.2 Hz, 2H), 9.13-8.95 (m, 2H), 8.33 (dd, J=17.8, 11.6 Hz, 1H), 7.66 (dd, J=36.3, 8.5 Hz, 1H), 6.50-6.33 (m, 1H), 6.27-6.07 (m, 2H), 4.92 (t, J=3.9 Hz, 1H), 4.72-4.22 (m, 5H), 4.00-3.90 (m, 3H), 3.88-3.67 (m, 4H), 3.62 (d, J=0.9 Hz, 4H), 3.55 (s, 4H), 3.33 (s, 24H), 2.53-2.47 (m, 4H), 2.20-1.99 (m, 1H), 1.84-1.62 (m, 8H), −2.25 (s, 1H), −2.41 (s, 1H). The product exists as a mixture of epimers which causes two sets of signals in an ˜3:1 ratio.

Step 2: A 3-necked 100 mL RBF was charged with phyllochlorin 2-deoxy-D-galactosamine-propylamide (100 mg, 0.149 mmol, 1 eq), 10% Pd/C (10 mg), THF (15 mL) and a stirrer bar (˜15 mm). A hydrogen balloon was connected to the middle joint of the flask via a short length air condenser and the side-arm was connected to a 3-way tap. The setup was evacuated and then re-filled with nitrogen (3 times), evacuated and refilled with hydrogen (2 times). The resulting solution was then stirred (600 RPM) under the hydrogen atmosphere with heating at 30° C. for 2 hours. The solution was filtered through Celite® (1×3 cm), washing with THF (˜10 mL) and the solvent then removed under reduced pressure to give 152 mg of crude product. The residual solid was purified by passage through a plug of silica gel (3×3 cm) eluting with 10% MeOH/DCM to give compound 24 as a dark blue solid (66 mg, 66%) (HPLC purity: 96.3%).

¹H NMR (400 MHz, DMSO-d₆) δ 9.73 (s, 1H), 9.61 (s, 1H), 9.05-8.92 (m, 2H), 7.67 (dd, J=26.0, 8.6 Hz, 1H), 6.51-6.33 (m, 1H), 6.24 (dd, J=4.4, 1.2 Hz, 1H), 4.91 (t, J=3.9 Hz, 1H), 4.69-4.40 (m, 3H), 4.37-4.21 (m, 1H), 4.16-4.02 (m, 1H), 4.02-3.90 (m, 4H), 3.86 (s, 1H), 3.84-3.73 (m, 2H), 3.69 (q, J=4.4, 3.9 Hz, 1H), 3.62 (d, J=1.0 Hz, 3H), 3.56-3.48 (m, 1H), 3.42 (s, 3H), 3.17 (d, J=3.4 Hz, 1H), 2.18-2.08 (m, 1H), 1.78-1.59 (m, 10H), −2.34 (s, 1H), −2.40 (s, 1H).

### Synthesis Example 25—Synthesis of Reduced Phyllochlorin (6-hexyl)triphenylphosphonium Chloride Propylamide (Compound 25)

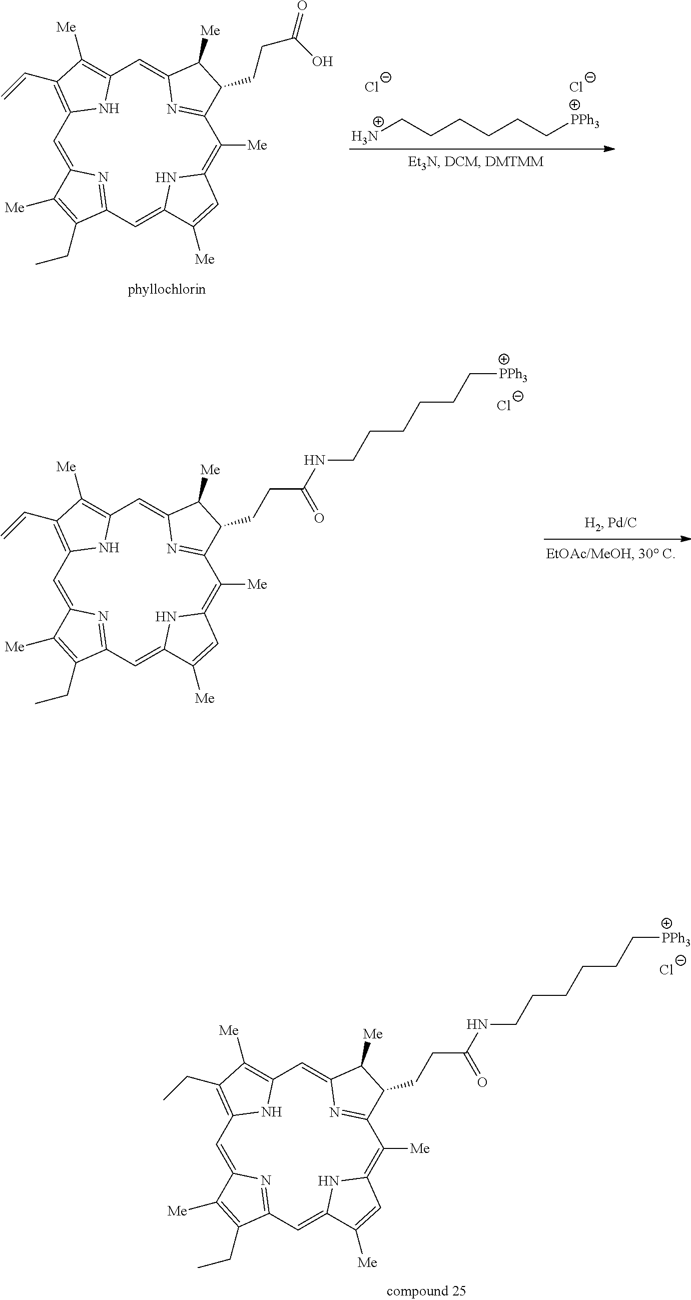

Step 1: To a 25 mL RBF was added phyllochlorin (100 mg, 0.1966 mmol, 1 eq), 4-(4,6-dimethoxy-1,3,5-triazin-2-yl)-4-methylmorpholin-4-ium chloride (DMTMM) (76 mg, 0.2752 mmol, 1.4 eq), DCM (5 mL), triethylamine (44 μL, 0.3145 mmol, 1.6 eq) and (6-aminohexyl)triphenylphosphonium chloride hydrochloride (120 mg, 0.2752 mmol, 1.4 eq). The resultant mixture was stirred (420 rpm) under nitrogen at ambient temperature for 30 minutes. The reaction mixture was concentrated using a rotary evaporator and the crude residue purified by column chromatography (silica, 5-7% MeOH/DCM) to give phyllochlorin (6-hexyl)triphenylphosphonium chloride propylamide as a dark blue/green solid (180 mg, quantitative).

¹H NMR (400 MHz, CDCl₃) δ 9.67 (m, 2H), 8.89 (s, 1H), 8.78 (s, 1H), 8.14 (dd, 1H), 7.83 (t, 1H), 7.62 (m, 6H), 7.48 (m, 9H), 6.35 (d, 1H), 6.10 (d, 1H), 4.70 (q, 1H), 4.51 (m, 1H), 3.98 (m, 7H), 3.82 (q, 2H), 3.60 (s, 3H), 3.49 (m, 5H), 3.35 (s, 3H), 3.26 (m, 2H), 2.88 (m, 1H), 2.61-2.48 (m, 2H), 1.90-1.80 (m, 1H), 1.78-1.70 (m, 6H), 1.60-1.40 (m, 8H), −2.11 (br s, 1H), −2.24 (br s, 1H).

Step 2: A 3-necked 100 mL RBF was charged with phyllochlorin (6-hexyl)triphenylphosphonium chloride propylamide (60 mg, 0.0675 mmol, 1 eq), 10% Pd/C (10 mg), ethyl acetate (7 mL), methanol (7 mL) and a stirrer bar (˜15 mm). A hydrogen balloon was connected to the middle joint of the flask via a short length air condenser and the side-arm was connected to a 3-way tap. The setup was evacuated and then re-filled with nitrogen (3 times), evacuated and refilled with hydrogen (2 times). The resulting solution was then stirred (600 RPM) under the hydrogen atmosphere with heating at 30° C. (heat block) for 4 hours. The solution was filtered through Celite® (1×3 cm), washing with methanol (˜10 mL) and the solvent was then removed under reduced pressure to give compound 25 as a dark blue solid (24 mg, 40%) (HPLC purity: 91.1%).

¹H NMR (400 MHz, CDCl₃) δ 9.67 (s, 1H), 9.50 (s, 1H), 8.77 (d, J=25.9 Hz, 2H), 7.89-7.61 (m, 8H), 7.59-7.39 (m, 9H), 4.66 (s, 1H), 4.51 (d, J=10.3 Hz, 1H), 4.01-3.87 (m, 6H), 3.83 (q, J=7.5 Hz, 2H), 3.66-3.48 (m, 5H), 3.44-3.33 (m, 7H), 3.32-3.29 (m, 1H), 3.28-3.16 (m, 3H), 2.77 (s, 1H), 2.65-2.51 (m, 1H), 2.46 (s, 1H), 1.99 (s, 1H), 1.73 (q, J=7.7 Hz, 10H), 1.46 (d, J=37.3 Hz, 10H), 1.32-1.16 (m, 2H), 0.95-0.76 (m, 2H), 0.06 (s, 1H), −2.24 (s, 1H).

### Synthesis Example 26—Synthesis of Reduced Phyllochlorin lysyl-lysyl-lysyl-lysyl-lysyl Dimethylamide Propylamide Pentahydrochloride (Compound 26)

### Synthesis of H-(Lys(Boc))—NMe₂

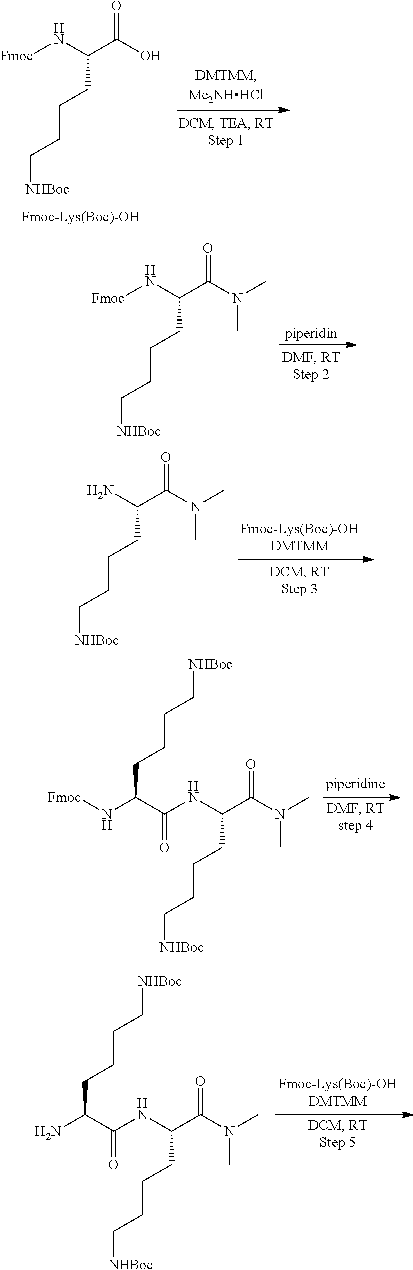

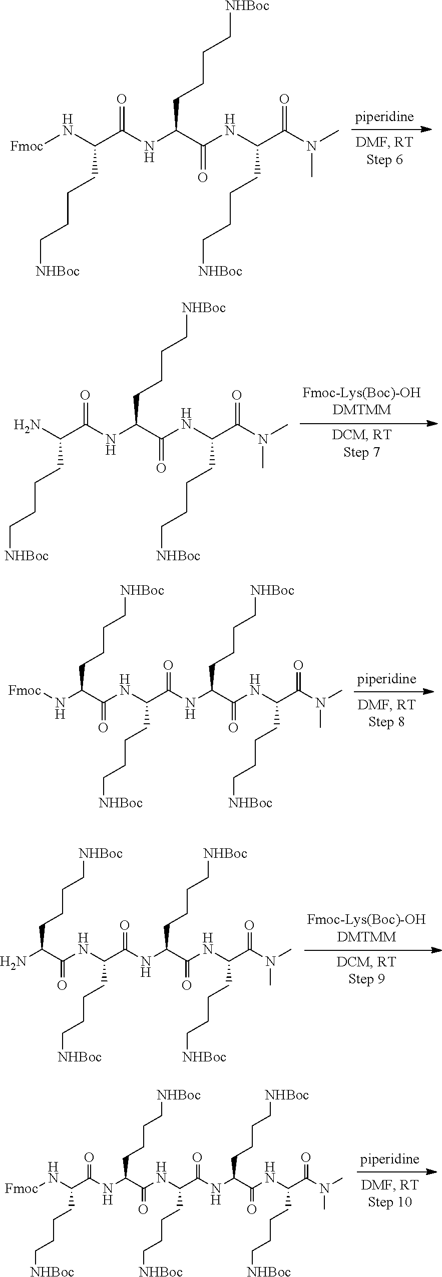

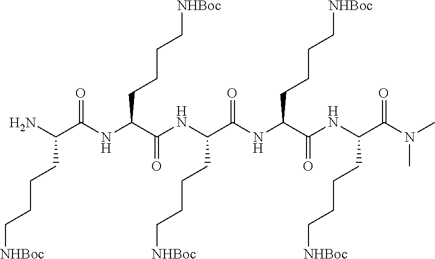 [SMILES: CN(C)C(=O)[C@H](CCCCNC(=O)OC(C)(C)C)NC(=O)[C@H](CCCCNC(=O)OC(C)(C)C)NC(=O)[C@H](CCCCNC(=O)OC(C)(C)C)NC(=O)[C@H](CCCCNC(=O)OC(C)(C)C)NC(=O)[C@@H](N)CCCCNC(=O)OC(C)(C)C]

Step 1: To a 500 mL RBF was added Fmoc-Lys(Boc)-OH (20.0 g, 42.7 mmol, 1 eq), DMTMM (14.19 g, 51.2 mmol, 1.2 eq), DCM (200 mL), triethylamine (5.62 g, 55.5 mmol, 1.3 eq) and dimethylamine hydrochloride (4.17 g, 51.2 mmol, 1.2 eq). The resultant mixture was stirred (420 rpm) under nitrogen at ambient temperature for 1 hour. The reaction mixture was transferred to a separatory funnel, washed with 0.5 M HCl (100 mL) and water (100 mL) and the organic layer dried (MgSO₄) and concentrated by rotary evaporation to give a pale yellow oil (25.32 g). The oil was subjected to column chromatography (9×18 cm). The crude product was dissolved in 2% MeOH/DCM and eluted using a gradient of 2-4% MeOH/DCM. Two major fractions containing the product were collected (Major UV active spot, R_{f}=0.5 in 5% MeOH/DCM) and were combined after concentration by rotary evaporation to give (S)-(9H-fluoren-9-yl)methyl tert-butyl (6-(dimethylamino)-6-oxohexane-1,5-diyl)dicarbamate as a light yellow solid (17.47 g, 83%).

¹H NMR (400 MHz, CDCl₃) δ 7.76 (d, 2H), 7.60 (m, 2H), 7.39 (t, 2H), 7.30 (t, 2H), 5.77 (d, 1H), 4.67 (m, 1H), 4.59 (m, 1H), 4.36 (m, 2H), 4.20 (t, 1H), 3.15-3.08 (m, 4H), 2.97 (s, 3H), 1.78-1.68 (m, 1H), 1.64-1.47 (m, 3H), 1.46-1.37 (m, 11H).

Step 2: A 250 mL RBF fitted with a stirrer bar and air condenser was loaded with (S)-(9H-fluoren-9-yl)methyl tert-butyl (6-(dimethylamino)-6-oxohexane-1,5-diyl)dicarbamate (12.00 g, 24.2 mmol, 1 eq). Piperidine (14.7 mL) and DMF (58.8 mL) were added to the RBF and the resultant solution was stirred at room temperature for 30 minutes. The solvent was then removed by rotary evaporation (65° C., 1 mbar). The residue was dissolved in DCM (200 mL) and the DCM solution then washed with 2% w/w NaHCO₃ aqueous solution (2×200 mL) and brine (200 mL). The organic layer was then dried over MgSO₄, filtered on a sintered glass frit and concentrated by rotary evaporation to give the crude product as a highly viscous light yellow syrup (10.70 g). The crude product was purified by column chromatography (6×20 cm silica gel, eluent gradient=1:1:98 Et₃N:MeOH:DCM to 1:3:96 Et₃N:MeOH:DCM to 1:5:94 Et₃N:MeOH:DCM, R_{f}=0.26, in 5% MeOH in DCM, visualised by ninhydrin stain/heating). The fractions containing the product were then combined and concentrated by rotary evaporation to give (S)-tert-butyl (5-amino-(6-dimethylamino)-6-oxohexyl)carbamate as a light yellow highly viscous syrup (2.98 g, 45%).

¹H NMR (400 MHz, CDCl₃) δ 4.59 (s, 1H), 3.65 (dd, J=7.5, 4.5 Hz, 1H), 3.10 (dd, J=13.3, 6.7 Hz, 2H), 3.02 (s, 3H), 2.96 (s, 3H), 1.67-1.54 (m, 1H), 1.52-1.31 (m, 13H).

Step 3: A 100 mL 1-neck RBF fitted with a stirrer bar and air condenser was loaded with Fmoc-Lys(Boc)-OH (3.81 g, 8.14 mmol, 1 eq), (S)-tert-butyl (5-amino-(6-dimethylamino)-6-oxohexyl)carbamate (2.47 g, 9.04 mmol, 1.11 eq), DMTMM (2.93 g, 10.6 mmol, 1.3 eq) and DCM (30 mL). The reaction mixture was stirred for 1 hour at room temperature and monitored by TLC. DCM (100 mL) was added to the reaction mixture and the reaction mixture was then washed with saturated aqueous NaHCO₃ solution (2×100 mL) and brine (100 mL). The organic phase was dried over MgSO₄, filtered and the solvent then removed by rotary evaporation of give the crude product as a colourless solid (9.47 g). The crude product was purified by column chromatography (2-5% MeOH in DCM, 6×20 cm silica gel, R_{f}=0.5, visualised by UV) to give Fmoc-(Lys(Boc))₂—NMe₂ as a colourless solid (5.81 g, 99%) (mp: 69-73° C.).

¹H NMR (400 MHz, CDCl₃) δ 7.80-7.71 (m, 2H), 7.60 (d, J=7.5 Hz, 2H), 7.43-7.37 (m, 2H), 7.31 (tdd, J=7.4, 2.1, 1.2 Hz, 2H), 6.82 (d, J=8.0 Hz, 1H), 5.56 (s, 1H), 4.94-4.85 (m, 1H), 4.79 (s, 1H), 4.71 (s, 1H), 4.38 (d, J=7.1 Hz, 2H), 4.21 (q, J=8.6, 7.9 Hz, 2H), 3.18-3.00 (m, 6H), 2.97 (d, J=4.9 Hz, 3H), 1.88-1.55 (m, 3H), 1.54-1.19 (m, 26H).

Step 4: A 100 mL RBF fitted with a stirrer bar and air condenser was loaded with Fmoc-(Lys(Boc))₂—NMe₂ (6.00 g, 8.29 mmol, 1 eq). Piperidine (7.35 mL) and DMF (29.4 mL) were added to the RBF and the resultant solution was stirred at room temperature for 30 minutes. The solvent was then removed by rotary evaporation (65° C., 1 mbar). The residue was dissolved in DCM (100 mL) and the DCM solution then washed with 2% w/w NaHCO₃ aqueous solution (2×100 mL) and brine (100 mL). The organic layer was then dried over MgSO₄, filtered on a sintered glass frit and concentrated by rotary evaporation to give the crude product as a highly viscous light yellow syrup (7.39 g). The crude product was purified by column chromatography (6×20 cm silica gel, eluent gradient=1:1:98 Et₃N:MeOH:DCM to 1:3:96 Et₃N:MeOH:DCM to 1:5:94 Et₃N:MeOH:DCM, R_{f}=0.26, in 5% MeOH in DCM, visualised by ninhydrin stain/heating). The fractions containing the product were then combined and concentrated by rotary evaporation to give H-(Lys(Boc))₂—NMe₂ as a light yellow highly viscous syrup (3.15 g, 76%).

¹H NMR (400 MHz, CDCl₃) δ 7.81 (d, J=8.6 Hz, 1H), 4.88 (td, J=8.3, 4.7 Hz, 1H), 4.68 (s, 2H), 3.32 (dd, J=7.8, 4.6 Hz, 1H), 3.13-3.01 (m, 7H), 2.94 (s, 3H), 1.84-1.66 (m, 1H), 1.64-1.28 (m, 26H).

Step 5: A 50 mL 1-neck RBF fitted with a stirrer bar and air condenser was loaded with Fmoc-Lys(Boc)-OH (1.90 g, 4.06 mmol, 1 eq), H-(Lys(Boc))₂—NMe₂ (2.26 g, 4.51 mmol, 1.11 eq), DMTMM (1.46 g, 5.28 mmol, 1.3 eq) and DCM (20 mL). The reaction mixture was stirred for 1 hour at room temperature and monitored by TLC. DCM (so mL) was added to the reaction mixture and the reaction mixture was then washed with saturated aqueous NaHCO₃ solution (2×50 mL) and brine (50 mL). The organic phase was dried over MgSO₄, filtered and the solvent then removed by rotary evaporation to give the crude product as a faint beige solid (5.23 g). The crude product was purified by column chromatography (2-5% MeOH in DCM, 6×20 cm silica gel, R_{f}=0.43, visualised by UV) to give Fmoc-(Lys(Boc))₃—NMe₂ as a faint beige solid (4.07 g, quant) (mp: 100-101° C.).

¹H NMR (400 MHz, CDCl₃) δ 7.75 (d, J=7.5 Hz, 2H), 7.59 (d, J=7.5 Hz, 2H), 7.38 (t, J=7.5 Hz, 2H), 7.29 (t, J=7.6 Hz, 2H), 7.11-6.76 (m, 2H), 5.78 (s, 1H), 5.01-4.65 (m, 4H), 4.49-4.28 (m, 2H), 4.20 (t, J=7.2 Hz, 2H), 3.06 (q, J=9.6, 7.4 Hz, 11H), 2.94 (d, J=2.4 Hz, 4H), 2.01 (s, 1H), 1.91-1.62 (m, 2H), 1.41 (d, J=7.3 Hz, 46H).

Step 6: A 100 mL RBF fitted with a stirrer bar and air condenser was loaded with Fmoc-(Lys(Boc))₃—NMe₂ (3.87 g, 4.06 mmol, 1 eq). Piperidine (5.00 mL) and DMF (20.0 mL) were added to the RBF and the resultant solution was stirred at room temperature for 30 minutes. The solvent was then removed by rotary evaporation (65° C., 1 mbar). The residue was dissolved in DCM (100 mL) and the DCM solution then washed with 2% w/w NaHCO₃ aqueous solution (2×100 mL) and brine (100 mL). The organic layer was then dried over MgSO₄, filtered on a sintered glass frit and concentrated by rotary evaporation to give the crude product as a highly viscous light yellow syrup (4.27 g). The crude product was purified by column chromatography (6×20 cm silica gel, eluent gradient=1:2:97 Et₃N:MeOH:DCM to 1:4:95 Et₃N:MeOH:DCM to 1:6:93 Et₃N:MeOH:DCM, R_{f}=0.26, in 5% MeOH in DCM, visualised by ninhydrin stain/heating). The fractions containing the product were then combined and concentrated by rotary evaporation to give H-(Lys(Boc))₃—NMe₂ as a light yellow highly viscous syrup (2.56 g, 87%).

¹H NMR (400 MHz, CDCl₃) δ 7.73 (d, J=7.5 Hz, 1H), 6.90 (d, J=8.0 Hz, 1H), 4.84 (dd, J=10.5, 5.8 Hz, 3H), 4.74 (s, 1H), 4.35 (td, J=8.0, 5.6 Hz, 1H), 3.37 (dd, J=7.8, 4.5 Hz, 1H), 3.15-2.99 (m, 9H), 2.94 (s, 3H), 1.87-1.60 (m, 3H), 1.59-1.23 (m, 38H).

Step 7: A 100 mL 1-neck RBF fitted with a stirrer bar and air condenser was loaded with Fmoc-Lys(Boc)-OH (1.22 g, 2.61 mmol, 1 eq), H-(Lys(Boc))₃—NMe₂ (2.12 g, 2.90 mmol, 1.11 eq), DMTMM (938 mg, 3.39 mmol, 1.3 eq) and DCM (20 mL). The reaction mixture was stirred for 1 hour at room temperature and monitored by TLC. DCM (so mL) was added to the reaction mixture and the reaction mixture was then washed with saturated aqueous NaHCO₃ solution (2×50 mL) and brine (50 mL). The organic phase was dried over MgSO₄, filtered and the solvent then removed by rotary evaporation to give the crude product as a faint beige solid (4.32 g). The crude product was purified by column chromatography (3-6% MeOH in DCM, 6×20 cm silica gel, R_{f}=0.35, visualised by UV) to give Fmoc-(Lys(Boc))₄—NMe₂ as a faint beige solid (3.14 g, quant) (mp: 152-158° C.).

¹H NMR (400 MHz, CDCl₃) δ 7.75 (d, J=7.5 Hz, 2H), 7.60 (d, J=7.4 Hz, 2H), 7.39 (t, J=7.5 Hz, 2H), 7.29 (t, J=7.5 Hz, 2H), 6.39-5.78 (m, 2H), 5.15-4.78 (m, 4H), 4.61-4.27 (m, 6H), 4.20 (t, J=7.2 Hz, 1H), 3.27-2.99 (m, 13H), 2.97-2.85 (m, 3H), 1.99-1.61 (m, 7H), 1.60-1.10 (m, 56H).

Step 8: A 100 mL RBF fitted with a stirrer bar and air condenser was loaded with Fmoc-(Lys(Boc))₄—NMe₂ (3.08 g, 2.61 mmol, 1 eq). Piperidine (5.00 mL) and DMF (20.0 mL) were added to the RBF and the resultant solution was stirred at room temperature for 30 minutes. The solvent was then removed by rotary evaporation (65° C., 1 mbar). The residue was dissolved in DCM (100 mL) and the DCM solution then washed with 2% w/w NaHCO₃ aqueous solution (2×100 mL) and brine (100 mL). The organic layer was then dried over MgSO₄, filtered on a sintered glass frit and concentrated by rotary evaporation to give the crude product as a colourless solid (4.15 g). The crude product was purified by column chromatography (4×20 cm silica gel, eluent gradient=1:2:97 Et₃N:MeOH:DCM to 1:4:95 Et₃N:MeOH:DCM to 1:6:93 Et₃N:MeOH:DCM, R_{f}=0.26, in 5% MeOH in DCM, visualised by ninhydrin stain/heating). The fractions containing the product were then combined and concentrated by rotary evaporation to give H-(Lys(Boc))₄—NMe₂ as a colourless solid (2.08 g, 83%) (mp: 57-63° C.).

¹H NMR (400 MHz, CDCl₃) δ 7.77 (s, 1H), 7.06-6.83 (m, 2H), 5.13-4.68 (m, 5H), 4.51-4.31 (m, 2H), 3.41-3.31 (m, 1H), 3.16-2.99 (m, 11H), 2.93 (s, 3H), 1.98-1.60 (m, 2H), 1.59-1.22 (m, 58H).

Step A: A 100 mL 1-neck RBF fitted with a stirrer bar and air condenser was loaded with Fmoc-Lys(Boc)-OH (439 mg, 0.937 mmol, 1 eq), H-(Lys(Boc))₄—NMe₂ (1.00 g, 1.04 mmol, 1.11 eq), DMTMM (338 mg, 1.22 mmol, 1.3 eq) and DCM (7 mL). The reaction mixture was stirred for 1 hour at room temperature and monitored by TLC. DCM (so mL) was added to the reaction mixture and the reaction mixture was then washed with saturated aqueous NaHCO₃ solution (2×50 mL) and brine (50 mL). The organic phase was dried over MgSO₄, filtered and the solvent then removed by rotary evaporation to give the crude product as a faint beige solid (1.39 g). The crude product was purified by column chromatography (2-6% MeOH in DCM, 4×20 cm silica gel, R_{f}=0.30, visualised by UV) to give Fmoc-(Lys(Boc))₅—NMe₂ as a faint beige solid (1.14 g, 86%) (mp: 200-204° C.).

¹H NMR (400 MHz, CDCl₃) δ 9.13 (s, 1H), 8.84 (s, 1H), 7.74 (d, J=7.6 Hz, 2H), 7.60 (d, J=7.5 Hz, 2H), 7.38 (t, J=7.5 Hz, 2H), 7.29 (d, J=7.5 Hz, 2H), 7.21-6.83 (m, 2H), 6.24-5.67 (m, 2H), 5.18-4.72 (m, 3H), 4.70-4.11 (m, 5H), 3.33-2.66 (m, 18H), 2.10 (s, 2H), 2.00-1.60 (m, 7H), 1.57-1.02 (m, 74H).

Step 10: A 50 mL RBF fitted with a stirrer bar and air condenser was loaded with Fmoc-(Lys(Boc))₅—NMe₂ (1.09 g, 0.774 mmol, 1 eq). Piperidine (2.00 mL) and DMF (8.00 mL) were added to the RBF and the resultant solution was stirred at room temperature for 30 minutes. The solvent was then removed by rotary evaporation (65° C., 1 mbar). The residue was dissolved in DCM (30 mL) and the DCM solution then washed with 2% w/w NaHCO₃ aqueous solution (2×30 mL) and brine (30 mL). The organic layer was then dried over MgSO₄, filtered on a sintered glass frit and concentrated by rotary evaporation to give the crude product as a colourless solid (1.40 g). The crude product was purified by column chromatography (4×20 cm silica gel, eluent gradient=1:2:97 Et₃N:MeOH:DCM to 1:4:95 Et₃N:MeOH:DCM, R_{f}=0.25, in 5% MeOH in DCM, visualised by ninhydrin stain/heating). The fractions containing the product were then combined and concentrated by rotary evaporation to give H-(Lys(Boc))₅—NMe₂ as a colourless solid (853 mg, 93%) (mp: 157-175° C.).

¹H NMR (400 MHz, CDCl₃) δ 8.83 (d, J=47.0 Hz, 1H), 8.27 (s, 1H), 7.83 (s, 1H), 7.21-6.84 (m, 2H), 6.10 (s, 1H), 6.00-5.70 (m, 1H), 5.26-4.63 (m, 2H), 4.63-4.07 (m, 2H), 3.50-3.18 (m, 1H), 3.18-2.96 (m, 9H), 2.93 (s, 3H), 1.92-1.61 (m, 4H), 1.60-1.17 (m, 63H).

### Synthesis of Reduced Phyllochlorin lysyl-lysyl-lysyl-lysyl-lysyl dimethylamide propylamide pentahudrochloride (Compound 26)

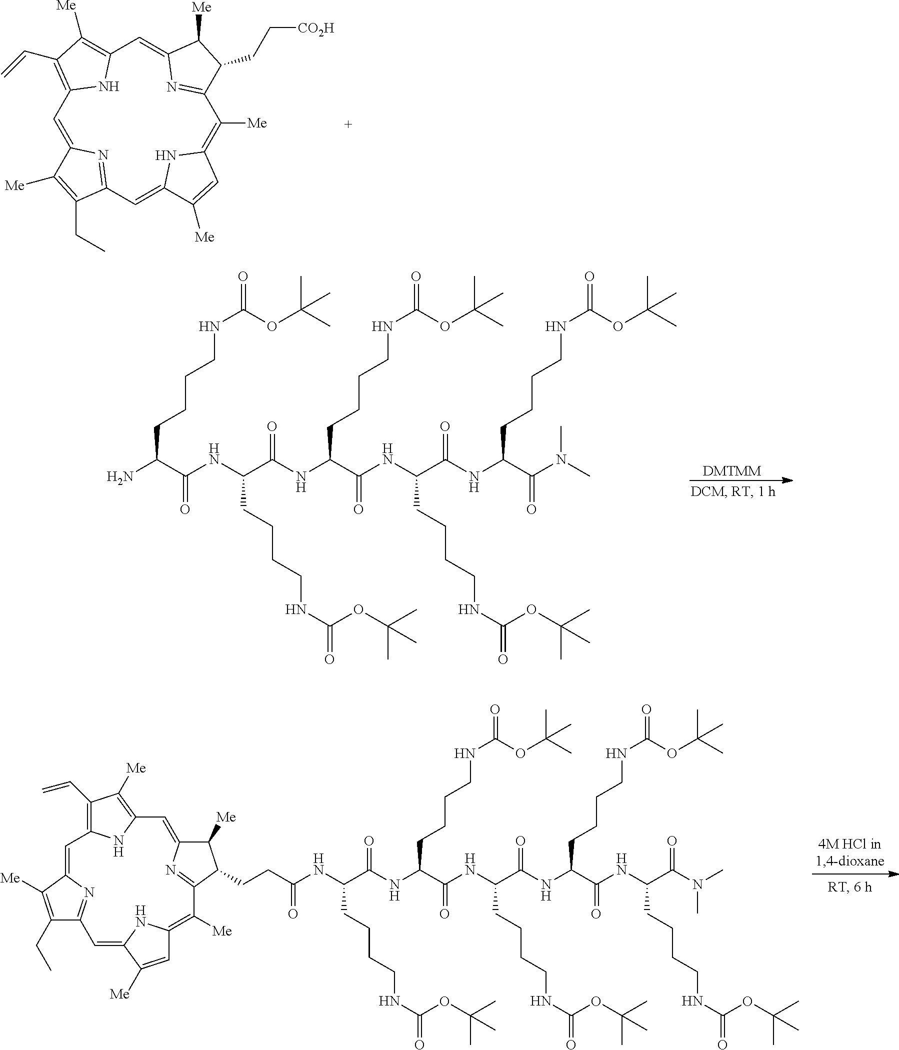

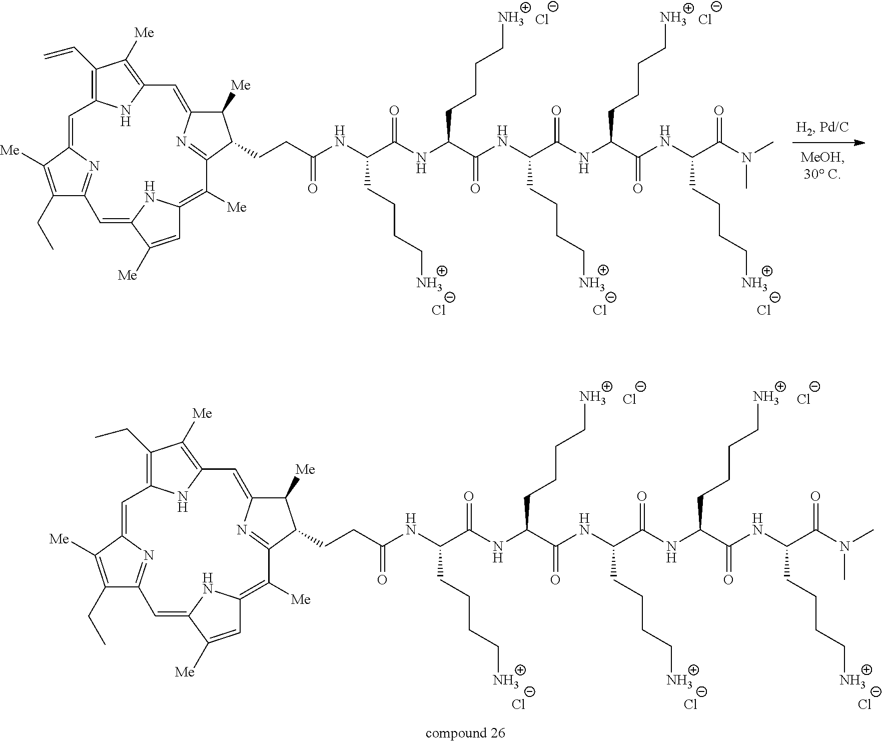

Step 1: To a 25 mL RBF containing a stirrer bar was added phyllochlorin (143 mg, 0.281 mmol, 1 eq), DMTMM (101 mg, 0.365 mmol, 1.3 eq), DMF (7 mL) and H-(Lys(Boc))₅—NMe₂ (400 mg, 0.472 mmol, 1.2 eq). The resultant mixture was stirred for 1 hour with monitoring by HPLC. The reaction mixture was diluted with the addition of DCM (13 mL) and then washed with saturated aqueous NaHCO₃ solution (2×20 mL) and brine (20 mL). The organic layer was then dried with Na₂SO₄, filtered on a sintered glass frit and concentrated by rotary evaporation to give the crude product a dark blue solid. The crude product was subjected to column chromatography (silica gel, 4×20 cm) using an eluent gradient of 2-6% MeOH in DCM, beginning to change the gradient in 2% MeOH increments when the first light blue-green band eluted from the column. Fractions containing the product (Major dark green spot, R_{f}=0.3 in 5% MeOH/DCM) were combined and the solvent removed by rotary evaporation. The solid was dried in a vacuum oven at 60° C. for 3 hours to give phyllochlorin-(Lys(Boc))₅—NMe₂ as a dark blue solid (378 mg, 80%).

¹H NMR (400 MHz, CDCl₃) δ 9.70 (d, J=3.3 Hz, 2H), 8.83 (d, J=4.0 Hz, 2H), 8.16 (dd, J=17.9, 11.6 Hz, 1H), 7.13-6.65 (m, 1H), 6.36 (d, J=17.8 Hz, 1H), 6.13 (dd, J=11.5, 1.5 Hz, 1H), 6.07-5.70 (m, 1H), 5.20-4.64 (m, 3H), 4.66-4.16 (m, 3H), 3.96 (d, J=4.0 Hz, 3H), 3.84 (q, J=7.5 Hz, 2H), 3.79 (s, 0H), 3.63 (s, 3H), 3.53 (s, 2H), 3.36 (s, 3H), 3.18-2.70 (m, 8H), 2.48 (s, 1H), 2.39-2.01 (m, 1H), 1.96-1.55 (m, 12H), 1.53-0.81 (m, 66H), −2.12 (s, 1H), −2.24 (s, 2H).

Step 2: To a 50 mL RBF containing a stirrer bar and an air condenser was added phyllochlorin-(Lys(Boc))₅—NMe₂ (442 mg, 0.305 mmol, 1 eq) and 4M HCl in 1,4-dioxane (10 mL). The mixture was stirred for 2 hours at room temperature. An additional portion of 4M HCl in 1,4-dioxane (10 mL) was added and the reaction stirred for another 4 hours before the solvent was removed by rotary evaporation. The residue was dried in a vacuum oven overnight at 60° C. to give phyllochlorin lysyl-lysyl-lysyl-lysyl-lysyl dimethylamide propylamide pentahydrochloride as a dark blue solid (312 mg, 135%).

¹H NMR (400 MHz, CD₃OD) δ 10.14 (s, 1H), 10.09 (s, 1H), 9.49 (s, 1H), 9.32-9.26 (m, 1H), 8.40-8.26 (m, 3H), 8.19 (dd, J=16.5, 7.5 Hz, 2H), 6.45 (dd, J=17.8, 1.3 Hz, 1H), 6.35 (dd, J=11.7, 1.3 Hz, 1H), 4.84-4.74 (m, 2H), 4.72 (s, 1H), 4.51 (s, 1H), 4.42-4.25 (m, 3H), 4.13 (d, J=4.9 Hz, 4H), 3.97 (p, J=7.2 Hz, 2H), 3.91-3.78 (m, 3H), 3.78-3.70 (m, 23H), 3.70-3.62 (m, 39H), 3.61-3.54 (m, 12H), 3.51 (s, 1H), 3.43 (s, 3H), 3.35 (s, 1H), 3.12 (s, 3H), 3.00-2.84 (m, 17H), 2.82-2.71 (m, 1H), 2.63-2.52 (m, 1H), 2.42-2.31 (m, 1H), 2.19-2.12 (m, 1H), 2.01-1.60 (m, 38H), 1.60-1.35 (m, 23H).

Step 3: A 3-necked 100 mL RBF was charged with phyllochlorin lysyl-lysyl-lysyl-lysyl-lysyl dimethylamide propylamide pentahydrochloride (50 mg, 0.0368 mmol, 1 eq), 10% Pd/C (10 mg), methanol (15 mL) and a stirrer bar (˜15 mm). A hydrogen balloon was connected to the middle joint of the flask via a short length air condenser and the side-arm was connected to a 3-way tap. The setup was evacuated and then re-filled with nitrogen (3 times), evacuated and refilled with hydrogen (2 times). The resulting solution was then stirred (600 RPM) under the hydrogen atmosphere with heating at 30° C. for 2 hours. The solution was filtered through a 0.45 μm syringe filter and then a 0.20 μm syringe filter before the solvent was removed under reduced pressure to give compound 26 as a dark blue solid (26 mg, 52%).

¹H NMR (400 MHz, CD₃OD) δ 10.19 (s, 1H), 10.05 (s, 1H), 9.40 (s, 1H), 9.26 (s, 1H), 4.40-4.23 (m, 5H), 4.16-4.07 (m, 5H), 4.02 (m, 2H), 3.90-3.78 (m, 2H), 3.77-3.70 (m, 13H), 3.70-3.62 (m, 19H), 3.61-3.55 (m, 5H), 3.53 (s, 2H), 3.49 (s, 3H), 3.11 (d, J=2.1 Hz, 4H), 3.00-2.85 (m, 16H), 2.80-2.68 (m, 1H), 2.63-2.50 (m, 1H), 2.40-2.26 (m, 1H), 2.16 (t, J=6.8 Hz, 1H), 2.01-1.58 (m, 22H), 1.58-1.35 (m, 14H).

### Synthesis Example 27—Synthesis of Reduced Phyllochlorin 3-triphenylphosphonium Propyl Iodide (Compound 27)

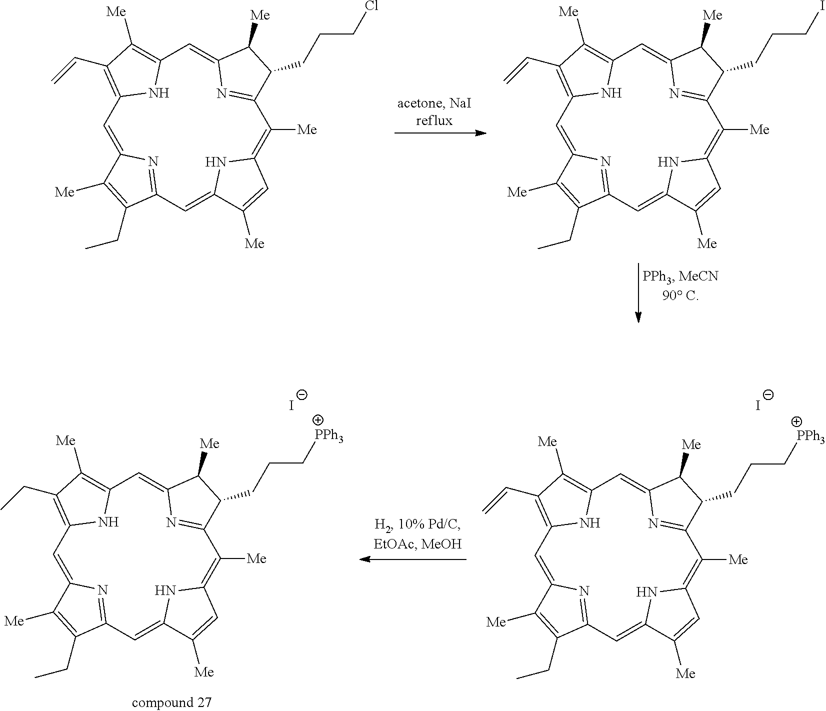

Step 1: To a 50 mL RBF containing phyllochlorin 3-chloropropyl (300 mg, 0.585 mmol, 1 eq) fitted with a reflux condenser and a stirrer bar was added acetone (15 mL) and NaI (263 mg, 1.754 mmol, 3 eq). The solution was heated at 65° C. under nitrogen with monitoring by HPLC. After 4 hours a further portion of NaI (263 mg, 1.754 mmol, 3 eq) was added and heating continued for a total of 30 hours. The mixture was cooled and the acetone removed by rotary evaporation. The residue was diluted with DCM (25 mL) and washed with water (25 mL), dried (Na₂SO₄) and concentrated by rotary evaporation to give the crude product as a dark green oil. The residue was purified by column chromatography (3×20 cm) using a gradient of 0-0.5% MeOH/DCM. Fractions containing the major dark green spot (R_{f}=0.50 in DCM) were combined to give phyllochlorin 3-propyl iodide as a dark green solid (270 mg, 76%) (HPLC purity: 93.3%) that was used directly in the next step.

Step 2: To a sealed tube (2.5×20 cm with Teflon screw cap) containing phyllochlorin 3-propyl iodide (120 mg, 0.198 mmol, 1 eq) and a stirrer bar was added MeCN (7 mL) and PPh₃ (57 mg, 0.218 mmol, 1.1 eq). After flushing the tube with nitrogen and sealing the tube, the dark green mixture was stirred (300 rpm) at 90° C. for 20 hours. The mixture was cooled, transferred to a RBF and the solvent removed by rotary evaporation to remove MeCN to give the crude product as a dark green oil. The residue was purified by column chromatography (3×13 cm) using 2-4% MeOH/DCM. Fractions containing the major dark green spot (R_{f}=0.15 in 5% MeOH/DCM) were combined to give phyllochlorin 3-triphenylphosphonium propyl iodide as a dark green solid (120 mg, 70%) (HPLC purity: 97.6%).

¹H NMR (400 MHz, CDCl₃) δ 9.73 (m, 2H), 8.82 (s, 1H), 8.69 (s, 1H), 8.18 (dd, 1H), 7.02 (m, 3H), 6.60 (m, 6H), 6.40 (m, 7H), 6.19 (d, 1H), 4.72 (m, 1H), 4.50 (m, 1H), 3.94 (s, 3H), 3.88 (q, 2H), 3.68 (s, 3H), 3.48 (s, 3H), 3.39 (s, 3H), 3.20 (m, 1H), 2.72 (m, 1H), 2.61 (m, 1H), 2.08 (m, 1H), 1.80-1.71 (m, 6H), 1.25-1.10 (m, 1H), 0.40-0.30 (m, 1H), −2.25 (brs, 1H), −2.35 (brs, 1H).

Step 3: A 3-neck 50 mL RBF was charged with phyllochlorin 3-triphenylphosphonium propyl iodide (50 mg, 0.058 mmol), 10% Pd/C (10 mg), ethyl acetate (3 mL), MeOH (2 mL) and a stirrer bar. A hydrogen balloon was connected to the middle joint of the flask via a short length air condenser and the side-arm was connected to a 3-way tap. The flask was evacuated and re-filled with nitrogen (3 times), and then evacuated and re-filled with hydrogen (2 times). The reaction mixture was then stirred under a hydrogen atmosphere with heating at 30° C. for 3 hours. The flask was evacuated and re-filled with nitrogen (3 times). Then the reaction mixture was filtered through Celite® (0.5×2 cm), washing with MeOH (˜5 mL). The solvent was then removed under reduced pressure to give a dark green residue. The residue was purified by column chromatography (3×14 cm) eluting with 2-4% MeOH/DCM. Fractions containing the major dark green spot (R_{f}=0.25 in 5% MeOH/DCM) were combined to give compound 27 as a dark green solid (37 mg, 74%).

¹H NMR (400 MHz, CDCl₃) δ 9.72 (s, 1H), 9.53 (s, 1H), 8.78 (s, 1H), 8.61 (s, 1H), 7.01 (m, 3H), 6.59 (m, 6H), 6.37 (m, 6H), 4.69 (m, 1H), 4.45 (m, 1H), 3.96 (q, 2H), 3.91-3.83 (m, 5H), 3.65 (s, 3H), 3.39 (s, 3H), 3.36 (s, 3H), 3.18-3.08 (m, 1H), 2.72-2.62 (m, 1H), 2.61-2.52 (m, 1H), 2.10-1.98 (m, 1H), 1.84-1.70 (m, 6H), 1.32-1.10 (m, 3H), 0.90-0.82 (m, 1H), 0.41-0.30 (m, 1H), −2.30 (brm, 2H).

### Synthesis Example 28—Synthesis of Reduced Phyllochlorin N-(2-(2-(2-picolinylethoxy)ethoxy)ethyl)-N-methyl-3-amide chloride (Compound 28)

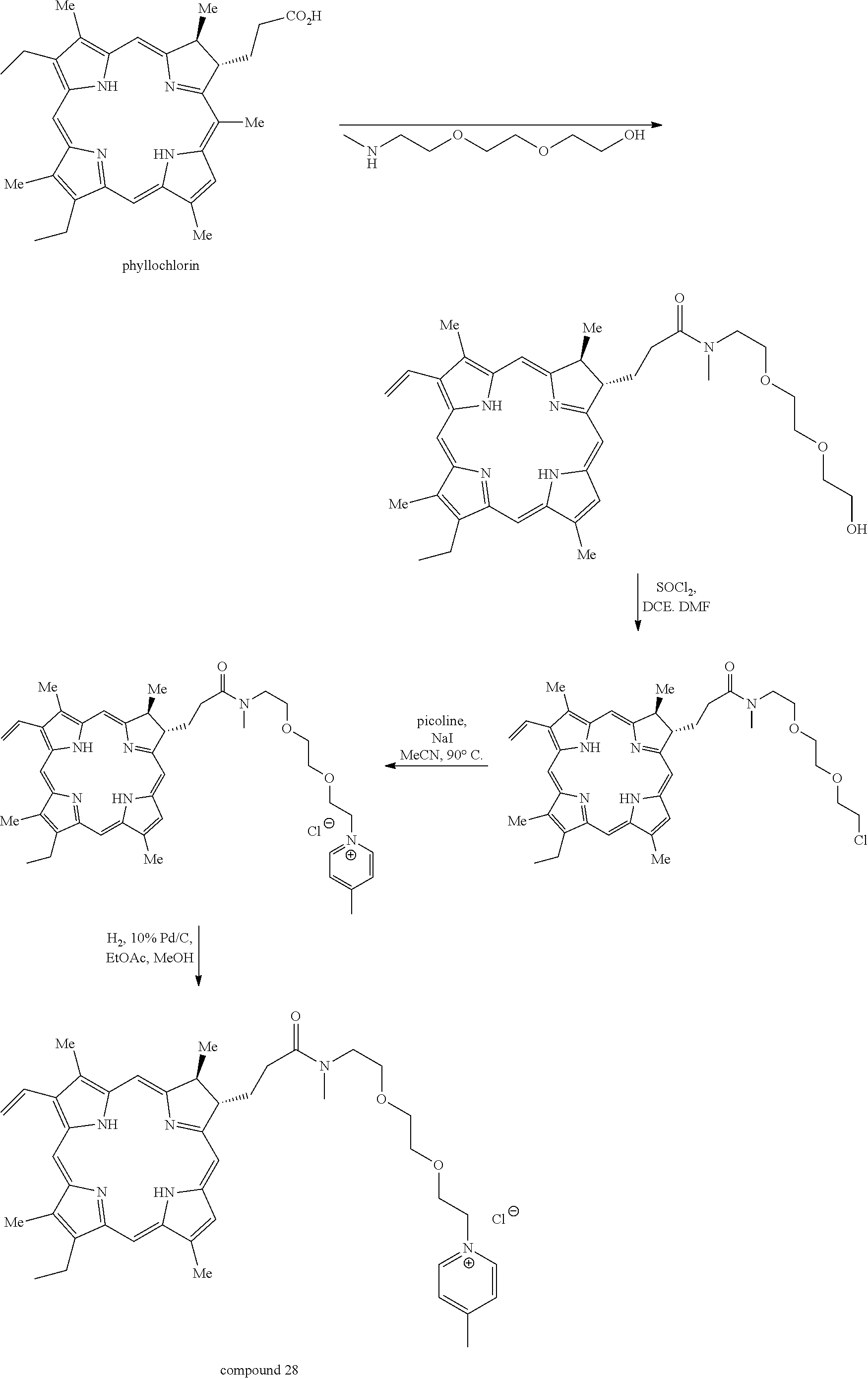

Step 1: Into a 50 mL RBF fitted with a nitrogen inlet and containing a small stirrer bar was added phyllochlorin (500 mg, 0.983 mmol, 1 eq), dichloromethane (15 mL), PyBOP (563 mg, 1.1 eq), triethylamine (409 μL, 3 eq) and 2-(2-(2-(methylamino)ethoxy)ethoxy)ethanol (193 mg, 1.2 eq). The mixture was stirred at room temperature for 1 hour. Analysis by HPLC showed the reaction to be complete. The reaction mixture was transferred to a separatory funnel and washed with water (2×10 mL) and the organic layer dried (Na₂SO₄) and concentrated by rotary evaporation to give the crude product as a blue/brown film (1.10 g). The crude mixture was loaded directly onto a silica column and eluted with 3-7% MeOH/DCM. Pure fractions containing a green/blue compound by TLC(R_{f}=0.20 in 5% MeOH/DCM) were combined to give phyllochlorin N-(2-(2-(2-hydroxyethoxy)ethoxy)ethyl)-N-methyl-3-amide.

¹H NMR (400 MHz, CDCl₃) δ 9.71 (m, 2H), 8.83 (m, 2H), 8.16 (dd, 1H), 6.38 (dd, 1H), 6.13 (dd, 1H), 4.66 (m, 1H), 4.54 (m, 1H), 4.01 (s, 3H), 3.84 (q, 2H), 3.63 (s, 3H), 3.55-3.50 (m, 4H), 3.47-3.30 (m, 10H), 3.04 (m, 1H), 2.77-2.50 (m, 6H), 2.40-2.20 (m, 4H), 1.93-1.35 (m, 1H), 1.30-1.22 (m, 6H), −2.10 (br, 1H), −2.24 (br, 1H).

Step 2: A 50 mL RBF was charged with phyllochlorin N-(2-(2-(2-hydroxyethoxy)ethoxy)ethyl)-N-methyl-3-amide (1.20 g, 1.84 mmol, 1 eq), dichloroethane (10 mL) and DMF (1 drop) and cooled using a water bath. Thionyl chloride (328 mg, 2.76 mmol, 1.5 eq) was dissolved in dichloroethane (2 mL) and the thionyl chloride solution added to the reaction over 1 minute. The resultant solution was stirred under a nitrogen atmosphere at room temperature for 2 hours with monitoring by HPLC. The reaction was then cooled using an ice/water bath and pH=7 phosphate buffer (30 mL) and water (30 mL) were added. The mixture was then extracted with DCM (2×30 mL), dried (Na₂SO₄) and the solvent was removed under reduced pressure to give 2.07 g of the crude product. The residual blue-green crude product was purified by column chromatography (4×18 cm of silica) using 1-2% MeOH in DCM as eluent. Fractions with spots at R_{f}=0.7 by TLC (5% MeOH/DCM eluent) were combined to give phyllochlorin N-(2-(2-(2-chloroethoxy)ethoxy)ethyl)-N-methyl-3-amide as a dark blue-green solid (924 mg, 75%) (HPLC purity: 97.2%).

¹H NMR (400 MHz, CDCl₃) δ 9.71 (m, 2H), 8.86 (m, 2H), 8.17 (dd, 1H), 6.38 (dd, 1H), 6.15 (dd, 1H), 4.66 (m, 1H), 4.54 (m, 1H), 4.01 (m, 3H), 3.86 (q, 2H), 3.64 (m, 3H), 3.59-3.50 (m, 4H), 3.48-3.45 (m, 2H), 3.44-3.39 (m, 2H), 3.40-3.30 (m, 5H), 3.22 (t, 1H), 3.09 (t, 1H), 2.77-2.65 (m, 3H), 2.62-2.50 (m, 2H), 2.48-2.43 (m, 2H), 2.43-2.15 (m, 2H), 1.98-1.83 (m, 1H), 1.80-1.72 (m, 6H), −2.07 (brs, 1H), −2.21 (brs, 1H).

Step 3: To a sealed tube (2.5×20 cm with Teflon screw cap) containing phyllochlorin N-(2-(2-(2-chloroethoxy)ethoxy)ethyl)-N-methyl-3-amide (0.400 g, 0.595 mmol, 1 eq) and a stirrer bar was added MeCN (10 mL), 4-picoline (277 mg, 2.975 mmol, 5 eq) and NaI (178 mg, 1.190 mmol, 2 eq). After flushing the tube with nitrogen and sealing the tube, the dark green mixture was stirred (300 rpm) at 90° C. for 40 hours. The mixture was cooled and diluted with DCM (25 mL), washed with 0.1 M HCl (20 mL), dried (Na₂SO₄) and concentrated by rotary evaporation to give the crude product as a dark green oil. The residue was purified by column chromatography (3×18 cm) using 5-8% MeOH/DCM. Fractions containing the major dark green spot (R_{f}<0.1 in 5% MeOH/DCM) were combined to give phyllochlorin N-(2-(2-(2-picolinylethoxy)ethoxy)ethyl)-N-methyl-3-amide chloride as a dark green solid (207 mg, 45%) (HPLC purity: 96.9%).

¹H NMR (400 MHz, CDCl₃) δ 9.70 (m, 2H), 8.88 (m, 2H), 8.14 (m, 1H), 7.39 (d, 1H), 6.91 (d, 1H), 6.48 (dd, 1H), 6.20 (m, 2H), 5.99 (m, 1H), 4.66-4.53 (m, 2H), 3.99 (m, 3H), 3.84 (m, 2H), 3.67 (s, 3H), 3.54 (m, 3H), 3.42 (m, 1H), 3.36 (m, 4H), 3.30-3.23 (m, 2H), 3.20 (m, 1H), 3.15-2.95 (m, 4H), 2.88 (m, 1H), 2.66 (m, 3H), 2.62-2.30 (m, 4H), 2.28-1.85 (m, 3H), 1.80-1.71 (m, 6H), −2.22-−2.49 (brm, 2H).

Step 4: A 3-neck 50 mL RBF was charged with phyllochlorin N-(2-(2-(2-picolinylethoxy)ethoxy)ethyl)-N-methyl-3-amide chloride (60 mg, 0.0 mmol), 10% Pd/C (10 mg), ethyl acetate (3 mL), MeOH (2 mL) and a stirrer bar. A hydrogen balloon was connected to the middle joint of the flask via a short length air condenser and the side-arm was connected to a 3-way tap. The flask was evacuated and re-filled with nitrogen (3 times), and then evacuated and re-filled with hydrogen (2 times). The reaction mixture was then stirred under a hydrogen atmosphere with heating at 30° C. After 4 days a further portion of 10% Pd/C (10 mg) was added and stirring under hydrogen continued overnight. The flask was evacuated and re-filled with nitrogen (3 times). Then the reaction mixture was filtered through Celite® (0.5×2 cm), washing with MeOH (˜5 mL). The solvent was then removed under reduced pressure to give a dark green residue. The residue was purified by column chromatography (3×15 cm) eluting with 3-7% MeOH/DCM. Fractions containing the major dark green spot (R_{f}=0.10 in 5% MeOH/DCM) were combined to give compound 28 as a dark green solid (37 mg, 62%).

¹H NMR (400 MHz, CDCl₃) δ 9.68 (m, 1H), 9.51 (m, 1H), 8.82 (m, 1H), 8.78 (m, 1H), 7.13 (d, 1H), 6.70 (d, 1H), 6.00 (d, 1H), 5.86 (d, 1H), 4.62-4.49 (m, 2H), 3.97 (m, 5H), 3.83 (m, 2H), 3.64 (s, 3H), 3.41 (m, 3H), 3.38 (m, 3H), 3.30-3.20 (m, 3H), 3.18-3.02 (m, 4H), 2.98-2.75 (m, 2H), 2.71 (s, 2H), 2.66 (s, 1H), 2.59 (m, 2H), 2.40-2.15 (m, 3H), 2.10-1.98 (m, 1H), 1.80-1.70 (m, 10H), −2.30 (brs, 1H), −2.44 (brm, 1H).

### Synthesis Example 29—Synthesis of Reduced Phyllochlorin N-methyl-N-(2-methoxy)ethyl Propylamide (Compound 29)

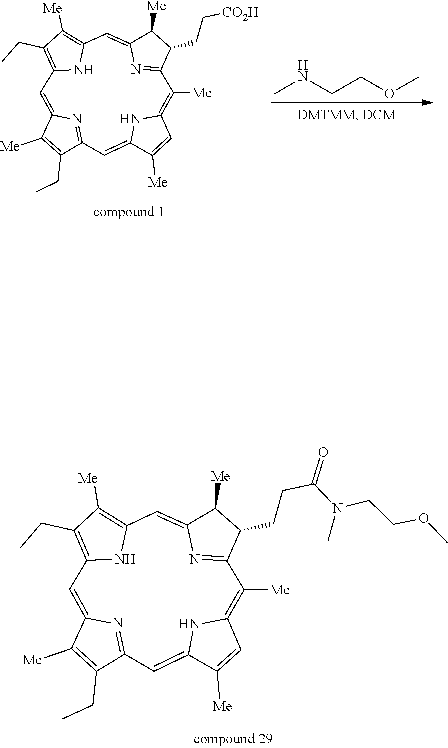

A 25 mL RBF was charged with reduced phyllochlorin (compound 1) (150 mg, 0.293 mmol, 1 eq), DMTMM (121 mg, 0.440 mmol, 1.5 eq), DCM (10 mL) and 2-methoxy-N-methylethan-1-amine (39 mg, 0.440 mmol, 1.5 eq). The reaction mixture was stirred under nitrogen at ambient temperature for 2 hours. The reaction mixture was transferred to a separatory funnel, diluted with DCM (15 mL) and washed with 0.5 M HCl (20 mL). The aqueous layer was re-extracted with DCM (2×5 mL). The combined organic layers were washed with pH=7 phosphate buffer (20 mL), dried (Na₂SO₄) and concentrated by rotary evaporation to give a blue-black film. The residue was purified by column chromatography (4×18 cm) using 0.5-2% MeOH/DCM, loaded as a solution in the eluent. Fractions containing the major dark green band (R_{f}=0.50 in 5% MeOH/DCM) were combined and concentrated by rotary evaporation to give compound 29 as a dark green solid (145 mg, 85%).

¹H NMR (400 MHz, CDCl₃) δ 9.72 (m, 1H), 9.52 (m, 1H), 8.88 (s, 1H), 8.75 (s, 1H), 4.62 (m, 1H), 4.50 (m, 1H), 4.05-3.82 (m, 9H), 3.80-3.68 (m, 8H), 3.64 (s, 3H), 3.57 (m, 3H), 3.42 (m, 3H), 3.40-3.35 (m, 8H), 3.22 (s, 3H), 3.17 (m, 1H), 2.92 (m, 1H), 2.88-2.75 (m, 2H), 2.62-2.42 (m, 2H), 2.22-2.10 (m, 1H), 2.10-1.75 (m, 1H), 1.81-1.72 (m, 9H), −2.21 (brs, 2H).

### Synthesis Example 30—Synthesis of Reduced Phyllochlorin N-(2-methoxy)ethyl propylamide (Compound 30)

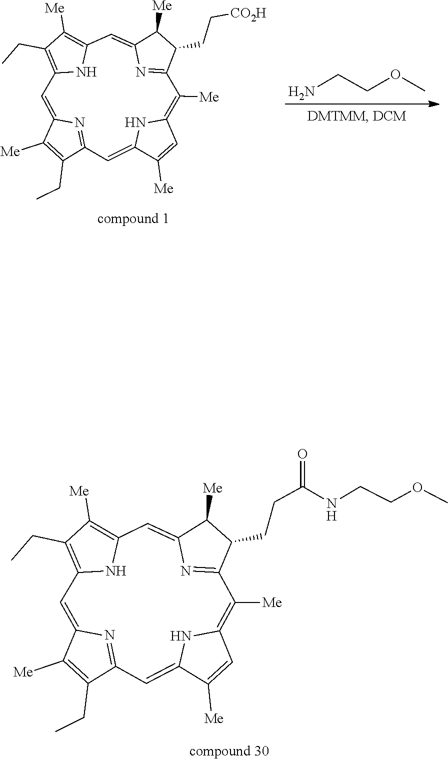

A 50 mL RBF was charged with reduced phyllochlorin (compound 1) (150 mg, 0.294 mmol, 1 eq), DMTMM (122 mg, 0.441 mmol, 1.5 eq), DCM (6 mL) and 2-methoxyethylamine (33 mg, 0.441 mmol, 1.5 eq). The reaction mixture was stirred under nitrogen at ambient temperature for 5 hours. The reaction mixture was transferred to a separatory funnel, diluted with DCM (15 mL) and washed with 0.5 M HCl (20 mL). The aqueous layer was re-extracted with DCM (2×5 mL). The combined organic layers were washed with pH=7 phosphate buffer (20 mL), dried (Na₂SO₄) and concentrated by rotary evaporation to give a blue-black film. The residue was purified by column chromatography (3×16 cm) eluting with 0.5-2% MeOH/DCM. Fractions containing the major dark green spot (R_{f}=0.40 in 5% MeOH/DCM) were combined to give compound 30 as a dark green solid (83 mg, 50%).

¹H NMR (400 MHz, CDCl₃) δ 9.73 (s, 1H), 9.55 (s, 1H), 8.84-8.76 (m, 2H), 5.06 (m, 1H), 4.61 (m, 1H), 4.48 (m, 1H), 4.07 (s, 3H), 3.97 (m, 6H), 3.87 (q, 2H), 3.65 (s, 3H), 3.40 (m, 6H), 3.20-3.03 (m, 3H), 3.00 (s, 3H), 2.58-2.48 (m, 1H), 2.22-2.08 (m, 2H), 1.83-1.73 (m, 9H), −2.16 (brs, 2H).

### Synthesis Example 31—Synthesis of Reduced Phyllochlorin N-Lysyl Dimethylamide Propylamide Hydrochloride (Compound 31)

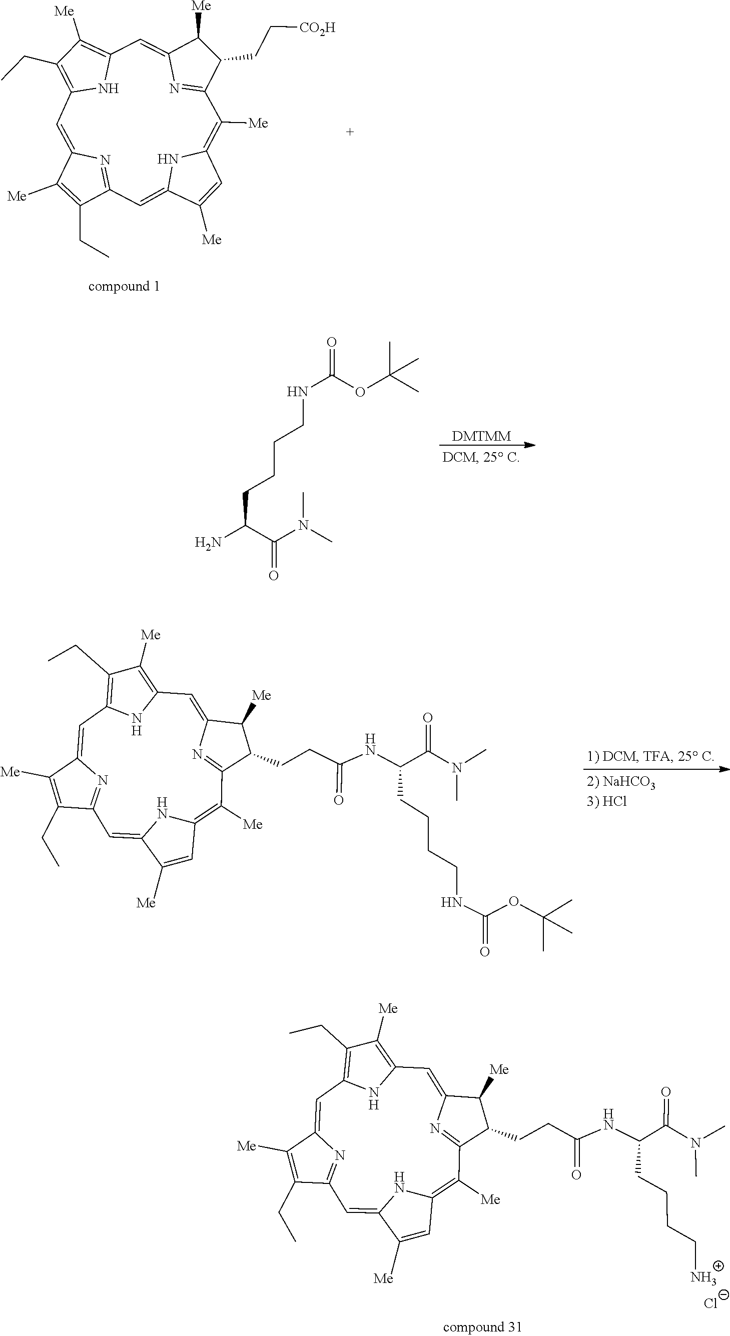

Step 1: A 50 mL RBF containing a stirrer bar was charged with reduced phyllochlorin (compound 1) (200 mg, 0.392 mmol, 1 eq), DMTMM (141 mg, 0.510 mmol, 1.3 eq), DCM (7 mL) and H-Lys(Boc)-NMe₂ (128 mg, 0.470 mmol, 1.2 eq). The reaction mixture was stirred at 25° C. for 1 hour and the reaction progress was monitored by HPLC. The reaction mixture was diluted with DCM (13 mL) and then washed with saturated aqueous NaHCO₃ solution (2×20 mL) and brine (20 mL). The organic layer was then dried (Na₂SO₄), filtered on a sintered glass frit and concentrated by rotary evaporation to give a dark blue residue. The residue was purified by column chromatography (4×20 cm) eluting with 1-3% MeOH/DCM, beginning to change the gradient in 1% MeOH increments when the first light blue-green band eluted from the column. Fractions containing a dark green spot (R_{f}=0.49 in 5% MeOH/DCM) were combined and the solvent removed by rotary evaporation to give reduced phyllochlorin-Lys(Boc)-NMe₂ as a dark blue solid (171 mg, 57%).

¹H NMR (400 MHz, CDCl₃) δ 9.69 (s, 1H), 9.52 (s, 1H), 8.79 (dd, J=2.1, 1.1 Hz, 1H), 8.74 (s, 1H), 5.96 (d, J=8.0 Hz, 1H), 4.69 (td, J=7.9, 4.7 Hz, 1H), 4.59-4.53 (m, 1H), 4.45 (q, J=7.2 Hz, 1H), 4.37 (s, 1H), 3.97-3.89 (m, 5H), 3.83 (q, J=7.6 Hz, 2H), 3.62 (s, 3H), 3.41 (s, 3H), 3.36 (s, 3H), 2.95 (s, 3H), 2.83 (s, 5H), 2.51-2.41 (m, 1H), 2.32-2.22 (m, 1H), 2.21-2.09 (m, 1H), 1.74 (dt, J=11.0, 7.7 Hz, 10H), 1.47-1.36 (m, 1H), 1.29 (s, 9H), 1.24-1.12 (m, 2H), 1.07-0.92 (m, 1H), −2.07-−2.40 (m, 2H).

Step 2: A 25 mL RBF containing a stirrer bar, air condenser and drying tube packed with silica beads was charged with reduced phyllochlorin-Lys(Boc)-NMe₂ (153 mg, 0.200 mmol, 1 eq), DCM (5 mL) and TFA (2 mL). The reaction mixture was stirred at 25° C. for 2 hours and the reaction progress was monitored by HPLC. The reaction mixture was concentrated by rotary evaporation to remove DCM and bulk TFA. The residue was then re-dissolved in DCM (20 mL) and washed with saturated aqueous NaHCO₃ solution (2×30 mL). The organic layer was then washed with H₂O (20 mL), dried (Na₂SO₄), filtered on a sintered glass funnel and concentrated by rotary evaporation. HCl (0.1017 M in H₂O, 1.47 mL, 0.150 mmol, 0.75 eq) was then added to the residue, followed by H₂O (30 mL). The mixture was then sonicated at 30° C. for 30 minutes at which point the pH of the mixture was 4.31. The mixture was then passed through a 0.2 μm syringe filter and freeze dried overnight to give compound 31 as a light green solid (141 mg, quantitative).

¹H NMR (400 MHz, DMSO-d₆) δ 9.85 (s, 1H), 9.74 (s, 1H), 9.09 (s, 2H), 8.20 (d, J=8.2 Hz, 1H), 7.80 (s, 3H), 4.69 (td, J=8.4, 5.4 Hz, 1H), 4.62 (q, J=7.1 Hz, 1H), 4.54 (dd, J=10.0, 2.8 Hz, 1H), 3.99 (q, J=7.7 Hz, 2H), 3.94 (s, 3H), 3.86 (q, J=7.6 Hz, 2H), 3.64 (s, 3H), 3.44 (s, 3H), 3.05 (s, 3H), 2.82 (s, 3H), 2.72-2.61 (m, 2H), 2.60-2.52 (m, 1H), 2.44-2.32 (m, 1H), 2.15-2.02 (m, 1H), 1.84-1.63 (m, 9H), 1.61-1.34 (m, 3H), 1.28-1.13 (m, 2H), −2.58 (s, 2H).

### Synthesis Example 32—Synthesis of Reduced Phyllochlorin N-Lysyl-Lysyl Dimethylamide Propylamide Dihydrochloride (Compound 32)

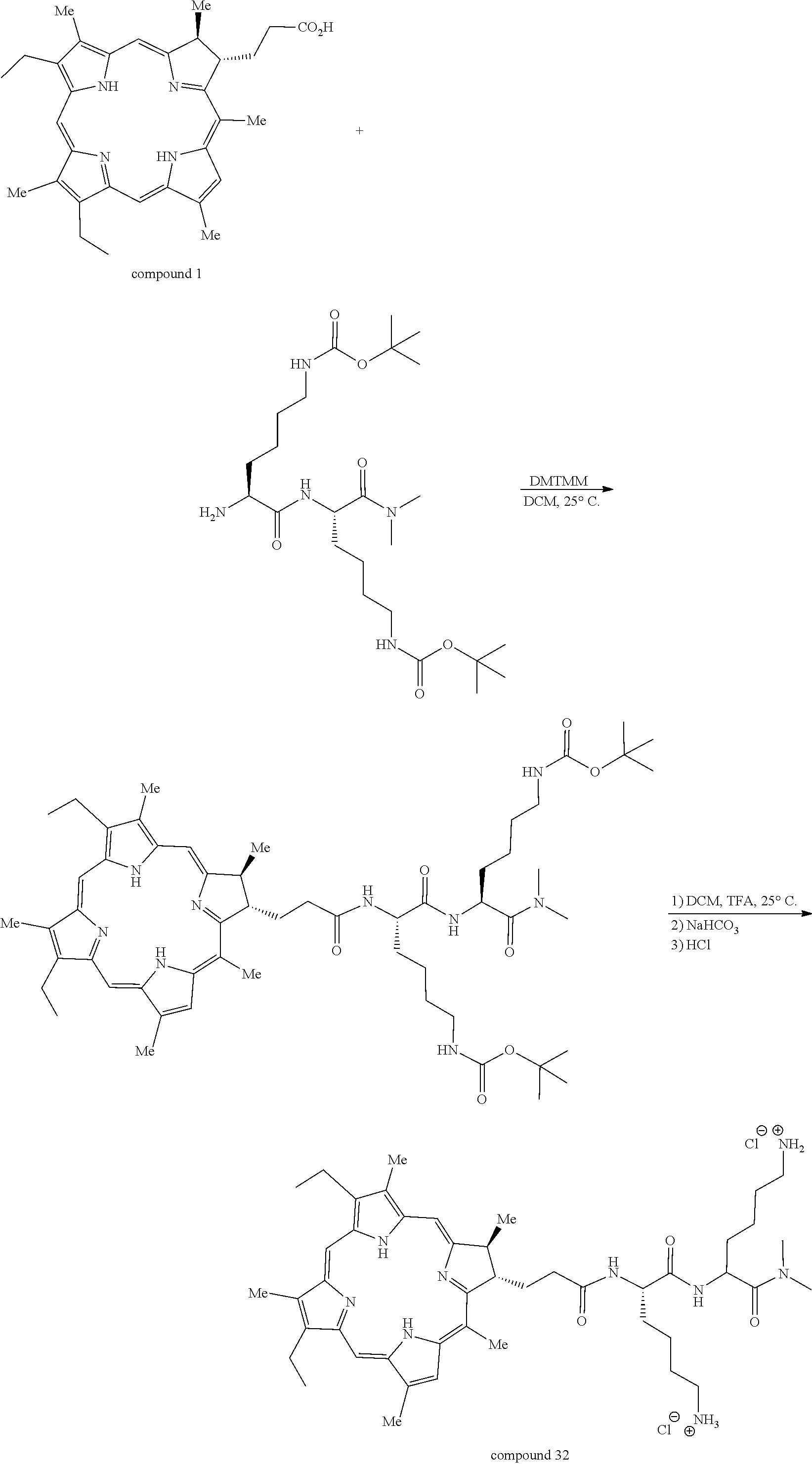

Step 1: A 25 mL RBF containing a stirrer bar was charged with reduced phyllochlorin (compound 1) (300 mg, 0.587 mmol, 1 eq), DMTMM (211 mg, 0.763 mmol, 1.3 eq), DCM (7 mL) and H-(Lys(Boc))₂—NMe₂ (351 mg, 0.700 mmol, 1.2 eq). The reaction mixture was stirred at 25° C. for 1 hour and the reaction progress was monitored by HPLC. The reaction mixture was diluted with DCM (25 mL) and then washed with saturated aqueous NaHCO₃ solution (2×30 mL) and brine (30 mL). The organic layer was then dried (Na₂SO₄), filtered on a sintered glass frit and concentrated by rotary evaporation to give a dark blue residue. The residue was purified by column chromatography (4×20 cm) eluting with 2-4% MeOH/DCM, beginning to change the gradient in 1% MeOH increments when the first light blue-green band eluted from the column. Fractions containing a dark green spot (R_{f}=0.34 in 5% MeOH/DCM) were combined and the solvent removed by rotary evaporation to give reduced phyllochlorin-(Lys(Boc))₂—NMe₂ as a dark blue solid (434 mg, 74%).

¹H NMR (400 MHz, CDCl₃) δ 9.70 (s, 1H), 9.52 (s, 1H), 8.82-8.78 (m, 1H), 8.76 (s, 1H), 6.61 (d, J=8.1 Hz, 1H), 5.52 (s, 1H), 4.75-4.67 (m, 1H), 4.66-4.61 (m, 1H), 4.61-4.54 (m, 1H), 4.48 (q, J=7.2 Hz, 1H), 4.03-3.90 (m, 5H), 3.84 (q, J=7.6 Hz, 2H), 3.63 (d, J=1.0 Hz, 3H), 3.41 (s, 3H), 3.37 (s, 3H), 2.96 (s, 3H), 2.95-2.90 (m, 2H), 2.87 (s, 3H), 2.84-2.71 (m, 1H), 2.52-2.41 (m, 1H), 2.25 (t, J=16.0 Hz, 3H), 1.80-1.71 (m, 9H), 1.68 (s, 3H), 1.51-1.39 (m, 1H), 1.36 (s, 9H), 1.29 (s, 9H), 1.26-1.15 (m, 1H), 1.11-0.95 (m, 2H), 0.87 (s, 2H), −2.09-−2.29 (m, 2H).

Step 2: A 25 mL RBF containing a stirrer bar, air condenser and drying tube packed with silica beads was charged with reduced phyllochlorin-(Lys(Boc))₂—NMe₂ (200 mg, 0.201 mmol, 1 eq), DCM (5 mL) and TFA (2 mL). The reaction mixture was stirred at 25° C. for 2 hours and the reaction progress was monitored by HPLC. The reaction mixture was concentrated by rotary evaporation to remove DCM and bulk TFA. The residue was then re-dissolved in DCM (30 mL) and washed with saturated aqueous NaHCO₃ solution (2×30 mL). The organic layer was then washed with H₂O (20 mL), dried (Na₂SO₄), filtered on a sintered glass funnel and concentrated by rotary evaporation. HCl (0.1017 M in H₂O, 3.17 mL, 0.322 mmol, 1.6 eq) was then added to the residue, followed by H₂O (30 mL). The mixture was then sonicated at 30° C. for 30 minutes at which point the pH of the mixture was 3.17. The mixture was then passed through a 0.2 μm syringe filter and freeze dried overnight to give compound 32 as a dark blue solid (146 mg, quantitative).

¹H NMR (400 MHz, DMSO-d₆) δ 9.98 (s, 1H), 9.88 (s, 1H), 9.30-9.10 (m, 2H), 8.24 (d, J=7.8 Hz, 1H), 8.15-7.87 (m, 7H), 4.75-4.57 (m, 3H), 4.24 (td, J=8.3, 4.9 Hz, 1H), 4.03 (d, J=7.8 Hz, 2H), 3.98 (s, 2H), 3.90 (t, J=7.7 Hz, 1H), 3.67 (s, 3H), 2.98 (s, 3H), 2.75 (s, 3H), 2.73-2.60 (m, 2H), 2.45-2.35 (m, OH), 2.21-2.08 (m, 1H), 1.78 (d, J=7.1 Hz, 3H), 1.71 (t, J=7.5 Hz, 3H), 1.67-1.43 (m, 11H), 1.42-1.14 (m, 6H), −2.61-−3.13 (m, 2H).

### Synthesis Example 33—Synthesis of Reduced Phyllochlorin N-Lysyl-Lysyl-Lysyl Dimethylamide Propylamide Trihydrochloride (Compound 33)

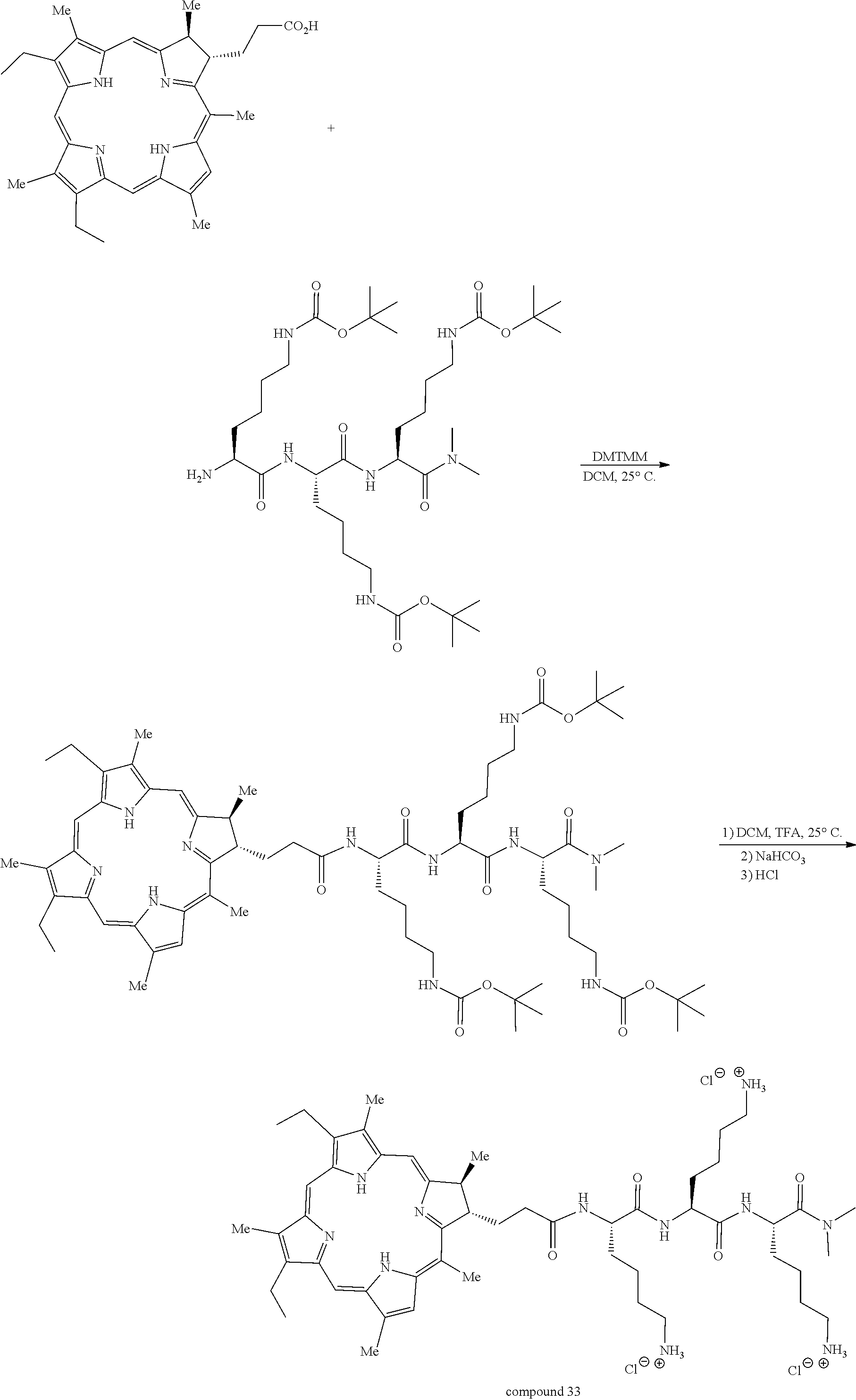

Step 1: A 50 mL RBF containing a stirrer bar was charged with reduced phyllochlorin (compound 1) (500 mg, 0.979 mmol, 1 eq), DMTMM (351 mg, 1.27 mmol, 1.3 eq), DCM (15 mL) and H-(Lys(Boc))₃—NMe₂ (854 mg, 1.17 mmol, 1.2 eq). The reaction mixture was stirred at 25° C. for 1 hour, at which point HPLC analysis showed the reaction to be complete. The reaction mixture was diluted with DCM (25 mL) and then washed with saturated aqueous NaHCO₃ solution (2×40 mL) and brine (40 mL). The organic layer was then dried (Na₂SO₄), filtered on a sintered glass frit and concentrated by rotary evaporation to give a dark blue residue. The residue was purified by column chromatography (5×20 cm) eluting with 2-6% MeOH/DCM, beginning to change the gradient in 1% MeOH increments when the first light blue-green band eluted from the column. Fractions containing a dark green spot (R_{f}=0.34 in 5% MeOH/DCM) were combined and the solvent removed by rotary evaporation to give reduced phyllochlorin-(Lys(Boc))₃—NMe₂ as a dark blue solid (1.12 g, 94%).

¹H NMR (400 MHz, CDCl₃) δ 9.70 (s, 1H), 9.52 (s, 1H), 8.79 (s, 1H), 8.75 (s, 1H), 6.92-6.64 (m, 2H), 5.77 (s, 1H), 4.90-4.68 (m, 4H), 4.62-4.43 (m, 2H), 4.26 (s, 1H), 4.12-3.98 (m, 1H), 3.97-3.89 (m, 6H), 3.84 (q, J=7.6 Hz, 2H), 3.63 (s, 3H), 3.42 (s, 3H), 3.37 (s, 3H), 3.09-2.76 (m, 14H), 2.56-2.40 (m, 1H), 2.35-2.17 (m, 2H), 1.82 (s, 3H), 1.79-1.70 (m, 9H), 1.63-1.48 (m, 1H), 1.41-1.27 (m, 35H), 1.23-1.07 (m, 12H), 1.02-0.87 (m, 2H), −2.20 (s, 2H).

Step 2: A 25 mL RBF containing a stirrer bar, air condenser and drying tube packed with silica beads was charged with reduced phyllochlorin-(Lys(Boc))₃—NMe₂ (200 mg, 0.164 mmol, 1 eq), DCM (5 mL) and TFA (2 mL). The reaction mixture was stirred at 25° C. for 2 hours and the reaction progress was monitored by HPLC. The reaction mixture was concentrated by rotary evaporation to remove DCM and bulk TFA. The residue was then re-dissolved in DCM (40 mL) and washed with saturated aqueous NaHCO₃ solution (40 mL), resulting in an emulsion. EtOH (15 mL) was added to the emulsion in ˜2 mL portions, gently swirling the mixture with each addition to aid separation of the DCM and aqueous layers. The aqueous layer was then extracted with DCM (30 mL), forming an emulsion. EtOH (5 mL) was added to the emulsion to aid separation of the DCM and aqueous layers. The organic layers were combined and washed with H₂O (50 mL), resulting in an emulsion. Brine (50 mL) and then MeOH (50 mL) were added to the emulsion and the DCM layer collected, dried (Na₂SO₄), filtered on a sintered glass funnel and concentrated by rotary evaporation to give the free base intermediate (96 mg). HCl (0.1017 M in H₂O, 2.43 mL, 0.247 mmol, 1.51 eq) was then added to the residue, followed by H₂O (30 mL). The mixture was then sonicated at 30° C. for 30 minutes at which point the pH of the mixture was 2.16. The mixture was then passed through a 0.2 μm syringe filter and freeze dried overnight to give compound 33 as a dark blue solid (88 mg, 52%).

¹H NMR (400 MHz, DMSO-d₆) δ 10.20 (s, 1H), 10.12 (s, 1H), 9.41 (s, 1H), 9.34 (s, 1H), 8.34 (d, J=7.7 Hz, 1H), 8.29-8.08 (m, 10H), 8.03 (d, J=8.0 Hz, 1H), 4.76 (q, J=7.3 Hz, 1H), 4.69 (dd, J=9.9, 2.7 Hz, 1H), 4.60 (td, J=8.3, 4.9 Hz, 1H), 4.25 (dtd, J=26.2, 8.4, 5.4 Hz, 2H), 4.15-3.93 (m, 6H), 3.71 (s, 3H), 3.48 (s, 4H), 3.46 (s, 3H), 2.93 (s, 3H), 2.76 (s, 3H), 2.75-2.59 (m, 8H), 2.47-2.36 (m, 1H), 2.23-2.10 (m, 1H), 1.81 (d, J=7.1 Hz, 3H), 1.70 (t, J=7.5 Hz, 3H), 1.66-1.45 (m, 12H), 1.43-1.16 (m, 6H), −3.05 (s, 1H), −3.39 (s, 1H).

### Synthesis Example 34—Synthesis of Reduced Phyllochlorin ((methylamino)-3-propyl)triphenylphosphonium Bromide Propylamide (Compound 34)

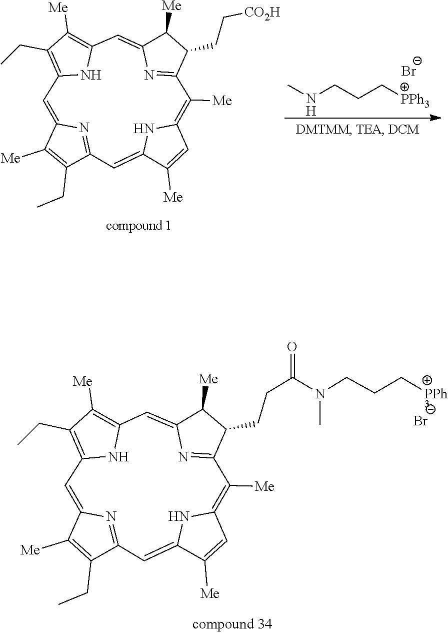

A 25 mL RBF was charged with reduced phyllochlorin (compound 1) (100 mg, 0.197 mmol, 1 eq), DMTMM (82 mg, 0.295 mmol, 1.5 eq), DCM (5 mL), TEA (34 mg, 0.295 mmol, 1.7 eq) and (3-(methylamino)propyl)triphenylphosphonium bromide hydrobromide (146 mg, 0.295 mmol, 1.5 eq). The reaction mixture was stirred under nitrogen at ambient temperature for 2 hours. The reaction mixture was transferred to a separatory funnel, diluted with DCM (10 mL) and washed with 0.5 M HCl (10 mL). The aqueous layer was re-extracted with DCM (2×5 mL). The combined organic layers were washed with pH=7 phosphate buffer (10 mL), dried (Na₂SO₄) and concentrated by rotary evaporation to give a blue-black film. The residue was purified by column chromatography (3×16 cm) eluting with 3-6% MeOH/DCM. Fractions containing the major dark green spot (R_{f}=0.20 in 7% MeOH/DCM) were combined to give compound 34 as a dark green solid (105 mg, 59%).

¹H NMR (400 MHz, CDCl₃) δ 9.70 (s, 1H), 9.52 (s, 1H), 8.79-8.75 (m, 2H), 7.71-7.64 (m, 6H), 7.62-7.58 (m, 3H), 7.55-7.48 (m, 6H), 4.55-4.45 (m, 2H), 3.98-3.90 (m, 5H), 3.84 (q, 2H), 3.80-3.70 (m, 1H), 3.64 (s, 3H), 3.57 (m, 2H), 3.39 (s, 3H), 3.37 (s, 3H), 2.83 (s, 3H), 2.58-2.37 (m, 2H), 2.08-1.94 (m, 2H), 1.88-1.66 (m, 20H), −2.18 (brs, 2H).

### Synthesis Example 35—Synthesis of Reduced Chlorin e4 Dimethyl Ester (Compound 35)

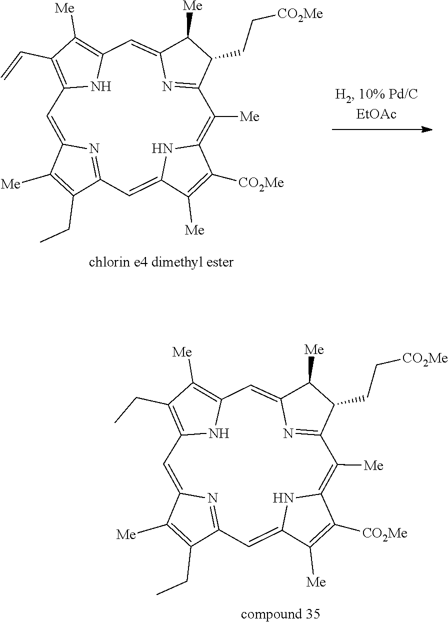 [SMILES: C=Cc1c(C)c2cc3nc(c(C)c4[nH]c(cc5nc(cc1[nH]2)C(C)=C5CC)c(C)c4C(=O)OC)[C@@H](CCC(=O)OC)[C@@H]3C ; CCC1=C(C)c2cc3[nH]c(cc4nc(c(C)c5[nH]c(cc1n2)c(C)c5C(=O)OC)[C@@H](CCC(=O)OC)[C@@H]4C)c(C)c3CC]

A 3-neck 100 mL RBF was charged with chlorin e4 dimethyl ester (200 mg, 0.344 mmol), 10% Pd/C (20 mg), ethyl acetate (40 mL) and a stirrer bar. A hydrogen balloon was connected to the middle joint of the flask via a short length air condenser and the side-arm was connected to a 3-way tap. The flask was evacuated and re-filled with nitrogen (3 times), and then evacuated and re-filled with hydrogen (2 times). The reaction mixture was then stirred under a hydrogen atmosphere for 45 minutes. The flask was evacuated and re-filled with nitrogen (3 times). Then the reaction mixture was filtered through Celite® (0.5×3 cm), washing with ethyl acetate (˜10 mL). The solvent was then removed under reduced pressure to give a green residue (0.25 g). The residue was purified by column chromatography (3×15 cm) eluting with 0.5-1% MeOH/DCM. Fractions containing the major dark green spot (R_{f}=0.70 in 3% MeOH/DCM) were combined to give compound 35 as a metallic blue/green solid (152 mg, 76%).

¹H NMR (400 MHz, CDCl₃) δ 9.68 (s, 1H), 9.39 (s, 1H), 8.67 (s, 1H), 4.50-4.40 (m, 2H), 4.29 (s, 3H), 3.91-3.85 (q, 2H), 3.80 (m, 5H), 3.61 (s, 3H), 3.58 (s, 3H), 3.35 (s, 3H), 3.31 (s, 3H), 2.60-2.52 (m, 1H), 2.48-2.38 (m, 1H), 2.25-2.16 (m, 1H), 1.98-1.88 (m, 1H), 1.78-1.69 (m, 9H), −1.50 (brs, 2H).

### Synthesis Example 36—Synthesis of Reduced Chlorin e4 (Compound 36)

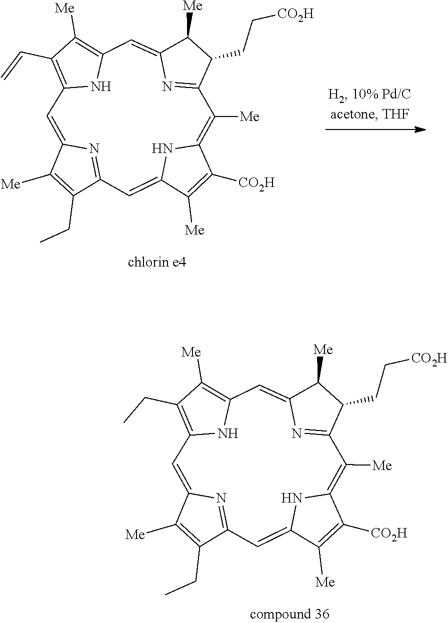 [SMILES: C=Cc1c(C)c2cc3nc(c(C)c4[nH]c(cc5nc(cc1[nH]2)C(C)=C5CC)c(C)c4C(=O)O)[C@@H](CCC(=O)O)[C@@H]3C ; CCC1=C(C)c2cc3[nH]c(cc4nc(c(C)c5[nH]c(cc1n2)c(C)c5C(=O)O)[C@@H](CCC(=O)O)[C@@H]4C)c(C)c3CC]

A 3-neck 250 mL RBF was charged with chlorin e4 (2.0 g, 3.619 mmol), 10% Pd/C (100 mg), acetone (so mL, THF (25 mL) and a stirrer bar. A hydrogen balloon was connected to the middle joint of the flask via a short length air condenser and the side-arm was connected to a 3-way tap. The flask was evacuated and re-filled with nitrogen (3 times), and then evacuated and re-filled with hydrogen (2 times). The reaction mixture was then stirred under a hydrogen atmosphere at ambient temperature for 3 hours. The flask was evacuated and re-filled with nitrogen (3 times). Then the reaction mixture was filtered through Celite® and washed through with THF (2×15 mL). The solvent was removed under reduced pressure to give a blue-black residue (˜2 g). The residue was purified by column chromatography (5×12 cm) eluting with 5-12% MeOH/DCM. Fractions containing the major dark green spot (R_{f}=0.20 in 10% MeOH/DCM) were combined to give compound 36 as a metallic blue/green solid (0.98 g, 49%).

¹H NMR (400 MHz, DMSO-d₆) δ 9.70 (s, 1H), 9.52 (s, 1H), 8.94 (s, 1H), 4.54 (q, 1H), 4.43 (m, 1H), 3.91 (q, 2H), 3.86-3.75 (m, 5H), 3.54 (s, 3H), (3.45-3.25 region partially obscured by H₂O signal), 3.30 (s, 3H), 2.65-2.55 (m, 1H), 2.33-2.20 (m, 2H), 1.73-1.64 (m, 9H), −1.88 (s, 1H), −2.06 (s, 1H).

### Synthesis Example 37—Synthesis of Reduced Chlorin e6 Triol-Triacetate (Compound 37)

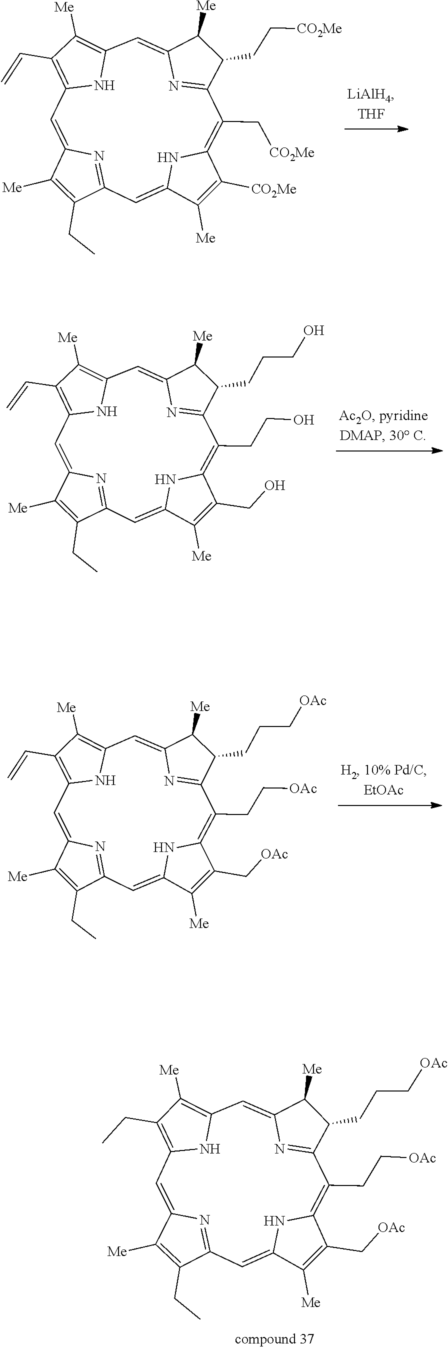 [SMILES: C=Cc1c(C)c2cc3nc(c(CC(=O)OC)c4[nH]c(cc5nc(cc1[nH]2)C(C)=C5CC)c(C)c4C(=O)OC)[C@@H](CCC(=O)OC)[C@@H]3C ; C=Cc1c(C)c2cc3nc(c(CCO)c4[nH]c(cc5nc(cc1[nH]2)C(C)=C5CC)c(C)c4CO)[C@@H](CCCO)[C@@H]3C ; C=Cc1c(C)c2cc3nc(c(CCOC(C)=O)c4[nH]c(cc5nc(cc1[nH]2)C(C)=C5CC)c(C)c4COC(C)=O)[C@@H](CCCOC(C)=O)[C@@H]3C ; CCC1=C(C)c2cc3[nH]c(cc4nc(c(CCOC(C)=O)c5[nH]c(cc1n2)c(C)c5COC(C)=O)[C@@H](CCCOC(C)=O)[C@@H]4C)c(C)c3CC]

Step 1: Into a 250 mL RBF was added chlorin e6 trimethyl ester (1.00 g, 1.566 mmol, 1 eq) and THF (100 mL) and the solution was stirred (350 rpm) and cooled using an ice/water bath for 10 minutes under nitrogen. Lithium aluminium hydride (625 mg, 15.65 mmol, 10 eq) was added in portions over 5 minutes and the mixture was then allowed to warm to room temperature and stirred for 1 hour. Further lithium aluminium hydride (310 mg, 5 eq) was added in portions and stirring was continued for 1 hour. The flask was cooled (ice/water bath) and water (1 mL) was added followed by 4M NaOH (1 mL). After stirring for 10 minutes further water (3 mL) was added and the solution was warmed to room temperature and stirred for 15 minutes. Sodium sulfate was added and the mixture stirred 10 minutes before being filtered, rinsing with DCM until no more colour eluted. The solvent was then removed under reduced pressure to give crude product (˜0.7 g) which was purified by column chromatography (4×20 cm) eluting with 4-6% MeOH/DCM. Fractions containing the major dark green spot (R_{f}=0.25 in 10% MeOH/DCM) were combined to give chlorin e6 triol as a dark green solid (158 mg, 18%).

¹H NMR (400 MHz, DMSO-d₆) δ 9.78 (s, 1H), 9.76 (s, 1H), 9.14 (s, 1H), 8.33 (dd, 1H), 6.45 (dd, 1H), 6.14 (dd, 1H), 5.74 (s, 3H), 5.25 (m, 1H), 5.18 (m, 1H), 4.68-4.58 (m, 2H), 4.37 (m, 1H), 4.19 (m, 3H), 3.82 (q, 2H), 3.63 (s, 3H), 3.54 (s, 3H), 3.31 (s, 3H), 2.03-1.91 (m, 1H), 1.81-1.67 (m, 8H), 1.58-1.40 (m, 2H), −1.86 (br, 1H), −2.28 (br, 1H).

Step 2: Into a 50 mL RBF was added chlorin e6 triol (60 mg, 0.108 mmol, 1 eq), pyridine (1 mL), acetic anhydride (0.11 g, 1.08 mmol, 10 eq) and DMAP (1 mg). The solution was stirred at 30° C. for 1 hour. Ethyl acetate (10 mL) and water (10 mL) were added and the mixture stirred vigorously for 10 minutes. The layers were separated and the ethyl acetate layer washed with 0.5 M HCl (3×10 mL), water (10 mL), saturated NaHCO₃ (10 mL), dried (Na₂SO₄) and concentrated to give a dark green solid (˜80 mg) which was purified by column chromatography (3×16 cm) eluting with 0.5-1% MeOH/DCM. Fractions containing the major dark green spot (R_{f}=0.40 in 1% MeOH/DCM) were combined to give chlorin e6 triol-triacetate as a dark green solid (50 mg, 68%).

¹H NMR (400 MHz, CDCl₃) δ 9.79 (s, 1H), 9.65 (s, 1H), 8.85 (s, 1H), 8.11 (dd, 1H), 6.50 (d, 1H), 6.37 (m, 2H), 6.14 (dd, 1H), 4.87 (m, 2H), 4.71 (m, 2H), 4.53 (m, 1H), 4.38 (m, 1H), 4.06 (m, 2H), 3.82 (q, 2H), 3.67 (s, 3H), 3.51 (m, 3H), 3.33 (m, 3H), 2.26 (s, 3H), 2.19 (s, 3H), 2.10-2.00 (m, 2H), 1.97 (m, 4H), 1.81-1.70 (m, 8H), 1.70-1.60 (m, 2H), −1.48 (brs, 1H), −1.74 (brs, 1H).

Step 0.2: A 3-neck 50 mL RBF was charged with chlorin e6 triol-triacetate (50 mg, 0.073 mmol), 10% Pd/C (10 mg), ethyl acetate (3 mL) and a stirrer bar. A hydrogen balloon was connected to the middle joint of the flask via a short length air condenser and the side-arm was connected to a 3-way tap. The flask was evacuated and re-filled with nitrogen (3 times), and then evacuated and re-filled with hydrogen (2 times). The reaction mixture was then stirred under a hydrogen atmosphere with heating at 35° C. overnight. The flask was evacuated and re-filled with nitrogen (3 times). Then the reaction mixture was filtered through Celite® (0.5×2 cm), washing with ethyl acetate (˜10 mL). The solvent was then removed under reduced pressure to give a dark green residue. The residue was purified by column chromatography (3×13 cm) eluting with 1-2% MeOH/DCM. Fractions containing the major dark green spot (R_{f}=0.80 in 5% MeOH/DCM) were combined to give compound 37 as a dark green solid (6 mg, 12%).

¹H NMR (400 MHz, CDCl₃) δ 9.79 (s, 1H), 9.48 (s, 1H), 8.77 (s, 1H), 6.49 (d, 1H), 6.37 (d, 1H), 4.86 (m, 2H), 4.70 (m, 2H), 4.51 (m, 1H), 4.38 (m, 1H), 4.06 (m, 2H), 3.91 (q, 2H), 3.83 (q, 2H), 3.67 (m, 3H), 3.40 (m, 3H), 3.35 (m, 3H), 2.26 (s, 3H), 2.20 (s, 3H), 2.10-2.00 (m, 2H), 1.97 (m, 4H), 1.81-1.70 (m, 11H), −1.53 (brs, 1H), −1.67 (brs, 1H).

### Synthesis Example 38—Synthesis of Reduced Chlorin K Methyl Ester (Compound 38)

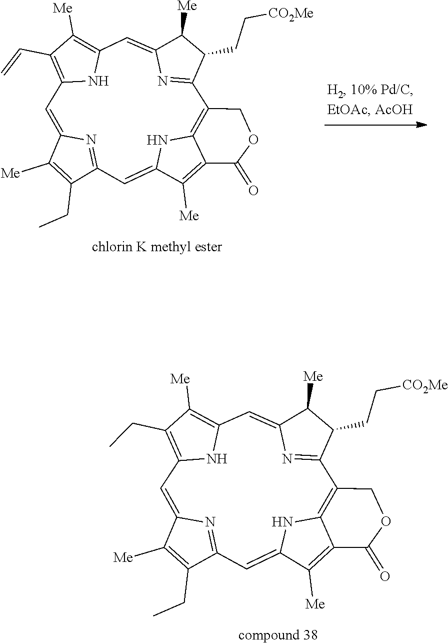 [SMILES: C=Cc1c(C)c2cc3nc(c4c5[nH]c(cc6nc(cc1[nH]2)C(C)=C6CC)c(C)c5C(=O)OC4)[C@@H](CCC(=O)OC)[C@@H]3C ; CCC1=C(C)c2cc3[nH]c(cc4nc(c5c6[nH]c(cc1n2)c(C)c6C(=O)OC5)[C@@H](CCC(=O)OC)[C@@H]4C)c(C)c3CC]

A 3-neck 100 mL RBF was charged with chlorin K methyl ester (100 mg, 0.177 mmol), 10% Pd/C (10 mg), EtOAc (15 mL), AcOH (1.5 mL) and a stirrer bar. A hydrogen balloon was connected to the middle joint of the flask via a short length air condenser and the side-arm was connected to a 3-way tap. The flask was evacuated and re-filled with nitrogen (3 times), and then evacuated and re-filled with hydrogen (2 times). The reaction mixture was then stirred under a hydrogen atmosphere at ambient temperature for 6 hours. The flask was evacuated and re-filled with nitrogen (3 times). Then the reaction mixture was filtered through Celite® and washed through with DCM (3×15 mL). The filtrate was transferred to a separating funnel and washed with saturated NaHCO₃ (30 mL), dried (Na₂SO₄) and the solvent was removed under reduced pressure to give a blue-black residue (0.1 g). The residue was purified by column chromatography (3×16 cm) eluting with 0-1% MeOH/DCM. Fractions containing the major dark green spot (R_{f}=0.55 in 5% MeOH/DCM) were combined to give compound 38 as a metallic blue/green solid (85 mg, 85%).

¹H NMR (400 MHz, CDCl₃) δ 9.67 (s, 1H), 9.35 (s, 1H), 8.60 (s, 1H), 6.79 (s, 2H), 4.45 (q, 1H), 4.34 (m, 1H), 3.85 (m, 5H), 3.74 (m, 2H), 3.61 (s, 3H), 3.32 (s, 3H), 3.29 (s, 3H), 2.65-2.55 (m, 1H), 2.50-2.40 (m, 1H), 2.28-2.18 (m, 1H), 2.05-1.95 (m, 1H), 1.78-1.69 (m, 9H), −0.92 (s, 1H), −1.66 (s, 1H).

### Synthesis Example 39—Synthesis of Reduced Chlorin K (Compound 39)

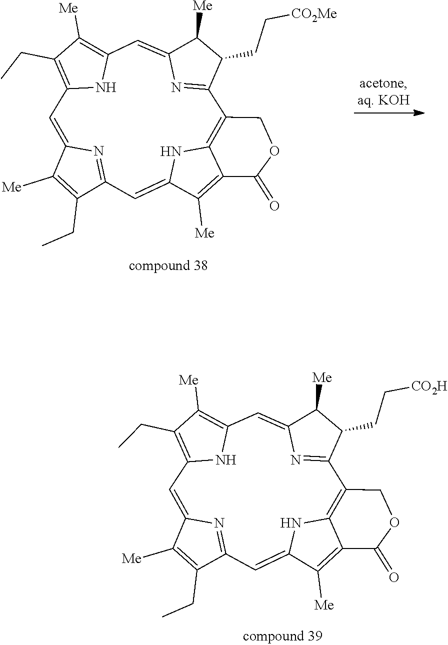 [SMILES: CCC1=C(C)c2cc3[nH]c(cc4nc(c5c6[nH]c(cc1n2)c(C)c6C(=O)OC5)[C@@H](CCC(=O)OC)[C@@H]4C)c(C)c3CC ; CCC1=C(C)c2cc3[nH]c(cc4nc(c5c6[nH]c(cc1n2)c(C)c6C(=O)OC5)[C@@H](CCC(=O)O)[C@@H]4C)c(C)c3CC]

A 250 mL RBF was charged with reduced chlorin K methyl ester (compound 38) (135 mg, 0.238 mmol), acetone (10 mL) and 15% w/v potassium hydroxide (10 mL). The reaction mixture was stirred under nitrogen at 30° C. for 1 hour. Then the reaction mixture was concentrated on a rotary evaporator to remove acetone, before DCM (25 mL) was added to the aqueous residue. The mixture was acidified to pH-5 using 2M HCl (˜20 mL) and the DCM layer was collected. The aqueous layer was further extracted with DCM (2×10 mL). The combined DCM layers were washed with water (30 mL), pH=7 phosphate buffer (20 mL), dried (Na₂SO₄) and concentrated by rotary evaporation to give a dark green residue. The residue was purified by column chromatography (3×12 cm) eluting with 5-6% MeOH/DCM. Fractions containing the major dark green spot (R_{f}=0.15 in 5% MeOH/DCM) were combined to give compound 39 as a dark green solid (104 mg, 79%).

¹H NMR (400 MHz, CDCl₃) δ 9.62 (s, 1H), 9.31 (s, 1H), 8.59 (s, 1H), 6.74 (m, 2H), 4.43 (q, 1H), 4.33 (m, 1H), 3.83 (m, 5H), 3.70 (m, 2H), 3.31 (s, 3H), 3.25 (s, 3H), 2.70-2.60 (m, 1H), 2.40-2.32 (m, 1H), 2.30-2.21 (m, 1H), 1.95-1.85 (m, 1H), 1.77-1.66 (m, 9H), −1.67 (s, 1H).

### Synthesis Example 40—Synthesis of Reduced Chlorin K β-D-1-thioglucose-N-methylpropylamide (Compound 40)

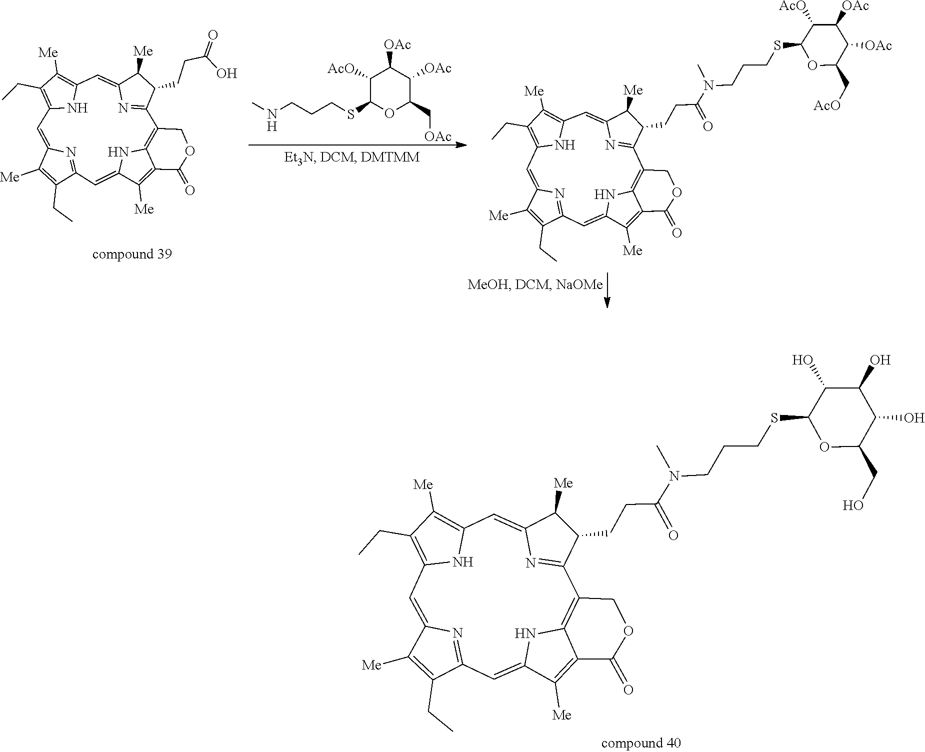 [SMILES: CCC1=C(C)c2cc3[nH]c(cc4nc(c5c6[nH]c(cc1n2)c(C)c6C(=O)OC5)[C@@H](CCC(=O)O)[C@@H]4C)c(C)c3CC ; CNCCCS[C@@H]1O[C@H](COC(C)=O)[C@@H](OC(C)=O)[C@H](OC(C)=O)[C@H]1OC(C)=O ; CCC1=C(C)c2cc3[nH]c(cc4nc(c5c6[nH]c(cc1n2)c(C)c6C(=O)OC5)[C@@H](CCC(=O)N(C)CCCS[C@@H]1O[C@H](COC(C)=O)[C@@H](OC(C)=O)[C@H](OC(C)=O)[C@H]1OC(C)=O)[C@@H]4C)c(C)c3CC ; CCC1=C(C)c2cc3[nH]c(cc4nc(c5c6[nH]c(cc1n2)c(C)c6C(=O)OC5)[C@@H](CCC(=O)N(C)CCCS[C@@H]1O[C@H](CO)[C@@H](O)[C@H](O)[C@H]1O)[C@@H]4C)c(C)c3CC]

Step 1: A 25 mL RBF was charged with reduced chlorin K (compound 39) (78 mg, 0.141 mmol, 1 eq), DMTMM (58 mg, 0.198 mmol, 1.4 eq), N-methyl-3-(((2S,3R,4S,5R,6R)-3,4,5-triacetoxy-6-(acetoxymethyl)tetrahydro-2H-pyran-2-yl)thio)propan-1-aminium 2,2,2-trifluoroacetate (109 g, 0.198 mmol, 1.4 eq), DCM (3 mL) and triethylamine (0.031 mL, 0.226 mmol, 1.6 eq). The reaction mixture was stirred under nitrogen at ambient temperature for 1 hour. The reaction mixture was transferred to a separatory funnel and washed with 0.5 M HCl (2×15 mL), then pH=7 buffer (1×15 mL). The organic phase was dried (Na₂SO₄) and concentrated by rotary evaporation to give a blue-black film. The residue was purified by column chromatography (3×18 cm) eluting with 1-2.5% MeOH/DCM. Fractions containing the major dark green spot (R_{f}=0.45 in 5% MeOH/DCM) were combined to give reduced chlorin K β-D-1-thioglucose-N-methylpropylamide peracetate as a dark green solid (140 mg).

Step 2: To a solution of reduced chlorin K β-D-1-thioglucose-N-methylpropylamide peracetate (140 mg, 0.144 mmol, 1 eq) in MeOH (1 mL) and DCM (1 mL) was added NaOMe (4.6 M in MeOH, 0.016 mL, 0.072 mmol, 0.5 eq). The reaction mixture was stirred under nitrogen for 1 hour. Then the reaction mixture was concentrated by rotary evaporation to give a black film. The residue was purified by column chromatography (3×13 cm) eluting with 5-10% MeOH/DCM. Fractions containing the major dark green spot (R_{f}=0.25 in 10% MeOH/DCM) were combined to give compound 40 as a dark green solid (59 mg, 52% over 2 steps).

¹H NMR (400 MHz, CDCl₃) δ 9.08 (d, 1H), 8.99 (d, 1H), 8.39 (s, 1H), 6.68-6.50 (m, 2H), 5.38 (m, 1H), 5.10 (m, 1H), 4.96 (m, 1H), 4.46 (m, 1H), 4.30-4.20 (m, 2H), 4.13 (m, 1H), 3.95-3.80 (m, 3H), 3.70 (m, 2H), 3.65-3.50 (m, 3H), 3.50-3.30 (m, 8H), 3.24 (m, 1H), 3.10-3.00 (m, 8H), 2.95-2.70 (m, 4H), 2.60-2.50 (m, 3H), 2.45-2.35 (m, 3H), 2.35-2.20 (m, 1H), 2.10-1.90 (m, 2H), 1.90-1.77 (m, 1H), 1.65-1.40 (m, 14H), −1.30 (s, 1H), −1.92 (s, 1H).

### Synthesis Example 41—Synthesis of Reduced Phyllochlorin N-(6-aminohexyl) propylamide (Compound 41)

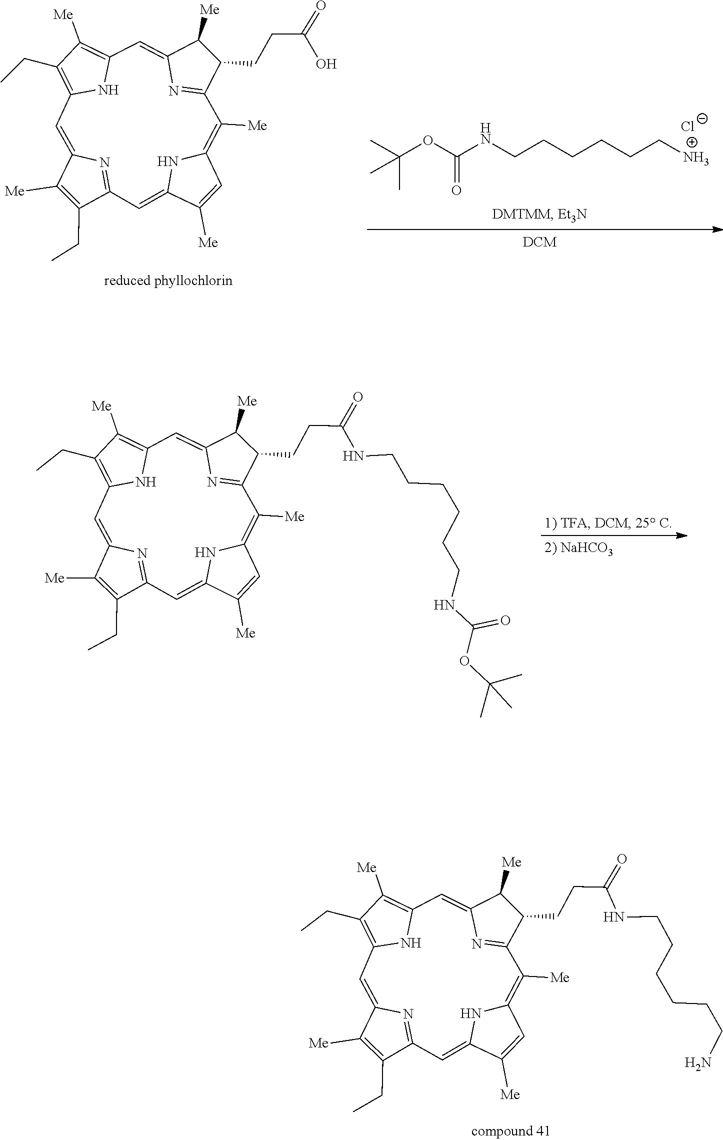

Step 1: To a 50 mL RBF containing a stirrer bar was added reduced phyllochlorin (300 mg, 0.587 mmol, 1 eq), DMTMM (211 mg, 0.763 mmol, 1.3 eq), DCM (7 mL), triethylamine (95 mg, 0.939 mmol, 1.6 eq) and N-Boc-1,6-diaminohexane hydrochloride (193 mg, 1.17 mmol, 1.2 eq). The resultant mixture was stirred for 1 hour. The reaction progress was monitored by HPLC. The reaction mixture was diluted with the addition of DCM (30 mL) and then washed with saturated aqueous NaHCO₃ solution (2×30 mL) and brine (30 mL). The organic layer was then dried (Na₂SO₄), filtered on a sintered glass frit and concentrated by rotary evaporation to give a dark blue solid. The residue was purified by column chromatography (silica gel) using an eluent gradient of 2% MeOH in DCM, beginning to change the gradient in 1% MeOH increments when the first light blue-green band eluted from the column. Fractions containing the product (major dark green spot, R_{f}=0.37 in 5% MeOH/DCM) were combined and the solvent removed by rotary evaporation to give reduced phyllochlorin N-(6-(tert-butylcarbamato)hexyl) propylamide as a dark green solid (426 mg, quantitative).

¹H NMR (400 MHz, CDCl₃) δ 9.71 (s, 1H), 9.53 (s, 1H), 8.82-8.71 (m, 2H), 4.60-4.52 (m, 1H), 4.52-4.32 (m, 3H), 3.98-3.92 (m, 2H), 3.91 (s, 3H), 3.84 (q, J=7.6 Hz, 2H), 3.63 (d, J=1.0 Hz, 3H), 3.40 (s, 3H), 3.37 (s, 3H), 3.08 (q, J=6.7 Hz, 0H), 2.90 (q, J=6.7 Hz, 2H), 2.75-2.64 (m, 1H), 2.59-2.49 (m, 1H), 2.49-2.36 (m, 1H), 2.22-2.09 (m, 1H), 1.92 (dt, J=15.5, 7.8 Hz, 1H), 1.79-1.70 (m, 9H), 1.69-1.51 (m, 2H), 1.44 (s, 2H), 1.38 (s, 8H), 1.17 (p, J=7.2 Hz, 2H), 1.00-0.90 (m, 2H), 0.88-0.73 (m, 4H), −2.08-−2.34 (m, 2H).

Step 2: To a 25 mL RBF containing a stirrer bar was added reduced phyllochlorin N-(6-(tert-butylcarbamato)hexyl) propylamide (400 mg, 0.564 mmol, 1 eq), DCM (5 mL) and TFA (1.5 mL). The mixture was stirred at 25° C. for 2 hours and the reaction progress was monitored by HPLC. The reaction mixture was concentrated by rotary evaporation and then re-dissolved in DCM (30 mL). The DCM solution was washed with saturated aqueous NaHCO₃ solution (2×30 mL) and brine (30 mL) before being dried (Na₂SO₄), filtered on a sintered glass frit and concentrated by rotary evaporation to give a dark green solid. The residue was purified by column chromatography using an eluent gradient of 10-20% MeOH in DCM. Fractions containing the product (major dark green spot, R_{f}=0.3 in 10% MeOH/DCM) were combined and the solvent removed by rotary evaporation to give compound 41 as a dark green solid (302 mg, 88%).

¹H NMR (400 MHz, CDCl₃) δ 9.65 (s, 1H), 9.49 (s, 1H), 8.69 (d, J=15.6 Hz, 2H), 4.30 (d, J=7.3 Hz, 1H), 4.22-4.11 (m, 2H), 3.88-3.73 (m, 3H), 3.70 (s, 3H), 3.50 (s, 3H), 3.31 (d, J=5.8 Hz, 6H), 2.47 (t, J=7.2 Hz, 2H), 2.00 (s, 1H), 1.84 (p, J=7.2 Hz, 2H), 1.72-1.60 (m, 6H), 1.56 (d, J=7.1 Hz, 3H), 1.48-1.37 (m, 1H), 1.30-1.10 (m, 2H), 1.08-0.96 (m, 1H), 0.93-0.77 (m, 1H), 0.75-0.60 (m, 2H), 0.43-0.30 (m, 2H), 0.29-0.15 (m, 2H), −2.30 (s, 1H).

### Synthesis Example 42—Synthesis of Reduced Phyllochlorin N-(6-((R)-2-amino-3-(1-methyl-1H-indol-3-yl)amido)hexyl) propylamide (Compound 42)

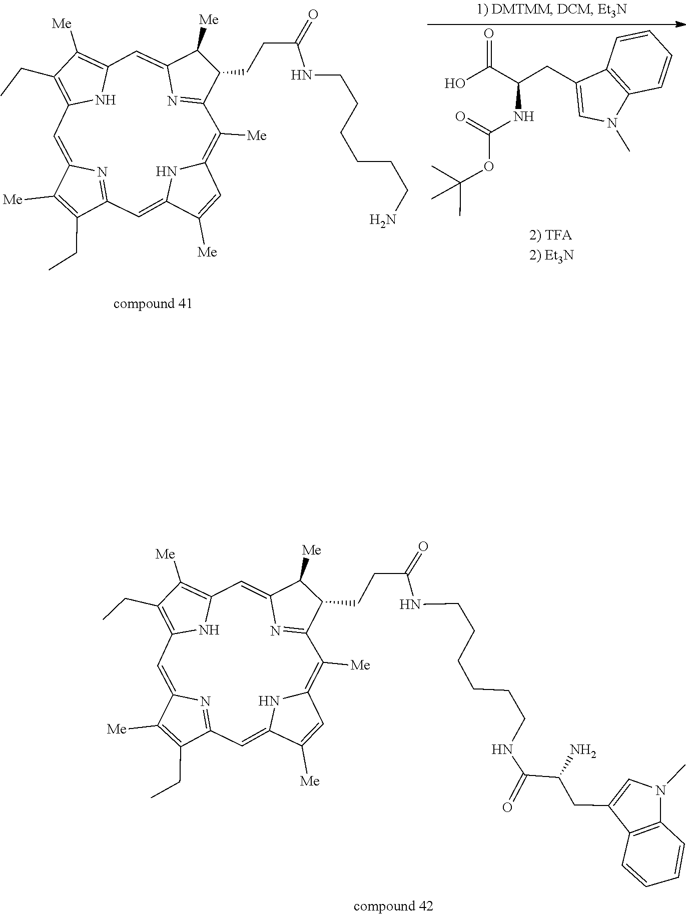

To a 25 mL RBF fitted with a stirrer bar was added reduced phyllochlorin N-(6-aminohexyl) propylamide (compound 41) (100 mg, 0.164 mmol, 1 eq), DMTMM (59 mg, 0.213 mmol, 1.3 eq), (R)-2-((tert)-butoxycarbonyl)amino)-3-(1-methyl-1H-indol-3-yl)propanoic acid (68 mg, 0.213 mmol, 1.3 eq), triethylamine (33 mg, 0.328 mmol, 2 eq) and DCM (3 mL). The reaction mixture was stirred for 2 hours and the reaction progress was monitored by HPLC. TFA (1.0 mL) was then added and the reaction stirred for a further 2 hours and the reaction progress was monitored by HPLC. The reaction mixture was then concentrated by rotary evaporation to remove bulk TFA. The reaction residue was re-dissolved in DCM (20 mL) and triethylamine (1 mL) was added dropwise to the solution while stirring. The resulting solution was then washed with H₂O (2×20 mL) and brine (20 mL) before being dried (Na₂SO₄), filtered on a sintered glass frit and concentrated by rotary evaporation to give a dark green solid. The residue was purified by column chromatography using 5-7% MeOH/DCM, loaded as a solution in the eluent. Fractions containing the major dark green band (R_{f}=0.25 in 5% MeOH/DCM) were concentrated by rotary evaporation to give compound 42 a dark green solid (73 mg, 55%).

¹H NMR (400 MHz, CDCl₃) δ 9.72 (s, 1H), 9.55 (s, 1H), 8.79 (s, 1H), 8.77 (s, 1H), 7.54 (d, J=8.0 Hz, 1H), 7.26 (s, 1H), 7.21-7.12 (m, 2H), 7.10-7.00 (m, 2H), 6.68 (s, 1H), 4.60-4.48 (m, 2H), 4.43 (q, J=7.2 Hz, 1H), 3.99-3.93 (m, 2H), 3.91 (s, 3H), 3.86 (q, J=7.6 Hz, 2H), 3.63 (s, 3H), 3.57 (s, 5H), 3.47 (dd, J=8.8, 4.2 Hz, 1H), 3.40 (d, J=8.1 Hz, 6H), 3.15 (dd, J=14.5, 4.2 Hz, 1H), 3.03 (q, J=6.7 Hz, 2H), 2.79-2.64 (m, 2H), 2.62-2.51 (m, 1H), 2.48-2.34 (m, 1H), 2.17-2.03 (m, 1H), 1.96-1.83 (m, 1H), 1.81-1.70 (m, 10H), 1.43-1.32 (m, 1H), 1.19 (p, J=7.0 Hz, 2H), 1.00-0.76 (m, 7H), −2.18 (s, 1H).

### Synthesis Example 43—Synthesis of Reduced Phyllochlorin ((methylamino)-3-propyl)triphenylphosphonium Bromide Propylamide (Compound 43)

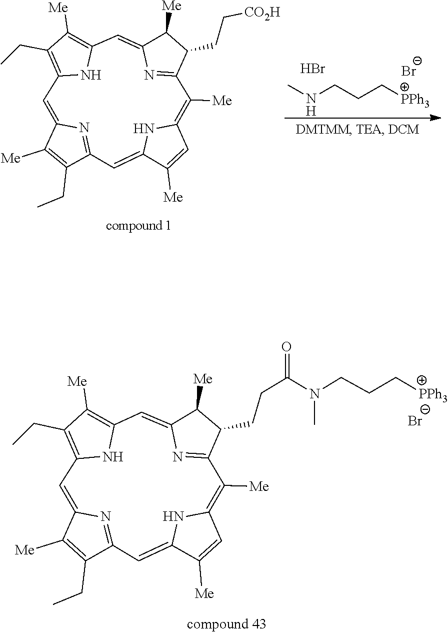

To a 25 mL RBF was added reduced phyllochlorin (compound 1) (100 mg, 0.197 mmol, 1 eq), DMTMM (82 mg, 0.295 mmol, 1.5 eq), DCM (5 mL), TEA (34 mg, 0.295 mmol, 1.7 eq) and (3-(methylamino)propyl)triphenylphosphonium bromide hydrobromide (146 mg, 0.295 mmol, 1.5 eq). The resultant mixture was stirred under nitrogen for 2 hours. The reaction mixture was transferred to a separatory funnel, diluted with DCM (10 mL) and washed with 0.5 M HCl (10 mL). The aqueous layer was re-extracted with DCM (2×5 mL) and the combined organic layers were washed with pH=7 phosphate buffer (10 mL), dried (Na₂SO₄) and concentrated by rotary evaporation to give a blue-black film. The residue was purified by column chromatography eluting with 3-6% MeOH/DCM. Fractions containing the major dark green spot (R_{f}=0.20 in 7% MeOH/DCM) were combined to give compound 43 as a dark green solid (105 mg, 59%).

¹H NMR (400 MHz, CDCl₃) δ 9.70 (s, 1H), 9.52 (s, 1H), 8.79-8.75 (m, 2H), 7.71-7.64 (m, 6H), 7.62-7.58 (m, 3H), 7.55-7.48 (m, 6H), 4.55-4.45 (m, 2H), 3.98-3.90 (m, 5H), 3.84 (q, 2H), 3.80-3.70 (m, 1H), 3.64 (s, 3H), 3.57 (m, 2H), 3.39 (s, 3H), 3.37 (s, 3H), 2.83 (s, 3H), 2.58-2.37 (m, 2H), 2.08-1.94 (m, 2H), 1.88-1.66 (m, 20H), −2.18 (brs, 2H).

### Biological Experimental Details

### Example 1—Determination of Solubility of Reduced Chlorin Analogues

Absorbance maxima were used as a surrogate measure of solubility. The relevant chlorin analogue was diluted to 50 μM in PBS (phosphate buffered saline) solutions containing decreasing amounts of DMSO from 100% to 0%.

Where required, polyvinylpyrrolidone (K30) was added to a final concentration of 1% w/v. Absorbance was measured using a Cytation 3 Multimode Plate Reader (Biotek) in spectral scanning mode, with spectra captured between 500-800 nm in 2 nm increments. Equivalent blank solutions were also measured and subtracted accordingly. Each spectrum was normalized to have a minimum signal of 0, and a maximum signal in pure DMSO solution (the most soluble state) of 100%.

### Example 2—Cytotoxicity, Phototoxicity and Therapeutic Index

### Preparation of Photosensitizer Stock Solutions

Photosensitizers (e.g. chlorin analogue, chlorin e4 disodium (provided by Advanced Molecular Technologies, Scoresby) or Talaporfin sodium (purchased from Focus Bioscience cat #HY-16477-5MG)) were resuspended in 100% dimethylsulfoxide (DMSO) at a concentration of 5.5 mM. Samples were stored at 4° C. protected from light.

### Preparation of Photosensitizers for In Vitro Studies

For in vitro experiments, photosensitizers (stock solution 5.5 mM in 100% DMSO) were diluted 1:100 in concentrated excipient solution (final 55 μM photosensitizer in 10% w/v Kollidon-12, 42.4% w/v polysorbate 80, 0.6% w/v citric acid anhydrous, 40% w/v ethanol, 1.0% DMSO). Serial dilutions were prepared in cell culture media (Dulbecco's Modified Eagle Medium/Nutrient Mixture F-12 (DMEM/F-12)) supplemented with 10% v/v Fetal Bovine Serum, 100 U/mL penicillin, 100 μg/mL streptomycin and the same excipient solution at a constant 1:55 dilution.

### Cell Culture

Human ovarian cancer cell line SKOV3 (ATCC #HTB-77) was maintained in Dulbecco's Modified Eagle Medium/Nutrient Mixture F-12 (DMEM/F-12), supplemented with 10% v/v Fetal Bovine Serum, 100 U/mL penicillin and 100 μg/mL streptomycin. Monolayer cultures were grown in a humidified incubator at 37° C. with 5% CO₂. Once cells had reached ˜80% confluence, spent media was replaced with media containing photosensitizer at the required concentration and cells were incubated for the desired period of time to allow photosensitizer uptake.

### Statistical Analyses

All data were analysed using GraphPad PRISM v8.3.1 (549) (GraphPad Software, CA). Spectral absorbance and viability measurements were normalized in the range 0-100%, with a minimum of 0 and a maximum value determined from the dataset. Dose response was determined using a sigmoidal four-point non-linear regression with variable slope, and IC10 or IC90 calculated for each compound. All data are shown as mean±SD (where appropriate).

### Cytotoxicity

SKOV3 cells were seeded in 96-well black wall plates (Greiner #655090) at a cell density of 5000 cells in 100 μl culture medium per well. On reaching ˜60% confluence, media was aspirated and replaced with fresh media containing the relevant reduced chlorin analogue from 0-100 μM in DMSO. Cells were incubated for a further 24 hours, allowing uptake of reduced chlorin analogues.

To test for inherent cytotoxicity (i.e. “dark toxicity”) of the reduced chlorin analogues, the culture media was replaced after 24 hours with fresh media containing 10% (v/v) AlamarBlue Cell Viability Reagent (ThermoFisher) and cells incubated at 37° C. for 6 hours. Untreated cells were used as a control. Fluorescence (Ex 555 nm/Em 596 nm) was measured using a Cytation 3 Cell Imaging Multi-Mode Reader (Biotek), and cytotoxicity assessed according to the % viable cells remaining. All measurements were made in quadruplicate.

### Phototoxicity

SKOV3 cells were seeded in 96-well black wall plates (Greiner #655090) at a cell density of 5000 cells in 100 μl culture medium per well. On reaching ˜60% confluence, media was aspirated and replaced with fresh media containing the relevant reduced chlorin analogue from 0-100 μM in DMSO. Cells were incubated for a further 24 hours, allowing uptake of reduced chlorin analogues.

To test for phototoxicity, cells incubated with reduced chlorin analogues (0-10 μM in DMSO) had culture media replaced after 24 hours (as above) and were then exposed to a 660 nm laser (Invion) or light-emitting diode (LED) panel (Invion) with optical power density at 50 mW/cm² for 5 mins (total 15 J/cm²). Laser and LED exposure induce an equivalent response in relation to phototoxicity. Following activation, cells were cultured for a further 24 hours. Media was then replaced with fresh media containing AlamarBlue, and % viable cells remaining assessed as above. Controls included cells treated with reduced chlorin analogues but not activated by laser light; cells without reduced chlorin analogue treatment but with laser light; and untreated controls. All measurements were made in quadruplicate.

### Toxicity Profile for Reduced Chlorin Analogues

The phototoxicity and inherent cytotoxicity (i.e. “dark toxicity”) of several reduced chlorin analogues were assessed as previously using SKOV3 ovarian cancer cells. For comparative purposes, reduced chlorin analogues were compared against phyllochlorin free acid, chlorin e4 disodium and Talaporfin sodium, a clinically approved photosensitizer used in the photodynamic treatment of lung cancers. Phototoxicity IC90 values and dark toxicity IC10 values were calculated using a log[inhibitor]-vs normalized response dose curve with variable slope, using the formula Y=100/(1+(IC90/X)\hat{ }HillSlope (phototoxicity IC90)) or Y=100/(1+(IC10/X)\hat{ }HillSlope (dark toxicity IC10)).

Phototoxicity and dark toxicity values are provided in Table 1. With only one exception (compound 36) all reduced chlorin analogues had phototoxicity IC90 values in the nM range; with thirteen compounds having an IC90 of 50 nM or below, and four compounds (compounds 11, 13, 18 and 27) having an IC90 of 10 nM or below (Table 1) indicating exceptional phototoxicity against SKOV3 cancer cells. These were substantially better than chlorin e4 disodium (IC90 21.32 μM) or Talaporfin sodium (IC90 22.83 μM); indeed, the best-performing compound (compound 11) achieved 4 orders of magnitude greater phototoxicity compared to Talaporfin sodium. Thus, reduced chlorin analogues achieved an up to ˜7,500-fold increase in phototoxicity compared to Talaporfin sodium, a clinically approved photosensitizer.

Substantial variation in the dark toxicity of the reduced chlorin analogues of the present invention was observed (Table 1). The greater phototoxicity afforded by the reduced chlorin analogues of the present invention, however, is expected to offset any dark toxicity issue through a decreased dose requirement in use.

### Therapeutic Index for Reduced Chlorin Analogues

To evaluate the therapeutic potential of reduced chlorin analogues, the therapeutic index (TI) was calculated. TI provides a quantitative measurement to describe relative drug safety, by comparing the drug concentration required for desirable effects versus the concentration resulting in undesirable off-target toxicity. TI was calculated using phototoxicity IC90 vs dark toxicity IC10.

TI values are provided in Table 1. Talaporfin sodium had a low therapeutic index (TI=0.49) with chlorin e4 disodium only marginally better (TI=1.89), indicating that whilst their relative cytotoxicity is low, the potential therapeutic window for their use is small. Most of the reduced chlorin analogues of the present invention had comparatively significantly improved TIs with substantially greater phototoxicity (Table 1).

Thus, the reduced chlorin analogues of the present invention have a desirable therapeutic index that is better than a clinically applied photosensitizer. Moreover the greater phototoxicity of the reduced chlorin analogues suggests their potential use at a greatly reduced dose in vivo. The reduced chlorin analogues therefore have an acceptable therapeutic profile for clinical application.

TABLE 1
Toxicity profile and therapeutic index
for reduced chlorin analogues:

| | Phototoxicity | Dark toxicity | Therapeutic |
|---|---|---|---|
| Species | μM (IC90) | μM (IC10) | Index |
| Compound 1 | 0.176 | 10.450 | 59.38 |
| Compound 2 | 0.071 | 47.840 | 673.80 |
| Compound 3 | 0.043 | 13.330 | 310.00 |
| Compound 4 | 0.068 | 2.780 | 40.88 |
| Compound 5 | 0.102 | 18.440 | 180.78 |
| Compound 6 | 0.371 | 11.650 | 31.40 |
| Compound 7 | 0.036 | 26.100 | 725.00 |
| Compound 9 | 0.098 | 12.780 | 130.41 |
| Compound 10 | 0.050 | 4.905 | 98.10 |
| Compound 11 | 0.003 | 4.347 | 1,449.00 |
| Compound 12 | 0.078 | 5.975 | 76.60 |
| Compound 13 | 0.006 | 1.260 | 210.00 |
| Compound 14 | 0.161 | 3.940 | 24.47 |
| Compound 15 | 0.070 | 5.400 | 77.14 |
| Compound 16 | 0.011 | 3.205 | 291.36 |
| Compound 17 | 0.174 | 4.752 | 27.31 |
| Compound 18 | 0.010 | 3.946 | 394.60 |
| Compound 19 | 0.061 | 15.940 | 261.31 |
| Compound 20 | 0.063 | 5.825 | 92.46 |
| Compound 21 | 0.022 | 1.465 | 66.59 |
| Compound 22 | 0.042 | 4.348 | 103.52 |
| Compound 23 | 0.064 | 4.540 | 70.94 |
| Compound 24 | 0.049 | 7.011 | 143.08 |
| Compound 26 | 0.494 | 14.670 | 29.70 |
| Compound 27 | 0.0055 * | 1.326 | 241.09 |
| Compound 28 | 0.0326 * | 1.510 | 46.32 |
| Compound 31 | 0.0110 * | 8.850 | 804.55 |
| Compound 36 | 4.362 | 12.200 | 2.80 |
| Compound 39 | 0.1413 * | 6.613 | 46.80 |
| Compound 40 | 0.2286 * | 3.222 | 14.09 |
| Chlorin e4 | 21.32 | 40.23 | 1.89 |
| disodium | | | |
| Talaporfin | 22.83 | 11.10 | 0.49 |
| sodium | | | |
| Phyllochlorin | 0.43 | 13.50 | 31.40 |
| free acid | | | |
| Phyllochlorin | 0.35 | 21.96 | 62.74 |
| sodium salt | | | |

* denotes that the phototoxicity was measured by LED

### Reduction of 13-Vinyl to 13-Ethyl (Meso-Substitution) Results in Enhanced Phototoxicity of Phyllochlorin Photosensitizers

One advantage of reduced phyllochlorins is that they are not as susceptible to oxidation by singlet oxygen and ROS by virtue of the removal of the reactive exocyclic alkene and as a result of this, more singlet oxygen and ROS should be available, for example, for the destruction of cancers.

The phototoxicity of meso-phyllochlorin free acid (compound 1) and meso-phyllochlorin sodium salt (compound 2) were compared to their non-meso analogues, phyllochlorin free acid and phyllochlorin sodium salt. In both cases, the phototoxicity afforded by the meso-compounds was increased between 2.4-fold to 4.9-fold respectively. Dark toxicity was largely unaffected by this chemical change, as was TI; although in the case of compound 2, the 4.9-fold increase in phototoxicity was reflected in a substantially improved TI (Table 1).

Thus, meso substitutions can be used to improve the phototoxicity of phyllochlorins.

### Example 3—Reduced Phyllochlorin is Non-Toxic when Injected into Mice

To assess acute toxicity, reduced phyllochlorin or chlorin e4 disodium (each prepared with 1% PVP in PBS+20 mM HEPES) were administered at a dose of 5 mg/kg to C57BL/6 mice by intraperitoneal injection.

As PBS buffer (pH 7.4) is used in the experiments and the carboxylic acid in reduced phyllochlorin (and the same carboxy group in other related chlorins) is estimated to have a pKa of less than 5, reduced phyllochlorin in the PBS buffer will exist predominantly as the sodium and potassium salts with very little to no free acid.

Mice were observed for 30 mins for any clinical signs of acute toxicity (e.g. squinting, piloerection, loss of motility, general behaviour). No side effects were noted following administration of either compound, suggesting that reduced phyllochlorin delivered at a dose of 5 mg/kg is safe for injection.

It will be understood that the present invention has been described above by way of example only. The examples are not intended to limit the scope of the invention. Various modifications and embodiments can be made without departing from the scope and spirit of the invention, which is defined by the following claims only.

1. A compound of formula (1) or a complex of formula (2): 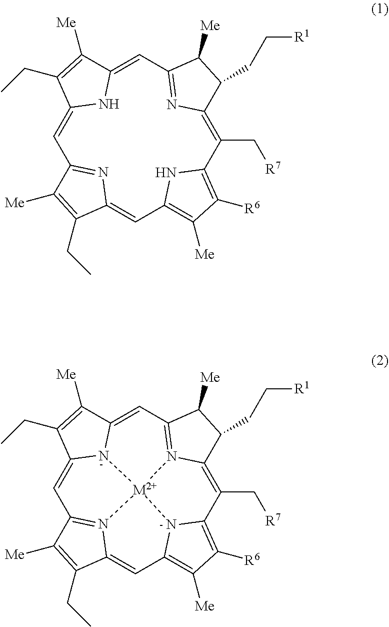
or a pharmaceutically acceptable salt thereof, wherein:
—R¹ is selected from —CH₂OR², —CH₂SR², —CH₂S(O)R², —CH₂S(O)₂R², —CH₂N(R²)₂, —R², —C(O)—OR³, —C(O)—SR³, —C(O)—N(R³)₂, —C(S)—OR³, —C(S)—SR³ or —C(S)—N(R³)₂;
—R², each independently, is selected from —H, —C(O)R⁴, —C(O)—OR⁴, —C(O)—SR⁴, —C(O)—N(R⁴)₂, —C(S)—OR⁴, —C(S)—SR⁴, —C(S)—N(R⁴)₂, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁸]Y, —R^{α}—[N(R⁵)₂(R^{5′})], —R^{α}—[P(R⁵)₂(R^{5′})] or —R^{α}—[R^{8′}];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁸]Y, —R^{α}—[N(R⁵)₂(R^{5′})], —R^{α}—[P(R⁵)₂(R^{5′})] or —R^{α}—[R^{8′}];
—R^{α}—, each independently, is selected from a C₁-C₄₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O, S, P and Se in its carbon skeleton;
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂⁻, wherein the phenyl or C₅-C₆ heteroaryl may optionally be further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁶ is selected from hydrogen, —C(O)—OR³, —C(O)—SR³, —C(O)—N(R³)₂, —C(S)—OR³, —C(S)—SR³ or —C(S)—N(R³)₂;
—R⁷ is selected from hydrogen, —C(O)—OR³, —C(O)—SR³, —C(O)—N(R³)₂, —C(S)—OR³, —C(S)—SR³ or —C(S)—N(R³)₂;
or —R⁶ and —R⁷ together form —C(O)—O—;
—R⁸ is —[NC₅H₅] optionally substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{8′} is —[NC₅H₅] substituted with —CO₂ and optionally further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
n is 1, 2, 3, 4, 5 or 6;
X is a halo group;
Y is a counter anion;
Z is a counter cation; and
M²⁺ is a metal cation.

2. A compound of formula (1) or a complex of formula (2): 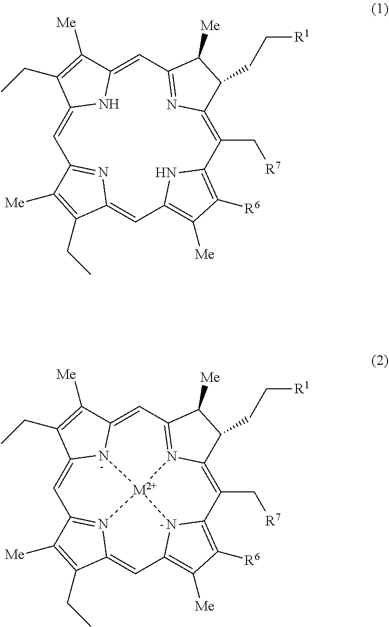
or a pharmaceutically acceptable salt thereof, for use in medicine, wherein:
—R¹ is selected from —CH₂OR², —CH₂SR², —CH₂S(O)R², —CH₂S(O)₂R², —CH₂N(R²)₂, —R², —C(O)—OR³, —C(O)—SR³, —C(O)—N(R³)₂, —C(S)—OR³, —C(S)—SR³ or —C(S)—N(R³)₂;
—R², each independently, is selected from —H, —C(O)R⁴, —C(O)—OR⁴, —C(O)—SR⁴, —C(O)—N(R⁴)₂, —C(S)—OR⁴, —C(S)—SR⁴, —C(S)—N(R⁴)₂, —R^{α}—H, —R^{β}, —R^{α}—R^{β}, —R^{α}—OH, —R^{α}—OR^{β}, —R^{α}—SH, —R^{α}—SR^{β}, —R^{α}—S(O)R^{β}, —R^{α}—S(O)₂R^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁸]Y, —R^{α}—[N(R⁵)₂(R^{5′})], —R^{α}—[P(R⁵)₂(R^{5′})] or —R^{α}—[R^{8′}];
—R³ and —R⁴, each independently, is selected from —H, —R^{α}—H, —R^{β}, —R^{α}—R^{β}, —R^{α}—OH, —R^{α}—OR^{β}, —R^{α}—SH, —R^{α}—SR^{β}, —R^{α}—NH₂, —R^{α}—NH(R^{β}), —R^{α}—N(R^{β})₂, —R^{α}—X, —R^{α}—[N(R⁵)₃]Y, —R^{α}—[P(R⁵)₃]Y, —R^{α}—[R⁸]Y, —R^{α}—[N(R⁵)₂(R^{5′})], —R^{α}—[P(R⁵)₂(R^{5′})] or —R^{α}—[R^{8′}];
—R^{α}—, each independently, is selected from a C₁-C₄₂ alkylene group, wherein the alkylene group may optionally be substituted with one or more C₁-C₄ alkyl, C₁-C₄ haloalkyl or halo groups, and wherein one or more carbon atoms in the backbone of the alkylene group may optionally be replaced by a heteroatom or group independently selected from O, S, NH or NMe;
—R^{β}, each independently, is a saturated or unsaturated hydrocarbyl group, wherein the hydrocarbyl group may be straight-chained or branched, or be or include cyclic groups, wherein the hydrocarbyl group may optionally be substituted, and wherein the hydrocarbyl group may optionally include one or more heteroatoms N, O, S, P and Se in its carbon skeleton;
—R⁵, each independently, is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, wherein the phenyl or C₅-C₆ heteroaryl may optionally be substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{5′} is selected from C₁-C₄ alkyl, C₁-C₄ haloalkyl, —(CH₂CH₂O)ₙ—H, —(CH₂CH₂O)ₙ—CH₃, phenyl or C₅-C₆ heteroaryl, each substituted with —CO₂, wherein the phenyl or C₅-C₆ heteroaryl may optionally be further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R⁶ is selected from hydrogen, —C(O)—OR³, —C(O)—SR³, —C(O)—N(R³)₂, —C(S)—OR³, —C(S)—SR³ or —C(S)—N(R³)₂;
—R⁷ is selected from hydrogen, —C(O)—OR³, —C(O)—SR³, —C(O)—N(R³)₂, —C(S)—OR³, —C(S)—SR³ or —C(S)—N(R³)₂;
or —R⁶ and —R⁷ together form —C(O)—O—;
—R⁸ is —[NC₅H₅] optionally substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
—R^{8′} is —[NC₅H₅] substituted with —CO₂ and optionally further substituted with one or more C₁-C₆ alkyl, C₁-C₆ haloalkyl, —O(C₁-C₆ alkyl), —O(C₁-C₆ haloalkyl), halo, —CO₂H, —CO₂Z, —CO₂NH₂, —O—(CH₂CH₂O)ₙ—H or —O—(CH₂CH₂O)ₙ—CH₃ groups;
n is 1, 2, 3, 4, 5 or 6;
X is a halo group;
Y is a counter anion;
Z is a counter cation; and
M²⁺ is a metal cation.

3. The compound or complex according to claim 1 or 2, wherein each —R^{α}— is independently selected from C₁-C₆ alkylene.

4. The compound or complex according to any preceding claim, wherein at least one of —R², —R³ and —R⁴ is selected from —R^{α}—OR^{β} or —R^{α}—SR^{β}, and —R^{β} is a saccharidyl group.

5. The compound or complex according to claim 4, wherein —R^{β} is a saccharidyl group selected from:

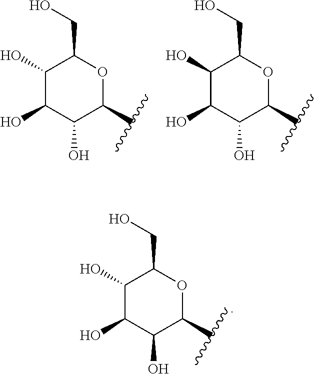 [SMILES: *[C@@H]1O[C@H](CO)[C@@H](O)[C@H](O)[C@H]1O ; *[C@@H]1O[C@H](CO)[C@H](O)[C@H](O)[C@H]1O ; *[C@@H]1O[C@H](CO)[C@@H](O)[C@H](O)[C@@H]1O]

6. The compound or complex according to claim 5, wherein the saccharidyl group is:

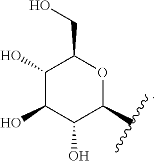 [SMILES: *[C@@H]1O[C@H](CO)[C@@H](O)[C@H](O)[C@H]1O]

7. The compound or complex according to claim 4, wherein —R^{β} is a saccharidyl group selected from: 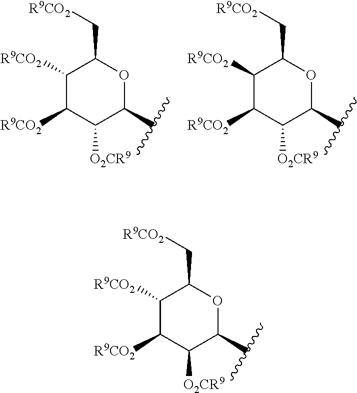
wherein —R⁹ is selected from C₁-C₄ alkyl.

8. The compound or complex according to claim 7, wherein —R⁹ is methyl.

9. The compound or complex according to any preceding claim, wherein —R¹ is —C(O)—N(R³)₂.

10. The compound or complex according to claim 9, wherein —R¹ is —C(O)—N(R³)(R^{3′}), wherein —R³ is selected from —R^{α}—OR^{β} or —R^{α}—SR^{β} and —R^{β} is a saccharidyl group, and —R^{3′} is H or C₁-C₄ alkyl.

11. The compound or complex according to any preceding claim, wherein —R⁶ is hydrogen.

12. The compound or complex according to any one of claims 1-10, wherein —R⁶ is —C(O)—OR³ and —R³ is C₁-C₄ alkyl.

13. The compound or complex according to any one of claims 1-10, wherein —R⁶ is selected from —C(O)—OR³, —C(O)—SR³ or —C(O)—N(R³)(R^{3′}), wherein —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl.

14. The compound or complex according to any preceding claim, wherein —R⁷ is hydrogen.

15. The compound or complex according to any one of claims 1-13, wherein —R⁷ is —C(O)—OR³ and —R³ is C₁-C₄ alkyl.

16. The compound or complex according to any one of claims 1-13, wherein —R⁷ is selected from —C(O)—OR³, —C(O)—SR³ or —C(O)—N(R³)(R^{3′}), wherein —R³ is selected from —R^{α}—OR^{β}, —R^{α}—SR^{β}, —R^{α}—S(O)R^{β} or —R^{α}—S(O)₂R^{β}, and —R^{β} is a saccharidyl group, and —R^{3′} is H or C₁-C₄ alkyl.

17. The compound or complex according to claim 1 or 2, wherein the compound or complex is: 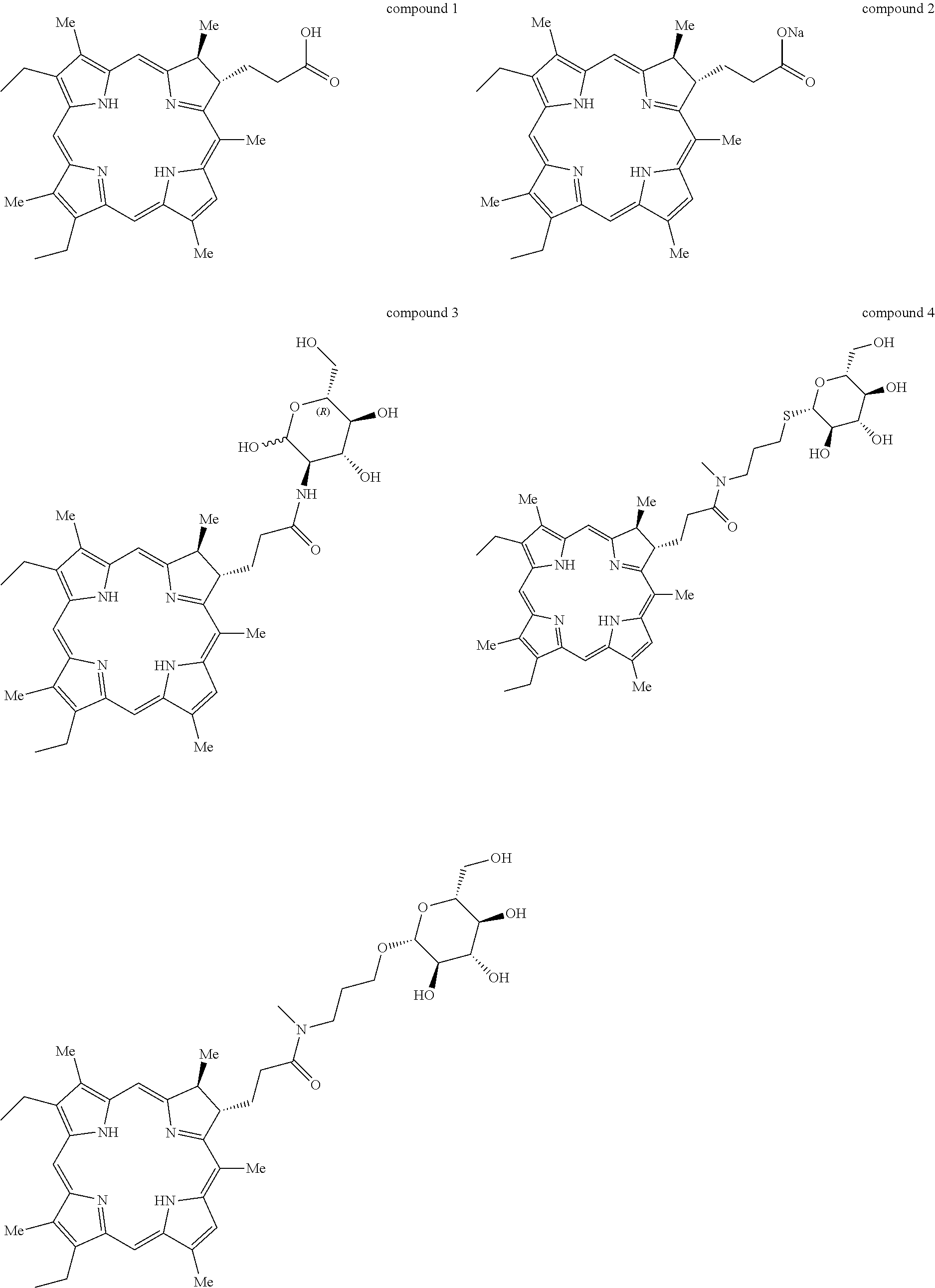 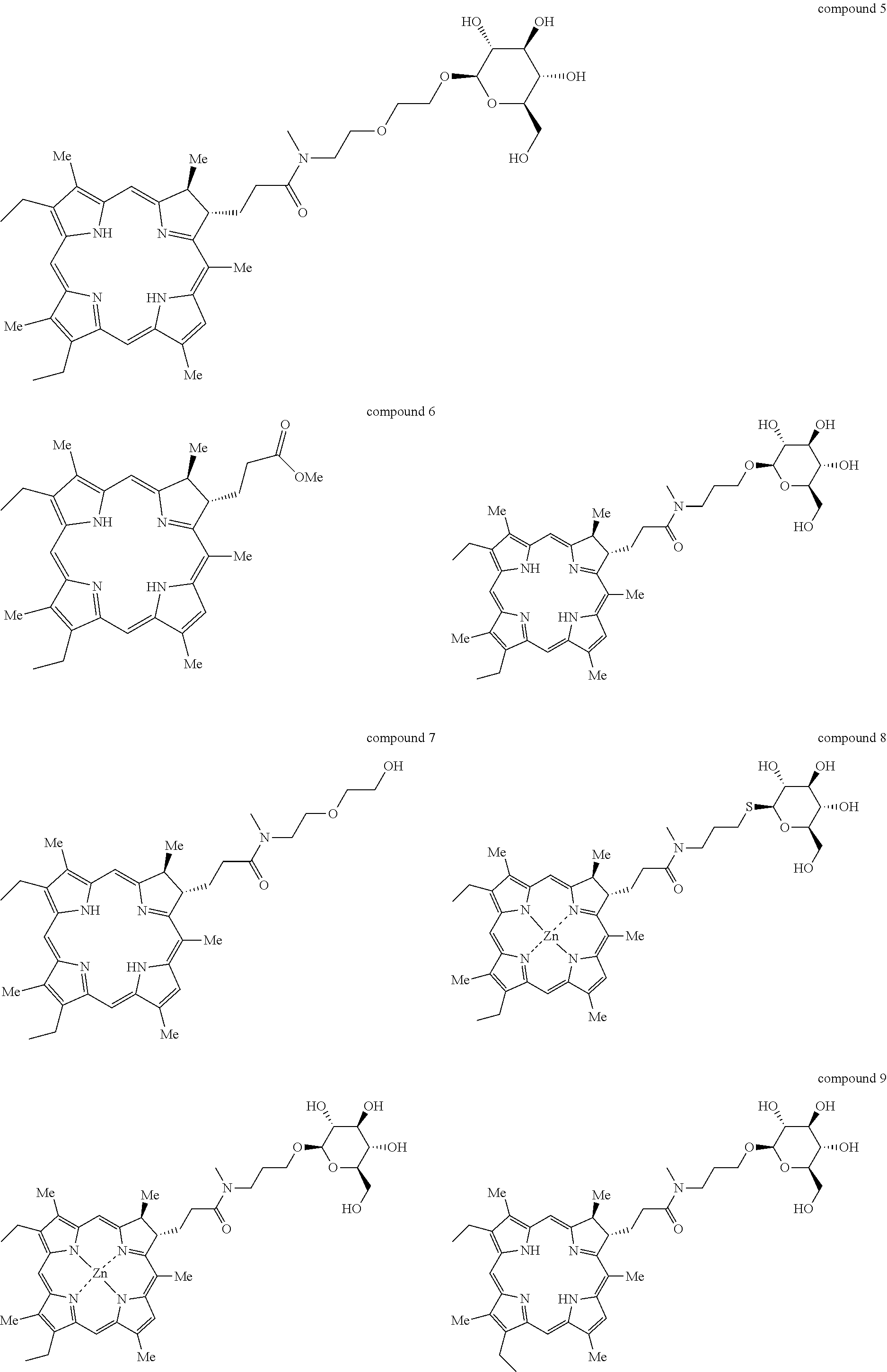 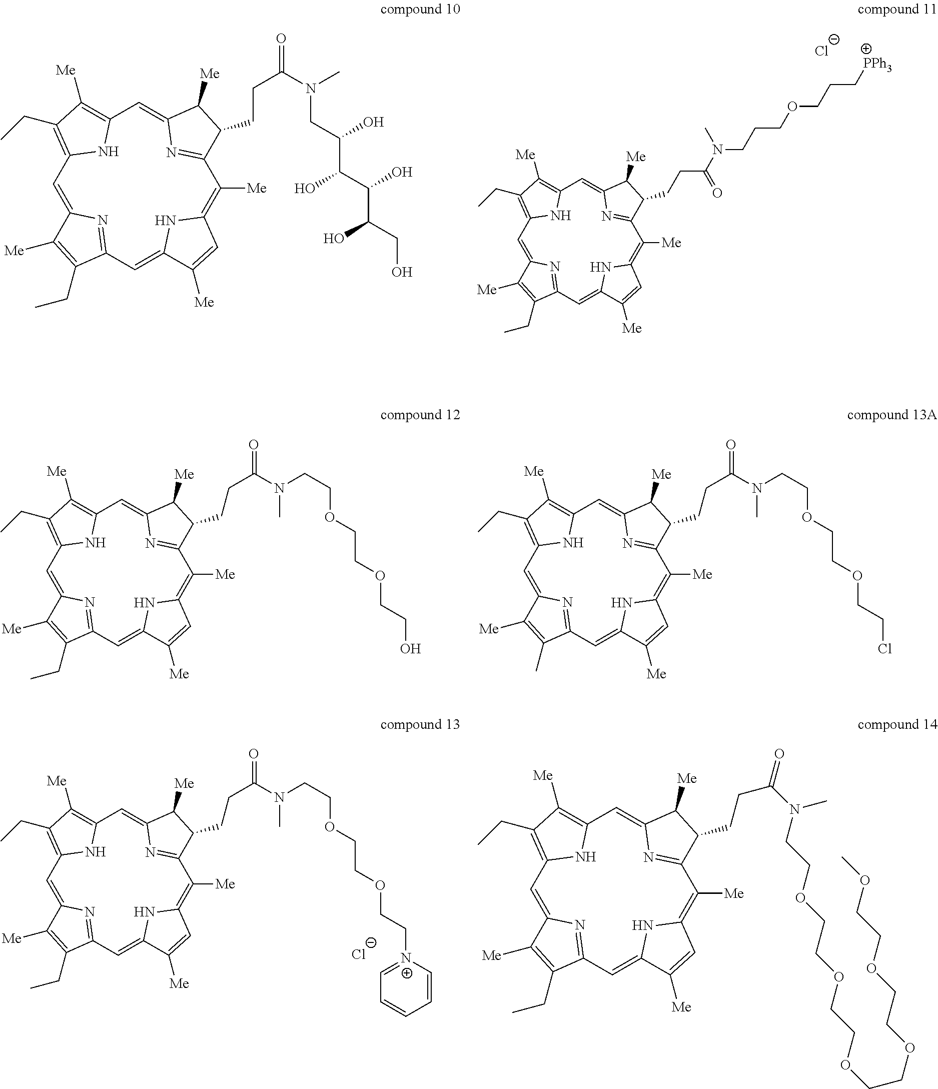 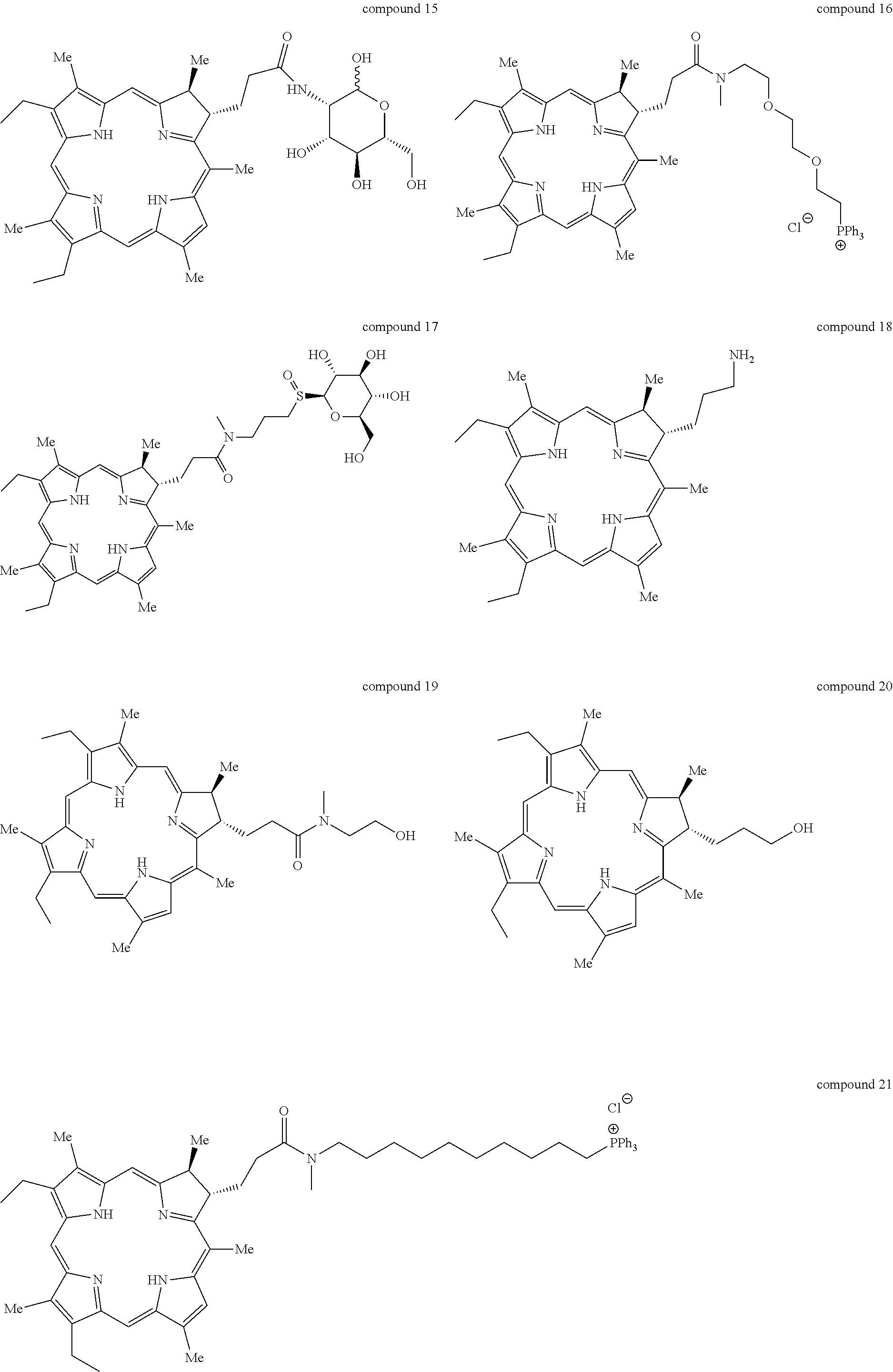 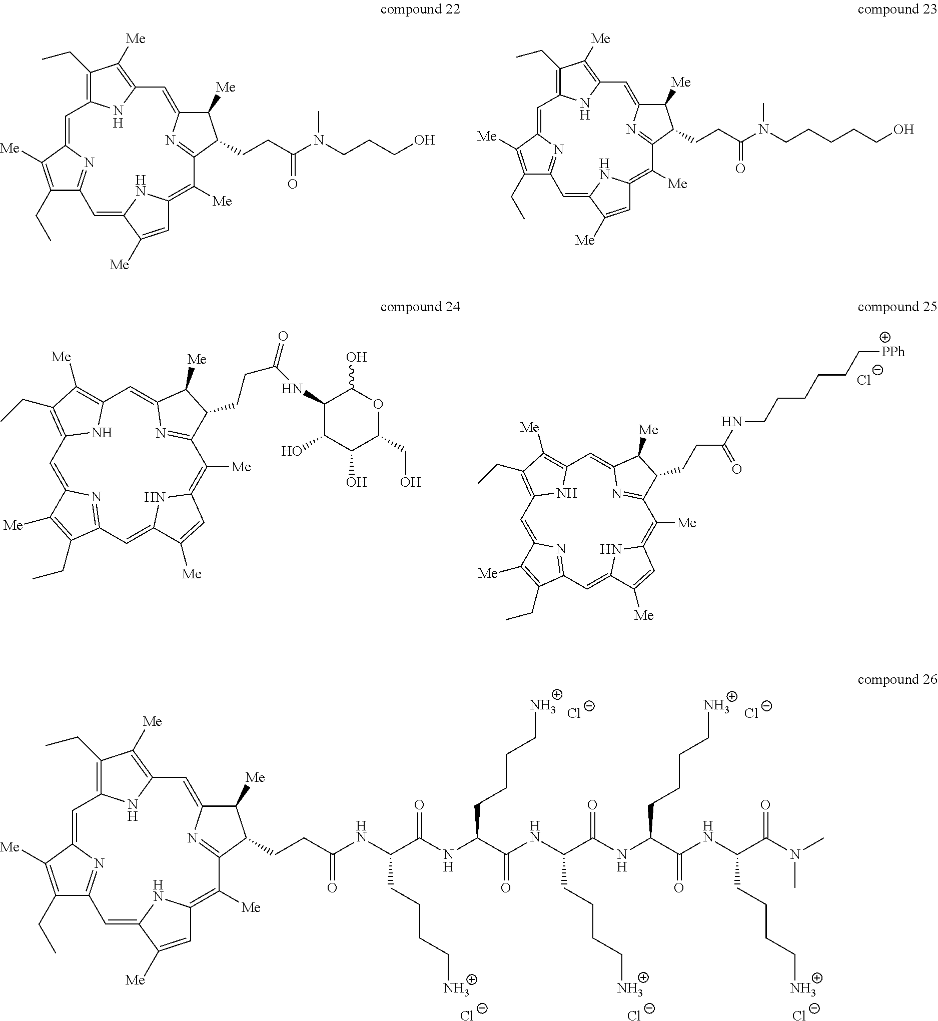 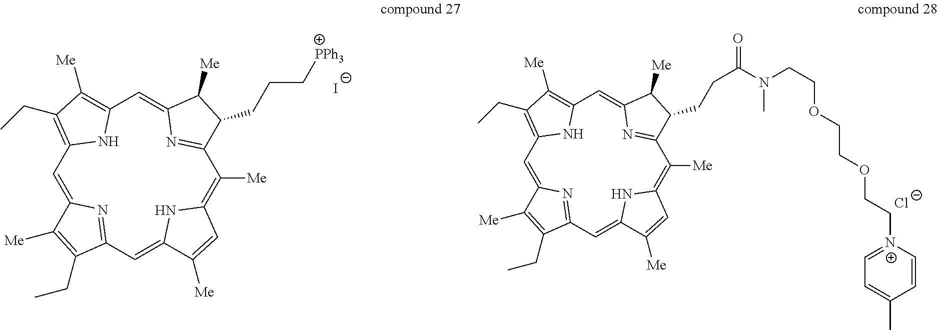 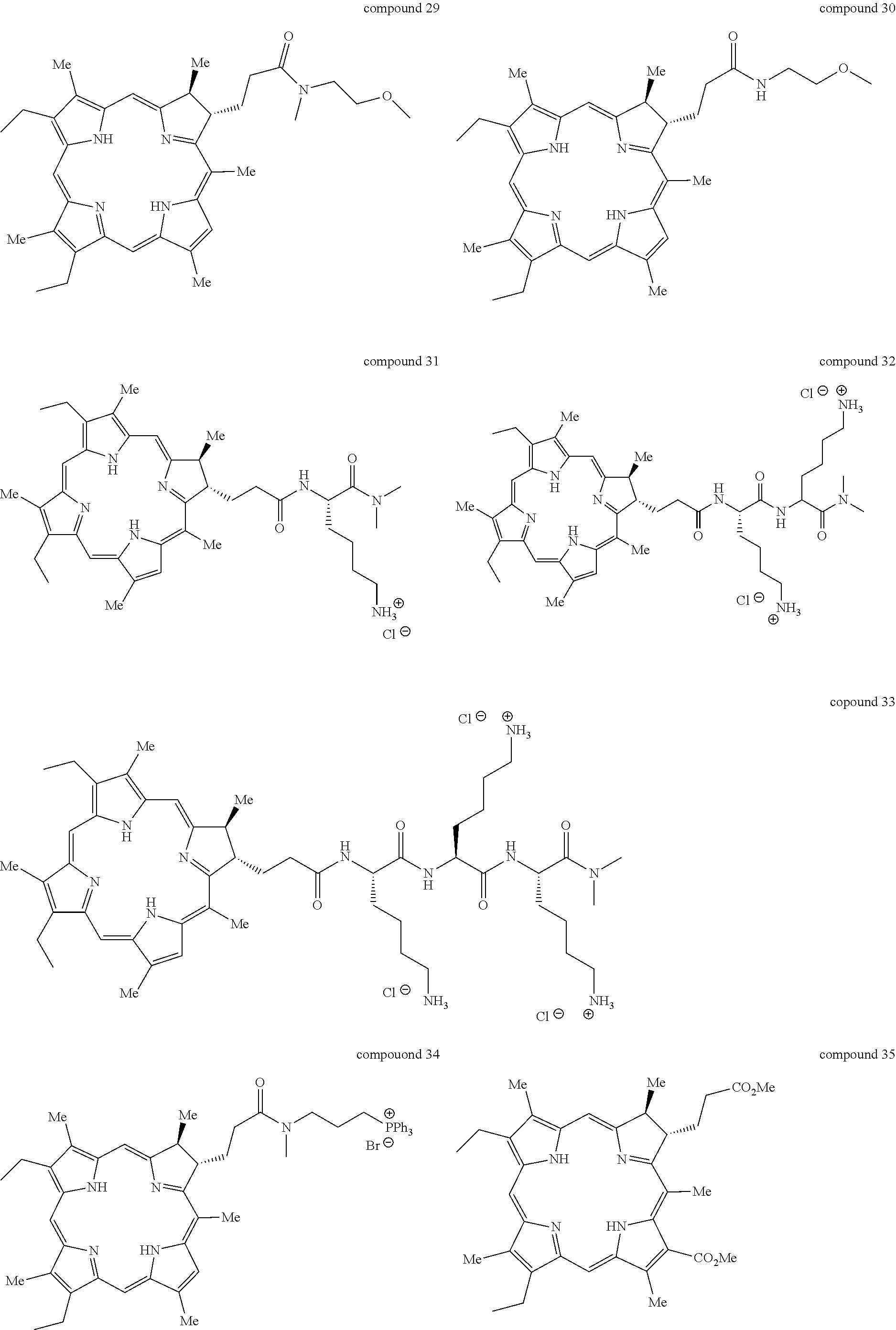 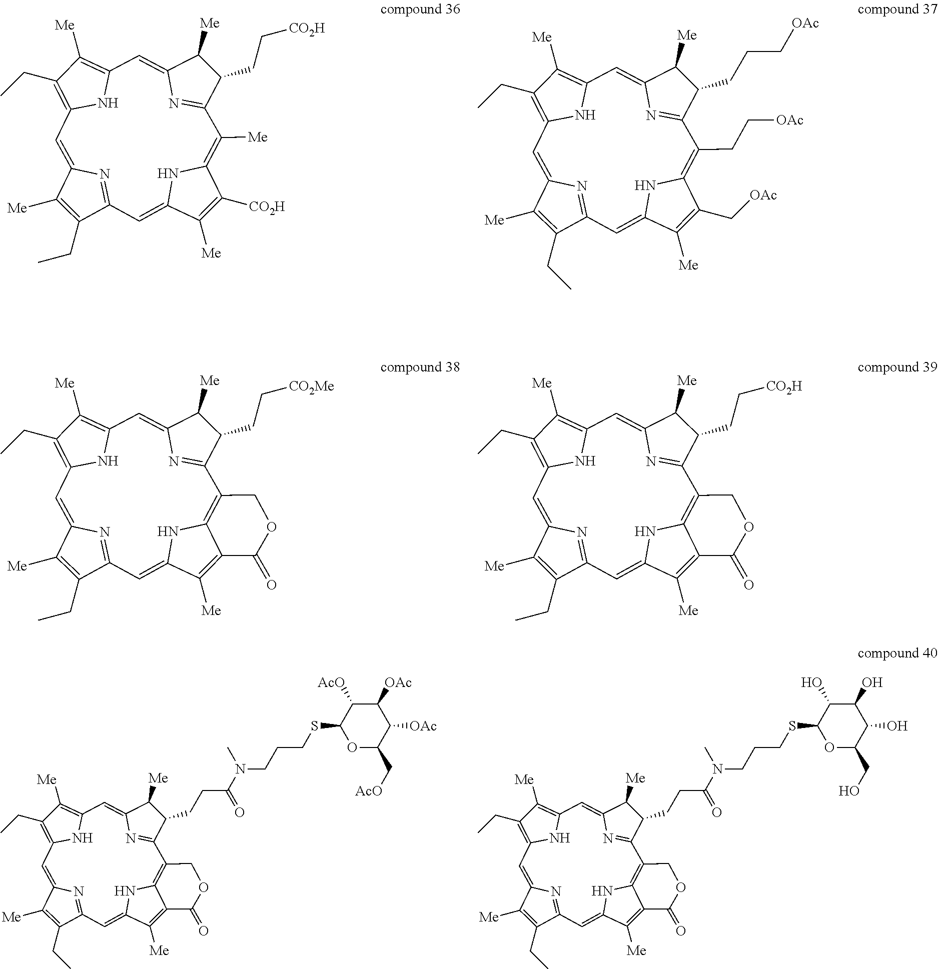 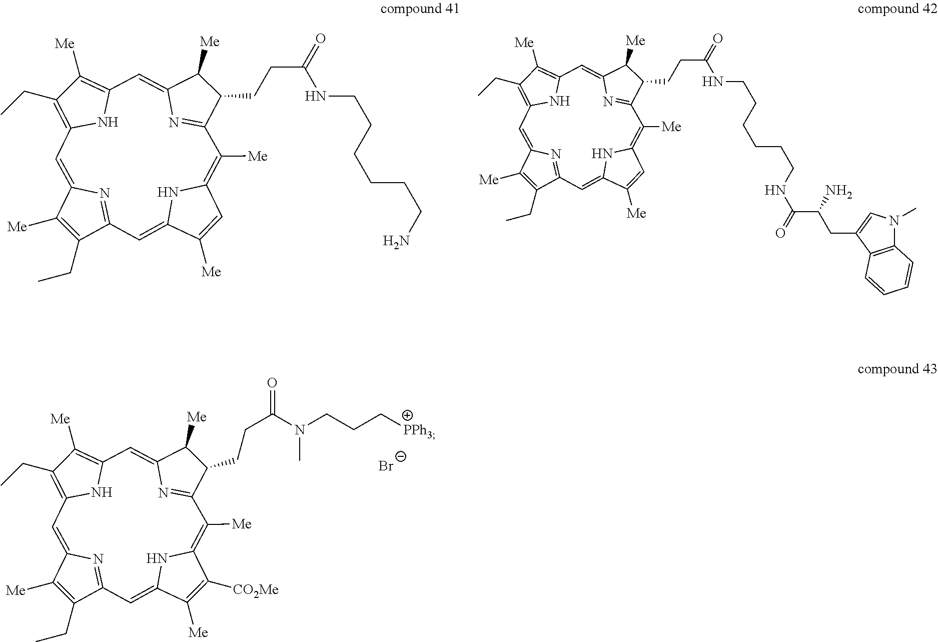
or a metal cation complex thereof, or a pharmaceutically acceptable salt thereof.

18. The compound or complex according to claim 1 or 2, wherein the compound is reduced phyllochlorin in the form of a pharmaceutically acceptable salt.

19. The compound or complex according to claim 18, wherein the compound is reduced phyllochlorin mono-sodium.

20. The compound or complex according to claim 2, wherein the compound is:
(1) reduced phyllochlorin free acid; or
(2) reduced phyllochlorin methyl ester.

21. The compound or complex according to any preceding claim, for use in photodynamic therapy or cytoluminescent therapy.

22. The compound or complex according to any preceding claim, for use in the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile so duct, intestine, colon, colorectum, rectum, bladder, ureter, kidney, liver, gall bladder, spleen, brain, lymphatic system, bones, skin or pancreas.

23. The compound or complex according to any preceding claim, for use in the treatment of a disease characterised by benign or malignant cellular hyperproliferation or by areas of neovascularisation.

24. The compound or complex according to any preceding claim, for use in the treatment of a benign or malignant tumour.

25. The compound or complex according to any preceding claim, for use in the treatment of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

26. The compound or complex according to any preceding claim, for use in photodynamic diagnosis.

27. The compound or complex according to any preceding claim, wherein the compound is adapted for administration prior to administration of irradiation.

28. The compound or complex according to claim 27, wherein the irradiation is electromagnetic radiation with a wavelength in the range of from 500 nm to 1000 nm.

29. A pharmaceutical composition comprising a compound or complex according to any preceding claim and a pharmaceutically acceptable carrier or diluent.

30. The pharmaceutical composition according to claim 29, further comprising polyvinylpyrrolidone.

31. The pharmaceutical composition according to claim 29 or 30, further comprising an immune checkpoint inhibitor.

32. The pharmaceutical composition according to claim 31, wherein the immune checkpoint inhibitor is selected from Pembrolizumab, Nivolumab, Cemiplimab, Atezolizumab, Avelumab, Durvalumab or Ipilimumab.

33. The pharmaceutical composition according to any one of claims 29-32, for use in photodynamic therapy or cytoluminescent therapy.

34. The pharmaceutical composition according to any one of claims 29-33, for use in the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

35. The pharmaceutical composition according to any one of claims 29-34, for use in the treatment of a disease characterised by benign or malignant cellular hyperproliferation or by areas of neovascularisation.

36. The pharmaceutical composition according to any one of claims 29-35, for use in the treatment of a benign or malignant tumour.

37. The pharmaceutical composition according to any one of claims 29-36, for use in the treatment of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

38. The pharmaceutical composition according to claim 29 or 30, for use in photodynamic diagnosis.

39. The pharmaceutical composition according to any one of claims 29-38, wherein the pharmaceutical composition is adapted for administration prior to administration of irradiation.

40. The pharmaceutical composition according to claim 39, wherein the irradiation is electromagnetic radiation with a wavelength in the range of from 500 nm to 1000 nm.

41. The pharmaceutical composition according to any one of claims 29-40, wherein the pharmaceutical composition is in a form suitable for oral, parenteral (including intravenous, subcutaneous, intramuscular, intradermal, intratracheal, intraperitoneal, intratumoral, intraarticular, intraabdominal, intracranial and epidural), transdermal, airway (aerosol), rectal, vaginal or topical (including buccal, mucosal and sublingual) administration.

42. The pharmaceutical composition according to claim 41, wherein the pharmaceutical composition is in a form suitable for oral or parenteral administration.

43. Use of a compound or complex according to any one of claims 1-28, in the manufacture of a medicament for the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

44. Use of a compound or complex according to any one of claims 1-28, in the manufacture of a phototherapeutic agent for use in photodynamic therapy or cytoluminescent therapy.

45. The use according to claim 44, wherein the phototherapeutic agent is for the treatment of atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

46. The use according to any one of claims 43-45, wherein the medicament or the phototherapeutic agent is for the treatment of a disease characterised by benign or malignant cellular hyperproliferation or by areas of neovascularisation.

47. The use according to any one of claims 43-46, wherein the medicament or the phototherapeutic agent is for the treatment of a benign or malignant tumour.

48. The use according to any one of claims 43-47, wherein the medicament or the phototherapeutic agent is for the treatment of early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

49. Use of a compound or complex according to any one of claims 1-28, in the manufacture of a photodiagnostic agent for use in photodynamic diagnosis.

50. The use according to any one of claims 43-49, wherein the medicament, the phototherapeutic agent or the photodiagnostic agent is adapted for administration prior to administration of irradiation.

51. The use according to claim 50, wherein the irradiation is electromagnetic radiation with a wavelength in the range of from 500 nm to 1000 nm.

52. A method of treating atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas; the method comprising administering a therapeutically effective amount of a compound or complex according to any one of claims 1-28 to a human or animal in need thereof.

53. A method of photodynamic therapy or cytoluminescent therapy of a human or animal disease, the method comprising administering a therapeutically effective amount of a compound or complex according to any one of claims 1-28 to a human or animal in need thereof.

54. The method according to claim 53, wherein the human or animal disease is atherosclerosis; multiple sclerosis; diabetes; diabetic retinopathy; arthritis; rheumatoid arthritis; a fungal, viral, chlamydial, bacterial, nanobacterial or parasitic infectious disease; HIV; Aids; infection with sars virus (preferably severe acute respiratory syndrome coronavirus 2 (SARS-CoV-2)), Asian (chicken) flu virus, Dengue virus, herpes simplex or herpes zoster; hepatitis; viral hepatitis; a cardiovascular disease; coronary artery stenosis; carotid artery stenosis; intermittent claudication; a dermatological condition; acne; psoriasis; a disease characterised by benign or malignant cellular hyperproliferation or by areas of neovascularisation; a benign or malignant tumour; early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

55. The method according to any one of claims 52-54, wherein the human or animal disease is characterised by benign or malignant cellular hyperproliferation or by areas of neovascularisation.

56. The method according to any one of claims 52-55, wherein the human or animal disease is a benign or malignant tumour.

57. The method according to any one of claims 52-56, wherein the human or animal disease is early cancer; cervical dysplasia; soft tissue sarcoma; a germ cell tumour; retinoblastoma; age-related macular degeneration; lymphoma; Hodgkin's lymphoma; head and neck cancer; oral or mouth cancer; or cancer of the blood, prostate, cervix, uterus, vaginal or other female adnexa, breast, naso-pharynx, trachea, larynx, bronchi, bronchioles, lung, hollow organs, esophagus, stomach, bile duct, intestine, colon, colorectum, rectum, bladder, ureter, kidney, liver, gall bladder, spleen, brain, lymphatic system, bones, skin or pancreas.

58. A method of photodynamic diagnosis of a human or animal disease, the method comprising administering a diagnostically effective amount of a compound or complex according to any one of claims 1-28 to a human or animal.

59. The method according to any one of claims 52-58, wherein the human or animal is subjected to irradiation after the administration of the compound or complex according to any one of claims 1-28.

60. The method according to claim 59, wherein the irradiation is electromagnetic radiation with a wavelength in the range of from 500 nm to 1000 nm.

61. A pharmaceutical combination or kit comprising:
(a) a compound or complex according to any one of claims 1-28; and
(b) an immune checkpoint inhibitor.

62. The pharmaceutical combination or kit according to claim 61, wherein the immune checkpoint inhibitor is selected from Pembrolizumab, Nivolumab, Cemiplimab, Atezolizumab, Avelumab, Durvalumab or Ipilimumab.
